(19) United States
(12) Patent Application Publication
(10) Pub. No.: US 2026/0308630 A1
(43) Pub. Date: Oct. 8, 2026

(54) CRISPR-CAS GENOME ENGINEERING VIA A MODULAR AAV DELIVERY SYSTEM

(71) Applicant: The Regents of the University of California, Oakland, CA (US)

(72) Inventors: Prashant Mali, La Jolla, CA (US); Dhruva Katrekar, La Jolla, CA (US); Ana Collado, La Jolla, CA (US)

(21) Appl. No.: 19/713,547

(22) Filed: Jun. 18, 2026

Related U.S. Application Data
(63) Continuation of application No. 16/325,679, filed on Aug. 18, 2017.
(60) Provisional application No. 62/376,855, filed on Aug. 18, 2016.
(60) Provisional application No. 62/415,858, filed on Nov. 1, 2016.
(60) Provisional application No. 62/481,589, filed on Apr. 4, 2017.

(51) Int. Cl. C12N 15/86; A61K 35/76; A61P 25/04; A61P 33/06; C12N 7/00; C12N 9/22; C12N 15/113

(52) U.S. Cl. CPC C12N 15/86; A61K 35/76; A61P 25/04; A61P 33/06; C12N 7/00; C12N 9/22; C12N 15/113; C12N 2310/20; C12N 2740/15043; C12N 2750/14143; C12N 2800/80

(57) ABSTRACT

The present disclosure relates to a novel delivery system with unique modular CRIPSR-Cas9 architecture that allows better delivery, specificity and selectivity of gene editing. It represents significant improvement over previously described split-Cas9 systems. The modular architecture is “regulatable”. Additional aspects relate to systems that can be both spatially and temporally controlled, resulting in the potential for inducible editing. Further aspects related to a modified viral capsid allowing conjugation to homing agents.

### CROSS-REFERENCE TO RELATED APPLICATIONS

The present application is a continuation of U.S. application Ser. No. 16/325,679, filed Feb. 14, 2019, which application is a U.S. national stage application under 35 U.S.C. § 371 of International Application No. PCT/US2017/047687, filed Aug. 18, 2017, which in turn claims priority to U.S. Ser. No. 62/376,855, filed Aug. 18, 2016, U.S. Ser. No. 62/415,858, filed Nov. 1, 2016, and U.S. Ser. No. 62/481,589, filed Apr. 4, 2017, the content of each of which is incorporated herein by reference in its entirety.

### SEQUENCE LISTING

The instant application contains a Sequence Listing which has been filed electronically in XML format and is hereby incorporated by reference in its entirety. Said XML file created on Jun. 18, 2026, and named “00015-362US2.xml” and is 541,641 bytes in size.

### BACKGROUND

The following discussion of the background of the invention is merely provided to aid the reader in the understanding the invention and is not admitted to describe or constitute prior art to the present invention.

The recent advent of RNA-guided effectors derived from clustered regularly interspaced short palindromic repeats (CRISPR)-CRISPR-associated (Cas) systems has transformed the ability to engineer the genomes of diverse organisms.

Currently, Adeno-Associated Viruses (AAVs) have been widely utilized for genetic therapy due to their overall safety, mild immune response, long transgene expression, high infection efficiency, and are already being used in clinical trials. A main drawback, however, is that AAVs have a limited packaging capacity of around 4.5 kb, making it difficult to deliver Streptococcus pyogenes Cas9 (SpCas9), with a size of around 4.2 kb, a single guide RNA vector, and other components necessary for gene editing.

Thus, a need exists in the art to overcome this technical limitation. This disclosure satisfies this need and provides related advantages as well.

### SUMMARY

Some of the key challenges currently faced by genome editing are: delivery, specificity, and product selectivity. Aspects of this disclosure relate to methods of overcoming these challenges (FIG. 1).

Thus, in one aspect, the present disclosure relate to a modular delivery system that enables programmable incorporation of CRISPR-effectors and facile pseudotyping with the goal of integrating the advantages of both viral and non-viral delivery approaches.

Coupled with the growing knowledge of the genetic and pathogenic basis of disease, development of safe and efficient gene transfer platforms for CRISPR based genome and epigenome engineering can transform the ability to target various human diseases and to also engineer disease resistance. In this regard a range of novel viral and non-viral approaches have been developed towards in vitro and in vivo delivery of CRISPR reagents.

The present disclosure relates to a novel delivery system with unique modular CRISPR-Cas9 architecture that allows better delivery, specificity and selectivity of gene editing. It represents significant improvement over previously described split-Cas9 systems. The modular architecture is “regulatable”. Additional aspects relate to systems that can be both spatially and temporally controlled, resulting in the potential for inducible editing. Further aspects relate to a modified viral capsid allowing conjugation to homing agents, i.e. agents that enable targeting and/or localization of the capsid to a cell, organ, or tissue.

Aspects of the disclosure relate to a recombinant expression system for CRISPR-based genome or epigenome editing. In some embodiments, the recombinant expression system comprises, or alternatively consists essentially of, or yet further consists of: (a) a first expression vector comprising (i) a polynucleotide encoding C-intein, (ii) a polynucleotide encoding C-Cas9, and (iii) a promoter sequence for the first vector; and (b) a second expression vector comprising (i) a polynucleotide encoding N-Cas9, (ii) a polynucleotide encoding N-intein, and (iii) a promoter sequence for the second vector, wherein, optionally, both the first and second expression vectors are adeno-associated virus (AAV) or lentivirus vectors, and wherein co-expression of the first and second expression vectors results in the expression of a whole Cas9 protein.

In some embodiments, the promoter sequence of the first expression vector comprises, or alternatively consists essentially of, or yet further consists of a CMV promoter.

In some embodiments, the promoter sequence of the second vector comprises, or alternatively consists essentially of, or yet further consists of a first promoter operatively linked to an gRNA sequence, optionally an sgRNA, and a second promoter. In some embodiments, the first promoter sequence is a U6 promoter. In some embodiments, the second promoter sequence is a CMV promoter.

In some embodiments, both the first and second expression vectors further comprise, or alternatively consist essentially of, or yet further consist of a poly-A tail.

In some embodiments, the first expression vector further comprises, or alternatively consists essentially of, or yet further consists of a tetracycline response element and/or the second expression vector further comprises, or alternatively consists essentially of, or yet further consists of a tetracycline regulatable activator, or wherein the first expression vector further comprises, or alternatively consists essentially of, or yet further consists of a tetracycline regulatable activator and/or the second expression vector further comprises, or alternatively consists essentially of, or yet further consists of a tetracycline response element. In some embodiments, the tetracycline response element comprises one or more repeats of tetO, optionally seven repeats of tetO. In some embodiments, the tetracycline regulatable activator comprises rtTa and, optionally, 2A.

In some embodiments, the C-Cas9 is dC-Cas9 and the N-Cas9 is dN-Cas9. In further embodiments, the first expression vector and/or second expression vector further comprises, or alternatively consists essentially of, or yet further consists of one or more of KRAB, DNMT3A, or DNMT3L. In further embodiments, recombinant expression system further comprises, or alternatively consists essentially of, or yet further consists of a gRNA for a gene targeted for repression, silencing, or downregulation. In other embodiments, the first expression vector and/or second expression vector further comprises, or alternatively consists essentially of, or yet further consists of one or more of VP64, RtA, or P65. In further embodiments, the recombinant expression system further comprises, or alternatively consists essentially of, or yet further consists of a gRNA for a gene targeted for expression, activation, or upregulation. In still further embodiments, the recombinant expression system further comprises, or alternatively consists essentially of, or yet further consists of a third expression vector encoding the gene targeted for expression, activation, or upregulation and, optionally, a promoter.

In some embodiments, the first expression vector and/or the second expression vector further comprises, or alternatively consists essentially of, or yet further consists of an miRNA circuit.

Further aspects relate to a composition comprising the disclosed recombinant expression system, wherein the first expression vector is encapsulated in a first viral capsid and the second expression vector is encapsulated in a second viral capsid, and optionally, wherein the first viral capsid and/or the second viral capsid is an AAV or lentivirus capsid. In some embodiments, the AAV is one of AAV1, AAV2, AAV3, AAV4, AAV5, AAV6, AAV7, AAV8, AAV9, AAV10, AAV11, or AAV-DJ.

In some embodiments, the first viral capsid and/or the second viral capsid is modified to comprise one or more of the group of: an unnatural amino acid, a SpyTag, or a KTag. In some embodiments, the unnatural amino acid is N-epsilon-((2-Azidoethoxy) carbonyl)-L-lysine.

In some embodiments, the first viral capsid and/or the second viral capsid is pseudotyped with one or more of a peptide, aptamer, oligonucleotide, affibody, DARPin, Kunitz domain, fynomer, bicyclic peptide, anticalin, or adnectin.

In some embodiments, the first viral capsid and/or second viral capsid is an AAV2 capsid. In further embodiments, the unnatural amino acid, a SpyTag, or a KTag is incorporated at amino acid residue R447, S578, N587 or S662 of VP1.

In some embodiments, the first viral capsid and/or second viral capsid is an AAV-DJ capsid. In further embodiments, the unnatural amino acid, a SpyTag, or a KTag is incorporated at amino acid residue N589 of VP1.

In some embodiments, the first viral capsid and second viral capsid are linked.

Some aspects of the disclosure relate to a method of pain management in a subject in need thereof, comprising administering an effective amount of the disclosed composition to the subject, wherein the composition comprises a vector encoding a gRNA targeting one or more of SCN9A, SCN10A, SCN11A, SCN3A, TrpV1, SHANK3, NR2B, IL-10, PENK, POMC, or MVIIA-PC.

Some aspects of the disclosure relate to a method of treating or preventing malaria in a subject in need thereof, comprising administering an effective amount of the disclosed composition to the subject, wherein the composition comprises a vector encoding a gRNA targeting one or more of CD81, MUC13, or SR-B1.

Some aspects of the disclosure relate to a method of treating or preventing hepatitis C in a subject in need thereof, comprising administering an effective amount of the disclosed composition to the subject, wherein the composition comprises a vector encoding a gRNA targeting one or more of CD81, MUC13, SR-B1, GYPA, GYPC, PKLR, or ACKR1.

Some aspects of the disclosure relate to a method of treating or preventing immune rejection of hematopoietic stem cell therapy in a subject in need thereof, comprising administering an effective amount of the disclosed composition to the subject, wherein the composition comprises a vector encoding a gRNA targeting CCR5.

Some aspects of the disclosure relate to a method of treating or preventing HIV in a subject in need thereof, comprising administering an effective amount of the disclosed composition to the subject, wherein the composition comprises a vector encoding a gRNA targeting CCR5.

Some aspects of the disclosure relate to a method of treating or preventing muscular dystrophy in a subject in need thereof, comprising administering an effective amount of the disclosed composition to the subject, wherein the composition comprises a vector encoding a gRNA targeting dystrophin.

Some aspects of the disclosure relate to a method of treating or improving treatment of a cancer in a subject in need thereof, comprising administering an effective amount of the disclosed composition to the subject, wherein the composition comprises a vector encoding a gRNA targeting one or more of PDCD-1, NODAL, or JAK-2.

Some aspects of the disclosure relate to a method of treating or a cytochrome p450 disorder in a subject in need thereof, comprising administering an effective amount of the disclosed composition to the subject, wherein the composition comprises a vector encoding a gRNA targeting CYP2D6.

Some aspects of the disclosure relate to a method of treating or preventing Alzheimer's in a subject in need thereof, comprising administering an effective amount of the disclosed composition of to the subject, wherein the composition comprises a vector encoding a gRNA targeting on LilrB2.

In some embodiments of any one or more of the disclosed method aspects, the subject is a mammal, optionally a murine, a canine, a feline, an equine, a bovine, a simian, or in a human patient.

Further aspects relate to a modified AAV2 capsid comprising an unnatural amino acid, a SpyTag, or a KTag at amino acid residue R447, S578, N587 or S662 of VP1. In some embodiments, the unnatural amino acid is N-epsilon-((2-Azidoethoxy) carbonyl)-L-lysine. In some embodiments, the modified AAV2 capsid is pseudotyped with one or more of a peptide, aptamer, oligonucleotide, affibody, DARPin, Kunitz domain, fynomer, bicyclic peptide, anticalin, or adnectin. In some embodiments, the modified AAV2 capsid is coated with lipofectamine.

Further aspects relate to a modified AAV-DJ capsid comprising an unnatural amino acid, a SpyTag, or a KTag at amino acid residue N589 of VP1. In some embodiments, the unnatural amino acid is N-epsilon-((2-Azidoethoxy) carbonyl)-L-lysine. In some embodiments, the modified AAV-DJ capsid is pseudotyped with one or more of a peptide, aptamer, oligonucleotide, affibody, DARPin, Kunitz domain, fynomer, bicyclic peptide, anticalin, or adnectin. In some embodiments, modified AAV-DJ capsid is coated with lipofectamine.

### BRIEF DESCRIPTION OF THE FIGURES

FIG. 1 is a chart depicting the challenges associated with CRISPR delivery and aspects addressed by the present application.

FIG. 2 depicts a schematic of an exemplary dual-AAV system, each delivering a split-intein, split-Cas9, which is reconstituted upon co-expression.

FIG. 3 depicts a schematic of an exemplary inducible Split-Cas9 system.

FIG. 4A-B shows (A) depicts an exemplary split-Cas9 system for Gene Repression, with a KRAB repressor domain and (B) is an exemplary split-Cas system for gene activation, with VP64 and Rta domains.

FIG. 5 depicts an exemplary schematic of dual AAV with miRNA circuit.

FIG. 6 depicts a schematic of the virus-aptamer-cell interaction.

FIG. 7A-B depicts (A) an exemplary TK-GFP vector schematic and (B) merged fluorescent and phase microscopy images for AAV-DJ TK-GFP transduction of HEK293T cells at various multiplicities of infection (MOIs).

FIG. 8A-B depicts (A) 3 mice administered with an AAV8 inducible dual-Cas9 system targeting ApoB, no Doxycycline administered (B) 3 mice administered with AAV8 inducible dual-Cas9 system targeting ApoB, administered with 200 mg Doxycycline, three times a week, for 4 weeks, showing a 1.7% indel formation when administered with Doxycycline.

FIG. 9 depicts in vitro repression targeting CXCR4. 293T cells were transduced with dual-AAVDJ split-Cas9 virus, cells were collected on day 3, RNA was extracted and RT-qPCR was done.

FIG. 10 depicts in vivo CD81 repression, 3 mice administered with pAAV8_gCD81_KRAB_dCas9 vectors, for in vivo repression. Liver was harvested 4 weeks after AAV administration, RNA was extracted, and RT-qPCR experiments were done. The results show a 35% repression of the CD81 gene from mice administered with the repression vectors vs. wild-type.

FIG. 11 depicts liver stained with anti-CD81. From top to bottom: no primary antibody control, mice administered with AAV8 gCD81 repression split-Cas9 vectors, wild-type control.

FIG. 12 depicts in vitro activation using dC-Cas9_V with showing evidence of in vitro RHOX activation as determined by RT-qPCR using AAVDJ_VR_dCas9 vectors. Controls consist of gRNAs targeting the AAVS1 locus; and showing evidence of in vitro ASCL1 activation as determined by RT-qPCR using AAVDJ_VR_dCas9 vectors.

FIG. 13A-B depicts (A) a histogram showing the number of GFP+ cells normalized wrt to the negative control (in the absence of UAA) while varying the UAA concentration and (B) histogram showing the number of GFP+ cells normalized wrt to the negative control while varying the synthetase concentration.

FIG. 14 depicts a histogram showing the % cells transduced by equal volumes of the different mutants.

FIG. 15 depicts a histogram showing the % of cells transduced by equal volumes of the different variants

FIG. 16A-J depicts versatile genome engineering via a modular split-Cas9 dual AAV system: (A) An exemplary schematic of intein-mediated split-Cas9 pAAVs for genome editing, left, and for temporal inducible genome engineering, right. (B) From left to right, indel frequency at the AAVS1 locus in vitro in HEK293T cells, ex vivo in CD34+ hematopoietic stem cells, and in vivo at the ApoB locus. (C) Relative activity of in vitro AAVS1 locus editing with Cas9 AAVs as compared to inducible-Cas9 (iCas9) AAVs, media supplied with doxycycline (dox: 200 μg/ml). (D) Relative activity of in vivo ApoB editing between Cas9 AAVs and inducible Cas9 AAVs. Mice transduced with iCas9 AAVs where administered saline with or without doxycycline, (dox: 200 mg; total of 12 injections; error bars are SEM). (E) An exemplary schematic of genome repression, through a dCas9-KRAB repressor fusion protein, and schematic of genome activation, through a dCas9-VP64-RTA fusion protein. (F) Evidence of in vitro CXCR4 repression in HEK293T cells, targeting two distinct spacers. (G) Evidence of in vivo CD81 repression in adult mice livers. (H) Evidence of in vitro ASCL1 activation using a dual-gRNA. (I) Evidence of in vivo Afp activation in adult mice livers. (J) Representative immunofluorescence stains of liver sections and corresponding quantitative analysis of relative expression levels is shown: DAPI (lower panels) and anti-CD81 (upper panels). Left panels are negative control (secondary antibody stained sections), middle panels are positive control (non-targeting AAV), and right panels are mice transduced with CD81 AAVs. (scale bars: 250 μm; error bars are SEM).

FIG. 17A-G depicts versatile capsid pseudotyping via UAA mediated incorporation of click-chemistry handles: (A) An exemplary schematic of approach for addition of a UAA to the virus capsid and subsequent click-chemistry based chemical linking of an effector to the UAA. (B) Locations of the surface residues assayed for replacement with UAAs (VP1 residues numbered). (C) Relative titers of the AAV2 mutants in the presence and absence of 2 mM UAA (0.4 mM lysine): 293T cells were transduced with equal amounts of virus and number of fluorescent cells was quantified; no virus assembly is seen in the absence of the UAA. (D) Fluorophore pseudotyping of AAVs via Alexa594 DIBO alkyne was performed: successful linking onto the virus was confirmed via fluorescence visualization of the virus 2 hours post transduction of 293 Ts (scale bars: 250 μm). (E) Oligonucleotide pseudotyping of AAVs via alkyne-tagged oligonucleotides was performed: the selective capture on DNA array spots of AAVs bearing corresponding complementary oligonucleotides was evidenced via specific viral transduction of 293T cells dispersed on those spots (scale bars: 250 μm). (F) Concept of the integrated modular AAV platform that combines programmability in genome engineering effectors and capsid effectors to generate fully programmable modular AAVs. (G) Confirmation that the mAAV integrated system is functional, i.e., UAA modified AAVs can incorporate the split-Cas9 based genome engineering payloads and effect robust genome editing: indel signature and representative NHEJ profiles are shown. FIG. 17G discloses SEQ ID NOS 316-328, respectively, in order of appearance.

FIG. 18A-C depicts in vivo and in vitro genome regulation via mAAVs: (A) An exemplary schematic of workflow for in vivo mAAV-mediated genome engineering: AAV plasmids are designed and constructed, followed by virus production and purification via iodixanol gradients. Mice are then injected with ~0.5E12-1E12 GC through tail-vein or intra-peritoneal routes and whole tissues are harvested for processing at 4 weeks. (B) In vivo CD81 repression: Mice received 1E12 GC of non-targeting or CD81 targeting AAVs by intra-peritoneal (IP) injections. ~40-60% repression of CD81 at the whole tissue level was observed in this experiment via quantitative RT-PCR. (C) Left: in vitro RHOXF2 activation in 293T cells via targeting of two distinct spacers, gRHOXF2_1 and gRHOXF2_2, as well as a combination of both, dual-gRHOXF2. ~1.25-7 fold activation was observed via quantitative RT-PCR under these different conditions. Right: in vivo Afp activation in the liver: mice received 1E12 GC of non-targeting or Afp AAVs by IP injections. ~1.25-3 fold activation of Afp at the whole tissue level was observed in this experiment via quantitative RT-PCR.

FIG. 19A-C depicts optimization of UAA incorporation: synthetase and UAA concentration: (A) UAA incorporation into a GFP reporter sequence bearing a TAG stop site at Y39: Fluorescence images of 293T cells 48 hours post transfection are depicted under different experimental conditions-negative control, wt-GFP transfection, and GFP-Y39TAG reporter cum tRNA-tRNA synthetase transfection in the absence or presence of 2 mM UAA (N-epsilon-((2-Azidoethoxy) carbonyl)-L-lysine; structure shown). UAA incorporation in the latter condition restores robust GFP expression. (B) Role of synthetase amount on UAA incorporation: optimization of the amount of the tRNA-tRNA synthetase plasmid relative to the reporter plasmid (under 2 mM UAA) was performed. A 5:1 ratio showed nearly a 5 fold higher UAA incorporation as compared to a 1:1 ratio. (C) Optimization of UAA concentration on UAA incorporation: A range of UAA concentrations in the presence of 5:1 ratio of tRNA-tRNA synthetase to the reporter plasmid was evaluated. No significant difference in incorporation efficiencies was observed, although at high concentrations of UAA there was greater cell death in the cultures.

FIG. 20A-D depicts versatile capsid pseudotyping via click-chemistry mediated facile linking of moieties to AAV surface. (A) Comparison of the viral titers of AAV2-N587UAA and AAV-DJ-N589UAA produced under identical culture conditions. (B) Confirmation that UAA incorporation does not affect AAV activity (experiments performed in 293 Ts). (C) Representation of a ‘shielded AAV’ resistant to antibody neutralization. (D) Relative activity (assayed via mCherry expression) of AAV-DJ-N589UAA viruses tethered to a range of small molecule and polymer moieties post exposure to pig serum.

FIG. 21A-B shows domain optimization for AAV-CRISPR repression and activation: (A) Domain optimization for AAV-CRISPR repression: Activity of multiple C terminal domain fusions: KRAB or DNA methyltransferase (DNMT3A or DNMT3L) were evaluated, but in transient repression assays no significant additional repression was observed. (error bars are SEM; cells: HEK293 Ts, locus: CXCR4) (B) Domain optimization for AAV-CRISPR activation: Activity of multiple N terminal domain fusions: VP64 and P65 were evaluated, and notably addition of a VP64 domain yielded ~4-fold higher gene expression. (error bars are SEM; p=0.0007; HEK293 Ts, locus: ASCL1).

FIG. 22A-D depicts (A) Schematic of intein-mediated split-dCas9 pAAVs for genome regulation. (B) Approach for modular usage of effector cassettes to enable genome repression via a KRAB-dCas9-Nrl repressor fusion protein, and genome activation via a dCas9-VP64-RTA fusion protein. (C) Evidence of in vivo Afp activation in adult mice livers. Control mice received non-targeting AAV8 virus at the same titers, 5E+11 vg/mouse. (error bars are SEM; p=0.0117). (D) After optimizing domains for activation in vitro, a VP64 activation domain was added onto the dNCas9 vector and the in vivo Afp activation experiments were repeated in mice receiving AAV8 5E+11 vg/mouse. Control mice received non-targeting AAV8 virus at the same titers, 5E+11 vg/mouse. A >6 fold activation was observed at the Afp with the additional VP64 domain. (error bars are SEM; p=0.0271).

FIG. 23A-D shows Split-Cas9 dual AAV system rescues dystrophin expression in mdx mice. (A) Mdx mouse models have a premature stop codon at exon 23. Two different approaches were utilized, using either a single or a dual-gRNA Cas9 system. The single-gRNA was designed to target the stop codon in exon 23. The dual-gRNAs were designed to target up and downstream of exon 23, leading to an excision of the mutated exon 23, and thus the reading frame of the dystrophin gene is recovered and protein expression restored. (B) Dystrophin immunofluorescence in mdx mice transduced with 1E+12 vg/mouse AAV8 split-Cas9 dual gRNA system for exon 23 deletion. (dystrophin, top 3 panels; nuclei, 4′,6′-diamidino-2-phenylindole (DAPI), bottom 3 panels; Scale bar: 250 μm). (C) List of target sequences for Dmd editing. gRNA-L and gRNA-R engineer excision of exon 23, and gRNA-T targets the premature stop codon in exon 23. PAM sequences are underlined; coding sequences are in upper case and intronic sequences in lower case. FIG. 23C discloses SEQ ID NOS 329-331, respectively, in order of appearance. (D) Western blot for dystrophin shows recovery of dystrophin expression. Comparison to protein from WT mice demonstrates restored dystrophin is about ~7-10% of normal amounts for both the dual-gRNA and single-gRNA methods.

FIG. 24 relates to pain Management: Mice were injected intrathecally with 1E+12 vg/mouse of AAV5 Nav 1.7 KRAB repression constructs (dCas9). As seen, about a 70% repression is seen in the SCN9A gene (Nav 1.7), and is shown to be specific, since Nav 1.8 shows no sign of repression. This demonstrates in vivo functionality of the constructs targeting the dorsal root ganglions (DRGs).

FIG. 25 shows mCherry Expression in mice injected intrathecally with 1E+12 vg/mouse of various serotypes (AAV5, AAV1, AAV8, AAV9, AAVDJ) expressing mCherry. A group of mice received intrathecal injections once a week for four weeks of 1E+12 vg/mouse AAV5 mCherry (AAV5_multiple above). As seen, AAV9 and AAVDJ show higher transduction efficiency as compared to other serotypes.

FIG. 26 is a schematic of linking two AAV capsids using SpyTag and KTag or pseudotyped hybridizing oligonucleotides.

FIG. 27 is a schematic showing the general paradigm of pseudotyping using unnatural amino acids with an azide-alkyne reaction or SpyTag and KTag.

FIG. 28A-B shows (A) comparison of the viral titers of AAV2-N587UAA and AAV-DJ-N589UAA (error bars are +/−SEM) and (B) confirmation that UAA incorporation does not negatively affect AAV activity (experiments performed in HEK 293 Ts at varying vg/cell) (error bars are +/−SEM).

FIG. 29A-C shows (A) Coomassie stain of SDS-PAGE resolved capsid proteins of AAVDJ and AAVDJ-N589UAA, (B) Coomassie stain of SDS-PAGE resolved capsid proteins of AAVDJ and AAVDJ-N589UAA following treatment with an alkyne-oligonucleotide (10 kDa), and (C) Western blot of the non-denatured AAV-DJ and AAV-DJN589UAA following treatment with an alkyne-oligonucleotide, and probed with a complementary oligonucleotide-biotin conjugate followed by streptavidin-HRP.

FIG. 30A-D shows versatile capsid pseudotyping via click-chemistry mediated linking of effectors to the AAV surface: (A) Representation of a ‘cloaked AAV’ resistant to antibody neutralization. (B) Relative activity of AAVDJ and AAVDJ-N589UAA viruses tethered to a range of small molecule and polymer moieties post exposure to pig serum assayed via AAV-mCherry based transduction of HEK 293T cells. (C) Relative activity of AAVDJ and AAVDJ-N589UAA viruses tethered to a range of small molecule and polymer moieties post exposure to pig serum assayed via AAV-mCherry based transduction of HEK 293T cells. (D) AAVS1 editing rates (% NHEJ events) of AAVDJ-N589UAA, AAVDJ-N589UAA+oligo, and AAVDJ-N589UAA+oligo+lipofectamine in HEK 293T cells (1E+5 vg/cell).

FIG. 31A-C shows optimization of UAA incorporation into AAVs: (A) Role of synthetase amount on UAA incorporation: optimization of the amount of tRNA and tRNA synthetase plasmid relative to the reporter plasmid (2 mM UAA) was performed. A 5:1 ratio showed nearly 5-fold higher UAA incorporation as compared to a 1:1 ratio. (B) Optimization of UAA concentration on UAA incorporation: a range of UAA concentrations in the presence of 5:1 ratio of tRNA and tRNA synthetase to the reporter plasmid were evaluated. No significant difference in incorporation efficiencies was observed, although at high concentrations of UAA there was greater cell death in the cultures. (C) In the presence of eTF1-E55D a 1.5-4-fold increase in UAA-AAV titers was observed.

FIG. 32 shows transduction efficiency of the ‘cloaked AAVs’ across cell lines: specifically, transduction efficiency of the AAV-DJ-N589UAA and AAV-DJ-N589UAA+oligo+lipofectamine in a variety of cell lines.

FIG. 33 shows a schematic of how gRNA constructs mediate simultaneous activation and repression at endogenous human genes via gRNA-M2M recruiting MCP-VP64 and gRNA-Com recruiting Com-KRAB.

FIG. 34 shows vector design for simultaneous activation and repression (two vector system).

FIG. 35 shows a three vector system for gene repression and gene overexpression. Mice will be injected intrathecally with our split-Cas9 system (vectors a and b) for gene repression (gRNA can be swapped to target different genes) and with a third vector containing a CMV promoter and gene of interest for overexpression (vector c).

FIG. 36 shows a schematic of a split-Cas system comprising a base editing model.

FIG. 37A is an exemplary sequence for one of two vectors in a dual AAV (pX600) system comprising the following elements: a CMV promoter, dCInteinCCas9, KRAB, and PolyA. FIG. 37A discloses SEQ ID NO: 332. FIG. 37B provides annotation information for each of the underlined and/or highlighted portions of the sequence in FIG. 37A. FIG. 37C is a graphical map of the construct encoded by FIG. 37A.

FIG. 38A is an exemplary sequence for one of two vectors in a dual AAV (pX600) system comprising the following elements: a CMV promoter, dCInteinCCas9, DNMT3L, and PolyA. FIG. 38A discloses SEQ ID NO: 333. FIG. 38B provides annotation information for each of the underlined and/or highlighted portions of the sequence in FIG. 38A. FIG. 38C is a graphical map of the construct encoded by FIG. 38A.

FIG. 39A is an exemplary sequence for one of two vectors in a dual AAV (pX600) system comprising the following elements: a CMV promoter, dCInteinCCas9, DNMT3A, and PolyA. FIG. 39A discloses SEQ ID NO: 334. FIG. 39B provides annotation information for each of the underlined and/or highlighted portions of the sequence in FIG. 39A. FIG. 39C is a graphical map of the construct encoded by FIG. 39A.

FIG. 40A is an exemplary sequence for one of two vectors in a dual AAV (Custom) system comprising the following elements: a U6 promoter followed by a guide RNA cloning site, CMV promoter, CP64, and dNCas9NIntein. FIG. 40A discloses SEQ ID NO: 335. FIG. 40B provides annotation information for each of the underlined and/or highlighted portions of the sequence in FIG. 40A. FIG. 40C is a graphical map of the construct encoded by FIG. 40A.

FIG. 41A is an exemplary sequence for one of two vectors in a dual AAV (Custom) system comprising the following elements: a U6 promoter followed by a guide RNA cloning site, CMV promoter, CP65, and dNCas9NIntein. FIG. 41A discloses SEQ ID NO: 336. FIG. 41B provides annotation information for each of the underlined and/or highlighted portions of the sequence in FIG. 41A. FIG. 41C is a graphical map of the construct encoded by FIG. 41A.

FIG. 42A is an exemplary sequence for one of two vectors in a dual AAV system comprising the following elements: an miRNA recognition site, Zac, iU6 promoter, gSa, CMV promoter, and tTRKRAB. FIG. 42A discloses SEQ ID NO: 337. FIG. 42B provides annotation information for each of the underlined and/or highlighted portions of the sequence in FIG. 42A. FIG. 42C is a graphical map of the construct encoded by FIG. 42A.

FIG. 43A is an exemplary sequence for one of two vectors in a dual AAV system comprising the following elements: tetO (Custom), U6 promoter followed by a guide RNA cloning site, CMV promoter, NCas9NIntein, and M2rtTA. FIG. 43A discloses SEQ ID NO: 338. FIG. 43B provides annotation information for each of the underlined and/or highlighted portions of the sequence in FIG. 43A. FIG. 43C is a graphical map of the construct encoded by FIG. 43A.

FIG. 44A is an exemplary sequence for one of two vectors in a dual AAV system comprising the following elements: tetO, CBL, and iCInteinCCas9. FIG. 44A discloses SEQ ID NO: 339. FIG. 44B provides annotation information for each of the underlined and/or highlighted portions of the sequence in FIG. 44A. FIG. 44C is a graphical map of the construct encoded by FIG. 44A.

FIG. 45A is an exemplary sequence for one of two vectors in a dual AAV (pX600) system comprising the following elements: a CMV promoter, CIntein-CCas9, BE3C, and PolyA. FIG. 45A discloses SEQ ID NO: 340. FIG. 45B provides annotation information for each of the underlined and/or highlighted portions of the sequence in FIG. 45A. FIG. 45C is a graphical map of the construct encoded by FIG. 45A.

FIG. 46A and FIG. 46B provide an exemplary sequence for one of two vectors in a dual AAV (Custom) system comprising the following elements: a U6 promoter followed by a guide RNA cloning site, CMV promoter, BE3N, and dNCas9NIntein. FIGS. 46A and 46B disclose SEQ ID NO: 341. FIG. 46C provides annotation information for each of the underlined and/or highlighted portions of the sequence in FIG. 46A and FIG. 46B. FIG. 46D is a graphical map of the construct encoded by FIG. 46A and FIG. 46B.

FIG. 47A and FIG. 47B provide an exemplary sequence for an AAV (pX601) vector comprising the following elements: a CMV promoter, Cas9Sa, U6 promoter, and gSa. FIGS. 47A and 47B disclose SEQ ID NO: 342. FIG. 47C provides annotation information for each of the underlined and/or highlighted portions of the sequence in FIG. 47A and FIG. 47B. FIG. 47D is a graphical map of the construct encoded by FIG. 47A and FIG. 47B.

FIG. 48A is an exemplary sequence for one of two vectors in a dual AAV (pX600) system comprising the following elements: a CMV promoter, dCInteinCCas9, VR, and PolyA. FIG. 48A discloses SEQ ID NO: 343. FIG. 48B provides annotation information for each of the underlined and/or highlighted portions of the sequence in FIG. 48A. FIG. 48C is a graphical map of the construct encoded by FIG. 48A.

FIG. 49A is an exemplary sequence for one of two vectors in a dual AAV (pX600) system comprising the following elements: a CMV promoter, dCInteinCCas9, EcoRV, and PolyA. FIG. 49A discloses SEQ ID NO: 344. FIG. 49B provides annotation information for each of the underlined and/or highlighted portions of the sequence in FIG. 49A. FIG. 49C is a graphical map of the construct encoded by FIG. 49A.

FIG. 50A is an exemplary sequence for one of two vectors in a dual AAV (Custom) system comprising the following elements: a U6 promoter followed by a guide RNA cloning site, CMV promoter, KRAB, and dNCas9NIntein. FIG. 50A discloses SEQ ID NO: 345. FIG. 50B provides annotation information for each of the underlined and/or highlighted portions of the sequence in FIG. 50A. FIG. 50C is a graphical map of the construct encoded by FIG. 50A.

FIG. 51A is an exemplary sequence for one of two vectors in a dual AAV (Custom) system comprising the following elements: a U6 promoter followed by a guide RNA cloning site, CMV promoter, EcoRV, and dNCas9. FIG. 51A discloses SEQ ID NO: 346. FIG. 51B provides annotation information for each of the underlined and/or highlighted portions of the sequence in FIG. 51A. FIG. 51C is a graphical map of the construct encoded by FIG. 51A.

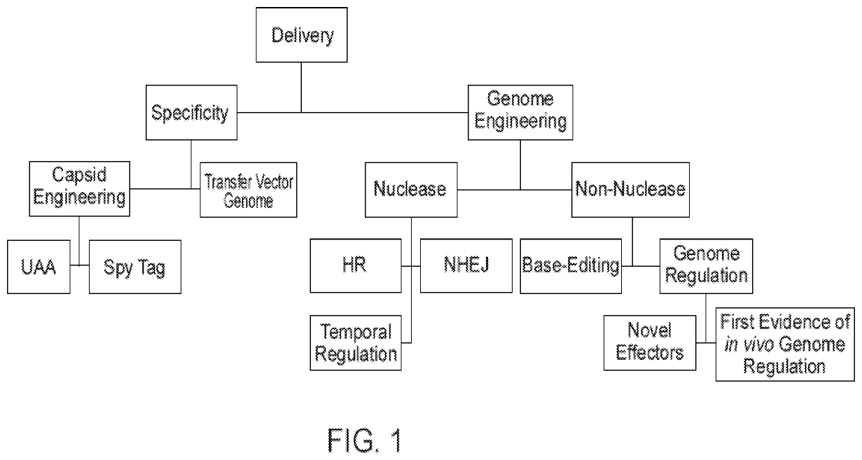

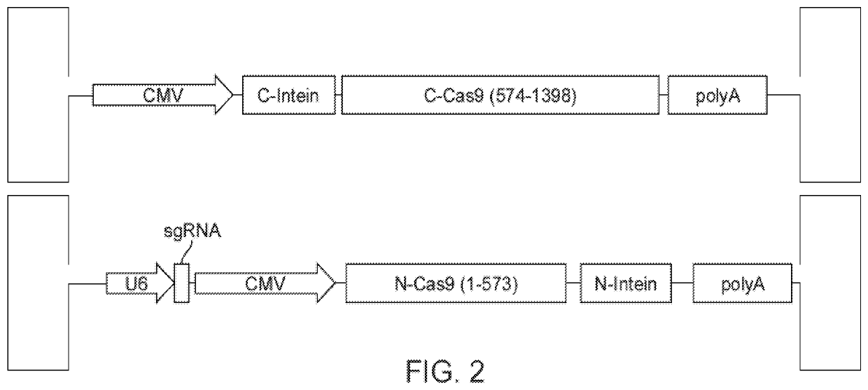

### TABLES

Table 1 lists the guide RNA spacer sequences used in Example 1. Table discloses SEQ ID NOS: 268-281, respectively, in order of appearance.

Table 2a lists the oligonucleotide sequences of the qPCR primers used in Example 1. Table discloses the forward primers as SEQ ID NOS: 282-291 and the reverse primers as SEQ ID NOS 292-301, respectively, in order of appearance.

Table 2b lists the oligonucleotide sequences of the NGS primers used in Example 1. Table discloses SEQ ID NOS: 302-311, respectively, in order of appearance.

Table 2c lists the oligonucleotide sequences of the oligonucleotides for AAV tethering used in Example 1. Table discloses SEQ ID NOS: 312-315, respectively, in order of appearance.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

The term “cell” as used herein may refer to either a prokaryotic or eukaryotic cell, optionally obtained from a subject or a commercially available source.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps and those that do not materially affect the basic and novel characteristics of the recited embodiment. Thus, the “term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

As used herein, the terms “nucleic acid sequence,” “oligonucleotide,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “isolated” as used herein refers to molecules or biologicals or cellular materials being substantially free from other materials.

As used herein, the term “organ” a structure which is a specific portion of an individual organism, where a certain function or functions of the individual organism is locally performed and which is morphologically separate. Non-limiting examples of organs include the skin, blood vessels, cornea, thymus, kidney, heart, liver, umbilical cord, intestine, nerve, lung, placenta, pancreas, thyroid and brain.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. Peptides can be defined by their configuration. For example, “bicyclic peptides” refer to a family of peptides comprising two cyclized portions, optionally engineered to function as an antibody mimetic.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

An “effective amount” or “efficacious amount” is an amount sufficient to achieve the intended purpose. In one aspect, the effective amount is one that functions to achieve a stated therapeutic purpose, e.g., a therapeutically effective amount. As described herein in detail, the effective amount, or dosage, depends on the purpose and the composition, and can be determined according to the present disclosure.

As used herein, the term “CRISPR” refers to a technique of sequence specific genetic manipulation relying on the clustered regularly interspaced short palindromic repeats pathway, which unlike RNA interference regulates gene expression at a transcriptional level. The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. See, e.g., Doench et al. (2014) Nature Biotechnol. 32 (12): 1262-7 and Graham et al. (2015) Genome Biol. 16:260, incorporated by reference herein. When used herein, gRNA can refer to a dual or single gRNA. Non-limiting exemplary embodiments of both are provided herein.

The term “Cas9” refers to a CRISPR associated endonuclease referred to by this name (UniProtKB G3ECR1 (CAS9_STRTR)) as well as dead Cas9 or dCas9, which lacks endonuclease activity (e.g., with mutations in both the RuvC and HNH domain). The term “Cas9” may further refer to equivalents of the referenced Cas9 having at least about 60%, 65%, 70%, 75%, 80%, 85%, 90%, 95%, or 99% identity thereto, including but not limited to other large Cas9 proteins.

The term “intein” refers to a class of protein that is able to excise itself and join the remaining portion(s) of the protein via protein splicing. A “split-intein” refers to an intein that comes from two genes. A non-liming example is the split intein in N. punctiforme disclosed herein as part of a split-Cas9 system. The prefixes N and C may be used in context of a split intein to establish which protein terminus the gene encoding the half of the intein comprises.

As used herein, the term “recombinant expression system” refers to a genetic construct for the expression of certain genetic material formed by recombination.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are disclosed in the prior art. Non-limiting exemplary serotypes useful in the methods disclosed herein include any of the 11 serotypes, e.g., AAV2 and AAV8, or variant serotypes, e.g. AAV-DJ.

The term “lentivirus” as used herein refers to a member of the class of viruses associated with this name and belonging to the genus lentivirus, family Retroviridae. While some lentiviruses are known to cause diseases, other lentivirus are known to be suitable for gene delivery. See, e.g., Tomás et al. (2013) Biochemistry, Genetics and Molecular Biology: “Gene Therapy-Tools and Potential Applications,” ISBN 978-953-51-1014-9, DOI: 10.5772/52534.

As used herein, the term “vector” intends a recombinant vector that retains the ability to infect and transduce non-dividing and/or slowly-dividing cells and integrate into the target cell's genome. The vector may be derived from or based on a wild-type virus. Aspects of this disclosure relate to an adeno-associated virus or lentiviral vector.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CMV promoter and U6 promoter. Non-limiting exemplary promoter sequences are provided herein below:

CMV promoter
(SEQ ID NO: 1)
ATACGCGTTGACATTGATTATTGACTAGTTATTAATAGTAATCAATT
ACGGGGTCATTAGTTCATAGCCCATATATGGAGTTCCGCGTTACATA
ACTTACGGTAAATGGCCCGCCTGGCTGACCGCCCAACGACCCCCGCC
CATTGACGTCAATAATGACGTATGTTCCCATAGTAACGCCAATAGGG
ACTTTCCATTGACGTCAATGGGTGGAGTATTTACGGTAAACTGCCCA
CTTGGCAGTACATCAAGTGTATCATATGCCAAGTACGCCCCCTATTG
ACGTCAATGACGGTAAATGGCCCGCCTGGCATTATGCCCAGTACATG
ACCTTATGGGACTTTCCTACTTGGCAGTACATCTACGTATTAGTCAT
CGCTATTACCATGGTGATGCGGTTTTGGCAGTACATCAATGGGCGTG
GATAGCGGTTTGACTCACGGGGATTTCCAAGTCTCCACCCCATTGAC
GTCAATGGGAGTTTGTTTTGGCACCAAAATCAACGGGACTTTCCAAA
ATGTCGTAACAACTCCGCCCCATTGACGCAAATGGGCGGTAGGCGTG
TACGGTGGGAGGTCTATATAAGCAGAGCTCGTTTAGTGAACCGTCAG
ATCGCCTGGAGACGCCATCCACGCTGTTTTGACCTCCATAGAAGACA
CCGGGACCGATCCAGCCTCCGGACTCTAGAGGATCGAACCCTT

or a biological equivalent thereof.

U6 promoter
(SEQ ID NO: 2)
GAGGGCCTATTTCCCATGATTCCTTCATATTTGCATATACGATACAAGGC
TGTTAGAGAGATAATTAGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACC

or a biological equivalent thereof.

A number of effector elements are disclosed herein for use in these vectors; e.g., a tetracycline response element (e.g., tetO), a tet-regulatable activator, T2A, VP64, RtA, KRAB, and a miRNA sensor circuit. The nature and function of these effector elements are commonly understood in the art and a number of these effector elements are commercially available. Non-limiting exemplary sequences thereof are disclosed herein and further description thereof is provided herein below.

The term “aptamer” as used herein refers to single stranded DNA or RNA molecules that can bind to one or more selected targets with high affinity and specificity. Non-limiting exemplary targets include by are not limited to proteins or peptides.

The term “affibody” as used herein refers to a type of antibody mimetic comprised of a small protein engineered to bind a large number of target proteins or peptides with high affinity. The general affibody structure is based on a three helix-bundle which can then be modified for binding to specific targets.

The term “DARPin” as used herein refers to a designed ankyrin repeat protein, a type of engineered antibody mimetic with high specificity and affinity for a target protein. In general. DARPins comprise at least three repeats of a protein motif (ankyrin), optionally four or five, and have a molecular weight of about 14 to 18 kDa.

The term “Kunitz domain” as used herein refers to a disulfide right alpha+beta fold domain found in proteins that function as a protease inhibitor. In general, Kunitz domains are approximately 50 to 60 amino acids in length and have a molecular weight of about 6 kDa.

The term “fynomers” as used herein refers to small binding proteins derived from human Fyn SH3 domains (described in GeneCards Ref. FYN), which can be engineered to be antibody mimetics.

The term “anticalin” as used herein refers to a type of antibody mimetic, currently commercialized by Pieris Pharmaceuticals, including artificial proteins capable of binding to antigens that are not structurally related to antibodies. Anticalins are derived from human lipcalins and modified to bind a particular target.

The term “adnectin” as used herein refers to a monobody, which is a synthetic binding protein serving as an antibody mimetic, which is constructed using a fibronectin type III domain (FN3).

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Modes of Carrying Out the Disclosure

The present disclosure relates to a novel delivery system with unique modular CRISPR-Cas9 architecture that allows better delivery, specificity and selectivity of gene editing. It represents significant improvement over previously described split-Cas9 systems. The modular architecture is “regulatable”. Additional aspects relate to systems that can be both spatially and temporally controlled, resulting in the potential for inducible editing. Further aspects relate to a modified viral capsid allowing conjugation to homing agents.

### Split-Cas System

In one aspect, the present disclosure relates to “split-Cas9” in which Cas9 is split into two halves—C-Cas9 and N-Cas9—and fused with a two intein moieties or a “split intein”. See, e.g., Volz et al. (2015) Nat Biotechnol. 33 (2): 139-42; Wright et al. (2015) PNAS 112 (10) 2984-89. A “split intein” comes from two genes. A non-limiting example of a “split-intein” are the C-intein and N-intein sequences originally derived from N. punctiforme. A non-limiting exemplary split-Cas9 has a C-Cas9 comprising residues 574-1398 and N-Cas9 comprising residues 1-573. An exemplary split-Cas9 for dCas9 involves two domains comprising these same residues of dCas9, denoted dC-Cas9 and dN-Cas9.

Non-limiting exemplary sequences for these split-Cas9 modules are provided herein below. The amino acid numbers are provided with respect to wild type Cas9.

Cintein (bold) +CCas9(normal) (H840, bold
underline, unmodified sequence)
(SEQ ID NO: 3)
MIKIATRKYLGKQNVYDIGVERDHNFALKINGFIASCFDSVEISGVEDRF
NASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGI
LQTVKVVDELVKVMGRHKPENIVIEMARENQTTQKGQKNSRERMKRIEEG
IKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDS
RMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGY
KEVKKDLIIKLPKYSLFELENGRKRMLASAGELQKGNELALPSKYVNFLY
LASHYEKLKGSPEDNEQKQLFVEQHKHYLDEIIEQISEFSKRVILADANL
DKVLSAYNKHRDKPIREQAENIIHLFTLTNLGAPAAFKYFDTTIDRKRYT
STKEVLDATLIHQSITGLYETRIDLSQLGGD

or a biological equivalent thereof.

Cintein (bold) +dCCas9 (normal) (H840A, bold
italics, modified sequence)
(SEQ ID NO: 4)
MIKIATRKYLGKQNVYDIGVERDHNFALKINGFIASCFDSVEISGVEDRF
NASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGI
LQTVKVVDELVKVMGRHKPENIVIEMARENQTTQKGQKNSRERMKRIEEG
IKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDS
RMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGY
KEVKKDLIIKLPKYSLFELENGRKRMLASAGELQKGNELALPSKYVNFLY
LASHYEKLKGSPEDNEQKQLFVEQHKHYLDEIIEQISEFSKRVILADANL
DKVLSAYNKHRDKPIREQAENIIHLFTLTNLGAPAAFKYFDTTIDRKRYT
STKEVLDATLIHQSITGLYETRIDLSQLGGD

or a biological equivalent thereof.

NCas9 (normal) (D10, bold underline, unmodified
sequence)+N-intein (bold)
(SEQ ID NO: 5)
MGPKKKRKVAAADYKDDDDKGIHGVPAADKKYSIGLDIGTNSVGWAVITD
EYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIE
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRVDNL
PN

or a biological equivalent thereof.

dNCas9(normal) (D10A, bold italic, modified
sequence)+N-intein (bold)
(SEQ ID NO: 6)
MGPKKKRKVAAADYKDDDDKGIHGVPAADKKYSIGLAIGTNSVGWAVITD
EYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIE
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRVDNL
PN

or a biological equivalent thereof.

Aspects of this disclosure relate to a recombinant expression system for CRISPR-based genome or epigenome editing comprising, or alternatively consisting essentially of, or yet further consisting of: (a) a first expression vector comprising (i) a polynucleotide encoding C-intein, (ii) a polynucleotide encoding C-Cas9, and (iii) a promoter sequence; and (b) a second expression vector comprising (i) a polynucleotide encoding N-Cas9, (ii) a polynucleotide encoding N-intein, and (iii) a promoter sequence, wherein co-expression of the first and second expression vectors results in the expression of a functional Cas9 protein.

In some embodiments, both the first and second expression vectors of the recombinant expression system are adeno-associated virus (AAV) vectors or lentiviral vectors.

The addition of effector elements to the vectors disclosed herein allows for the regulation of Cas9 expression to tailor the recombinant expression system for a particular use in CRISPR-based genome or epigenome editing. Non-limiting exemplary effector elements and their use in context of the disclosed “split-Cas9” and/or the recombinant expression system are provided below. It should be appreciated that each of the effector elements described below are described in context of a particular function in the recombinant expression system. Therefore, where more than one of these functions is desired, these effector elements may be used in combination in the recombinant expression system. In contrast, where only one of these functions is desired, only the corresponding effector element may be used in the recombinant expression system.

### Effector Elements for Temporal Regulation

In one aspect, the first and/or second vector of the recombinant expression system comprise, or alternatively consist essentially of, or yet further consist of, an effector element that allows for inducible expression, where introduction of a specific external agent allows induces the expression of a vector. In general, such induction is achieved due to the interaction between the specific agent and a effector element allows for completion of transcription or translation.

A non-limiting example of such an inducible switch is a tetracycline dependent system referred to herein as a “Tet-ON” system. The Tet-ON system comprises a tetracycline response element (“TRE”), which acts as a transcriptional repressor of the genes downstream of the TRE, and a corresponding tetracycline-regulatable activator (“tet-regulatable activator”, which binds to the TRE and allows for expression of the genes downstream of the TRE. The tet-regulatable activator requires the presence of tetracycline or its derivatives (such as but not limited to doxycycline) in order to bind to the TRE. Thus, by using a Tet-ON system, expression of the genes downstream of the TRE can be “turned on” by the addition of tetracycline or its derivatives (such as but not limited to doxycycline) provided that the tet-regulatable element has also been transcribed.

In some embodiments, the TRE comprises TetO, or optionally one or more repeating units thereof or seven repeating units thereof. The canonical nucleic acid sequence for TetO is: ACTCCCTATCAGTGATAGAGAA (SEQ ID NO: 7). The TRE may further comprise a promoter sequence. A non-limiting example of such a TRE, comprising seven repeating units of TetO and a minimal CMV promoter is the nucleic acid sequence:

tetO7-minCMV promoter
(SEQ ID NO: 8)
TTTACTCCCTATCAGTGATAGAGAACGTATGAAGAGTTTACTCCCTATCA
GTGATAGAGAACGTATGCAGACTTTACTCCCTATCAGTGATAGAGAACGT
ATAAGGAGTTTACTCCCTATCAGTGATAGAGAACGTATGACCAGTTTACT
CCCTATCAGTGATAGAGAACGTATCTACAGTTTACTCCCTATCAGTGATA
GAGAACGTATATCCAGTTTACTCCCTATCAGTGATAGAGAACGTATAAGC
TTTAGGCGTGTACGGTGGGCGCCTATAAAAGCAGAGCTCGTTTAGTGAAC
CGTCAGATCGCCTGGAGCAATTCCACAACACTTTTGTCTTATACCAACTT
TCCGTACCACTTCCTACCCTCGTAAA

or a biological equivalent thereof.

A further exemplary sequence comprises seven repeating units of TetO:

tetO7
(SEQ ID NO: 9)
TTTACTCCCTATCAGTGATAGAGAACGTATGAAGAGTTTACTCCCTATCA
GTGATAGAGAACGTATGCAGACTTTACTCCCTATCAGTGATAGAGAACGT
ATAAGGAGTTTACTCCCTATCAGTGATAGAGAACGTATGACCAGTTTACT
CCCTATCAGTGATAGAGAACGTATCTACAGTTTACTCCCTATCAGTGATA
GAGAACGTATATCCAGTTTACTCCCTATCAGTGATAGAGAACGTATAA

or a biological equivalent thereof.

In some embodiments, the tet-regulatable activator comprises rtTA, also known as “reverse tetracycline-controlled transactivator.” See, e.g., Gossen et al. (1995) Science 268 (5218): 1766-1769. Where the tet-regulatable activator is provided in a vector encoding more than gene (i.e. a multicistronic vector), the tet-regulatable activator can further comprise a “self-cleaving” peptide that allows for its dissociation from the other vector products. A non-limiting example of such a self-cleaving peptide is 2A, which is a short protein sequences first discovered in picornaviruses. Peptide 2A functions by making ribosomes skip the synthesis of a peptide bond at the C-terminus of a 2A element, resulting in a separation between the end of the 2A sequence and the peptide downstream thereof. This “cleavage” occurs between the Glycine and Proline residues at the C-terminus. A non-limiting exemplary amino acid sequence of tet-regulatable activator comprising both 2A and rtTA is provided below:

2A (bold) +M2rtTA (normal) (tet activator)
(SEQ ID NO: 10)
GSGATNFSLLKQAGDVEENPGPMSRLDKSKVINGALELLNGVGIEGLTTR
KLAQKLGVEQPTLYWHVKNKRALLDALPIEMLDRHHTHFCPLEGESWQDF
LRNNAKSFRCALLSHRDGAKVHLGTRPTEKQYETLENQLAFLCQQGFSLE
NALYALSAVGHFTLGCVLEEQEHQVAKEERETPTTDSMPPLLRQAIELFD
RQGAEPAFLFGLELIICGLEKQLKCESGGPADALDDFDLDMLPADALDDF
DLDMLPADALDDFDLDMLPG

or a biological equivalent thereof.

In some embodiments, Tet-ON system may be integrated into a split Cas-9 system, such as the recombinant expression system disclosed herein.

In some embodiments, the first vector comprises a tetracycline response element (“TRE”) and the second vector comprises the tetracycline-regulatable activator “tet-regulatable activator”). In some embodiments, the second vector comprises a TRE and the first vector comprises the tet-regulatable activator.

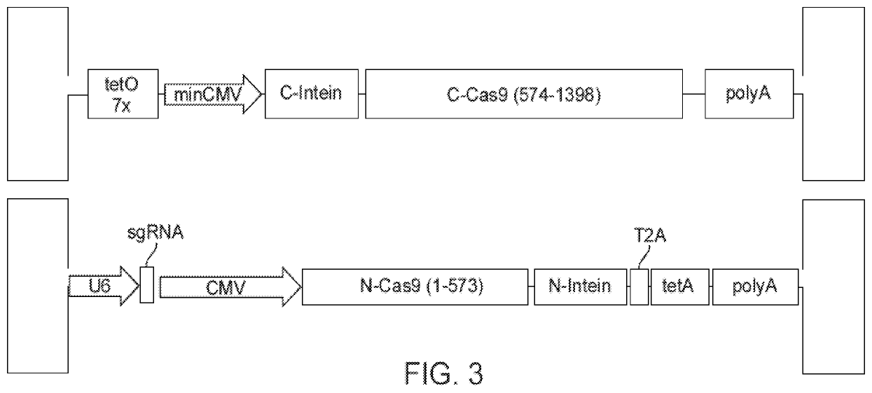

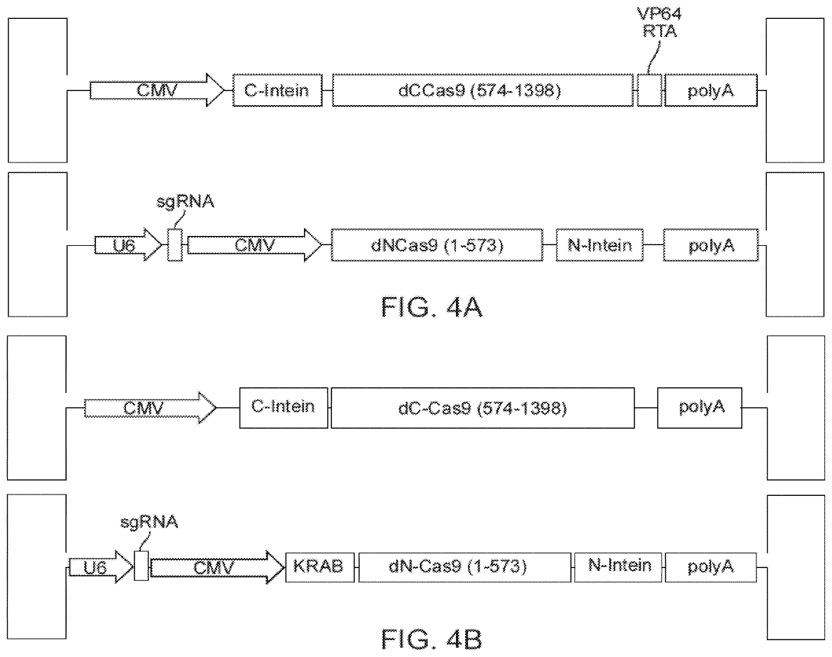

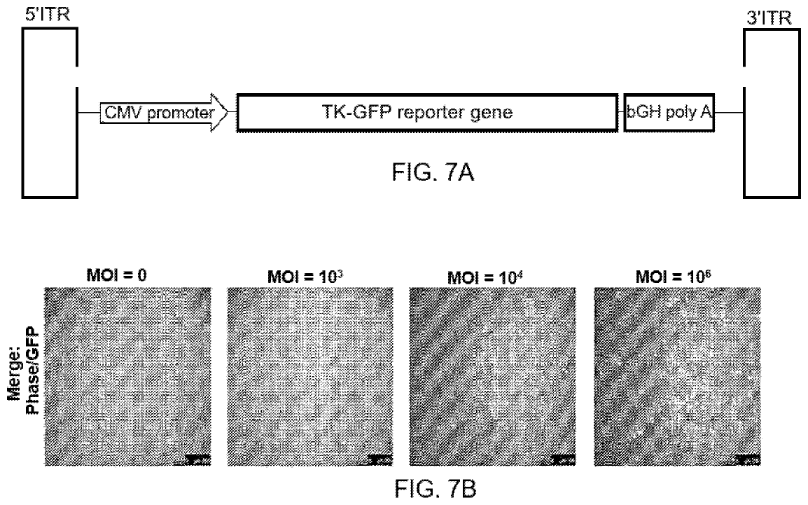

A non-limiting example is depicted in the Figures: for the C-Cas9 vector, a TRE comprising Tet operator (TetO) and a minimal CMV promoter, for the N-Cas9 vector, a tet-regulatable activator comprising rtTA can optionally be added. The introduction of doxycycline to the system allows rtTa to bind to TetO and initiate transcription of C-Cas9, allowing gene editing. (FIG. 3). Applicants have tested this non-limiting exemplary system in vivo and demonstrated that editing is seen in the presence of DOX+ mice, but not in DOX-mice (FIG. 7).

### Effector Elements for Tissue Specificity

In one aspect, the first and/or second vector of the recombinant expression system comprise, or alternatively consist essentially of, or yet further consist of, an effector element or “circuit” that provides for tissue specific expression, i.e. where the expression of the vector is induced by one or more agents, such as proteins, oligonucleotides, or other biological components, present in one or more specific tissues.

A non-limiting example of such as circuit is a tunable microRNA (“miRNA”) circuit or switch. An miRNA switch is a repressor or activator of gene expression that can be designed to be positively or negatively regulated by microRNA.

MircoRNA are small non-coding RNA molecules that silence mRNA by pairing to a target mRNA and causing one or more of cleavage of the mRNA strand into two pieces, destabilization of the mRNA through shortening of the poly(A)tail, and/or decreasing efficiency of mRNA translation. Specific miRNA that are expressed in specific tissues are catalogued in a variety of databases, for example in miRmine (guanlab.ccmb.med.umich.edu/mirmine/) and MESAdb (konulab.fen.bilkent.edu.tr/mirna/mirna.php). Non-limiting examples of miRNA and corresponding miRNA targets that may be relevant herein are provided:

HeLa:
miR-21-5p:
(SEQ ID NO: 11)
uagcuuaucagacugauguuga
Inserted target:
(SEQ ID NO: 12)
TCAACATCAGTCTGATAAGCTAAGATCTA
HUVEC:
miR-126-3p:
(SEQ ID NO: 13)
ucguaccgugaguaauaaugcg
Inserted target:
(SEQ ID NO: 14)
CGCATTATTACTCACGGTACGAAGATCAC
Heart:
miR-la-3p:
(SEQ ID NO: 15)
uggaauguaaagaaguauguau
Inserted target:
(SEQ ID NO: 16)
ATACATACTTCTTTACATTCCAAGATCAC
Liver:
miR-122a-5p:
(SEQ ID NO: 17)
uggagugugacaaugguguuug
inserted target:
(SEQ ID NO: 18)
CAAACACCATTGTCACACTCCAAGATCAC

or a biological equivalent each thereof. By selecting a tissue specific miRNA and generating an miRNA circuits targeted by this miRNA, vector expression can be calibrated to be highly tissue specific.

For example, an exemplary vector may contain an miRNA circuit comprised of a repressor of expression which is negatively regulated by a miRNA target site in its 5′ UTR. Thus, if the vector is delivered to a target tissue type which expresses the miRNA, the repressor is repressed, and the corresponding vector is activated. In contrast, if the vector is delivered to the incorrect tissue type which doesn't contain the miRNA site, the vector is repressed.

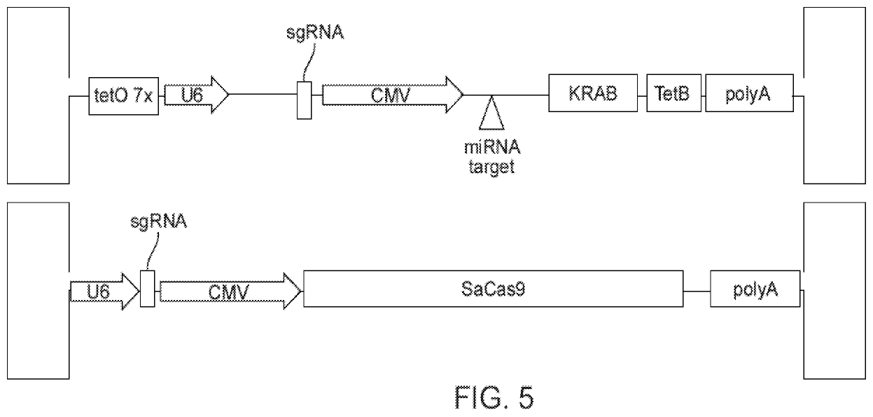

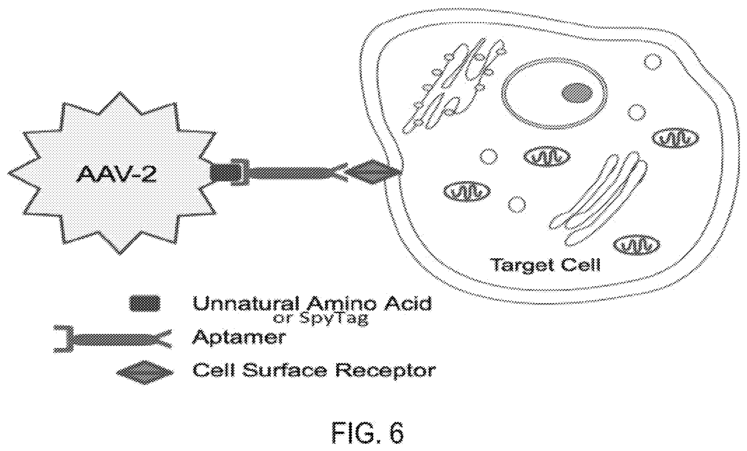

In some embodiments, the first and/or second vector incorporate an miRNA switch which targets specific tissues. A non-limiting exemplary schematic of such incorporation is provided in FIG. 5. In some embodiments, the miRNA switch comprises repressor of expression which is negatively regulated by a miRNA target.

### Effector Elements for Gene Editing

As the recombinant expression system disclosed herein can employ either active or dead Cas9, a variety of optional effector elements may be incorporated to facilitate genome editing along the lines described herein.

Knock-outs and Knock-ins: The recombinant expression system disclosed herein is designed for CRISPR-based genome or epigenome editing. In general, CRISPR-based genome or epigenome editing relies on the function of Cas9 to facilitate the pairing between a gRNA and a target sequence. The gRNA is generally designed target a specific target gene and can further comprise CRISPR RNA (crRNA) and trans-activating CRIPSPR RNA (tracrRNA). Upon pairing of the Cas9-gRNA complex to the target gene, an active Cas9 enzyme can trigger target specific cleavage to disrupt the gene and, optionally, known out or knock in a gene. This is the traditional approach taken to CRISPR-Cas9 gene editing and proves exceedingly useful for therapeutic applications, specifically with genetic diseases.

Alternatively, if dead Cas9 (“dCas9”) is used, the Cas9-gRNA complex can be configured for different editing effects, including but not limited to editing; downregulating, repressing, or silencing; upregulating, overexpressing, or activating; or altering the methylation of target gene.

Base Editing: In some embodiments, a base editing approach may be incorporated into the recombinant expression system, e.g. a split-Cas9 dual AAV system, employing dCas9.

For example, a cytidine deaminase enzyme that directs the conversion of a cytidine to uridine, therefore being useful to fix point-mutations, can be incorporated into the first and/or second vector. This approach does not require double-strand breaks and is efficient at gene correction with point mutations without introducing random indels, as risk posed by traditional CRISPR-Cas9 gene editing. Therefore, this system increases product selectivity by minimizing off-target random indel formations. A non-limiting example of this approach employs the third-generation base editor, APOBEC-XTEN-dCas9 (A840H)-UGI (disclosed in Komor et al. (2016) Nature 533:420-424 and Supplementary Materials), which nicks the non-edited strand containing a G opposite of the edited U. An construct for a Cas9 comprising APOBEC1 from Komor et al. that may be adapted into the recombinant expression system, e.g. split-Cas9 system, disclosed herein is provided below:

BE3 (rAPOBEC1 (bold, underline)-XTEN-Cas9n-
UGI-NLS)
(SEQ ID NO: 19)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGSETPGTSESATPESDKKYS
IGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKE
DIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVI
TLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLY
ETRIDLSQLGGDSGGSTNLSDIIEKETGKQLVIQESILMLPEEVEEVIGN
KPESDILVHTAYDESTDENVMLLTSDAPEYKPWALVIQDSNGENKIKMLS
GGSPKKKRKV

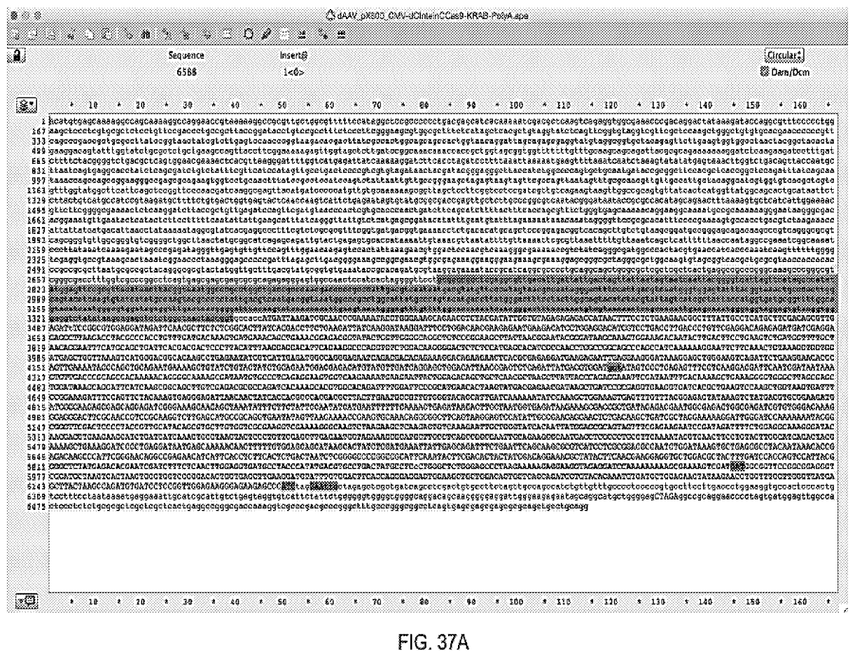

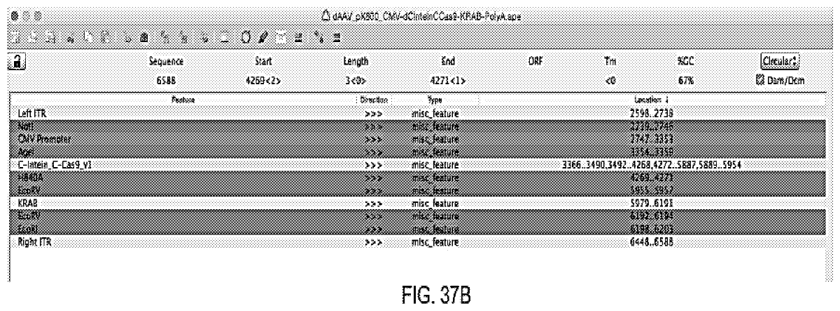

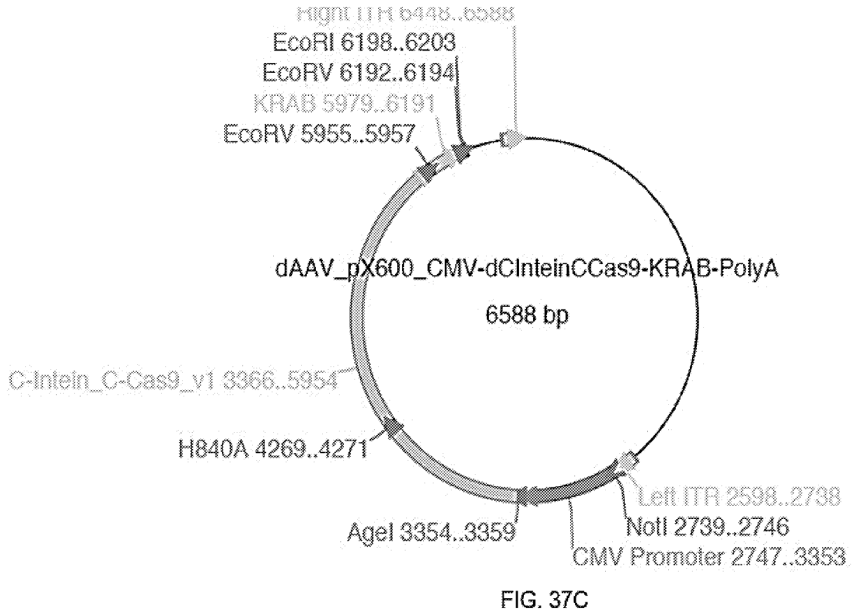

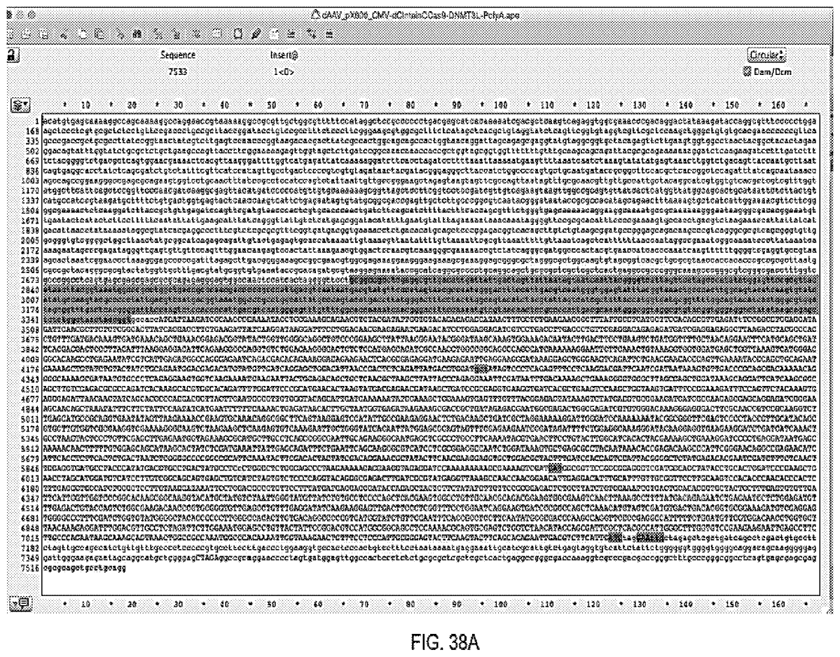

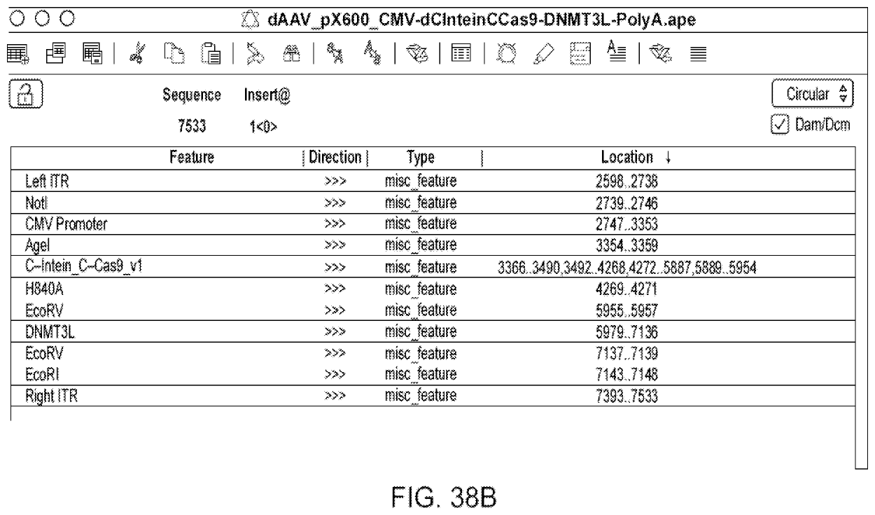

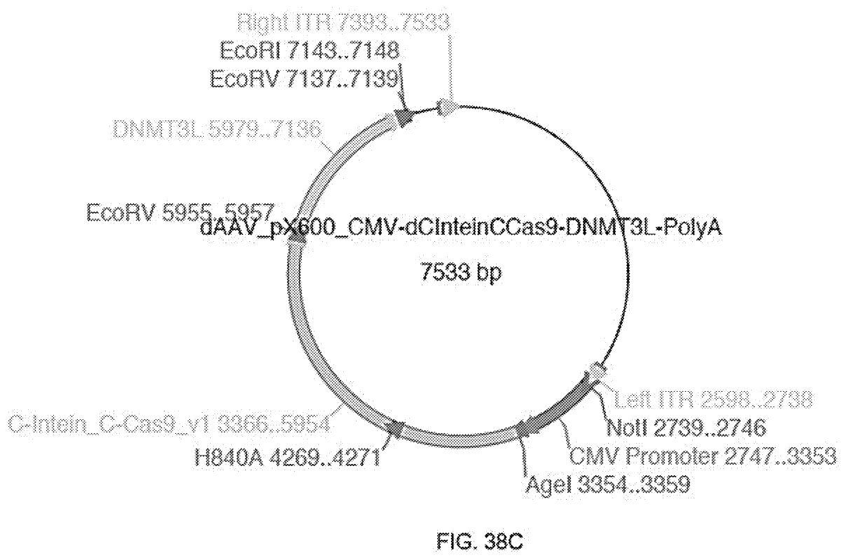

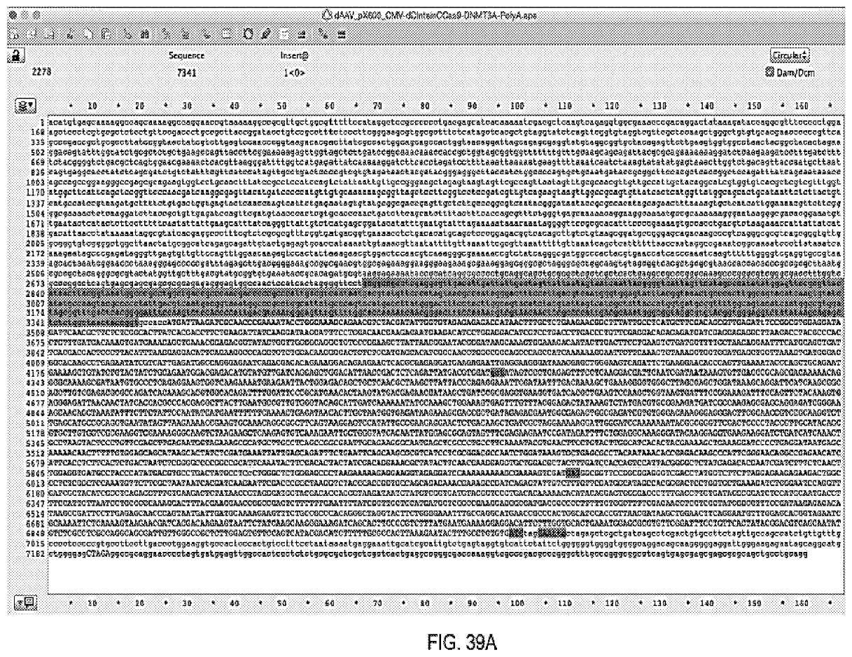

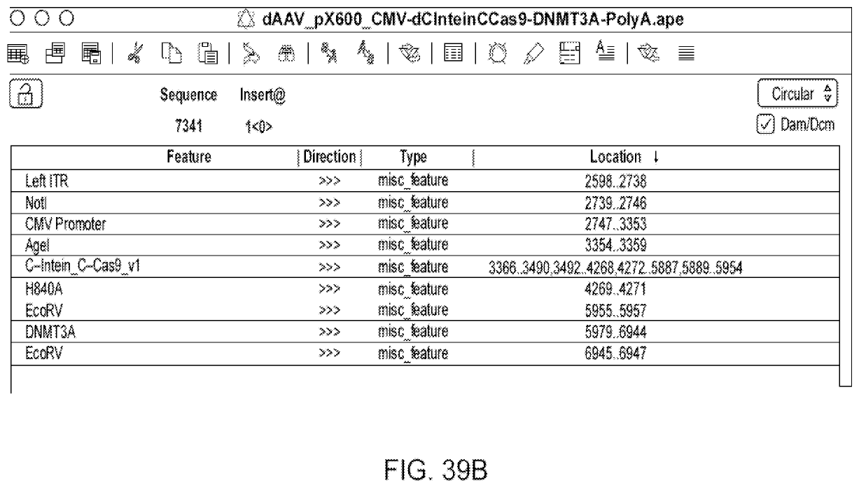

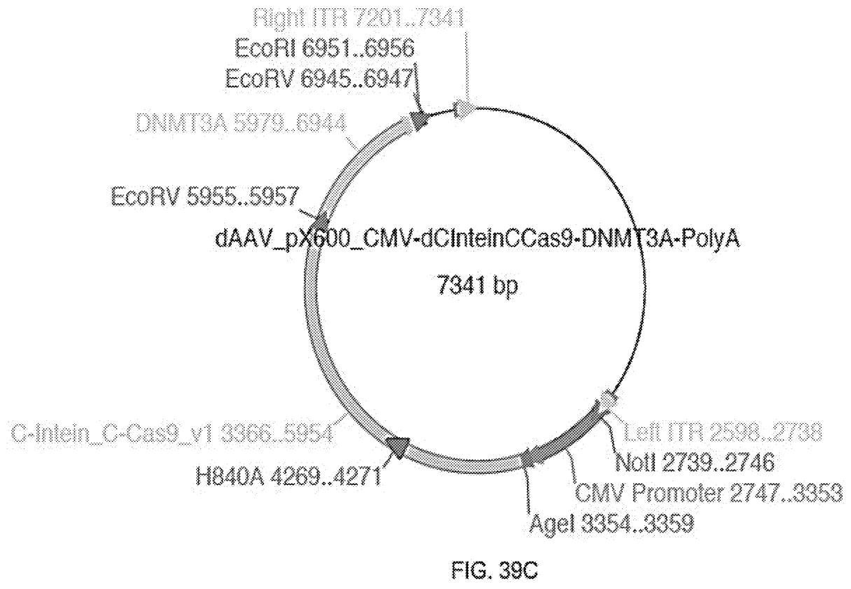

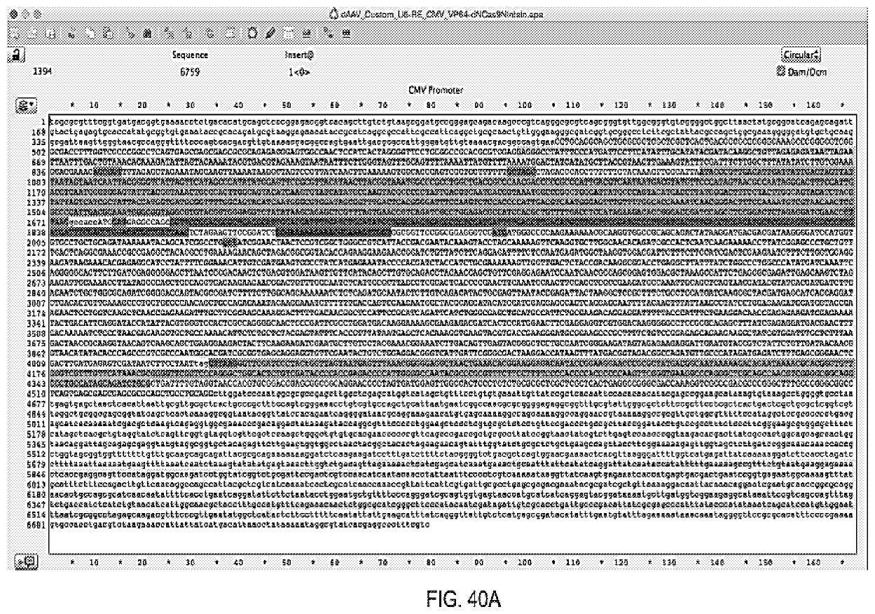

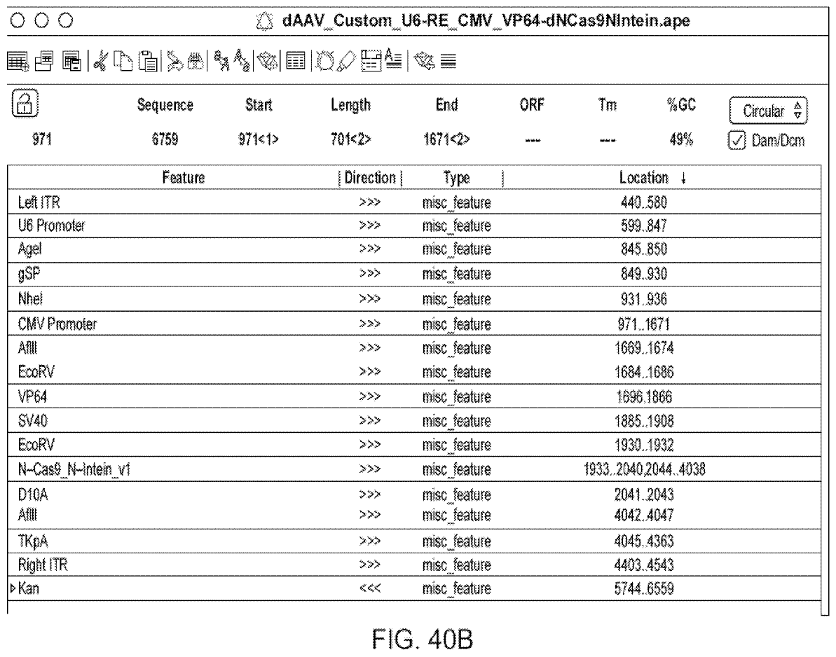

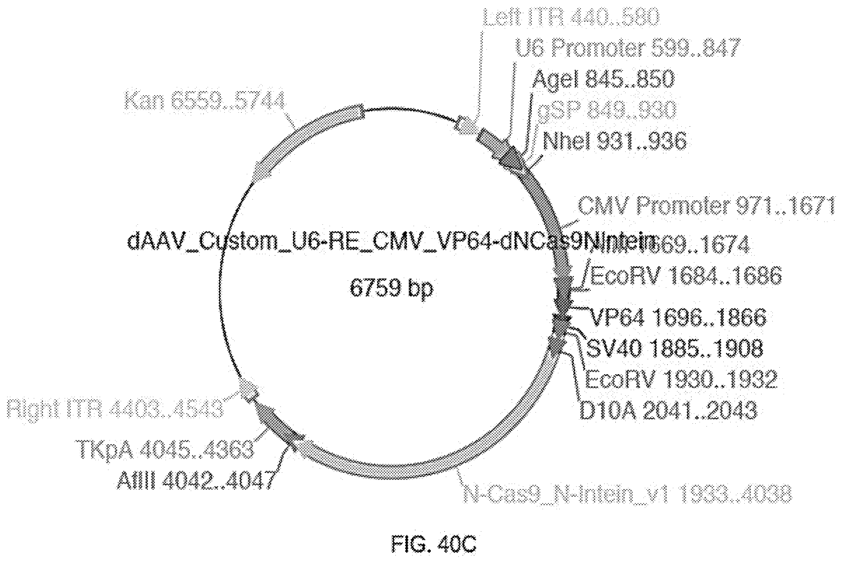

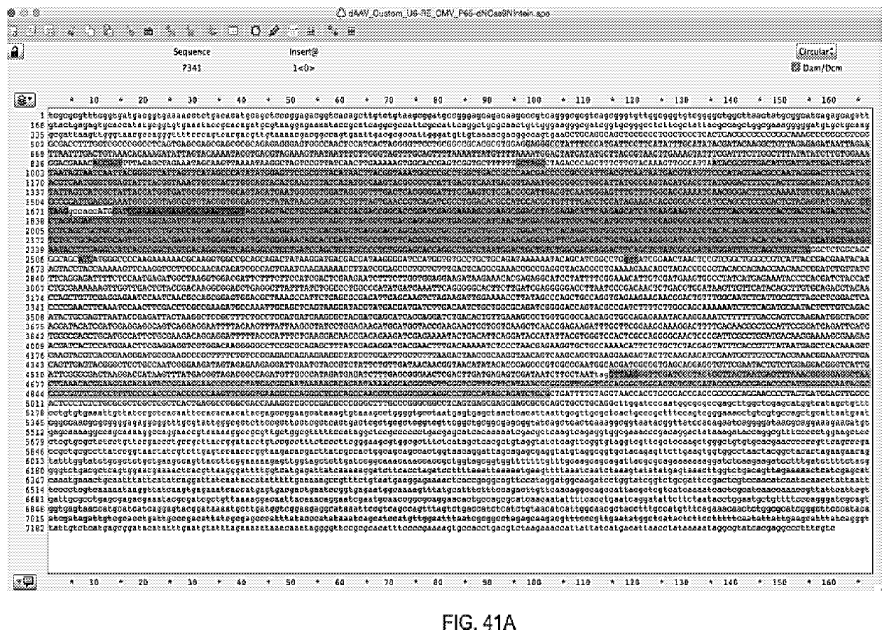

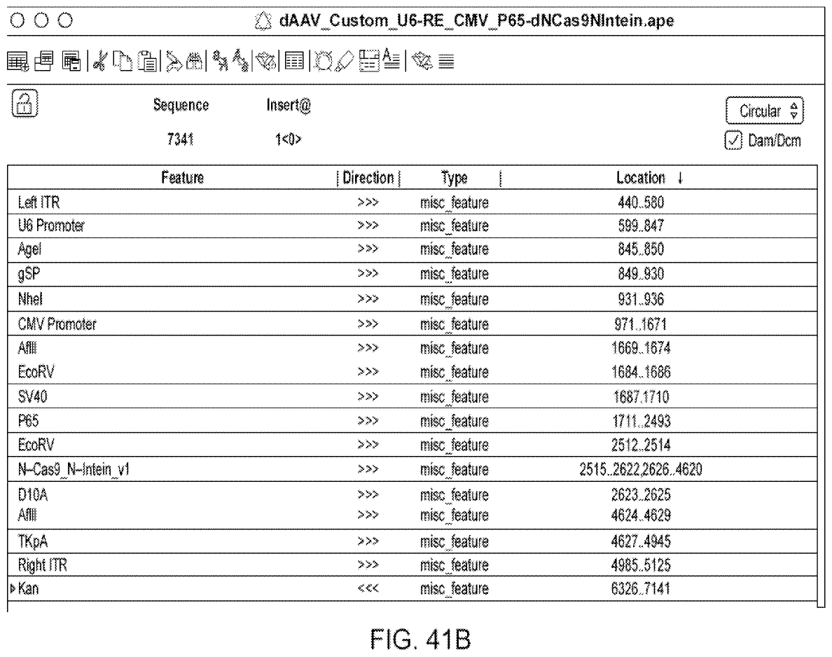

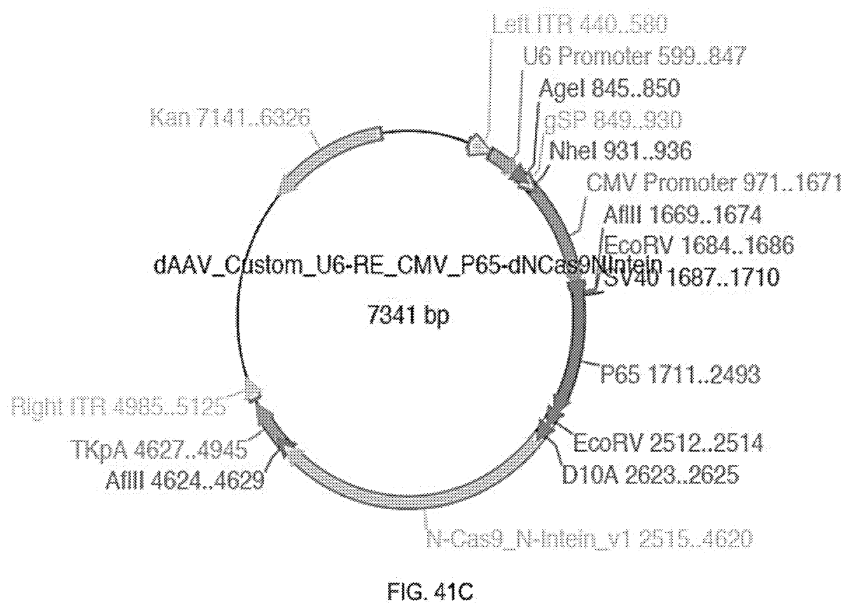

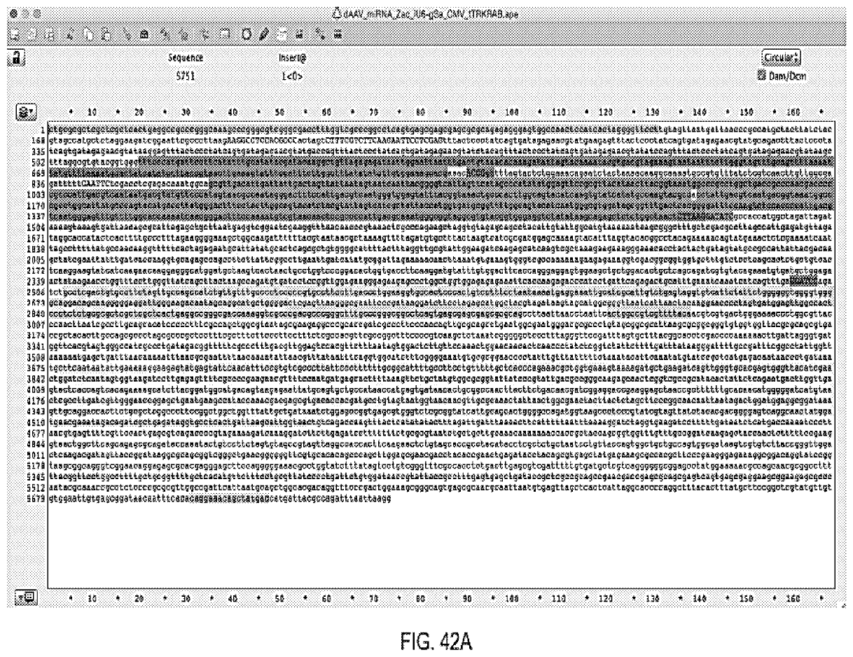

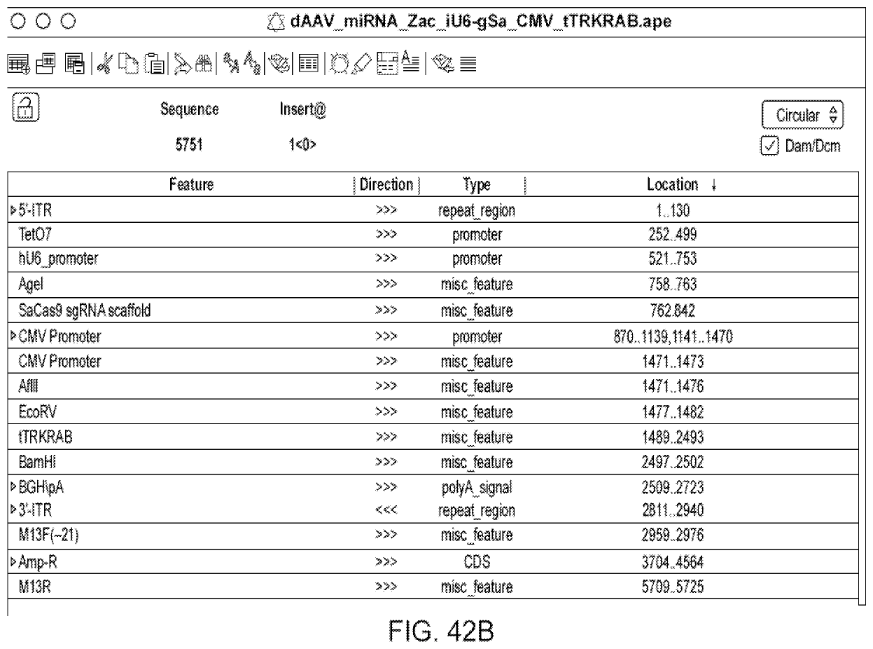

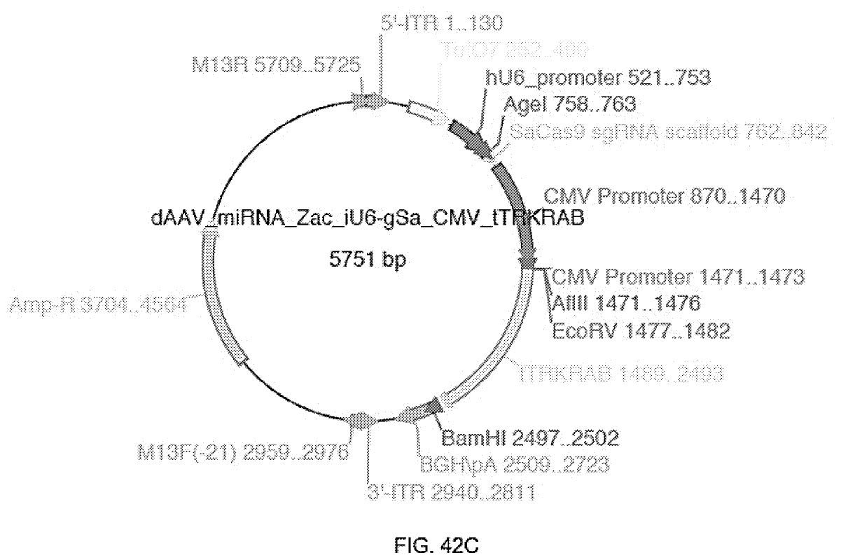

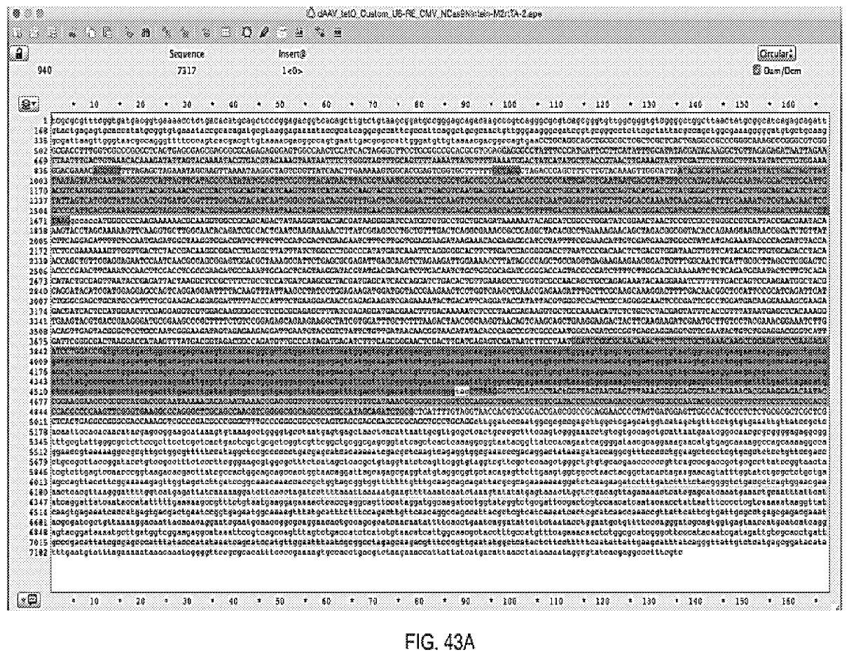

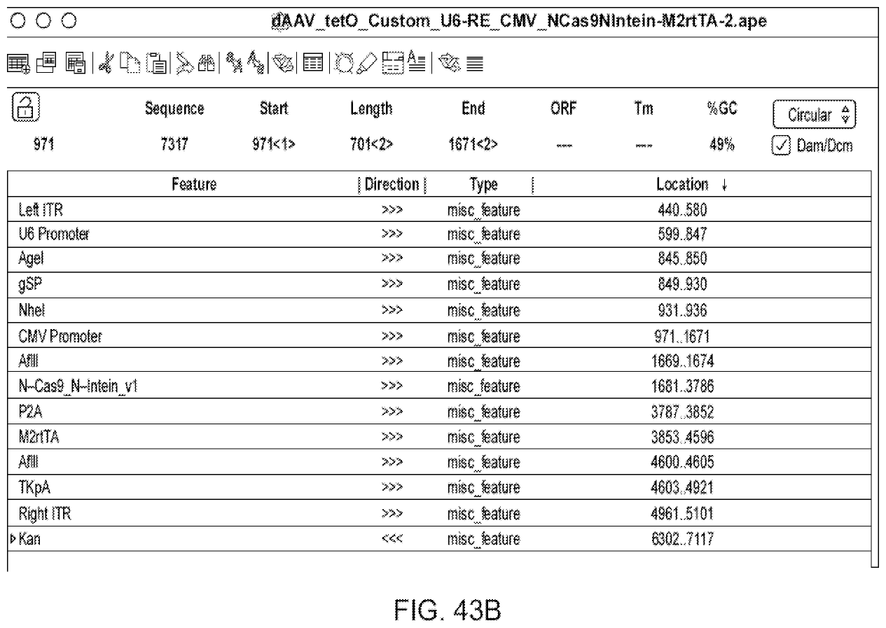

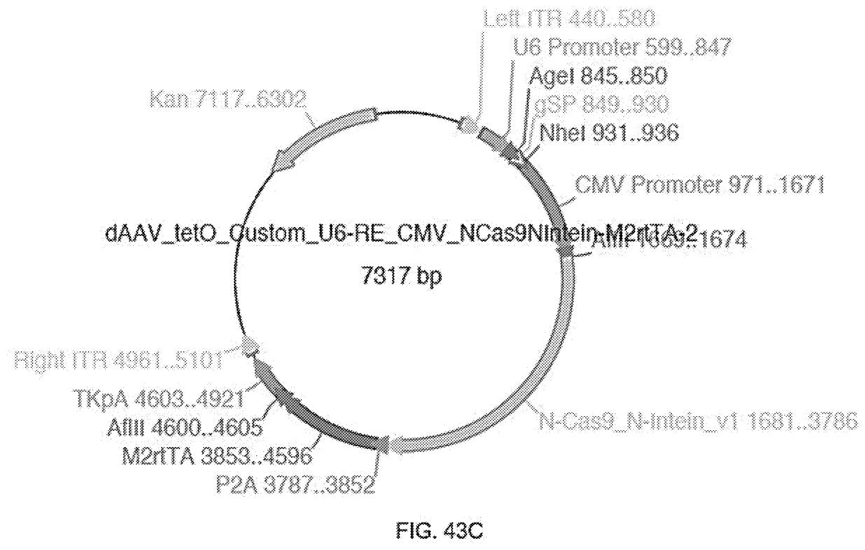

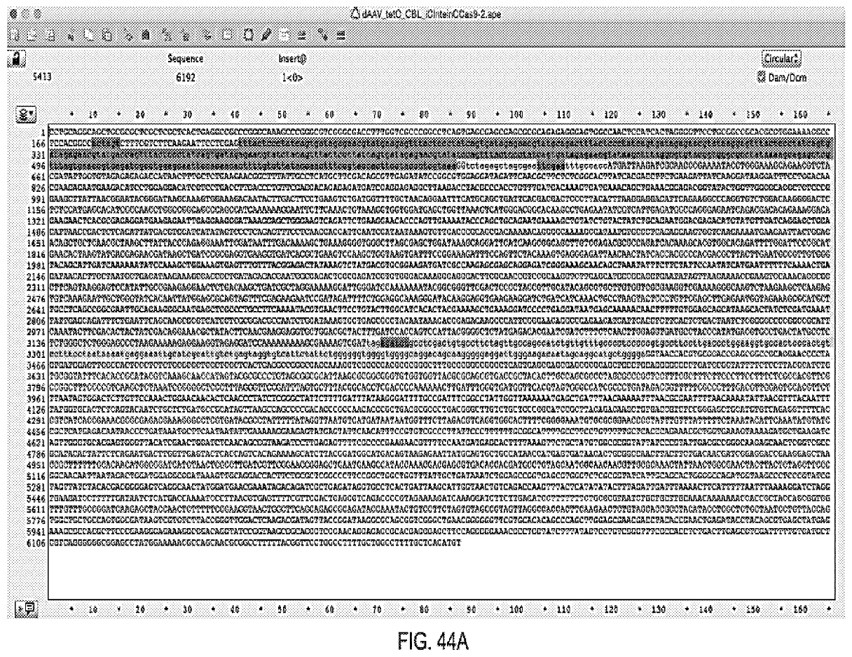

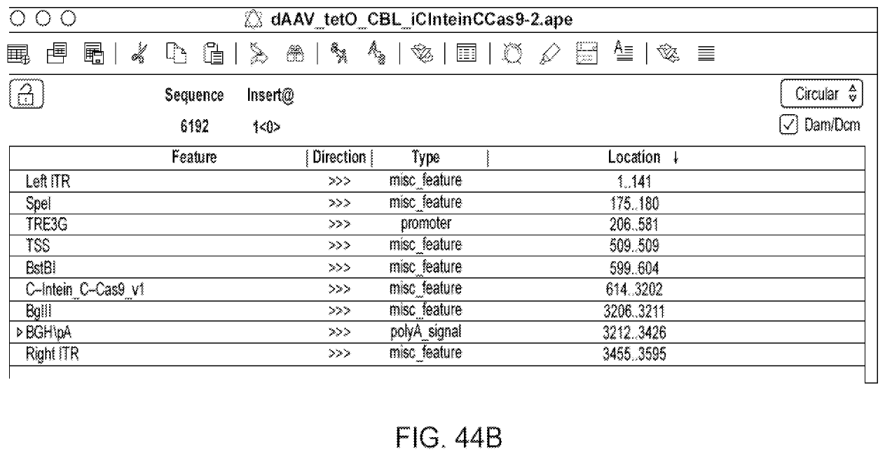

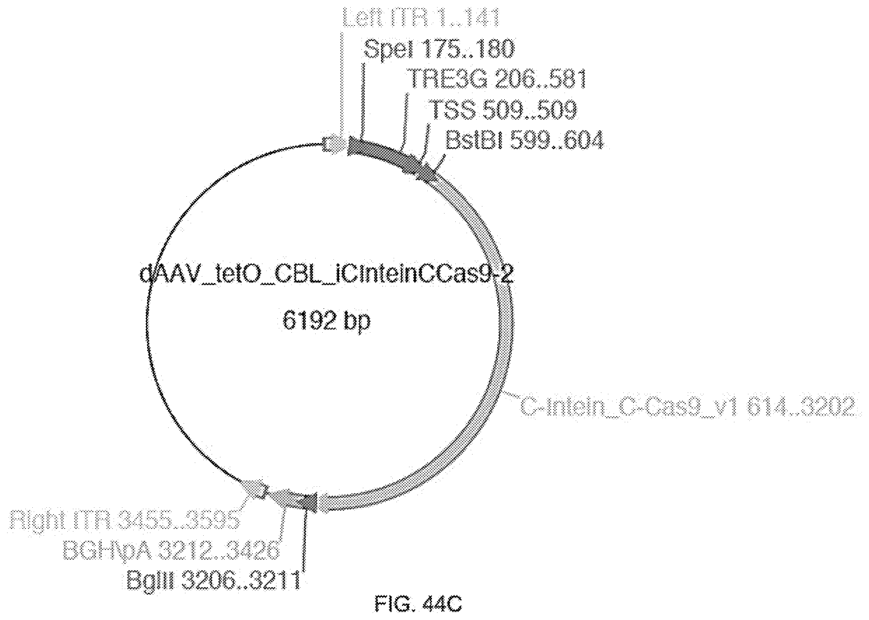

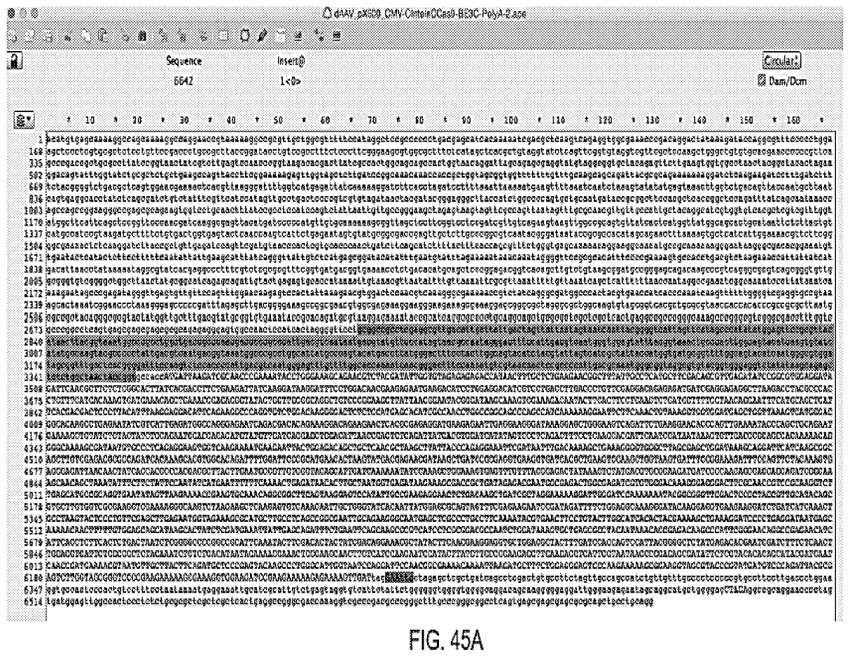

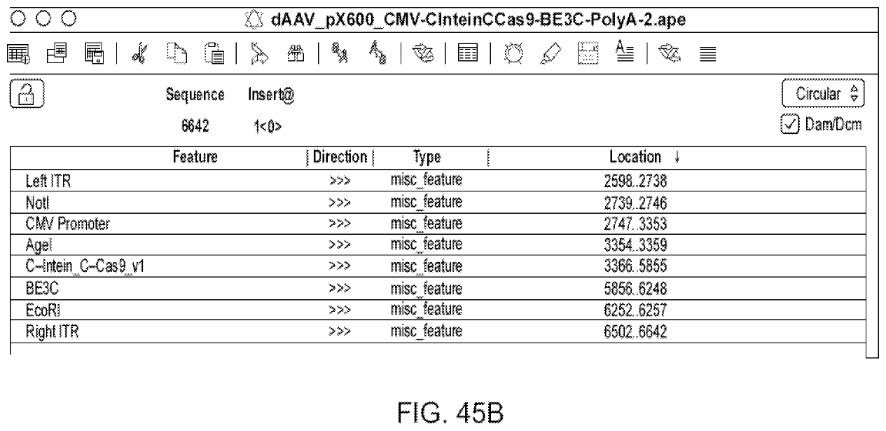

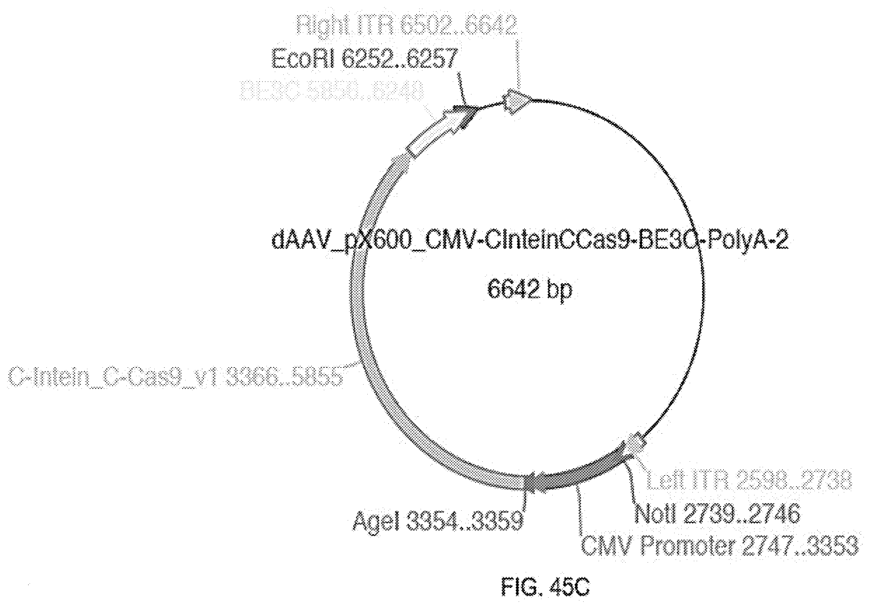

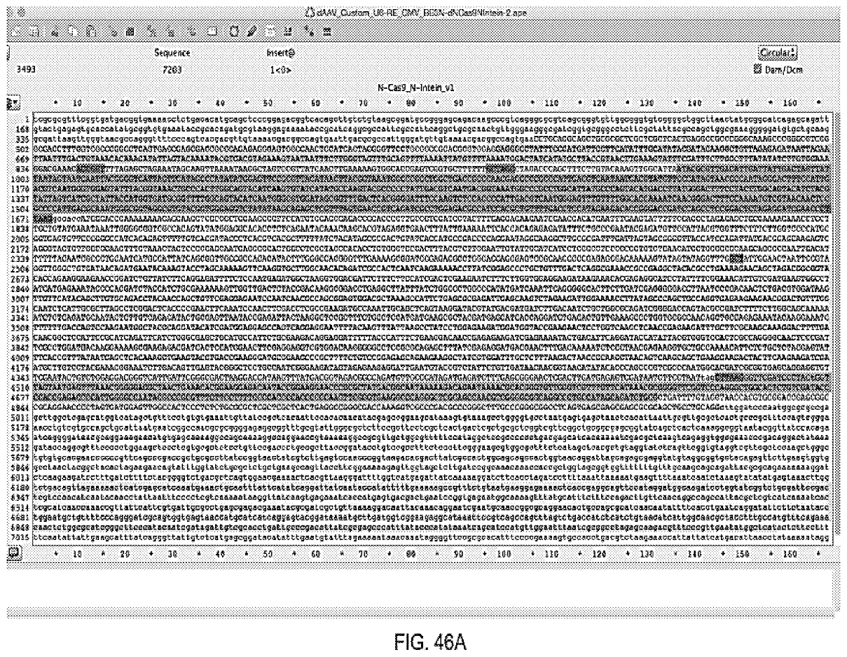

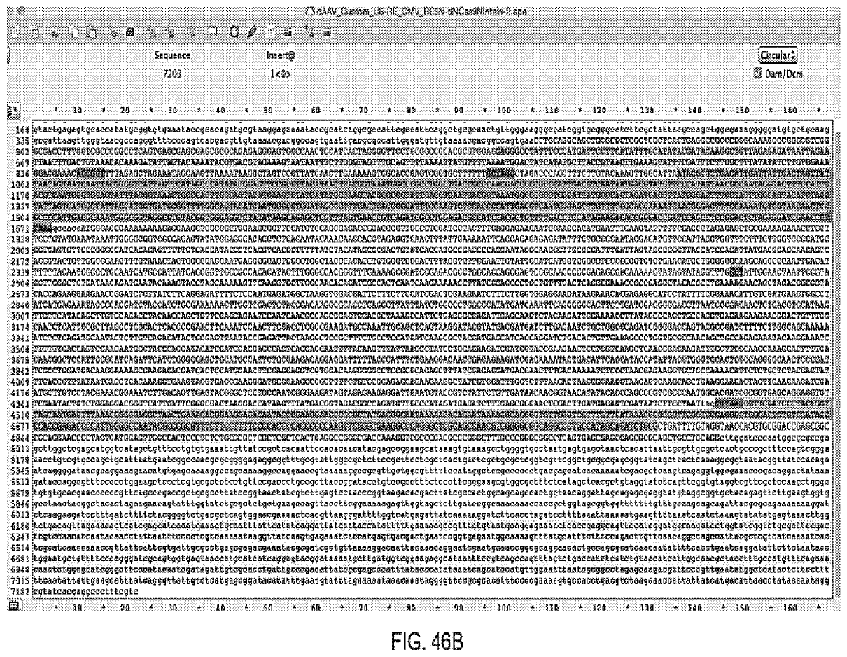

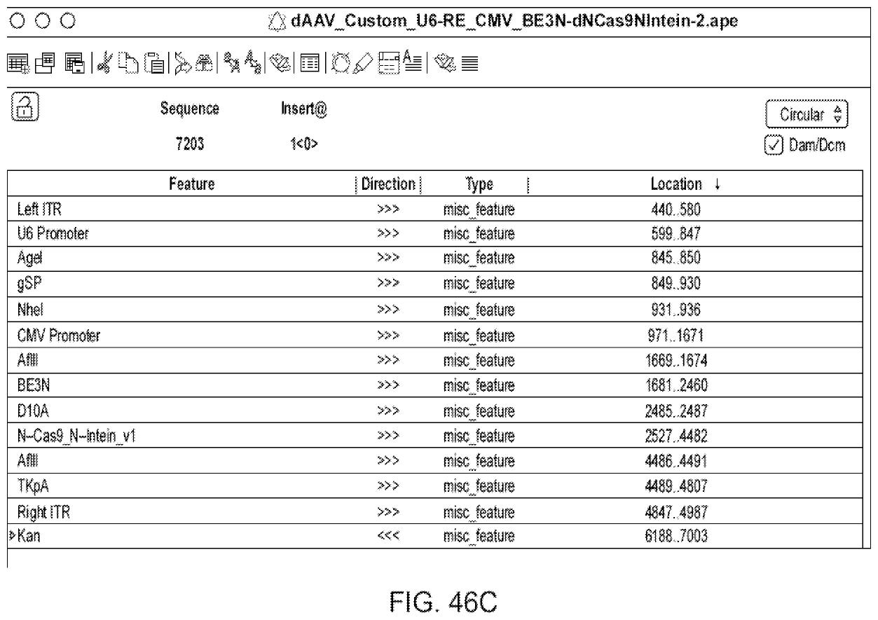

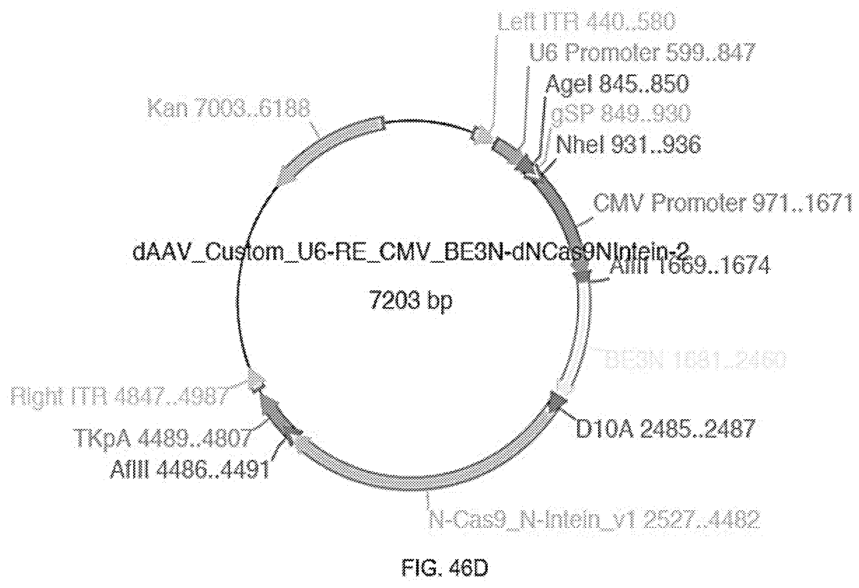

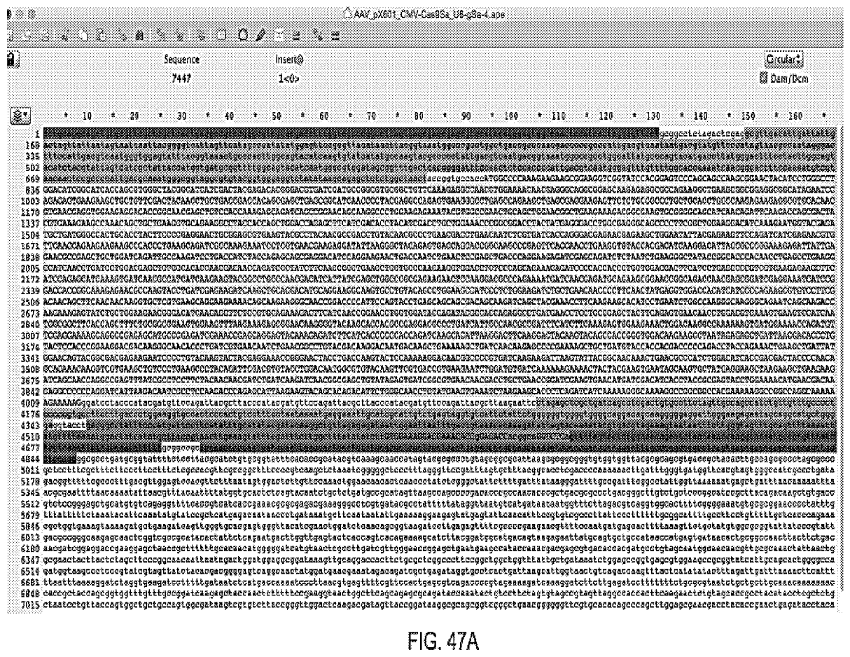

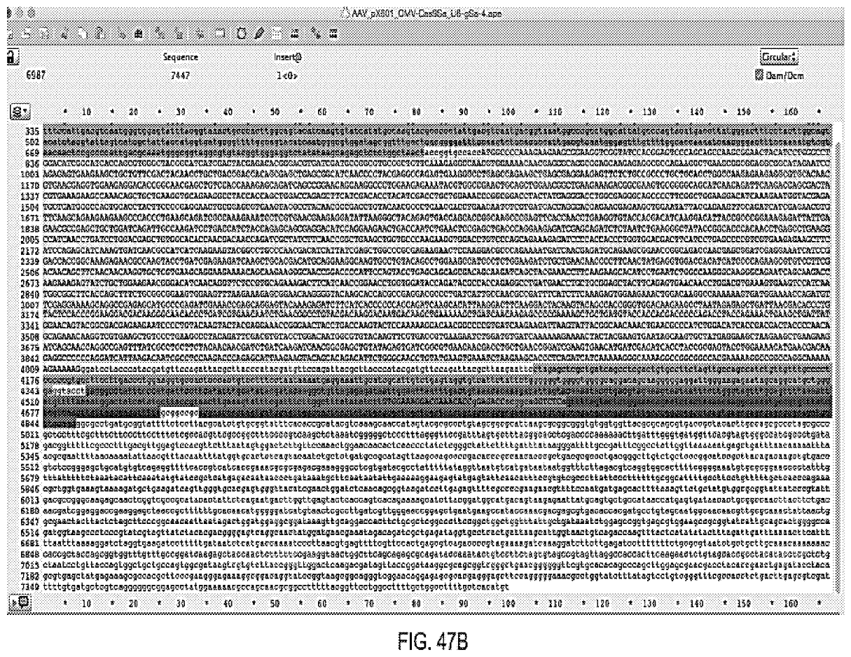

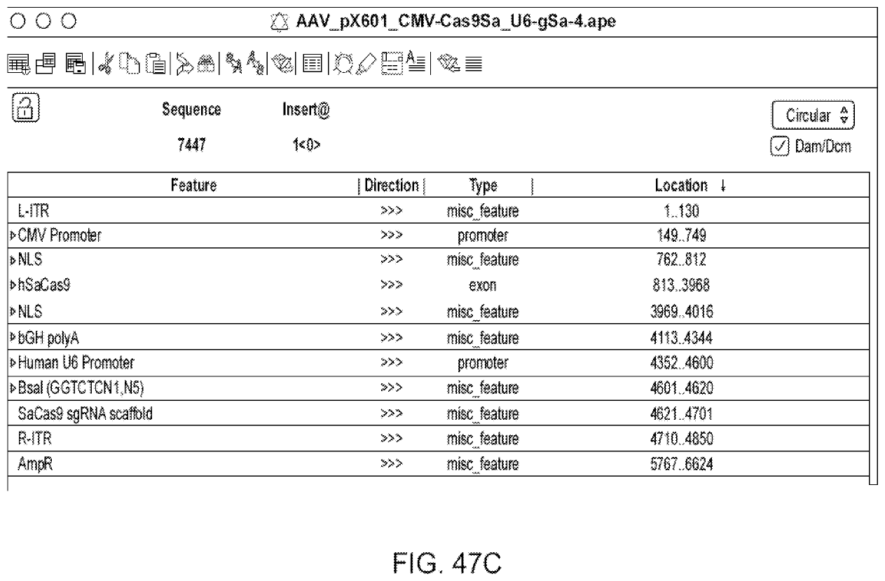

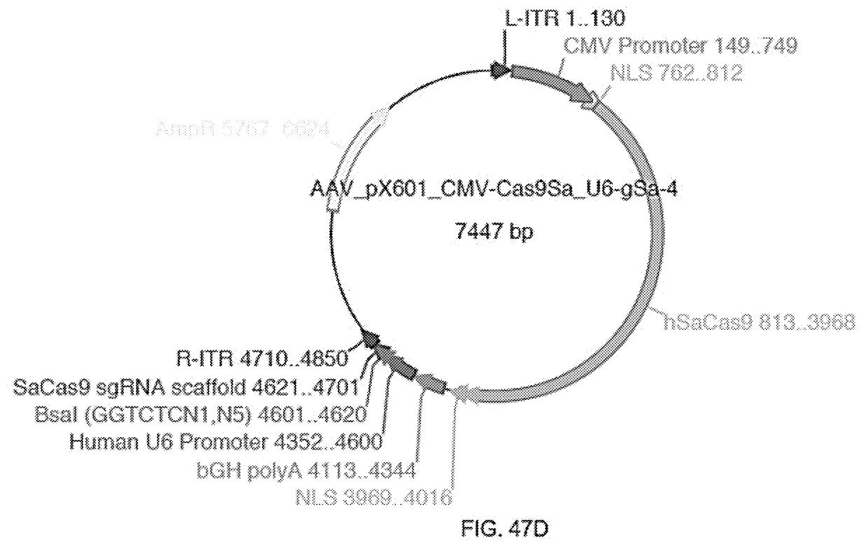

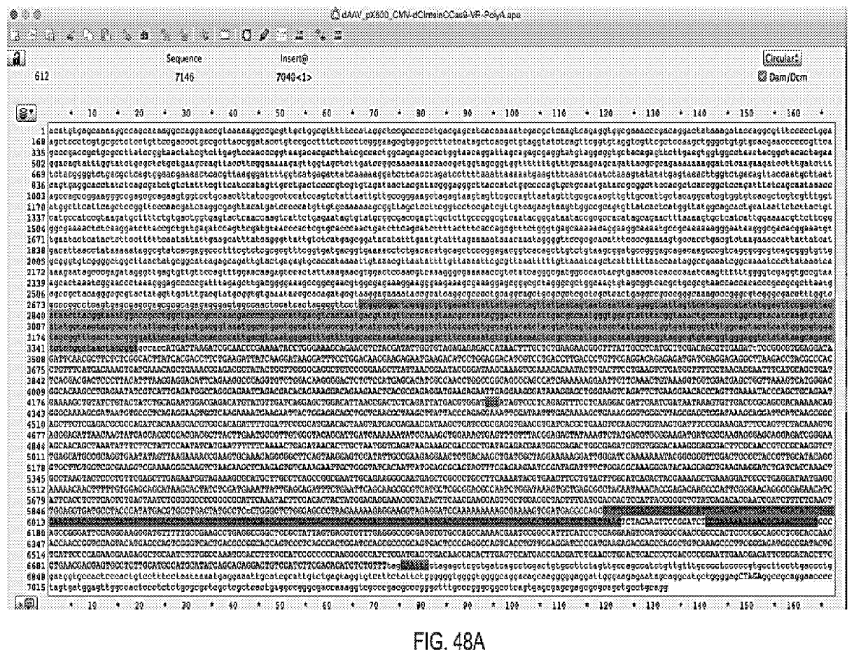

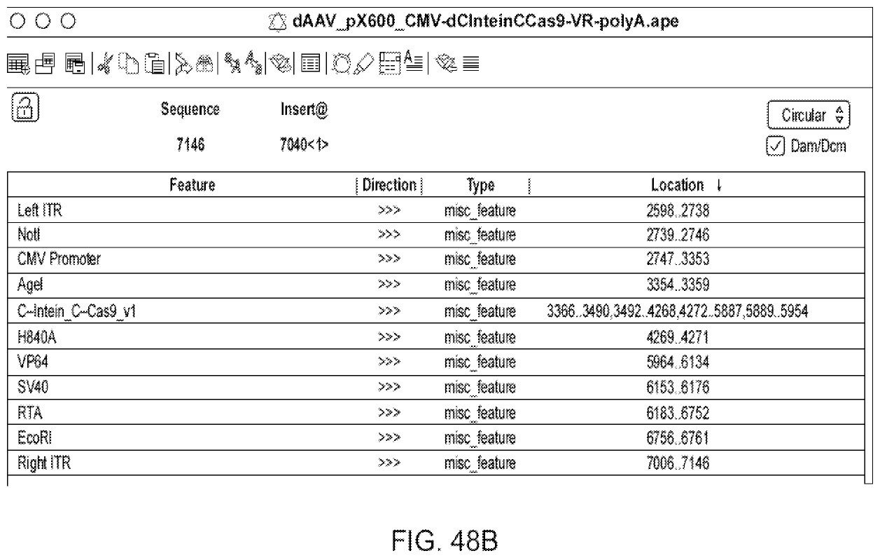

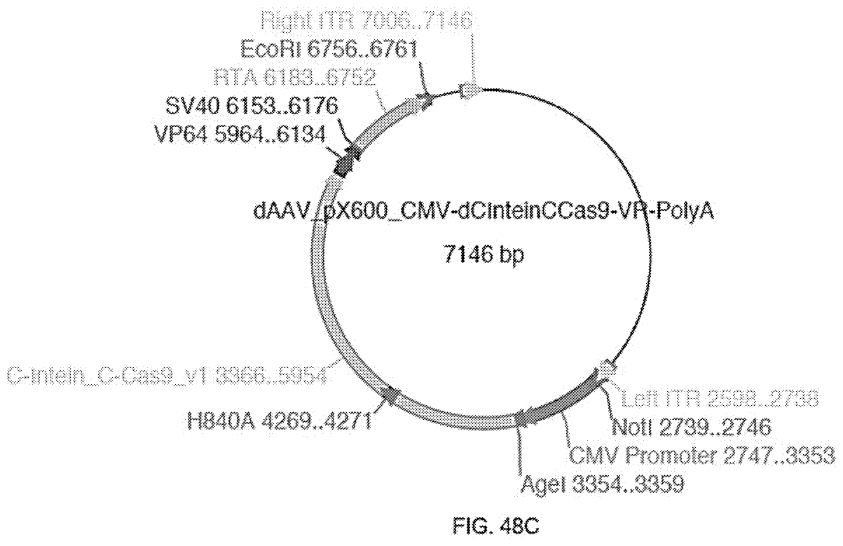

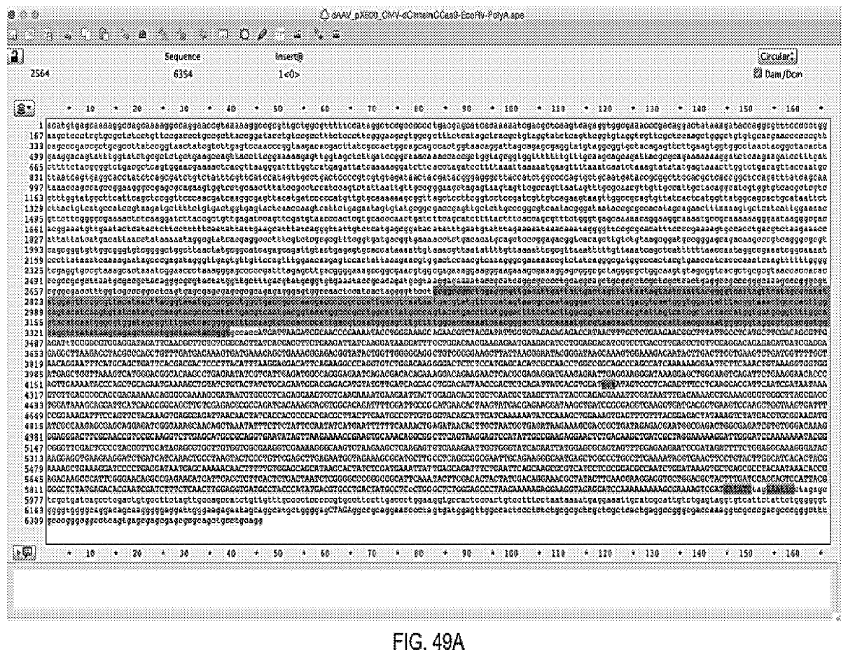

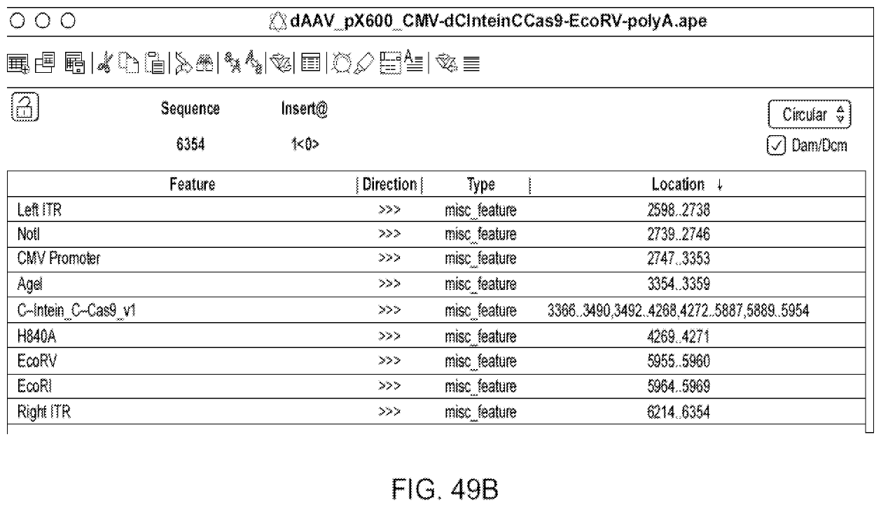

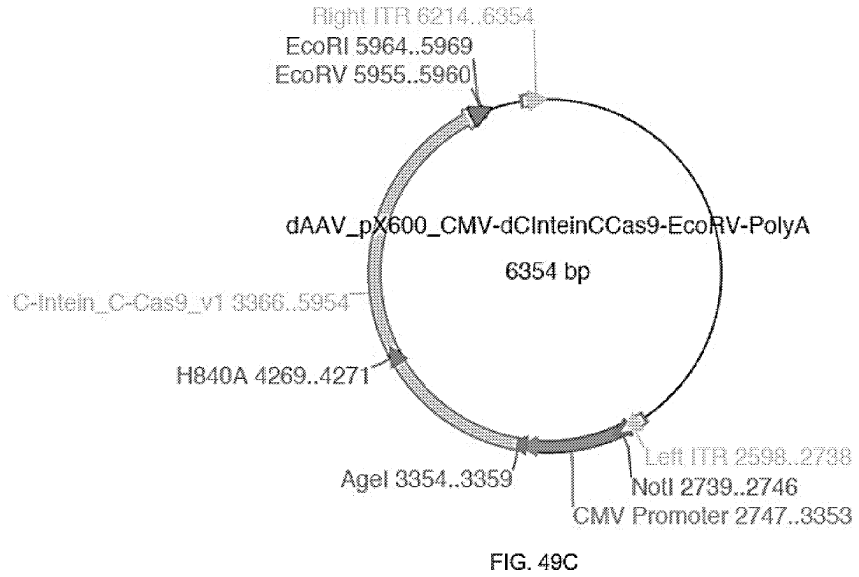

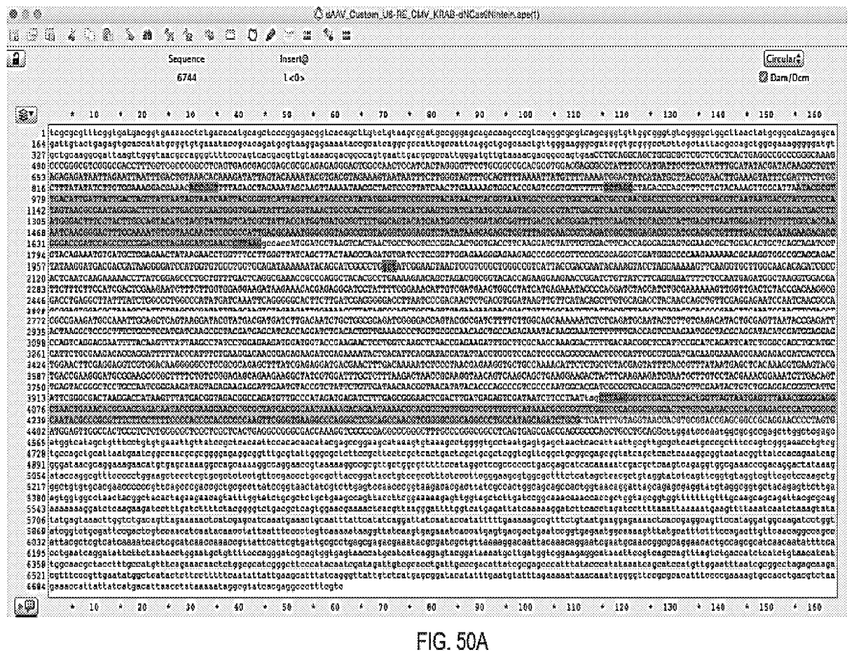

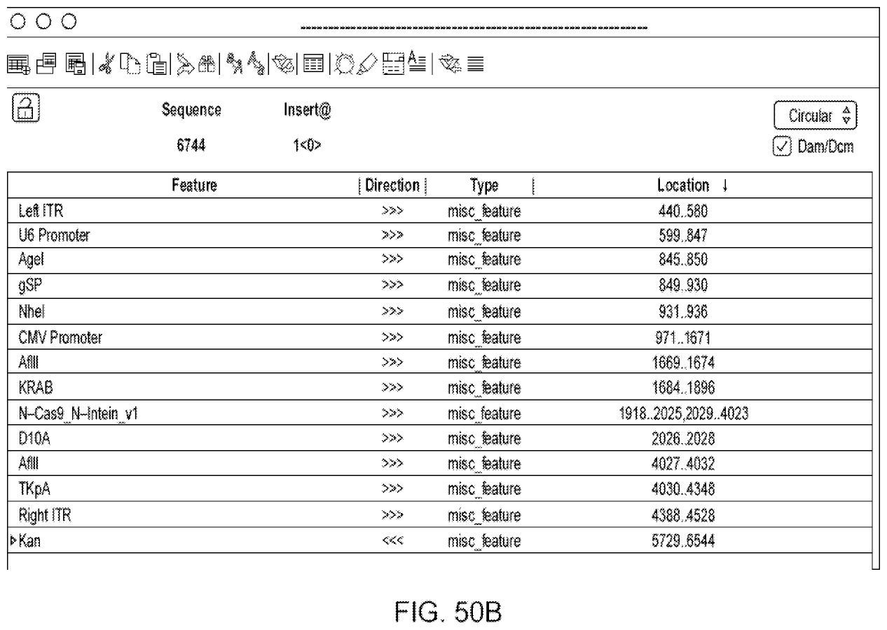

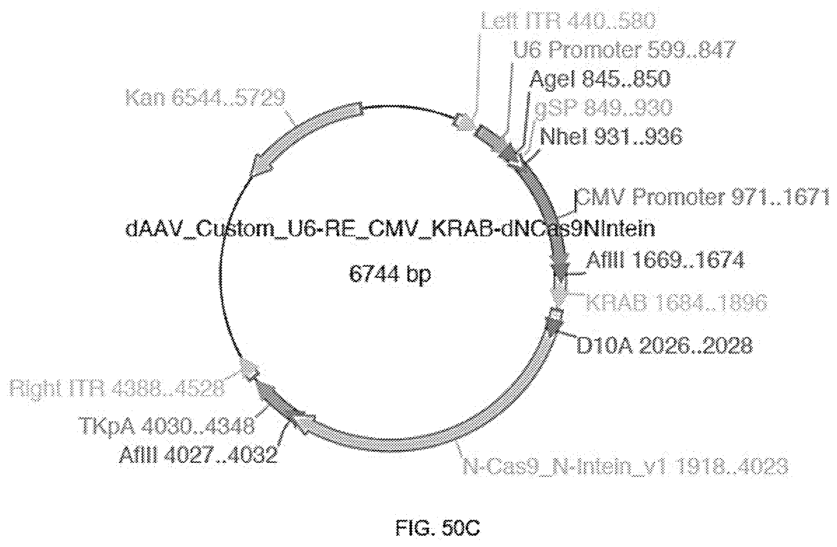

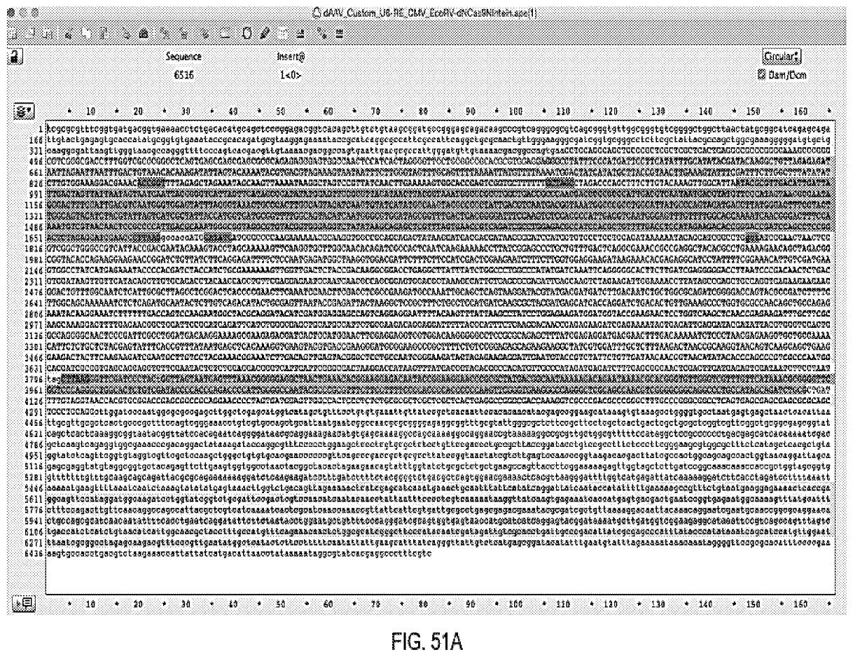

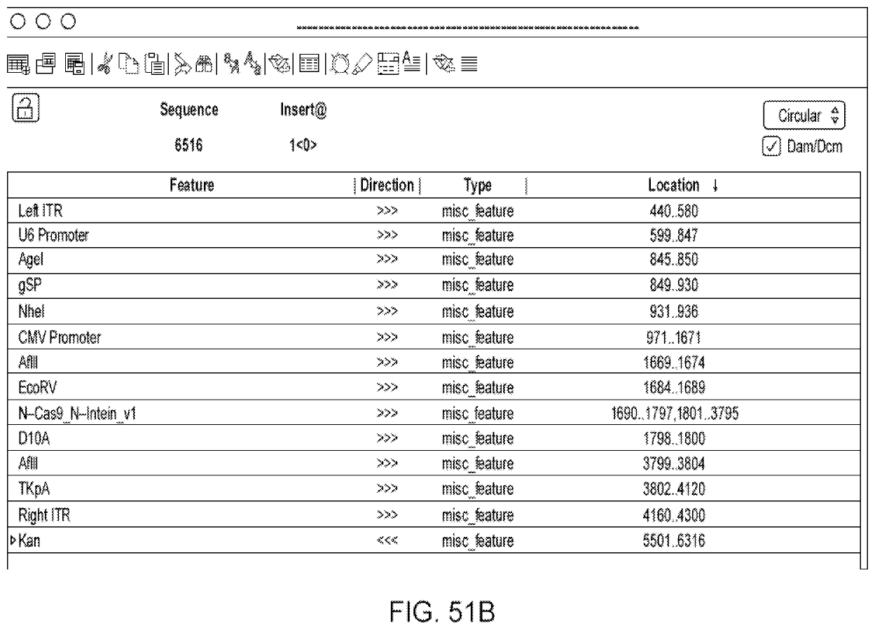

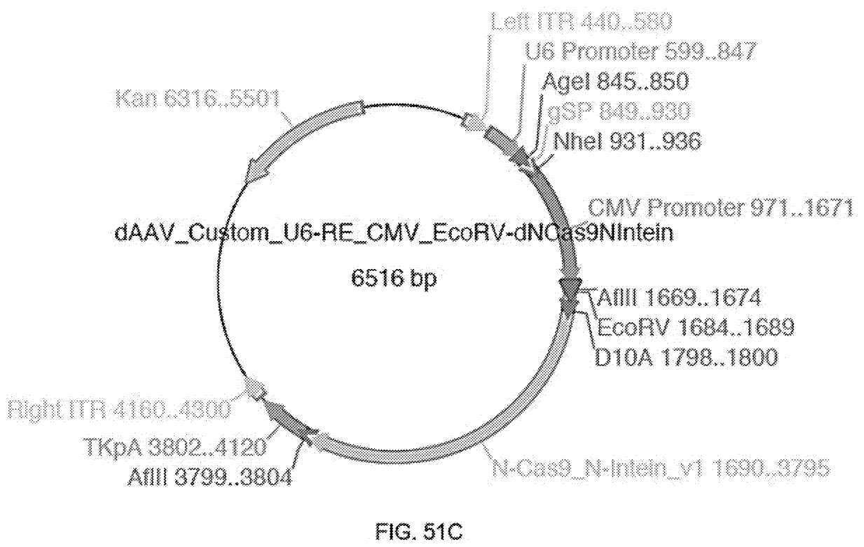

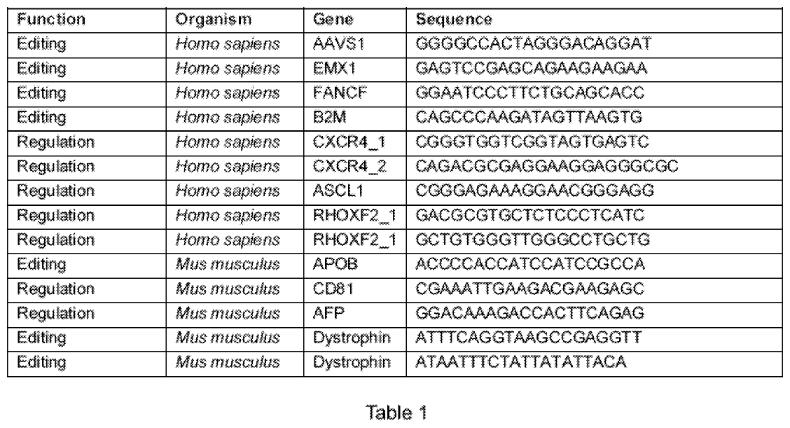

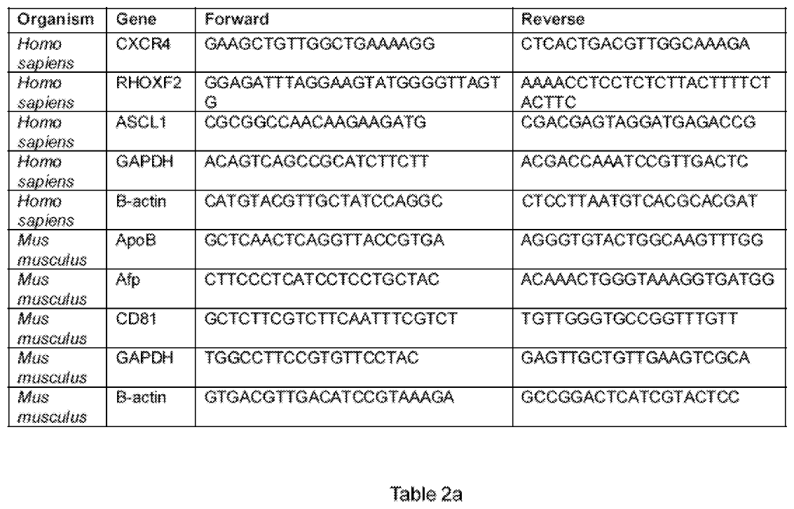

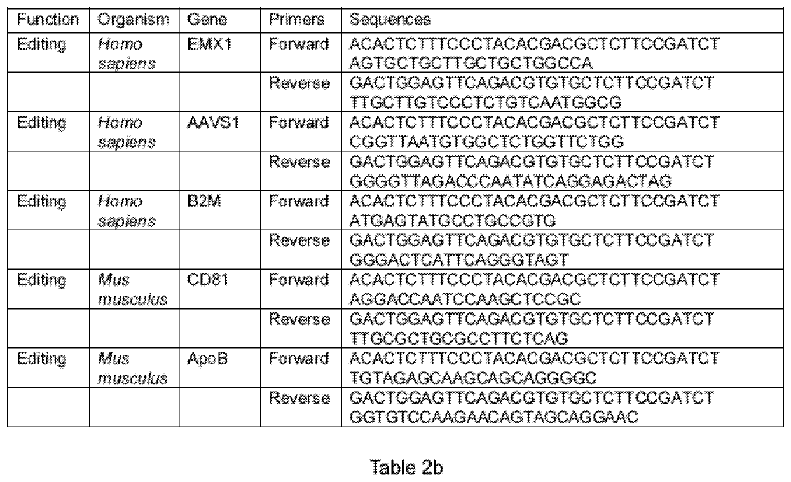

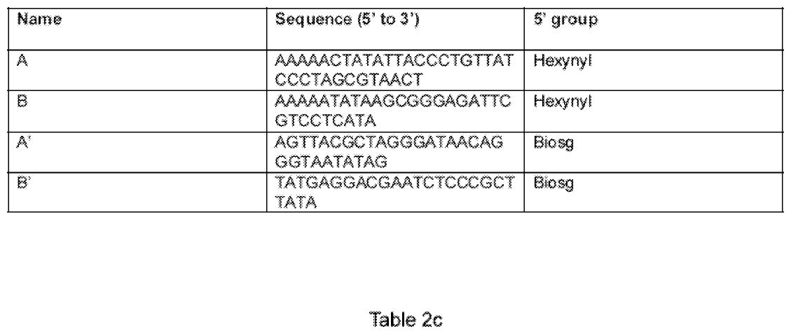

Further examples include but are not limited to human AID (UniProt Ref No. Q7Z599), human APOBEC3G (UniProt Ref No. Q9HC16), rat APOBEC1 (UniProt Ref. No. P38483), and lamprey CDA1 (GenBank Ref No. EF094822). In base editing embodiments, the base-editor utilizes a Cas9nickase. This results in only one of Cas9's two cleavage domains being mutated while retaining the ability to create a single-stranded break. For example, the exemplary base editing construct provided in FIG. 37 will contain a D10A mutation in the Cas9 cleavage domain. In some embodiments, this approach may be used in an in vivo setting.

In some embodiments, the first and/or second vector in the recombinant expression system encodes a cytidine deaminase enzyme that directs the conversion of a cytidine to uridine, therefore being useful to fix point-mutations.

Repression and Activation: Some aspects relate to the use of the recombinant expression system employing dCas9 for genome regulation. One concern with gene editing according to the traditional CRISPR-Cas9 model is the unknown effects that can arise after permanently editing a gene. This is a concern, as there are many genes with unknown functions and promiscuous activities associated with enzymes. For this reason, genome regulation is an attractive alternative, as it allows control of gene expression without the possible consequences that can come from editing genes. In some embodiments, the system is configured for controlled gene expression.

In some embodiments, a transcriptional activator or a transcriptional repressor is optionally incorporated into the recombinant expression system, e.g. a split-Cas9 dual AAV system, employing dCas9. In such embodiments, a gRNA is designed to target the promoter of the target gene.

A non-limiting exemplary transcriptional repressor is the Krüppel-associated box (“KRAB”), which is a highly conserved transcription repression module in higher vertebrates, an exemplary sequence of which is provided below:

KRAB
(SEQ ID NO: 20)
DAKSLTAWSRTLVTFKDVFVDFTREEWKLLDTAQQIVYRNVMLENYKNL
VSLGYQLTKPDVILRLEKGEEP

or a biological equivalent thereof.

A non-limiting exemplary transcriptional activators are VP74, RTa, and p65, exemplary sequences of which are provided below:

VP64
(SEQ ID NO: 21)
GSGRADALDDFDLDMLGSDALDDFDLDMLGSDALDDFDLDMLGSDALDD
FDLDMLIN
RTa
(SEQ ID NO: 22)
RDSREGMFLPKPEAGSAISDVFEGREVCQPKRIRPFHPPGSPWANRPLPA
SLAPTPTGPVHEPVGSLTPAPVPQPLDPAPAVTPEASHLLEDPDEETSQA
VKALREMADTVIPQKEEAAICGQMDLSHPPPRGHLDELTTTLESMTEDLN
P65
(SEQ ID NO: 23)
SQYLPDTDDRHRIEEKRKRTYETFKSIMKKSPFSGPTDPRPPPRRIAVPS
RSSASVPKPAPQPYPFTSSLSTINYDEFPTMVFPSGQISQASALAPAPPQ
VLPQAPAPAPAPAMVSALAQAPAPVPVLAPGPPQAVAPPAPKPTQAGEGT
LSEALLQLQFDDEDLGALLGNSTDPAVFTDLASVDNSEFQQLLNQGIPVA
PHTTEPMLMEYPEAITRLVTGAQRPPDPAPAPLGAPGLPNGLLSGDEDFS
SIADMDFSALL

or a biological equivalent each thereof.

In some embodiments, the first and/or second vector in the recombinant expression system comprises KRAB. In further embodiments, this recombinant expression system is used to silence, repress, or downregulate a target gene. In still further embodiments, the recombinant expression system comprises gRNA targeting the promoter for the target gene.

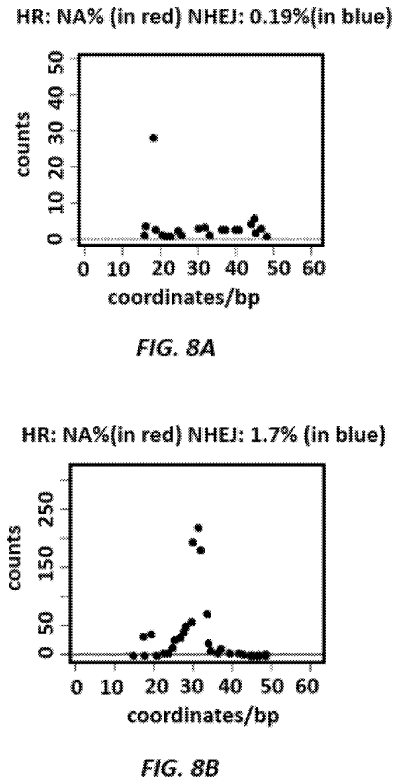

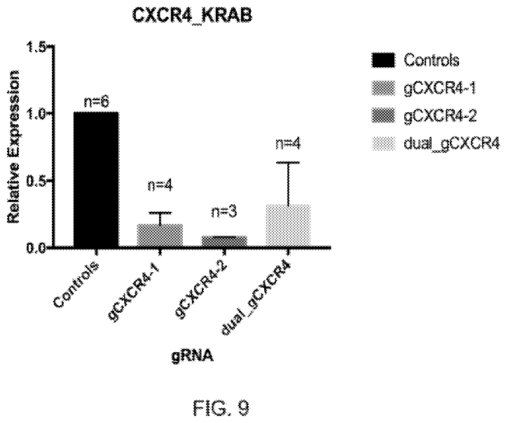

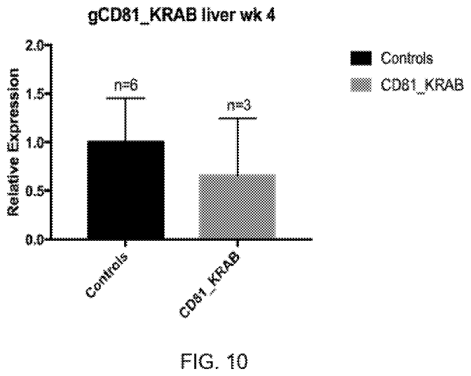

Applicants have tested this system in vitro and in vivo, and have showed up to 90% repression in vitro and 35% repression in vivo (FIGS. 8 and 9, respectively).

In some embodiments, the first and/or second vector in the recombinant expression system comprises VP64, RTa, and/or p65. In further embodiments, this recombinant expression system may be used to activate, overexpress, or upregulate a target gene. In still further embodiments, the recombinant expression system comprises gRNA targeting the promoter for the target gene. In embodiments relating to activation, overexpression, or upregulation of a target gene, the recombinant expression system may further comprise a third vector encoding the target gene for activation, overexpression, or upregulation.

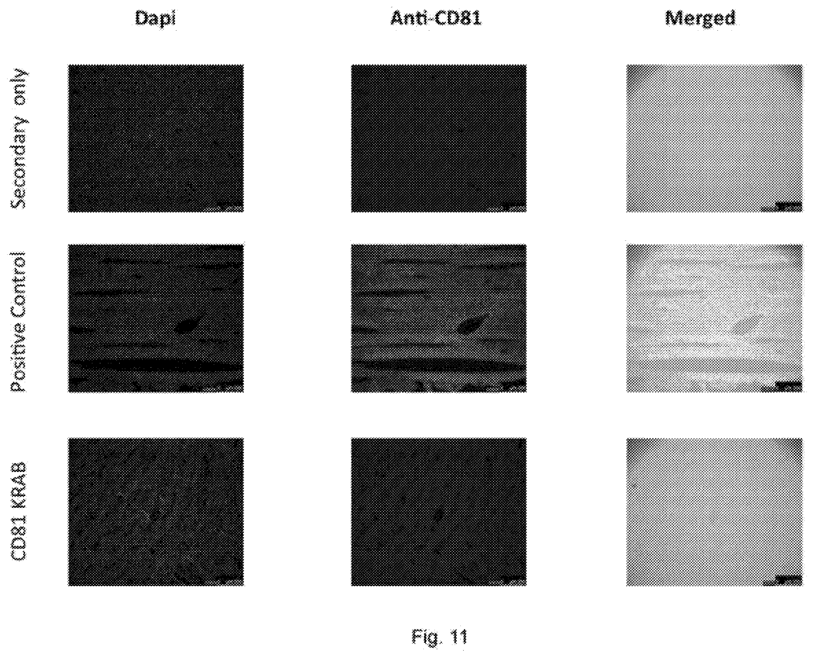

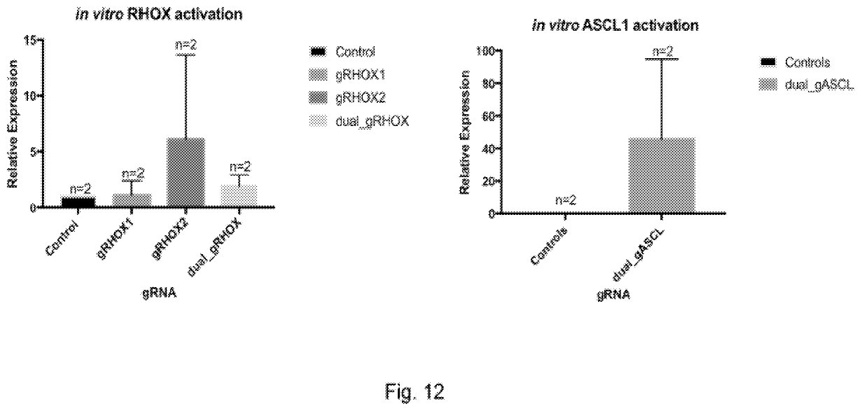

Applicants have measured an increase in relative expression in vitro of up to 40-fold (FIG. 11).

Methylation: In some embodiments, a regulator of methylation is optionally incorporated into the recombinant expression system; thus, allowing the epigenetic modification of a target gene. In such embodiments, a gRNA may be designed to target the promoter of the target gene.

Non-limiting examples of such regulators of methylation include but are not limited to DNMT3A and DNMT3L; exemplary sequences of which are provided below:

DNMT3A
(SEQ ID NO: 24)
TYGLLRRREDWPSRLQMFFANNHDQEFDPPKVYPPVPAEKRKPIRVLSLF
DGIATGLLVLKDLGIQVDRYIASEVCEDSITVGMVRHQGKIMYVGDVRSV
TQKHIQEWGPFDLVIGGSPCNDLSIVNPARKGLYEGTGRLFFEFYRLLHD
ARPKEGDDRPFFWLFENVVAMGVSDKRDISRFLESNPVMIDAKEVSAAHR
ARYFWGNLPGMNRPLASTVNDKLELQECLEHGRIAKFSKVRTITTRSNSI
KQGKDQHFPVFMNEKEDILWCTEMERVFGFPVHYTDVSNMSRLARQRLLG
RSWSVPVIRHLFAPLKEYFACV
DNMT3L
(SEQ ID NO: 25)
GSSELSSSVSPGTGRDLIAYEVKANQRNIEDICICCGSLQVHTQHPLFEG
GICAPCKDKFLDALFLYDDDGYQSYCSICCSGETLLICGNPDCTRCYCFE
CVDSLVGPGTSGKVHAMSNWVCYLCLPSSRSGLLQRRRKWRSQLKAFYDR
ESENPLEMFETVPVWRRQPVRVLSLFEDIKKELTSLGFLESGSDPGQLKH
VVDVTDTVRKDVEEWGPFDLVYGATPPLGHTCDRPPSWYLFQFHRLLQYA
RPKPGSPRPFFWMFVDNLVLNKEDLDVASRFLEMEPVTIPDVHGGSLQNA
VRVWSNIPAIRSRHWALVSEEELSLLAQNKQSSKLAAKWPTKLVKNCFLP
LREYFKYFSTELTSSL

or a biological equivalent each thereof.

In some embodiments, the first and/or second vector in the recombinant expression system comprises one or more of DNMT3A and DNMT3L. In further embodiments, this recombinant expression system is optionally used to silence, repress, or downregulate a target gene by altering the methylation thereof. In still further embodiments, the recombinant expression system comprises gRNA targeting the promoter for the target gene.

gRNAs for Specific Uses

In some embodiments, the recombinant expression system comprises a gRNA and is tailored to particular use based on the gRNA employed therein. Accordingly, in some embodiments, the first or second vector of the recombinant expression system encodes the gRNA. In other embodiments, the recombinant expression system comprises a third vector encoding the gRNA. In some embodiments, the gRNA is a dual gRNA (dgRNA) or a single gRNA (sgRNA).

Non-limiting exemplary method aspects for which gRNA are tailored are disclosed herein. Where exemplary gRNA are given, the uppercase lettering indicates exonic regions and the lowercase lettering indicates intronic regions.

It is appreciated that while the disclosed gRNA may be designed for a particular mammalian species, e.g. mouse or human, homologous genes and gRNAs thereto may be found using techniques and tools known in the art, such as protein and gene databases including but not limited to GenBank, BLAST, UniProt, SwissProt, KEGG, and GeneCards. Furthermore, validated gRNA sequences for a particular target and species can be found in one of many gRNA databases, such as the Cas database (rgenome.net/cas-database/) or through AddGene (addgene.org/crispr/reference/grna-sequence/) or GeneScript (genscript.com/gRNA-database.html). It should be further appreciated that the gRNA and/or target genes can be targeted by the recombinant expression system for these non-limiting exemplary methods and/or for any other disease or disorder associated with the gRNA and/or target genes.

It should be understood that when the term “repress” is used herein it intends reference to use with the recombinant expression system employing a transcriptional repressor, such as but not limited to KRAB; dCas9; and one or more disclosed gRNA; the term intends an effect on a target gene that reduces or eliminates its expression such as downregulation, repression, and/or silencing thereof. Similarly, when the term “activate” or “overexpress” is used herein it intends the recombinant expression system employing a transcriptional activator, such as but not limited to VP64, RTa, and p65; dCas9; and one or more disclosed gRNA; the term intends an effect on a target gene that increases its expression such as upregulation, activation, and/or overexpression thereof. More generally, “regulation” can be used in reference to gRNAs for use with a recombinant expression system employing dCas9, whereas “editing” can be used in reference to gRNAs for use with a recombinant expression system employing an active (or “live”) Cas9.

Pain Management: In some embodiments, gRNAs are employed in the recombinant expression system to target pain management. Long-term opioid usage has been linked to drug addiction and drug abuse, with an estimated 32.4 million people abusing opioids worldwide. In addition, 16% of first-time drug rehabilitation patients are seeking treatment for opioid abuse in Western and Central Europe, 45% in Asia, and 22% in North America. Furthermore, a recent report linked the use of morphine with doubling the duration of chronic constriction injury and predicted that prolonged pain is a consequence of the abundant use of opioids for chronic pain. For this reason, finding alternative ways of targeting pain could greatly be beneficial to the worldwide population. It is known that there are humans and mice with a loss of function mutation in the SCN9A gene (encoding voltage-gated sodium channel Nav 1.7), in conjunction with an increased expression in genes responsible for opioid peptides, that have low to high pain insensitivity. Humans and mice have point mutations in SCN9A resulting in this phenotype, including 18 missense mutations which cause substitution of a single amino acid and one in-frame deletion. Provided below are exemplary gRNA sequences that target SCN9A:

### Human SCN9a Designs

1:
(SEQ ID NO: 26)
GGAAAGCCGACAGCCGCCGC
2:
(SEQ ID NO: 27)
GGCGCGGGCCTCTCCTTCCC
3:
(SEQ ID NO: 28)
GAGCACGGGCGAAAGACCGA
4:
(SEQ ID NO: 29)
GTGTGCTCTTAAGGGGTGCG
5:
(SEQ ID NO: 30)
GTGGCGGTTGAGGCGAGCAC

### Mouse SCN9 Designs

Mouse SCN9 designs
1:
(SEQ ID NO: 31)
GACCCATGTAACAACTCCAC
2:
(SEQ ID NO: 32)
GTGTATATTGTTGAACCCGT
3:
(SEQ ID NO: 33)
AACAACTCCACTGGAGTAGA
4:
(SEQ ID NO: 34)
CAAACTGTTAAGAAACGGGC
5:
(SEQ ID NO: 35)
GGTTCTGGCAAAATTGCTGT

or a biological equivalent each thereof.

Not to be bound by theory, Applicants believe that using active Cas9 poses a risk to pain management to the extent that it may cause permanent insensitivity to pain and/or loss of olfactory sense. Specifically, Applicants are aware that mutation in the SCN9A gene can also cause a loss of functional NAV1.7 sodium channels in olfactory neurons resulting in a loss of olfactory sense. Accordingly, the exemplary gRNAs provided above are designed to target the promoter region of the SCN9A and can be employed in the embodiments of the recombinant expression system disclosed herein that employ dCas9. The intent of using these gRNA would be to silence or downregulate SCN9A.

For example, Applicants in one aspect, a disclosed recombinant expression system, e.g. a dual pAAV9_gSCN9a_dCas9 system, employing dCas9 is utilized (i) for prevention of pain during surgery, where the patient is administered the recombinant expression system before a surgery, or (ii) for the use of chronic pain. Not to be bound by theory, the amount of the recombinant expression system can be effective for the patient to have lowered pain for about a month at a time.

Additional genes that can be targeted for pain management include other sodium channels such as Nav 1.8 (SCN10A gene), 1.9 (SCN11A gene) and 1.3 (SCN3A gene), as well as the transient receptor potential cation channel subfamily V member 1 (TrpV1), also known as the capsaicin receptor and the vanilloid receptor 1. Other genes of interest include that will also be repressed or activated are as follows.

| | Effect of Recombinant |
|---|---|
| Gene | Expression System |
| SHANK3 (e.g. Accession No. JX122810.1) | Repress/Knock Out |
| NMDA receptor antagonists (including NR2B | Repress/Knock Out |
| (e.g. Accession No. NM_000834.4)) | |
| IL-10 (e.g. Accession No. NM_000572.2) | Activate (overexpress) |
| Penk (e.g. Accession No. NM_001135690.2) | Activate (overexpress) |
| Pomc (e.g. Accession No. NM_001035256.2) | Activate (overexpress) |
| MVIIA-PC (e.g. Accession No. FJ959111) | Activate (overexpress) |

Non-limiting examples of gRNAs that can be used for some of the named targets include: gRNA for Knockout:

Nav 1.3:
(SEQ ID NO: 36)
TCGTGGATTTCTATCACTTT
Nav 1.8:
(SEQ ID NO: 37)
CTTGGTAACGTCTTCTCTTG
Nav 1.9:
(SEQ ID NO: 38)
CGATGGTTCCACGTGCAATA
TrpV1:
(SEQ ID NO: 39)
TAAGCTGAATAACACCGTTG

gRNA for Repression:

Nav 1.3:
(SEQ ID NO: 40)
CCGCTTCCTGTTCTGAGATC
Nav 1.8:
(SEQ ID NO: 41)
GTCACGAGTTCCACCCTGCC
Nav 1.9:
(SEQ ID NO: 42)
CAGCCTGGATGGCTTACCTC
TrpV1:
(SEQ ID NO: 43)
GGGACTTACCAGCTAGGTGC

or a biological equivalent each thereof. Still further exemplary gRNAs are provided herein below:

| | | | | |
|---|---|---|---|---|
| sgID | gene | transcript | protospacer sequence | SEQ ID NO |

gRNA for Repression, in humans

| | | | | |
|---|---|---|---|---|
| SCN3A_+_166060543.23- | SCN3A | P1P2 | GATCTCAGAACAGGAAGCGG | 44 |
| P1P2 | | | | |
| SCN3A_+_166060199.23- | SCN3A | P1P2 | GTGTAAATTACAGGAACCAA | 45 |
| P1P2 | | | | |
| SCN3A_−_166060301.23- | SCN3A | P1P2 | GACCTGGTAGCTAGGTTCTA | 46 |
| P1P2 | | | | |
| SCN3A_+_166060552.23- | SCN3A | P1P2 | GATAGAGTGAATCTCAGAAC | 47 |
| P1P2 | | | | |
| SCN3A_+_166060129.23- | SCN3A | P1P2 | GAATAGAGCCTGTCTGGAAA | 48 |
| P1P2 | | | | |
| SCN3A_+_166060346.23- | SCN3A | P1P2 | GTGTTATGCTGTAATTCATA | 49 |
| P1P2 | | | | |
| SCN3A_+_166060119.23- | SCN3A | P1P2 | GGTCTGGAAATGGTGATTTA | 50 |
| P1P2 | | | | |
| SCN3A_+_166060135.23- | SCN3A | P1P2 | GAAAGAAAATAGAGCCTGTC | 51 |
| P1P2 | | | | |
| SCN3A_+_166060371.23- | SCN3A | P1P2 | GCCTAACCATCTTGGATGCT | 52 |
| P1P2 | | | | |
| SCN3A_+_166060281.23- | SCN3A | P1P2 | GACCATAGAACCTAGCTACC | 53 |
| P1P2 | | | | |
| SCN9A_+_167232419.23- | SCN9A | P1P2 | GGCGGTCGCCAGCGCTCCAG | 54 |
| P1P2 | | | | |
| SCN9A_+_167232052.23- | SCN9A | P1P2 | GCCACCTGGAAAGAAGAGAG | 55 |
| P1P2 | | | | |
| SCN9A_+_167232416.23- | SCN9A | P1P2 | GGTCGCCAGCGCTCCAGCGG | 56 |
| P1P2 | | | | |
| SCN9A_+_167232010.23- | SCN9A | P1P2 | GCCAGCAATGGGAGGAAGAA | 57 |
| P1P2 | | | | |
| SCN9A_−_167232085.23- | SCN9A | P1P2 | GTTCCAGGTGGCGTAATACA | 58 |
| P1P2 | | | | |
| SCN9A_+_167232476.23- | SCN9A | P1P2 | GGCGGGGCTGCTACCTCCAC | 59 |
| P1P2 | | | | |
| SCN9A + 167232437.23- | SCN9A | P1P2 | GGGCGCAGTCTGCTTGCAGG | 60 |
| P1P2 | | | | |
| SCN9A_+_167232409.23- | SCN9A | P1P2 | GGCGCTCCAGCGGCGGCTGT | 61 |
| P1P2 | | | | |
| SCN9A_+_167232021.23- | SCN9A | P1P2 | GACCGGGTGGTTCCAGCAAT | 62 |
| P1P2 | | | | |
| SCN9A_+_167232018.23- | SCN9A | P1P2 | GGGGTGGTTCCAGCAATGGG | 63 |
| P1P2 | | | | |
| SCN10A_−_38835462.23- | SCN10A | ENST00000449082.2 | GTGACTCCGGAGTAAAGCGA | 64 |
| ENST00000449082.2 | | | | |
| SCN10A_−_38835311.23- | SCN10A | ENST00000449082.2 | GGGAGCTCACCATAGAACTT | 65 |
| ENST00000449082.2 | | | | |
| SCN10A_−_38835269.23- | SCN10A | ENST00000449082.2 | GACGGATCTAGATCCTCCAG | 66 |
| ENST00000449082.2 | | | | |
| SCN10A_+_38835213.23- | SCN10A | ENST00000449082.2 | GCCGGGTAAGAGCTACTAGT | 67 |
| ENST00000449082.2 | | | | |
| SCN10A_−_38835251.23- | SCN10A | ENST00000449082.2 | GCCCGGTGTGTGCTGTAGAA | 68 |
| ENST00000449082.2 | | | | |
| SCN10A_+_38835434.23- | SCN10A | ENST00000449082.2 | GTTTACTCCGGAGTCACTGG | 69 |
| ENST00000449082.2 | | | | |
| SCN10A_−_38835449.23- | SCN10A | ENST00000449082.2 | GCTATCTCCACCAGTGACTC | 70 |
| ENST00000449082.2 | | | | |
| SCN10A_−_38835156.23- | SCN10A | ENST00000449082.2 | GACATCACCCAGGGCCAAGG | 71 |
| ENST00000449082.2 | | | | |
| SCN10A_−_38835491.23- | SCN10A | ENST00000449082.2 | GTAGTTTCGAGGGATCCAAT | 72 |
| ENST00000449082.2 | | | | |
| SCN10A_+_38835272.23- | SCN10A | ENST00000449082.2 | GCTCCCAGCAGAACTGATCG | 73 |
| ENST00000449082.2 | | | | |
| SCN11A_−_38991624.23- | SCN11A | ENST00000302328.3, | GATGGGTCCAAGTCTTCCAG | 74 |
| ENST00000302328.3, | | ENST00000450244.1 | | |
| ENST00000450244.1 | | | | |
| SCN11A_+_38992032.23- | SCN11A | ENST00000302328.3, | GGTTCCTGCTATACCCACAG | 75 |
| ENST00000302328.3, | | ENST00000450244.1 | | |
| ENST00000450244.1 | | | | |
| SCN11A_−_38991801.23- | SCN11A | ENST00000302328.3, | GCCAGAGAGTCGGAAGTGAA | 76 |
| ENST00000302328.3, | | ENST00000450244.1 | | |
| ENST00000450244.1 | | | | |
| SCN11A_+_38992029.23- | SCN11A | ENST00000302328.3, | GCCTGCTATACCCACAGTGG | 77 |
| ENST00000302328.3, | | ENST00000450244.1 | | |
| ENST00000450244.1 | | | | |
| SCN11A_+_38991609.23- | SCN11A | ENST00000302328.3, | GGGAAAGCCTCTGGAAGACT | 78 |
| ENST00000302328.3, | | ENST00000450244.1 | | |
| ENST00000450244.1 | | | | |
| SCN11A_−_38992040.23- | SCN11A | ENST00000302328.3, | GGAAGAGATGACCACCACTG | 79 |
| ENST00000302328.3, | | ENST00000450244.1 | | |
| ENST00000450244.1 | | | | |
| SCN11A_−_38991666.23- | SCN11A | ENST00000302328.3, | GGAATGTCGCCATAGAGCTT | 80 |
| ENST00000302328.3, | | ENST00000450244.1 | | |
| ENST00000450244.1 | | | | |
| SCN11A_+_38991618.23- | SCN11A | ENST00000302328.3, | GGAGCTCATAGGAAAGCCTC | 81 |
| ENST00000302328.3, | | ENST00000450244.1 | | |
| ENST00000450244.1 | | | | |
| SCN11A_+_38991924.23- | SCN11A | ENST00000302328.3, | GCTTTAAGACTGGAATCCTA | 82 |
| ENST00000302328.3, | | ENST00000450244.1 | | |
| ENST00000450244.1 | | | | |
| SCN11A_+_38991653.23- | SCN11A | ENST00000302328.3, | GGGAAGTTGCCCAAGCTCTA | 83 |
| ENST00000302328.3, | | ENST00000450244.1 | | |
| ENST00000450244.1 | | | | |
| SHANK3_+_51135959.23- | SHANK3 | P1P2 | GGAATTCGAATACAGCTCCT | 84 |
| P1P2 | | | | |
| SHANK3_+_51136404.23- | SHANK3 | P1P2 | GCTTCAGGCAGAGACCCCCG | 85 |
| P1P2 | | | | |
| SHANK3_+_51136356.23- | SHANK3 | P1P2 | GGAGCCTCCGTGGTGACACA | 86 |
| P1P2 | | | | |
| SHANK3_+_51136302.23- | SHANK3 | P1P2 | GCACGGCAGGAACCTTCCCC | 87 |
| P1P2 | | | | |
| SHANK3_+_51136319.23- | SHANK3 | P1P2 | GAGCACCGGAGGGACCCGCA | 88 |
| P1P2 | | | | |
| SHANK3_+_51136333.23- | SHANK3 | P1P2 | GGCCCGGAACGACAGAGCAC | 89 |
| P1P2 | | | | |
| SHANK3_+_51136329.23- | SHANK3 | P1P2 | GGGAACGACAGAGCACCGGA | 90 |
| P1P2 | | | | |
| SHANK3_−_51136143.23- | SHANK3 | P1P2 | GACcgcggcgaggccgtgaa | 91 |
| P1P2 | | | | |
| SHANK3_−_51136336.23- | SHANK3 | P1P2 | GCCTGCCGTGCGGGTCCCTC | 92 |
| P1P2 | | | | |
| SHANK3_+_51135950.23- | SHANK3 | P1P2 | GTACAGCTCCTGGGCGCGCC | 93 |
| P1P2 | | | | |
| TRPV1_+_3500355.23- | TRPV1 | P1P2 | GAGCGACTCCTGCTAGTGCA | 94 |
| P1P2 | | | | |
| TRPV1_+_3500317.23- | TRPV1 | P1P2 | GCGGGCCCGGGACCCCACGG | 95 |
| P1P2 | | | | |
| TRPV1_+_3499964.23- | TRPV1 | P1P2 | GCTCCTTGGAAGCACCTGGG | 96 |
| P1P2 | | | | |
| TRPV1_−_3500391.23- | TRPV1 | P1P2 | GAGTCGCTGTGGACGCCCTT | 97 |
| P1P2 | | | | |
| TRPV1_−_3500224.23- | TRPV1 | P1P2 | GGGACTCACCAGCTAGACGC | 98 |
| P1P2 | | | | |
| TRPV1_−_3500327.23- | TRPV1 | P1P2 | GTGGTCTCCCCGCCTCCGTG | 99 |
| P1P2 | | | | |
| TRPV1_−_3500298.23- | TRPV1 | P1P2 | GGGGAGAGCTGGGCTCGTGT | 100 |
| P1P2 | | | | |
| TRPV1_+_3500017.23- | TRPV1 | P1P2 | Gtgcctcaaaggtggtcgtg | 101 |
| P1P2 | | | | |
| TRPV1_+_3499899.23- | TRPV1 | P1P2 | GCTGCATCAGCCGTCCTCGG | 102 |
| P1P2 | | | | |
| TRPV1_−_3500400.23- | TRPV1 | P1P2 | GGGACGCCCTTCGGCACTCA | 103 |
| P1P2 | | | | |
| GRIN2B_−_14133341.23- | GRIN2B | P1P2 | GGATTCGCGTGTCCCCCGGA | 104 |
| P1P2 | | | | |
| GRIN2B_+_14132929.23- | GRIN2B | P1P2 | GGATATGCAAGCGAGAAGAA | 105 |
| P1P2 | | | | |
| GRIN2B_−_14132903.23- | GRIN2B | P1P2 | GCTCTAGACGGACAGATTAA | 106 |
| P1P2 | | | | |
| GRIN2B_−_14133316.23- | GRIN2B | P1P2 | GGGGGAAAAAGAGGCGGTCA | 107 |
| P1P2 | | | | |
| GRIN2B_+_14132924.23- | GRIN2B | P1P2 | GGCAAGCGAGAAGAAGGGAC | 108 |
| P1P2 | | | | |
| GRIN2B_−_14133295.23- | GRIN2B | P1P2 | GCCAAAGCGTCCCCTTCCTA | 109 |
| P1P2 | | | | |
| GRIN2B_−_14133298.23- | GRIN2B | P1P2 | GAAGCGTCCCCTTCCTAAGG | 110 |
| P1P2 | | | | |
| GRIN2B_+_14132855.23- | GRIN2B | P1P2 | GGCTTCTACAAACCAAGGTA | 111 |
| P1P2 | | | | |
| GRIN2B_+_14133247.23- | GRIN2B | P1P2 | GACCATGCTCCACCGAGGGA | 112 |
| P1P2 | | | | |
| GRIN2B_+_14133252.23- | GRIN2B | P1P2 | GGAATGACCATGCTCCACCG | 113 |
| P1P2 | | | | |

gRNA for Repression, in mice

| | | | | |
|---|---|---|---|---|
| Scn3a_+_65567459.23-P1P2 | Scn3a | P1P2 | GTGAATCTCAGAACAGGAAG | 114 |
| Scn3a_+_65567442.23-P1P2 | Scn3a | P1P2 | GAGCGGAGGCATAAGCAGAA | 115 |
| Scn3a_−_65567234.23-P1P2 | Scn3a | P1P2 | GATCTGGTGGCTAGATTCTA | 116 |
| Scn3a_−_65567301.23-P1P2 | Scn3a | P1P2 | GAGGAATCACAGCTCAACAA | 117 |
| Scn3a_−_65567522.23-P1P2 | Scn3a | P1P2 | GATCAGAAAACGGCCCTGGA | 118 |
| Scn3a_−_65567271.23-P1P2 | Scn3a | P1P2 | GGTTTTGTCAGCTTACCTGA | 119 |
| Scn3a_−_65567326.23-P1P2 | Scn3a | P1P2 | GGCATCCAAGATGGTTAGAA | 120 |
| Scn3a_+_65567264.23-P1P2 | Scn3a | P1P2 | GATTCCTAAGGCTCTCCATC | 121 |
| Scn3a_+_65567031.23-P1P2 | Scn3a | P1P2 | GCAATACAGACTAGGAATTA | 122 |
| Scn9a_+_66634758.23-P1P2 | Scn9a | P1P2 | GAGCTCAGGGAGCATCGAGG | 123 |
| Scn9a_−_66634675.23-P1P2 | Scn9a | P1P2 | GAGAGTCGCAATTGGAGCGC | 124 |
| Scn9a_−_66634637.23-P1P2 | Scn9a | P1P2 | GCCAGACCAGCCTGCACAGT | 125 |
| Scn9a_−_66634689.23-P1P2 | Scn9a | P1P2 | GAGCGCAGGCTAGGCCTGCA | 126 |
| Scn9a_−_66634610.23-P1P2 | Scn9a | P1P2 | GCTAGGAGTCCGGGATACCC | 127 |
| Scn9a_+_66634478.23-P1P2 | Scn9a | P1P2 | GAATCCGCAGGTGCACTCAC | 128 |
| Scn9a_−_66634641.23-P1P2 | Scn9a | P1P2 | GACCAGCCTGCACAGTGGGC | 129 |
| Scn9a_+_66634731.23-P1P2 | Scn9a | P1P2 | GCGACGCGGTTGGCAGCCGA | 130 |
| Scn10a_+_119719110.23-P1P2 | Scn10a | P1P2 | GGCAGGGTGGAACTCGTGAC | 131 |
| Scn10a_+_119719123.23-P1P2 | Scn10a | P1P2 | GCACCATCCAGCAAGCAGGG | 132 |
| Scn10a_−_119719078.23-P1P2 | Scn10a | P1P2 | GCGTCACTCAAGGATCTACA | 133 |
| Scn10a_+_119719086.23-P1P2 | Scn10a | P1P2 | GATGGGAATGGCACCCACGA | 134 |
| Scn10a_+_119718921.23-P1P2 | Scn10a | P1P2 | GCCTTTAGACGGAGAACAGA | 135 |
| Scn10a_+_119719051.23-P1P2 | Scn10a | P1P2 | GAGATCCTTGAGTGACGGAC | 136 |
| Scn10a_−_119719025.23-P1P2 | Scn10a | P1P2 | GCGGGGCTCCTCCACGAAGG | 137 |
| Scn10a_−_119719095.23-P1P2 | Scn10a | P1P2 | GCAAGGAATCACGCCTTCGT | 138 |
| Scn10a_+_119718881.23-P1P2 | Scn10a | P1P2 | GGCCATGCGCGAATGCTGAG | 139 |
| Scn10a_+_119719014.23-P1P2 | Scn10a | P1P2 | GGCAAGCCCAGCCACCTTCG | 140 |
| Scn11a_+_119825404.23-P1P2 | Scn11a | P1P2 | GAGGTAAGCCATCCAGGCTG | 141 |
| Scn11a_−_119825450.23-P1P2 | Scn11a | P1P2 | GTTCCTGCTAGGGAGGCTCA | 142 |
| Scn11a_−_119825400.23-P1P2 | Scn11a | P1P2 | GCCTGAAACGACAGAGGATG | 143 |
| Scn11a_+_119825277.23-P1P2 | Scn11a | P1P2 | GTCAGAGGTGGAGACCAGGT | 144 |
| Scn11a_−_119825394.23-P1P2 | Scn11a | P1P2 | GCCCCAGCCTGAAACGACAG | 145 |
| Scn11a_+_119825463.23-P1P2 | Scn11a | P1P2 | GGCCAAGAGCGAGAATCTCC | 146 |
| Scn11a_+_119825246.23-P1P2 | Scn11a | P1P2 | GGTCAGGTGTCAGAGCCCAT | 147 |
| Scn11a_+_119825242.23-P1P2 | Scn11a | P1P2 | GGGTGTCAGAGCCCATCGGT | 148 |
| Scn11a_+_119825431.23-P1P2 | Scn11a | P1P2 | GTGCCCTGAGCCTCCCTAGC | 149 |
| Scn11a_−_119825253.23-P1P2 | Scn11a | P1P2 | GTCTGTGAGAACCGACCGAT | 150 |
| Shank3_+_89499659.23-P1P2 | Shank3 | P1P2 | GGGCTCCGCAGGCGCAGCGG | 151 |
| Shank3_+_89499688.23-P1P2 | Shank3 | P1P2 | GgggccagcgcgggggACAG | 152 |
| Shank3_+_89499943.23-P1P2 | Shank3 | P1P2 | GCCGCTAGCGGGCCACACAG | 153 |
| Shank3_+_89499679.23-P1P2 | Shank3 | P1P2 | GcgggggACAGCGGCTCCGG | 154 |
| Shank3_+_89499612.23-P1P2 | Shank3 | P1P2 | GCATCGGCCCCGGCTTCGAG | 155 |
| Shank3_+_89499924.23-P1P2 | Shank3 | P1P2 | GGGGTACGGCGAGATCGCAA | 156 |
| Shank3_+_89499878.23-P1P2 | Shank3 | P1P2 | GATGCCGACGCGCACGACCA | 157 |
| Shank3_−_89499676.23-P1P2 | Shank3 | P1P2 | GGCCGCCGCCGCTGCGCCTG | 158 |
| Shank3_+_89499818.23-P1P2 | Shank3 | P1P2 | GGGGCCCGGACTGTTCCCGG | 159 |
| Shank3_+_89499938.23-P1P2 | Shank3 | P1P2 | GAGCGGGCCACACAGGGGTA | 160 |
| Trpv1_+_73234353.23-P1P2 | Trpv1 | P1P2 | GGGACTTACCAGCTAGGTGC | 161 |
| Trpv1_−_73234330.23-P1P2 | Trpv1 | P1P2 | GCCCACAAAGAACAGCTCCA | 162 |
| Trpv1_−_73234384.23-P1P2 | Trpv1 | P1P2 | GGCTGGTAAGTCCTTCTCAT | 163 |
| Trpv1_+_73234339.23-P1P2 | Trpv1 | P1P2 | GGGTGCAGGCACACTCCAAA | 164 |
| Trpv1_−_73234537.23-P1P2 | Trpv1 | P1P2 | GACTTAACTTGGCTGACTGT | 165 |
| Trpv1_+_73234478.23-P1P2 | Trpv1 | P1P2 | GTCAGCCTCCCAGAAGTCCA | 166 |
| Trpv1_−_73234495.23-P1P2 | Trpv1 | P1P2 | GGCTGCCTTGGACTTCTGGG | 167 |
| Trpv1_+_73234635.23-P1P2 | Trpv1 | P1P2 | GCCACGGAAGGCCTCCAGAT | 168 |
| Trpv1_−_73234346.23-P1P2 | Trpv1 | P1P2 | GCCAAGGCACTTGCTCCATT | 169 |
| Trpv1_+_73234280.23-P1P2 | Trpv1 | P1P2 | GGGCTGCTGTGTGGTAAGAG | 170 |
| Grin2b_−_136172154.23-P1P2 | Grin2b | P1P2 | GCCAACCTGAATGGAAGAGA | 171 |
| Grin2b_−_136172179.23-P1P2 | Grin2b | P1P2 | GAGGGAAGTGGAAAGCAAGG | 172 |
| Grin2b_−_136172123.23-P1P2 | Grin2b | P1P2 | GTGGGACAGGCATGGATGAA | 173 |
| Grin2b_+_136172089.23-P1P2 | Grin2b | P1P2 | GCCTGTCCCAGGAACGGCAT | 174 |
| Grin2b_−_136172145.23-P1P2 | Grin2b | P1P2 | GTGAGAAAAGCCAACCTGAA | 175 |
| Grin2b_−_136171934.23-P1P2 | Grin2b | P1P2 | GGATTCGAGTGTCTCCCGGA | 176 |
| Grin2b_−_136171999.23-P1P2 | Grin2b | P1P2 | GACCAAGTCGTTATAAGGAA | 177 |
| Grin2b_−_136172002.23-P1P2 | Grin2b | P1P2 | GAAGTCGTTATAAGGAAAGG | 178 |
| Grin2b_+_136171844.23-P1P2 | Grin2b | P1P2 | GGAATGACCACGCTCCACGG | 179 |
| Grin2b_+_136172019.23-P1P2 | Grin2b | P1P2 | GCCTCTGGTGTGTACTCTGT | 180 |

or a biological equivalent each thereof.

gRNA for Editing, in mouse

| | Target | | | Position of | | |
|---|---|---|---|---|---|---|
| Target | Gene | Target | Genomic | Base After | | |
| Gene ID | Symbol | Transcript | Sequence | Cut (1-based) | Strand | sgRNA Target Sequence |
| 20269 | Scn3a | NM_018732.3 | NC_000068.7 | 65495200 | sense | AAAGTGATAGAAATCCACGA |
| 20269 | Scn3a | NM_018732.3 | NC_000068.7 | 65497546 | sense | GTGTGTTTGCAAGATCAATG |
| 20269 | Scn3a | NM_018732.3 | NC_000068.7 | 65514506 | sense | CTGGATGGGAACCCGCTGAG |
| 20269 | Scn3a | NM_018732.3 | NC_000068.7 | 65507153 | sense | TATCCTGACCAACACGATGG |
| 20274 | Scn9a | NM_001290674.1 | NC_000068.7 | 66565145 | antisense | GCCAGTTCCAAGGGTCACGG |
| 20274 | Scn9a | NM_001290674.1 | NC_000068.7 | 66501680 | antisense | GTGTCCGTAGAGATTTAATG |
| 20274 | Scn9a | NM_001290674.1 | NC_000068.7 | 66526832 | sense | TATCTCAAACCGTACCCTTG |
| 20274 | Scn9a | NM_001290674.1 | NC_000068.7 | 66543284 | sense | CTGAGTACACGAGTTTAGGG |
| 20264 | Scn10a | NM_001205321.1 | NC_000075.6 | 119648039 | antisense | CAAGAGAAGACGTTACCAAG |
| 20264 | Scn10a | NM_001205321.1 | NC_000075.6 | 119669980 | antisense | GATCCATTGCCACACAACAA |
| 20264 | Scn10a | NM_001205321.1 | NC_000075.6 | 119661277 | antisense | CCAGCAATATGGAACTTCGA |
| 20264 | Scn10a | NM_001205321.1 | NC_000075.6 | 119635553 | sense | CATCACTGATCCTAACGTGT |
| 24046 | Scn11a | NM_011887.3 | NC_000075.6 | 119805789 | antisense | TATTGCACGTGGAACCATCG |
| 24046 | Scn11a | NM_011887.3 | NC_000075.6 | 119783806 | sense | GAGGACGATATGGAATGTTG |
| 24046 | Scn11a | NM_011887.3 | NC_000075.6 | 119795782 | antisense | TTTGTTTGCTCAAGGAGTTG |
| 24046 | Scn11a | NM_011887.3 | NC_000075.6 | 119790225 | antisense | CTTAATGAGAGTGTTTAATG |
| 58234 | Shank3 | NM_021423.3 | NC_000081.6 | 89548242 | sense | GAACCCTCTCCGACGCACCG |
| 58234 | Shank3 | NM_021423.3 | NC_000081.6 | 89525264 | sense | AGATGCGACAGTATGACACC |
| 58234 | Shank3 | NM_021423.3 | NC_000081.6 | 89547884 | antisense | CGTGCTCGGATCATACAGGC |
| 58234 | Shank3 | NM_021423.3 | NC_000081.6 | 89543866 | antisense | GTACCTACAGATTTGGTCCG |
| 193034 | Trpv1 | NM_001001445.2 | NC_000077.6 | 73246001 | sense | TAAGCTGAATAACACCGTTG |
| 193034 | Trpv1 | NM_001001445.2 | NC_000077.6 | 73250757 | antisense | AAGCCACATACTCCTTGCGA |
| 193034 | Trpv1 | NM_001001445.2 | NC_000077.6 | 73239324 | antisense | CCTGCGATCATAGAGCCTTG |
| 193034 | Trpv1 | NM_001001445.2 | NC_000077.6 | 73244214 | antisense | GCTCCACGAGAAGCATGTCG |
| 14812 | Grin2b | NM_008171.3 | NC_000072.6 | 135733840 | sense | TATCCTACGCTTGCTCCGAA |
| 14812 | Grin2b | NM_008171.3 | NC_000072.6 | 135774815 | antisense | GGCACCGGTTGTAACCCACA |
| 14812 | Grin2b | NM_008171.3 | NC_000072.6 | 135923390 | sense | ACATCATGGAAGAATACGAC |
| 14812 | Grin2b | NM_008171.3 | NC_000072.6 | 135923120 | sense | TGACTGGCTACGGCTACACA |
| Target | SEQ | | | | PAM | |

| Gene ID | ID NO | Target Context Sequence | SEQ ID NO | Sequence | Exon Number |
|---|---|---|---|---|---|
| 20269 | 181 | GCCGAAAGTGATAGAAATCCACGAA | 209 | AGG | 17 |
| | | GGGAA | | | |
| 20269 | 182 | AGGAGTGTGTTTGCAAGATCAATGA | 210 | AGG | 16 |
| | | GGACT | | | |
| 20269 | 183 | CTCCCTGGATGGGAACCCGCTGAGC | 211 | CGG | 11 |
| | | GGCGA | | | |
| 20269 | 184 | CCAGTATCCTGACCAACACGATGGA | 212 | AGG | 13 |
| | | GGGTA | | | |
| 20274 | 185 | TCCAGCCAGTTCCAAGGGTCACGGA | 213 | AGG | 5 |
| | | GGAAG | | | |
| 20274 | 186 | CTCAGTGTCCGTAGAGATTTAATGG | 214 | GGG | 21 |
| | | GGCCA | | | |
| 20274 | 187 | ACTATATCTCAAACCGTACCCTTGC | 215 | CGG | 17 |
| | | GGAGA | | | |
| 20274 | 188 | GCTGCTGAGTACACGAGTTTAGGGC | 216 | CGG | 11 |
| | | GGAGC | | | |
| 20264 | 189 | TGGCCAAGAGAAGACGTTACCAAGC | 217 | CGG | 15 |
| | | GGAAG | | | |
| 20264 | 190 | ATCAGATCCATTGCCACACAACAAG | 218 | GGG | 8 |
| | | GGATC | | | |
| 20264 | 191 | CTGCCCAGCAATATGGAACTTCGAC | 219 | CGG | 12 |
| | | GGCTT | | | |
| 20264 | 192 | ACTTCATCACTGATCCTAACGTGTG | 220 | GGG | 17 |
| | | GGTCT | | | |
| 24046 | 193 | GTTTTATTGCACGTGGAACCATCGG | 221 | GGG | 9 |
| | | GGCAG | | | |
| 24046 | 194 | AGAAGAGGACGATATGGAATGTTGT | 222 | TGG | 16 |
| | | GGTGA | | | |
| 24046 | 195 | TCGTTTTGTTTGCTCAAGGAGTTGT | 223 | TGG | 12 |
| | | GGCTG | | | |
| 24046 | 196 | TGATCTTAATGAGAGTGTTTAATGT | 224 | TGG | 15 |
| | | GGGCC | | | |
| 58234 | 197 | ACGAGAACCCTCTCCGACGCACCGC | 225 | CGG | 21 |
| | | GGGCC | | | |
| 58234 | 198 | GTGCAGATGCGACAGTATGACACCC | 226 | CGG | 12 |
| | | GGCAT | | | |
| 58234 | 199 | GAGGCGTGCTCGGATCATACAGGCC | 227 | CGG | 21 |
| | | GGCGG | | | |
| 58234 | 200 | AGCCGTACCTACAGATTTGGTCCGT | 228 | TGG | 20 |
| | | GGAAT | | | |
| 193034 | 201 | CCTATAAGCTGAATAACACCGTTGG | 229 | GGG | 9 |
| | | GGACT | | | |
| 193034 | 202 | ATGGAAGCCACATACTCCTTGCGAT | 230 | TGG | 11 |
| | | GGCTG | | | |
| 193034 | 203 | TGCTCCTGCGATCATAGAGCCTTGG | 231 | GGG | 3 |
| | | GGGCG | | | |
| 193034 | 204 | AAGGGCTCCACGAGAAGCATGTCGT | 232 | TGG | 8 |
| | | GGCGG | | | |
| 14812 | 205 | CCAATATCCTACGCTTGCTCCGAAC | 233 | CGG | 15 |
| | | GGCCA | | | |
| 14812 | 206 | GCTAGGCACCGGTTGTAACCCACAG | 234 | GGG | 10 |
| | | GGCTG | | | |
| 14812 | 207 | CTCAACATCATGGAAGAATACGACT | 235 | TGG | 5 |
| | | GGTAC | | | |
| 14812 | 208 | GGGCTGACTGGCTACGGCTACACAT | 236 | TGG | 5 |
| | | GGATC | | | |

or a biological equivalent each thereof.

Gene constructs for Activation (Overexpression)

| | |
|---|---|
| Insert_mll10 | gcagagctctctggctaactaccggtgccaccATGCCTGGCTCAGCACTGCTATGCTGCCTGC |
| | TCTTACTGACTGGCATGAGGATCAGCAGGGGCCAGTACAGCCGGGAAGACAATAACTGCACCC |
| | ACTTCCCAGTCGGCCAGAGCCACATGCTCCTAGAGCTGCGGACTGCCTTCAGCCAGGTGAAGA |
| | CTTTCTTTCAAACAAAGGACCAGCTGGACAACATACTGCTAACCGACTCCTTAATGCAGGACT |
| | TTAAGGGTTACTTGGGTTGCCAAGCCTTATCGGAAATGATCCAGTTTTACCTGGTAGAAGTGA |
| | TGCCCCAGGCAGAGAAGCATGGCCCAGAAATCAAGGAGCATTTGAATTCCCTGGGTGAGAAGC |
| | TGAAGACCCTCAGGATGCGGCTGAGGCGCTGTCATCGATTTCTCCCCTGTGAAAATAAGAGCA |
| | AGGCAGTGGAGCAGGTGAAGAGTGATTTTAATAAGCTCCAAGACCAAGGTGTCTACAAGGCCA |
| | TGAATGAATTTGACATCTTCATCAACTGCATAGAAGCATACATGATGATCAAAATGAAAAGCT |
| | AAgaattcctagagctcgctgatcagcc (SEQ ID NO: 237) |
| Insert_mPenk | gcagagctctctggctaactaccggtgccaccATGGCGCGGTTCCTGAGGCTTTGCACCTGGC |
| | TGCTGGCGCTTGGGTCCTGCCTCCTGGCTACAGTGCAGGCGGAATGCAGCCAGGACTGCGCTA |
| | AATGCAGCTACCGCCTGGTTCGCCCAGGCGACATCAATTTCCTGGCGTGCACACTGGAATGTG |
| | AAGGACAGCTGCCTTCTTTCAAAATCTGGGAGACCTGCAAGGATCTCCTGCAGGTGTCCAGGC |
| | CCGAGTTCCCTTGGGATAACATCGACATGTACAAAGACAGCAGCAAACAGGATGAGAGCCACT |
| | TGCTAGCCAAGAAGTACGGAGGCTTCATGAAACGGTACGGAGGCTTCATGAAGAAGATGGACG |
| | AGCTATATCCCATGGAGCCAGAAGAAGAAGCGAACGGAGGAGAGATCCTTGCCAAGAGGTATG |
| | GCGGCTTCATGAAGAAGGATGCAGATGAGGGAGACACCTTGGCCAACTCCTCCGATCTGCTGA |
| | AAGAGCTACTGGGAACGGGAGACAACCGTGCGAAAGACAGCCACCAACAAGAGAGCACCAACA |
| | ATGACGAAGACATGAGCAAGAGGTATGGGGGCTTCATGAGAAGCCTCAAAAGAAGCCCCCAAC |
| | TGGAAGATGAAGCAAAAGAGCTGCAGAAGCGCTACGGGGGCTTCATGAGAAGGGTGGGACGCC |
| | CCGAGTGGTGGATGGACTACCAGAAGAGGTATGGGGGCTTCCTGAAGCGCTTTGCTGAGTCTC |
| | TGCCCTCCGATGAAGAAGGCGAAAATTACTCGAAAGAAGTTCCTGAGATAGAGAAAAGATACG |
| | GGGGCTTTATGCGGTTCTGAgaattcctagagctcgctgatcagcc (SEQ ID NO: 238) |
| Insert_mPomc | gcagagctctctggctaactaccggtgccaccATGCCGAGATTCTGCTACAGTCGCTCAGGGG |
| | CCCTGTTGCTGGCCCTCCTGCTTCAGACCTCCATAGATGTGTGGAGCTGGTGCCTGGAGAGCA |
| | GCCAGTGCCAGGACCTCACCACGGAGAGCAACCTGCTGGCTTGCATCCGGGCTTGCAAACTCG |
| | ACCTCTCGCTGGAGACGCCCGTGTTTCCTGGCAACGGAGATGAACAGCCCCTGACTGAAAACC |
| | CCCGGAAGTACGTCATGGGTCACTTCCGCTGGGACCGCTTCGGCCCCAGGAACAGCAGCAGTG |
| | CTGGCAGCGCGGCGCAGAGGCGTGCGGAGGAAGAGGCGGTGTGGGGAGATGGCAGTCCAGAGC |
| | CGAGTCCACGCGAGGGCAAGCGCTCCTACTCCATGGAGCACTTCCGCTGGGGCAAGCCGGTGG |
| | GCAAGAAACGGCGCCCGGTGAAGGTGTACCCCAACGTTGCTGAGAACGAGTCGGCGGAGGCCT |
| | TTCCCCTAGAGTTCAAGAGGGAGCTGGAAGGCGAGCGGCCATTAGGCTTGGAGCAGGTCCTGG |
| | AGTCCGACGCGGAGAAGGACGACGGGCCCTACCGGGTGGAGCACTTCCGCTGGAGCAACCCGC |
| | CCAAGGACAAGCGTTACGGTGGCTTCATGACCTCCGAGAAGAGCCAGACGCCCCTGGTGACGC |
| | TCTTCAAGAACGCCATCATCAAGAACGCGCACAAGAAGGGCCAGTGAgaattcctagagctcg |
| | ctgatcagcc (SEQ ID NO: 239) |
| Insert_MVIIA-PC | gcagagctctctggctaactaccggtgccaccATGAGTGCATTGCTCATCCTGGCCCTGGTCG |
| | GGGCTGCCGTGGCTTGTAAAGGCAAAGGAGCTAAATGCAGTAGACTTATGTATGATTGTTGCA |
| | CGGGTTCATGTAGATCAGGGAAGTGCATCGACTATAAAGACGACGATGACAAACTGGCAGCTG |
| | CCGGTAACGGTAATGGGAATGGGAACGGCAACGGGAACGGTAACGGAGACGGCACGAGGGTAG |
| | CAGTAGGACAGGACACGCAAGAGGTAATCGTTGTACCGCATAGTCTCCCCTTCAAGGTAGTAG |
| | TGATCAGTGCTATACTGGCGCTGGTGGTTCTCACAATTATTAGTCTGATAATTTTGATAATGC |
| | TGTGGCAAAAAAAGCCCCGGAGAATCCGAATGGTCAGTAAGGGTGAAGAAGACAATATGGCCA |
| | TAATTAAGGAGTTCATGCGATTCAAGGTACATATGGAGGGTAGCGTCAATGGTCACGAGTTCG |
| | AAATAGAAGGCGAAGGCGAGGGGAGACCCTATGAAGGAACACAGACAGCTAAACTTAAGGTAA |
| | CGAAAGGCGGCCCACTCCCGTTCGCCTGGGATATTCTTAGTCCGCAGTTCATGTACGGTTCAA |
| | AGGCGTATGTCAAACATCCAGCGGACATCCCCGATTACCTGAAATTGAGCTTCCCAGAGGGAT |
| | TTAAATGGGAGCGGGTCATGAATTTCGAAGATGGGGGAGTTGTGACAGTAACTCAAGACTCCA |
| | GTCTCCAGGATGGTGAATTCATATACAAAGTCAAACTCAGGGGCACCAATTTCCCCAGCGACG |
| | GCCCCGTCATGCAAAAGAAAACCATGGGATGGGAGGCCAGCTCCGAGCGCATGTATCCTGAGG |
| | ATGGAGCTCTTAAAGGAGAGATCAAACAGCGCCTGAAGTTGAAGGATGGAGGCCACTACGATG |
| | CCGAGGTTAAGACAACCTATAAGGCCAAAAAGCCAGTGCAGCTTCCGGGAGCGTACAATGTAA |
| | ACATCAAGCTGGATATTACGAGCCACAACGAGGACTACACGATAGTAGAACAGTACGAGAGAG |
| | CAGAGGGACGGCACTCCACTGGTGGTATGGACGAATTGTATAAGTAAgaattcctagagctcg |
| | ctgatcagcc (SEQ ID NO: 240) |

or a biological equivalent each thereof.

Liver Disease: In some embodiments, gRNAs are designed to target liver disease and conditions related to liver malfunction, such as but not limited to malaria and hepatitis. Malaria is a life-threatening mosquito-borne disease caused by a parasite, with an estimated 3.3 billion people in 106 countries and territories at risk—nearly half the world's population. As a consequence, finding a way to prevent infection could be very beneficial. Malaria is associated with three host genes in the liver, CD81, Sr-b1, and MUC13. CD81 is also a known receptor for hepatitis C virus. Not to be bound by theory, it is believe that targeting one or more of these genes would impede the ability of one or more of these diseases to infect a host. Therefore, use of the disclosed recombinant expression system comprising gRNAs tailored for the regulation or editing of these gene targets may be useful in the treatment and/or prevention thereof. In some embodiments, this may include prophylactic administration of a recombinant expression system comprising these gRNAs. Non-limiting examples of gRNAs for use in liver diseases, such as but not limited to malaria, hepatitis C, or any other disease in which these genes are implicated, include:

CD81:
(SEQ ID NO: 241)
CGAAATTGAAGACGAAGAGC
MUC13:
(SEQ ID NO: 242)
GGAGACTGAGAGAGAGAAGC
Sr-b1:
(SEQ ID NO: 243)
TGATGAGGGAGGGCACCATG

or a biological equivalent each thereof.

Hematopoietic Stem Cell Therapy and HIV: In some embodiments, gRNAs are designed to prevent immune rejection of hematopoietic stem cells (HSC) and/or to prevent HIV from entering a host cell. HSC gene therapy can potentially cure a variety of human hematopoietic diseases, such as sickle cell anemia. The current process of HSC gene therapy, however, is very complex and expensive. Currently, the hematopoietic stem cell transplantation process involves taking HSCs from one person (donor) and transfusing them into another (recipient). Some drawbacks to this method include an immune response due to the cells being from a foreign body (or graft rejection). In order to prevent rejection, many patients also require chemotherapy and/or radiation therapy, which in itself weakens the patients. Another drawback is Graft versus Host Disease (GVHD), where mature T-cells from the donor perceive the recipient's tissue as foreign and attack these tissues. In this case, the recipient must take medication to suppress inflammation and T-cell activation. Interestingly, the CCR5 co-receptor is associated with the rejection of HSC transplants and the ability of HIV to enter a host cell. Indeed, people who are resistant to HIV, which have a mutation in the CCR5 gene, called CCR5-delta 32, which results in a truncated protein that does not allow HIV to infect the cells. Accordingly, for both applications, a recombinant expression system with a gRNA targeting CCR5 can be utilized. A non-limiting exemplary gRNA is provided:

CCR5 gRNA:
(SEQ ID NO: 244)
GGTCCTGCCGCTGCTTGTCA

or a biological equivalent thereof.

Cancer immunotherapy: Cancer immunotherapy uses the components of the immune system to combat cancers, usually by enhancing the body's own immune response against cancerous cells using either antibodies or engineered T-cells. Typically, T-cell based therapy involves extraction of the immune cells from a patient followed by re-infusion after enrichment, editing or treatment. Since PDCD-1 plays an important role in halting the T-cell immune response, knocking it out may improve the ability of the T-cells to eliminate cancer cells and, treatments using these engineered immune cells have generated some remarkable responses in patients with advanced cancer. Further non-cancer related immune responses may also be modulated with this approach. An exemplary recombinant expression system with a gRNA targeting PDCD-1 for this purpose is disclosed herein. Non-limiting exemplary gRNA are provided:

### PDCD-1 Target Sequences:

1.
(SEQ ID NO: 245)
AGCCGGCCAGTTCCAAACCC
2.
(SEQ ID NO: 246)
AGGGCCCGGCGCAATGACAG

or a biological equivalent each thereof.

Abnormal activity of signaling pathways can lead to cancer. For example, it has been demonstrated that downregulation of nodal (part of TGF-β family, e.g. Uniprot Ref No. Q96S42) may cause downregulation of molecules that are associated with metastatic melanoma and that blocking the hedgehog pathway can prevent tumor growth. Thus, the recombinant expression system may be used to downregulate target genes within these pathways could therefore be used to treat cancer by designing specific gRNAs to these targets.

A large fraction of myeloproliferative cancers show a V617F mutation in JAK-2 (e.g. Uniprot Ref No. O60674). However this mutation persists in the HSC population of the individual too gRNAs to target the V617F mutation in the HSC population are also within the scope of this disclosure.

Blood Diseases: Clinical symptoms of malaria occur during the blood stage of the life-cycle of the plasmodium parasites that invade and reside within erythrocytes, making use of host proteins and resources towards their own needs, leading to a transformation of the host cell. Certain cell surface receptors such as Duffy, Glycophorin A/C, etc. have been shown to be essential for the entry of parasites into the erythrocytes. In addition the parasite is heavily reliant on the Pyruvate Kinase in the erythrocytes. Knocking out these genes is believed to confer resistance to plasmodium invasion. The following non-limiting exemplary gRNAs are provided for constructs for this purpose:

### GYPA

1.
(SEQ ID NO: 247)
TCTTCAAATAACCACTCCTG
2.
(SEQ ID NO: 248)
TCAGCAACAATGTCAACACC

### GYPC

1.
(SEQ ID NO: 249)
GGCAATCTCCATAATGCCGT
2.
(SEQ ID NO: 250)
TATCCACAGAGCCTAACCCA

### PKLR

1.
(SEQ ID NO: 251)
TGTACGAAAAGCCAGTGATG
2.
(SEQ ID NO: 252)
GGGTTCACTCCAGACCTGTG

### ACKR1 (Duffy)

1.
(SEQ ID NO: 253)
AAGGTCTGAGAATCGCGAAG
2.
(SEQ ID NO: 254)
CATTCTGGCAGAGTTAGCAG

or a biological equivalent each thereof.

Muscular dystrophy: Aberrant dystrophin has been associated with muscular dystrophy, among other genes. Disclosed in Table 1 are exemplary gRNA for use in muscular dystrophy and other neurodegenerative diseases.

In utero fetus specific targeting: Specific gRNAs may be designed to a carrier mutation, for example from the father of a fetus, which would enable a recombinant expression system to specifically target a fetus and not the mother in utero. Thus, if a fetus presents with a diseased genotype that is not present in the mother, it could be resolved in utero without affecting the mother's genome.

Cytochrome P450-based disorders: Cytochrome P450 enzyme CYP2D6 (e.g. UniProt Ref No. P10635) is known to be associated with varied drug metabolism. Polymorphisms of this enzyme expressed by a percentage of certain populations (e.g. Caucasians) prevent the conversion of codeine to morphine, a pain-relieving drug. At least two active or functional copies of CYP2D6 are required in rapid and complete metabolism of codeine. For patients having 2 inactive copies of CYP2D6, providing a gRNA in the recombinant expression system that activates or overexpresses at least 1 active copy of CYP2D6 in the patient allows for metabolism of codeine.

In the presence of certain substrates or exposure to certain physiological conditions, cytochrome P450s (CYP), may produce reactive oxidative species (ROS) or give rise to metabolites disrupting normal metabolism or damaging tissues in the body. Being able to induce activation or repression of CYP genes may thus prevent toxicity not only from drug-drug interactions but also from conditions that result in abnormal levels of metabolic cofactors.

More generally, inconsistent drug responses may be addressed using targeted gRNA, designed to elicit a next generation drug-drug interactions that are beneficial to patients.

Reprogramming Macrophages: Macrophages contain different subpopulations polarized by chemokines and cytokines and ultimately affect whether an immune response is pro-inflammatory or pro-regenerative. Specific gRNA may be used in the recombinant expression system to target macrophages and drive phenotypes toward M2 macrophages for pro-regenerative conditions.

Repelling Mosquitoes: Although the cause seems to be largely unknown, mosquitoes and other insects have a preference for biting certain people yet avoiding others. A twin study showed that there seems to be a genetic component to this attraction, but the specific gene is unknown. Another factor that influences mosquito attraction is odors given off by the host. Through selecting a gRNA that could alter the gene that causes this attraction or cause the person to produce a substance that repels mosquitoes, the recombinant expression system could provide term protection for people visiting areas known to have disease-carrying insects. gRNAs targeting HSCs in the bone marrow, which may in turn defend against mosquitoes are also within the scope of this disclosure.

Alzheimer's: Researchers have shown that the binding of B-Amyloids to LilrB2 (e.g. UniProt Ref No. Q8N423) is one of the first steps leading to Alzheimer's. Thus, gRNAs are contemplated herein for use in the recombinant expression system, which in turn would be capable of causing point mutations in the DID2 region of LilrB2 such that it affects the B-Amyloid binding could prevent the onset of Alzheimer's. D1 is associated with Uniprot Ref No. P21728. D2 is associated with Uniprot Ref No. 14416. Non-limiting exemplary sequences thereof are provided herein below:

### Dopamine Receptor D1

```
(SEQ ID NO: 255)
                10 20 30 40
MRTLNTSAMD GTGLVVERDF SVRILTACFL SLLILSTLLG
                50 60 70 80
NTLVCAAVIR FRHLRSKVTN FFVISLAVSD LLVAVLVMPW
                90 100 110 120
KAVAEIAGFW PFGSFCNIWV AFDIMCSTAS ILNLCVISVD
              130 140 150 160
RYWAISSPFR YERKMTPKAA FILISVAWTL SVLISFIPVQ
              170 180 190 200
LSWHKAKPTS PSDGNATSLA ETIDNCDSSL SRTYAISSSV
              210 220 230 240
ISFYIPVAIM IVTYTRIYRI AQKQIRRIAA LERAAVHAKN
            250 260 270 280
CQTTTGNGKP VECSQPESSF KMSFKRETKV LKTLSVIMGV
            290 300 310 320
FVCCWLPFFI LNCILPFCGS GETQPFCIDS NTFDVFVWFG
            330 340 350 360
WANSSLNPII YAFNADFRKA FSTLLGCYRL CPATNNAIET
            370 380 390 400
VSINNNGAAM FSSHHEPRGS ISKECNLVYL IPHAVGSSED
            410 420 430 440
LKKEEAAGIA RPLEKLSPAL SVILDYDTDV SLEKIQPITQ
NGQHPT
```

### Dopamine Receptor D2

```
(SEQ ID NO: 256)
                10 20 30 40
MDPLSLSWYD DDLERQNWSR PFNGSDGKAD RPHYNYYATL
                50 60 70 80
LTLLIAVIVF GNVLVCMAVS REKALQTTTN YLIVSLAVAD
                90 100 110 120
LLVATLVMPW VVYLEVVGEW KFSRIHCDIF VTLDVMMCTA
              130 140 150 160
SILNLCAISI DRYTAVAMPM LYNTRYSSKR RVTVMISIVW
              170 180 190 200
VLSFTISCPL LFGLNNADQN ECIIANPAFV VYSSIVSFYV
              210 220 230 240
PFIVTLLVYI KIYIVLRRRR KRVNTKRSSR AFRAHLRAPL
              250 260 270 280
KGNCTHPEDM KLCTVIMKSN GSFPVNRRRV EAARRAQELE
              290 300 310 320
MEMLSSTSPP ERTRYSPIPP SHHQLTLPDP SHHGLHSTPD
              330 340 350 360
SPAKPEKNGH AKDHPKIAKI FEIQTMPNGK TRTSLKTMSR
              370 380 390 400
RKLSQQKEKK ATQMLAIVLG VFIICWLPFF ITHILNIHCD
              410 420 430 440
CNIPPVLYSA FTWLGYVNSA VNPIIYTTFN IEFRKAFLKI
LHC
```

Thyroid hormone production: Thyroid disorders (both hyper and hypothyroidism) affect a large set of human population. gRNAs are selected for use in the recombinant expression system which would allow for regulation of thyroid hormones and result in treatment or prevention of these disorders.

### Ordering of Effector Elements

It should be appreciated that the effector elements disclosed herein may be configured in a variety of ways depending on the space available in each of the two vectors in the recombinant expression system disclosed herein, e.g. a split-Cas9 system. Further, it is understood that the effector elements disclosed herein may optionally be used in a Cas9 system that comprises one vector encoding a full Cas9 protein and another encoding the requisite gRNA for CRISPR-based genomic or epigenomic editing. FIG. 5 provides an exemplary schematic of an miRNA circuit employed in this manner. The Figures provide non-limiting exemplary schematics and ordering of the various effector elements disclosed herein.

For example, effector elements used for activation (e.g. VP64, RTA, P65), repression (e.g. KRAB), and/or altering methylation (e.g. DNMT3A, DNMT3L) can be placed on either the first expression vector or the second expression vector of the recombinant expression system, e.g. a split-Cas9 system.

The TRE and tet-regulatable activator must be encoded in two different vectors in the recombinant expression system. In some embodiments, the tet-regulatable activator is encoded in the N-Cas9 encoding vector and the TRE is encoded in the C-Cas9 encoding vector. In some embodiments, this may be reversed wherein the TRE is encoded in the N-Cas9 encoding vector and the tet-regulatable element is encoded in the C-Cas9 encoding vector.

Promoter placement also is a consideration in the disclosed constructs. In one aspect, a construct comprising gRNA should have a promoter, optionally a U6 promoter, encoded upstream thereof. Similarly, a construct comprising Cas9 or either of the two halves of split-Cas9 should have a promoter, optionally a CMV promoter, encoded upstream thereof.

### Capsid Engineering

Aspects of this disclosure relate to a viral capsid engineered to impart favorable characteristics, such as but not limited to the addition of one or more unnatural amino acids and/or a SpyTag sequence or the corresponding KTag sequence. In some embodiments, the viral capsid is an AAV capsid or a lentiviral capsid.

A variety of sites can be modified on the capsid to incorporate one or more unnatural amino acid, SpyTag sequence, or KTag sequence. In some embodiments, a surface exposed site is identified as the appropriate site for incorporation of one or more unnatural amino acid, SpyTag sequence, or KTag sequence. A non-limiting example of such sites in the AAV2 capsid are residues 447, 578, 87, and 662 of the VP1 in AAV2. In some embodiments, sites for incorporation of the one or more unnatural amino acid, SpyTag sequence, or KTag sequence are those that do not compromise AAV function. With respect to AAV2, certain surface residues are known to perfect assembly, e.g. residues 509-522 and 561-565, confer HSPG binding, e.g. 586-591, 484, 487, and K532. Residues 138 and 139 are surface exposed and found at the N-terminal of VP2, which is comprised in the AAV2 capsid. Up to 15 amino acids can be inserted at positions 139, 161, 459, 584, and 587.

An unnatural amino acid (also referred to as “UAA” or a “non-canonical amino acid”) is an amino acid that may occur naturally or be chemically synthesized but is not one of the 22 canonical amino acids that are used in native eukaryote and prokaryote protein synthesis. Non-limiting examples of such include β-amino acids, homo-amino acids, proline and pyruvic acid derivatives, 3-substituted alanine derivatives, glycine derivatives, ring-substituted phenylalanine and tyrosine derivatives, linear core amino acids, and N-methyl amino acids. Non-limiting exemplary unnatural amino acids are described and commercially available through Sigma Aldrich (sigmaaldrich.com/chemistry/chemistry-products.html?TablePage=16274965). Further non-limiting examples include N-epsilon-((2-Azidoethoxy) carbonyl)-L-Lysine, pyrrolysine, and other lysine derivatives.

In some embodiments, the unnatural amino acid comprises an azide or an alkyne. The selection of functional groups comprised in the unnatural amino acid can facilitate the use of click chemistry to add further moieties to the viral capsid. For example, azide-alkyne addition provides a straightforward way to incorporate additional functional groups onto the amino acid.

In some embodiments, the unnatural amino acid is charged or uncharged or polar or nonpolar. In some embodiments, the unnatural amino acid is highly negatively or positively charged. The selection of charge and polarity of the unnatural amino acid is dependent on the next steps to be taken with the viral capsid. For example, if the viral capsid will be encapsulated with lipofectamine, a highly negatively charged unnatural amino acid may be desirable.

Methods of unnatural amino acids incorporation into proteins are known in the art and include the use of an orthogonal translational system making use of reassigned stop codons, e.g. amber suppression. Non-limiting examples of orthogonal tRNA synthetase for carrying out such additions include but are not limited to MbPylRS, MmPylRS, and AcKRS. Incorporation of unnatural amino acids may be further enhanced by the use of additional agents. A non-limiting example is eTF1, an exemplary sequence of which is provided below:

eTF1 (normal)-E55D (bold italic, modified
sequence)
(SEQ ID NO: 257)
MADDPSAADRNVEIWKIKKLIKSLEAARGNGTSMISLIIPPKDQISRVA
KMLADDFGTASNIKSRVNRLSVLGAITSVQQRLKLYNKVPPNGLVVYCG
TIVTEEGKEKKVNIDFEPFKPINTSLYLCDNKFHTEALTALLSDDSKFG
FIVIDGSGALFGTLQGNTREVLHKFTVDLPKKHGRGGQSALRFARLRME
KRHNYVRKVAETAVQLFISGDKVNVAGLVLAGSADFKTELSQSDMFDQR
LQSKVLKLVDISYGGENGFNQAIELSTEVLSNVKFIQEKKLIGRYFDEI
SQDTGKYCFGVEDTLKALEMGAVEILIVYENLDIMRYVLHCQGTEEEKI
LYLTPEQEKDKSHFTDKETGQEHELIESMPLLEWFANNYKKFGATLEIV
TDKSQEGSQFVKGFGGIGGILRYRVDFQGMEYQGGDDEFFDLDDY

Similar methods may be used to incorporate a SpyTag or KTag on the viral capsid. SpyTag is a known sequence AHIVMVDAYKPTK (SEQ ID NO: 258) that pairs with a corresponding KTag sequence ATHIKFSKRD (SEQ ID NO: 259) and ligate in the presence of SpyLigase—a commercially available enzyme available through AddGene and associated with GenBank Ref No. KJ401122—and in some instances spontaneously.

The below AAV sequences from AAV2 and AAV-DJ provide exemplary positions at which an unnatural amino acid, SpyTag, or KTag sequence can be incorporated.

AAV2 VP1 (normal) (R447 (bold); S578 (bold
underline); N587 (bold italic); S662 (bold,
double underline))
(SEQ ID NO: 260)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISQSGASNDNHYFGYSTPWGYFDFNRFHCHFSPRD
WQRLINNNWGFRPKRLNFKLFNIQVKEVTQNDGTTTIANNLTSTVQVFT
DSEYQLPYVLGSAHQGCLPPFPADVFMVPQYGYLTLNNGSQAVGRSSFY
CLEYFPSQMLRTGNNFTFSYTFEDVPFHSSYAHSQSLDRLMNPLIDQYL
YYLSRTNTPSGTTTQSRLQFSQAGASDIRDQSRNWLPGPCYRQQRVSKT
SADNNNSEYSWTGATKYHLNGRDSLVNPGPAMASHKDDEEKFFPQSGVL
IFGKQGSEKTNVDIEKVMITDEEEIRTTNPVATEQYGSVSTNLQRGNRQ
AATADVNTQGVLPGMVWQDRDVYLQGPIWAKIPHTDGHFHPSPLMGGFG
LKHPPPQILIKNTPVPANPSTTFSAAKFASFITQYSTGQVSVEIEWELQ
KENSKRWNPEIQYTSNYNKSVNVDFTVDTNGVYSEPRPIGTRYLTRNL
AAV-DJ VP1 (normal) (N589 (bold underline))
(SEQ ID NO: 261)
MAADGYLPDWLDETLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRLLEPLGLVEEAAKTAPGKKRPVE
HSPVEPDSSSGTGKAGQQPARKRLNFGQTGDADSVPDPQPIGEPPAAPS
GVGSLTMAAGGGAPMADNNEGADGVGNSSGNWHCDSTWMGDRVITTSTR
TWALPTYNNHLYKQISNSTSGGSSNDNAYFGYSTPWGYFDFNRFHCHFS
PRDWQRLINNNWGFRPKRLSFKLFNIQVKEVTQNEGTKTIANNLTSTIQ
VFTDSEYQLPYVLGSAHQGCLPPFPADVFMIPQYGYLTLNNGSQAVGRS
SFYCLEYFPSQMLRTGNNFQFTYTFEDVPFHSSYAHSQSLDRLMNPLID
QYLYYLSRTQTTGGTTNTQTLGFSQGGPNTMANQAKNWLPGPCYRQQRV
SKTSADNNNSEYSWTGATKYHLNGRDSLVNPGPAMASHKDDEEKFFPQS
GVLIFGKQGSEKTNVDIEKVMITDEEEIRTTNPVATEQYGSVSTNLQRG
NRQAATADVNTQGVLPGMVWQDRDVYLQGPIWAKIPHTDGHFHPSPLMG
GFGLKHPPPQILIKNTPVPADPPTTFNQSKLNSFITQYSTGQVSVEIEW
ELQKENSKRWNPEIQYTSNYYKSTSVDFAVNTEGVYSEPRPIGTRYLTR
NL

Unless otherwise provided, references to amino acid positions in the AAV2 or AAV-DJ VP1 sequence are based the position of the residues in the above disclosed sequences. Further, when the VP1 of each AAV are referred to, the intent is to also encompass biological equivalents thereof.

In some embodiments, the one or more unnatural amino acids, SpyTag, or KTag incorporated into the capsid is used to introduce additional moieties or “pseudotype” the surface of the capsid. The moieties include but are not limited peptides, aptamers, oligonucleotides, affibodies, DARPins, Kunitz domains, fynomers, bicyclic peptides, anticalin, and adnectin. The various moieties may be useful for a number of functions, including isolation of the virus, linking of the virus with another virus, and/or allowing homing of the virus to a particular target cell, organ, or tissue.

Such pseudotyping can be achieved through click chemistry. Where a SpyTag is incorporated onto the capsid, the click chemistry involves the conjugation of a KTag to the moiety to be pseudotyped. By adapting the reactions to facilitate the ligation of SpyTag to KTag (e.g. through the introduction of SpyLigase), the moiety is added to the surface of the capsid. A non-limiting example of sequences for such pseudotyping are KTag conjugated to Substance-P and RVG, two agents for neuronal homing in pain management:

KTag-SubstanceP:
(SEQ ID NO: 262)
ATHIKFSKRD GSGSGS RPKPQQFFGLM
SubstanceP-KTag:
(SEQ ID NO: 263)
RPKPQQFFGLM GSGSGS ATHIKFSKRD
RVG-Ktag:
(SEQ ID NO: 264)
YTIWWMPENPRPGTPCDIFTNSRGKRASNG GGK GG GSGSGS
ATHIKFSKRD
KTag-RVG:
(SEQ ID NO: 265)
ATHIKFSKRD GSGSGS GGK GG YTIWMPENPRPGTPCDIFTNSRGKR
ASNG

or a biological equivalent each thereof.

It should be appreciated, while the above exemplary embodiment shows the use of SpyTag on the capsid and KTag on the moiety, the reverse may also be accomplished but incorporating a KTag into the capsid and conjugating the SpyTag to the moiety. With respect to unnatural amino acid, azide-alkyne reactions-optionally catalyzed by copper—can be used to add moieties with the corresponding functional group (e.g. the unnatural amino acid comprises an azide and the moiety comprises an alkyne or vice versa).

In some embodiments, the engineered capsid can be used to link to viruses for joint delivery. Such linking is especially useful for the delivery of the recombinant expression system disclosed herein, where Cas9 is encoded as a split-Cas9 i.e. in two vectors. For example, one capsid may comprise a SpyTag and the other a KTag; thus, the viruses may be linked by catalyzing the ligation of SpyTag to KTag. Similarly, the azide-alkyne reaction can be used to facilitate the linking of the viruses where one comprises an azide containing unnatural amino acid and another comprises an alkyne containing unnatural amino acid. Further embodiments of linked viruses may be developed using one or more of the pseudotyped moieties where two viruses express moieties that hybridize to one another or may be linked spontaneously or through catalysis.

In further embodiments, the capsid may be engineered for immune shielding. Widespread exposure to viral capsids such as AAV has led to subjects harboring neutralizing antibodies against many natural virus serotypes. In some embodiments, the capsid may be modified through deletion or shuffling to evade the immune system; in some embodiments, the capsid may be associated with exosomes. In some embodiments, specific reagents are incorporated or used to coat the capsid for immune shielding. For example, the addition of polymers such as poly(lactic-co-glycolic acid), PEG, VSVG coating, and/or a lipid/amine (e.g. lipofectamine) coating may be used.

A non-limiting example of immune shielding is lipofectamine coating. For example, an alkyne-oligonucleotide may be linked to an unnatural amino acid comprising capsid. The modified virus is then washed with lipofectamine, which in turn forms a coating.

Further modifications may be made to the capsid in the interest of targeting specific tissues. As noted above, “homing” moieties can be used in pseudotyping to assure localization of the capsid to a particular target cell, organ, or tissue.

It is appreciated that further modifications may be made to the capsid that are known in the art to render it suitable for particular method aspects, such as but not limited to those described in U.S. Pat. Nos. 7,867,484; 7,892,809; 9,012,224; 8,632,764; 9,409,953; 9,402,921; 9,186,419; 8,889,641; 7,790,154; 7,465,583; 7,923,436; 7,301,898; 7,172,893; 7,071,172; 8,784,799; 7,235,235; 6,541,010; 6,531,135; 6,531,235; 5,792,462; 6,982,082; 6,008,035; 5,792,462; 9,617,561; 9,593,346; 9,587,250; 9,567,607; 9,493,788; 9,382,551; 9,359,618; 9,315,825; 9,217,159; 9,206,238; 9,198,984; 9,163,260; 9,133,483; 8,999,678; 8,962,332; 8,962,233; 8,940,290; 8,906,675; 8,846,031; 8,834,863; 8,685,387; US Patent Publication No. 2016/120960; 2017/0096646; 2017/0081392; 2017/0051259; 2017/0043035; 2017/0028082; 2017/0021037; 2017/0000904; 2016/0271192; 2016/0244783; 2916/0102295; 2016/0097040; 2016/0083748; 2016/0083749; 2016/0051603; 2016/0040137; 2016/0000887; 2015/0352203; 2015/0315612; 2015/0230430; 2015/0159173; 2014/0271550, and other family members associated with these patents and patent publications or the assignees or inventors thereof.

### Combinations and Methods

Aspects disclosed herein relate to the use of the recombinant expression system (split-Cas9) and the viral capsid engineered to impart favorable characteristics, such as but not limited to the addition of one or more unnatural amino acids and/or a SpyTag sequence or the corresponding KTag sequence alone or in combination with one another, e.g. in the form of a composition.

For example, the two vectors comprised in the recombinant expression system disclosed herein can each be packaged in a viral capsid engineered to incorporate one or more unnatural amino acid, SpyTag sequence, or KTag sequence. Alternatively, one or more of the vectors can be packaged in an unmodified viral capsid.

The combination offers advantages as noted above, particularly the ability to link the two portions of the split-Cas9 system to assure delivery of both vectors. Further, in embodiments in which the viral capsid is pseudotyped, tissue specific delivery may be achieved through the use of homing moieties.

In some embodiments, the recombinant expression system, the viral capsid engineered as disclosed herein, and/or the recombinant expression system wherein the two vectors comprising the split-Cas9 system are comprised in two viral capsids engineered as disclosed herein may be delivered to a subject. In some embodiments, the route and dose may be determined based on the subject or condition being treated.

Disclosed herein are gRNAs tailored to specific uses including but not limited to pain management, liver disease, HSC therapy, HIV, cancer immunotherapy, blood diseases, muscular dystrophy, in utero fetal targeting, cytochrome p450 based disorders, reprogramming macrophages, repelling mosquitos, Alzheimer's, and thyroid hormone production. The effector elements employed in the recombinant expression system as well as the pseudotyping of the viral capsid can be optimized for each of these uses.

For example, for pain management, the homing peptides disclosed herein above allow the viral capsid to target neurons, thereby conferring tissue specificity. Further aspects to convey such tissue specificity disclosed herein include but are not limited to the use of an miRNA circuit specific to neurons and/or the use of the specifically disclosed gRNAs in the recombinant expression system.

Another example in cancer immunotherapy is the regulation of signaling pathways. Since only a small number of pathways that regulate gene expression throughout the body, tissue specificity in this application is critical. The use of miRNA circuits, tissue specific promotes, and the incorporation of homing peptides specific to the target cancer in the viral capsid could ensure that the treatment would only affect the gene in the desired target.

With respect to HSC therapy and blood diseases implicating HSC, Applicants believe the route of delivery may be important and, thus, propose delivery of the virus in situ or in vivo introduction, such as but not limited to direct injection, of the disclosed recombinant expression system or composition into the bone marrow—where a reservoir of Hematopoietic stem cells (HSCs) or the thymus where T-cells mature. Similar bone marrow delivery can be used for in situ or in vivo T-cell editing and/or HSC editing for immune disorders, e.g. using PDCD-1 targeting gRNA and/or for cancer treatment. The HSCs and/or T-cells can be specifically edited based on the selection of tissue specific gRNA or other effector elements; thereby treating and/or preventing the immune disorder. It is believed that this in situ or in vivo approach is more effective approach than current treatments which rely heavily on ex vivo modification and transplantation cells (e.g. HSC and T cells) and are associated with a high possibility of HSC transplantation or T-cell transplantation. Further, in situ or in vivo delivery has great potential to reduce the cost of such cell therapies.

Alternatively, in these and cancer related embodiments relating to HSCs and/or T-cells, patient HSCs and/or T-cells may be modified ex vivo and delivered to the patient (e.g. via direct injection into the bone marrow). The modified cells can then expand in vivo. In some embodiments, the patient is administered these modified cells after eliminating the preexisting population of cells responsible for the disease.

In thyroid related embodiments, a dCas9 system with temporal regulation and optionally a viral capsid modified for homing to the thyroid can be utilized.

Further method aspects may comprise delivery of the recombinant expression system and/or viral capsid may employ a hydrogel. Hydrogels have been used as a drug-delivery biomaterial in vivo. Optimizing the entrapment and release of drugs in certain conditions has been widely studied. By tuning the hydrogel release properties, specific delivery of the recombinant expression system and/or viral capsid may be controlled according to discrete pH levels, temperature, or physiological conditions. For example, the recombinant expression system and/or viral capsid may be delivered, for example, to inflamed areas by tuning them to contract and release the recombinant expression system and/or viral capsid at a lower pH levels. Furthermore and without being bound by theory, optimized hydrogels can hold the recombinant expression system and/or viral capsid in place and prevent non-specific targeting—giving subjects more protection from undesired side effects. This delivery system can increase the specificity of the recombinant expression system and/or viral capsid.

In methods employing the split-Cas9 system, equal titer of both halves of the Cas9 is important to assure functional Cas9 is generated upon delivery. This may be assured by the pairing of the viral capsids comprising the two vectors and/or utilizing qPCR to target unique regions in each of the vectors to determine the titer of each vector relative to a titer control (e.g. ATCC-VR-1616).

Method aspects are also contemplated herein for using the disclosed viral capsid to test biocompatibility. One common method for testing a material's biocompatibility is to use animal models and perform histology and immunohistochemistry to characterize the cells present in each tissue. In addition to being expensive, this is also time and work intensive, and can be difficult to quantify. One possible alternative would be to introduce viral capsids packaging TK-GFP to the area of interest. Macrophages that phagocytose the TK-GFP AAV would then glow and express the reporter gene. Taking advantage of cell surface receptors on B and T cells may also allow transduction by TK-GFP AAVs to quantify lymphocytes in vivo. Facilitating macrophage phagocytosis or manipulating lymphocyte specific cell receptors would allow for quantification of innate and/or acquired immune responses. Ultimately, biomaterial testing will become more efficient and accessible.

Doses suitable for uses herein may be delivered via any suitable route, e.g. intravenous, transdermal, intranasal, oral, mucosal, or other delivery methods, and/or via single or multiple doses. It is appreciated that actual dosage can vary depending on the recombinant expression system used (e.g. AAV or lentivirus), the target cell, organ, or tissue, the subject, as well as the degree of effect sought. Size and weight of the tissue, organ, and/or patient can also affect dosing. Doses may further include additional agents, including but not limited to a carrier. Non-limiting examples of suitable carriers are known in the art: for example, water, saline, ethanol, glycerol, lactose, sucrose, dextran, agar, pectin, plant-derived oils, phosphate-buffered saline, and/or diluents. Additional materials, for instance those disclosed in paragraph of WO 2017/070605 may be appropriate for use with the compositions disclosed herein. Paragraphs through of WO 2017/070605 also provide non-limiting examples of dosing conventions for CRISPR-Cas systems which can be used herein. In general, dosing considerations are well understood by those in the art.

### EXAMPLES

The following examples are non-limiting and illustrative of procedures which can be used in various instances in carrying the disclosure into effect. Additionally, all reference disclosed herein below are incorporated by reference in their entirety.

### Example 1—Generation of Exemplary Modular AAV Systems

### Vector Design and Construction

Briefly, the split-Cas9 mAAV vectors were constructed by sequential assembly of corresponding gene blocks (Integrated DNA Technologies) into a custom synthesized rAAV2 vector backbone. For the UAA experiments, four gene blocks were synthesized with ‘TAG’ inserted in place of the nucleotides coding for the surface residues R447, S578, N587 and S662, and were inserted into the pAAV-RC2 vector (Cell Biolabs) using Gibson assembly. For ETF1-E55D, the gene block encoding the protein sequence was synthesized and inserted downstream of a CAG promoter via Gibson assembly.

### Mammalian Cell Culture

HEK293T cells were grown in Dulbecco's Modified Eagle Medium (10%) supplemented with 10% FBS and 1% Antibiotic-Antimycotic (ThermoFisher Scientific) in an incubator at 37° C. and 5% CO₂ atmosphere, and were plated in 24-well plates for AAV transductions. 293T cells transfected with pAAV inducible-Cas9 vectors were supplemented with 200 μg/ml of Doxycycline. Hematopoietic stem cells expressing CD34 (CD34+ cells) were grown in serum free StemSpan™ SFEM II with StemSpan™ CD34+ Expansion Supplement (10×) (all from StemCell Technologies). CD34+ cells were plated in 96-well plates for AAV transductions.

### Production of AAV Virus

AAV8 virus was utilized for all in vivo studies, AAVDJ was utilized for all in vitro studies in HEK293T cells, AAV6 was utilized for ex vivo studies in CD34+ cells, and AAV2 was utilized for the UAA incorporation studies.

Large-scale production: Virus was either prepared by the Gene Transfer, Targeting and Therapeutics (gT3) core at the Salk Institute of Biological Studies (La Jolla, CA), or in house. Briefly, AAV2/8, AAV2/2, AAV2/6, AAV2/DJ virus particles were produced using HEK293T cells transfected with 7.5 ug of pXR-capsid (pXR-8, pXR-2, pXR-6, pXR-DJ), 7.5 of ug recombinant transfer vector, and 22.5 ug of pAd5 helper vector using PEI in 15 cm plates at 80-90% confluency. The virus was harvested after 72 hours and purified using an iodixanol gradient. The virus was concentrated using 100 kDA filters (Millipore), to a final volume of ~1 mL and quantified by qPCR using primers specific to the ITR region, against a standard (ATCC VR-1616).

AAV-ITR-F:
(SEQ ID NO: 266)
5′-CGGCCTCAGTGAGCGA-3′
and
AAV-ITR-R:
(SEQ ID NO: 267)
5′-GGAACCCCTAGTGATGGAGTT-3′.

UAA incorporation: From two hours prior to transfection until harvesting, 293T cells were grown in DMEM containing 0.4 mM lysine (as opposed to the 0.8 mM lysine usually present in DMEM), and supplemented with 10% FBS and 2 mM N-epsilon-((2-Azidoethoxy) carbonyl)-L-lysine. The plasmid pAcBac1.tR4-MbPyl (gift from Peter Schultz, Addgene #50832) containing the pyrrolysyl-tRNA and tRNA synthetase was co-transfected into 293T cells along with the capsid vector pAAV-RC2 (and mutants thereof), recombinant transfer vector, and pAd5 helper vector at a 5:1 ratio with the capsid vector. The same protocol, as above, was followed for harvesting, purification and quantification of the virus. To further quantify functional activity, flow cytometry analysis of UAA AAVs was performed 48 hours post transduction and 20,000 cells were analyzed using a FACScan Flow Cytometer and the Cell Quest software (both Becton Dickinson).

Small-scale production: Small-scale AAV preps were prepared using 6-well plates containing HEK293T cells, which were co-transfected with 0.5 ug pXR-capsid, 0.5 ug recombinant transfer vector, and 1.5 μg pAd5 helper vector using PEI. The cells and supernatant were harvested after 72 hours, and the crude extract was utilized to transduce cells.

### Animal Experiments

AAV Injections: All animal procedures were performed in accordance with protocols approved by the Institutional Animal Care and Use Committee (IACUC) of the University of California, San Diego. All mice were acquired from Jackson labs. AAV injections were done in either adult C57BL/6J mice (10 weeks) through tail-vein injections or in neonates (4 weeks) through IP injections, using 0.5E+12-1E+12. Four weeks post-injection, mice were humanely sacrificed by CO2. Tissues were harvested and frozen in RNAlater stabilization solution (ThermoFisher Scientific).

Doxycycline administration: Mice transduced with pAAV inducible-Cas9 vectors were given IP injections of 200 mg Doxycyline in 10 mL 0.9% NaCl with 0.4 mL of IN HCl, three times a week for four weeks.

Histology: Mice were humanely sacrificed by CO₂. Livers were frozen in molds containing OCT compound (VWR) and frozen in a dry ice/2-methyl butane slurry. Histology was performed by the Moores Cancer Center Histology and Imaging Core Facility (La Jolla, CA). Liver sections were stained with hematoxylin and eosin (H&E) for pathology, and with anti-CD81 (BD Biosciences, No. 562240).

### Genomic DNA Extraction and NGS Preps

gDNA from cells and tissues was extracted using DNeasy Blood and Tissue Kit (Qiagen), according to the manufacturer's protocol. Next generation sequencing libraries were prepared as follows. Briefly, 4-10 μg of input gDNA was amplified by PCR with primers that amplify 150 bp surrounding the sites of interest (Table 2b) using KAPA Hifi HotStart PCR Mix (Kapa Biosystems). PCR products were gel purified (Qiagen Gel Extraction kit), and further per purified (Qiagen PCR Purification Kit) to eliminate byproducts. Library construction was done with NEBNext Multiplex Oligos for Illumina kit (NEB). 10-25 ng of input DNA was amplified with indexing primers. Samples were then purified and quantified using a qPCR library quantification kit (Kapa Biosystems, KK4824). Then, samples were pooled and loaded on an Illumina Miseq (150 bp paired-end run or 150 single-end run) at 4 nM concentrations. Data analysis was performed using CRISPR Genome Analyzer44.

Gene Expression Analysis and qRT-PCR

RNA from cells was extracted using RNeasy kit (Qiagen), and from tissue using RNeasy Plus Universal Kit (Qiagen). 1 μg of RNA was reverse-transcribed using a Protoscript II Reverse Transcriptase Kit (NEB). Real-time PCR (qPCR) reactions were performed using the KAPA SYBR Fast qpcr Kit (Kapa Biosystems), with gene specific primers (Table 2a). Data was normalized to GAPDH or B-actin.

### AAV Pseudotyping

Alexa 594 DIBO alkyne tethering: The AAV2 wild type and AAV2-S578UAA were incubated with Alexa 594 DIBO alkyne in TBS (both ThermoFisher Scientific) for 1 hour at room temperature. The excess label was washed off with PBS. The virus particles were added to 293T cells and the cells were imaged 2 hours post transduction.

Oligonucleotide tethering and DNA array: Oligos A′ and B′ (5 μM) were spotted on a streptavidin functionalize array (ArrayIt: SMSFM48) and incubated at room temperature for 30 minutes 45. Meanwhile, oligo A was linked to AAV2-N587UAA_mCherry via the process of click chemistry (Click-iT—ThermoFisher Scientific, C10276) and then washed with PBS. Next, the array was washed with PBS and the modified AAV2-N587UAA_mCherry was added to each well, incubated at room temperature for 30 minutes and then washed with PBS. Finally, 293T cells were added to each well. Cells were imaged for mCherry expression 48 hours post transduction.

### DISCUSSION

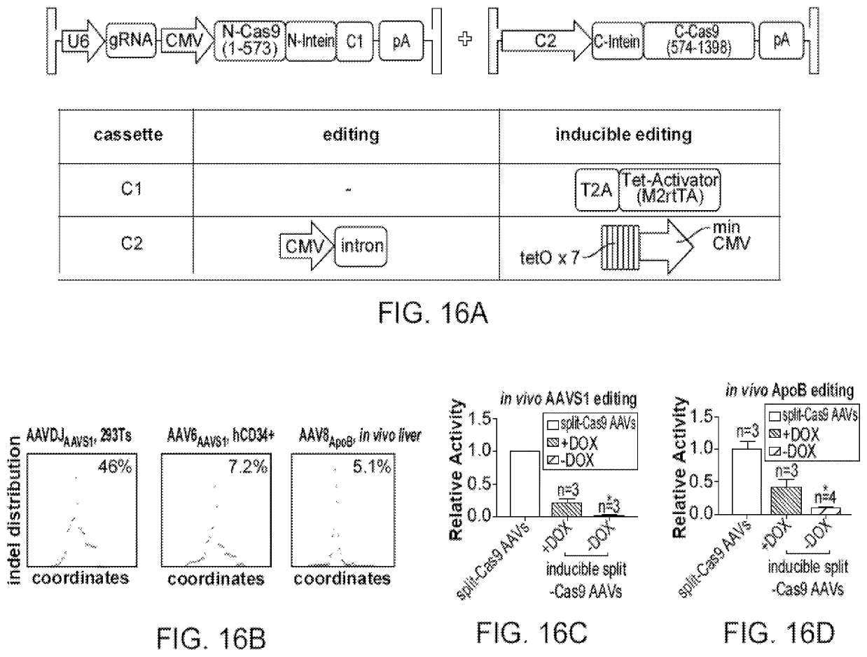

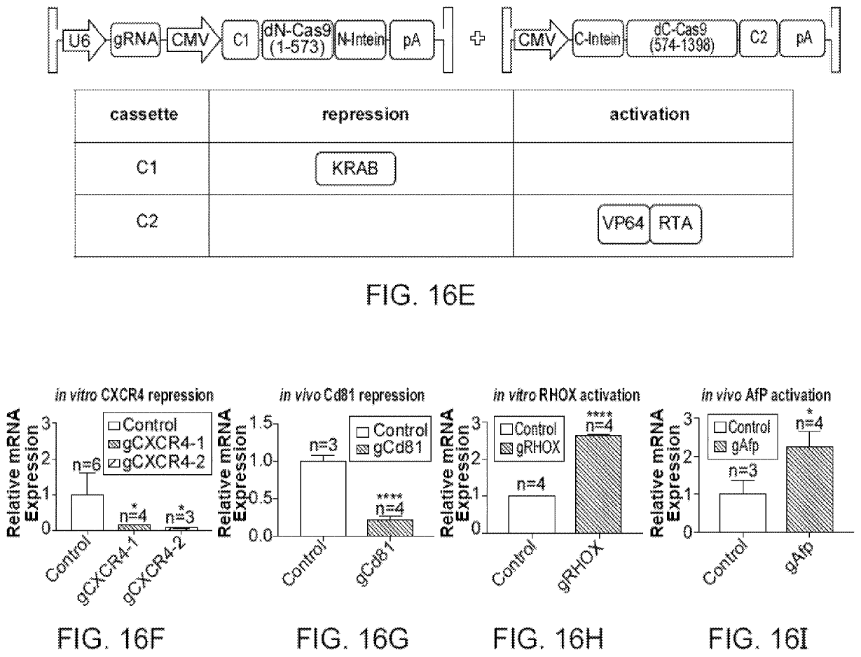

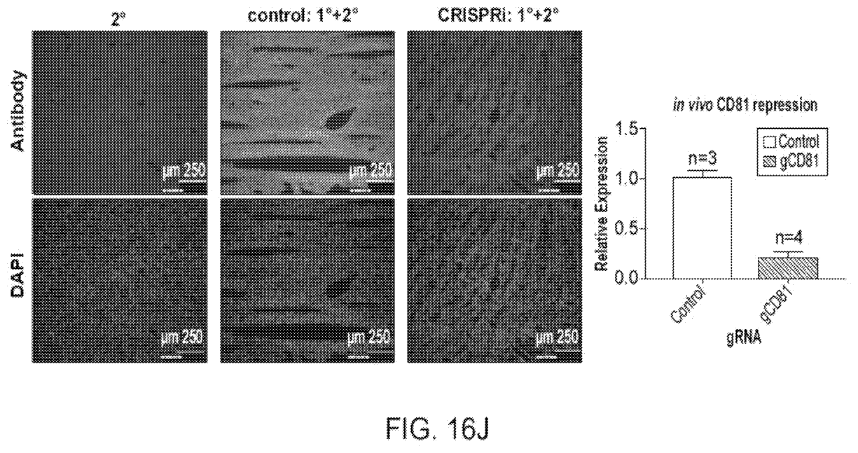

The exemplary platform is built using adeno-associated viruses (AAV) as the core delivery agent as AAVs are highly preferred for gene transfer due to their mild immune response, long-term transgene expression, ability to infect a broad range of cells, and favorable safety profile. However, AAVs have a limited packaging capacity (~4.7 kb), making it difficult to incorporate the large Cas9-like effector proteins and fusions thereof, and also the components necessary for efficacious gene and guide-RNA expression. Applicants thus leveraged split-Cas9 systems to bypass this limitation. In Applicants' delivery format the Staphylococcus pyogenes Cas9 (SpCas9) protein is split in half by utilizing split-inteins, originally derived from N. punctiforme, whereby each Cas9 half is fused to its corresponding split-intein moiety and upon co-expression the full Cas9 protein is reconstituted. This format of delivery utilizes two rAAVs and by appropriately designing the corresponding vectors Applicants leveraged the resulting residual packaging capacity to enable the full range of CRISPR-Cas genome engineering functionalities (FIG. 16).

Applicants first confirmed targeted genome editing across a range of cell types and genomic loci in in vitro and in vivo scenarios (FIG. 16a, 16b) and notably, also demonstrated robust AAV6 mediated editing in human CD34+ hematopoietic stem cells. As a hit and run approach suffices for genome editing and is in fact preferable over long-term nuclease expression, Applicants next engineered the incorporation of a synthetic circuit to enable small-molecule regulation of CRISPR-Cas editing activity. Here one rAAV construct was designed to bear a minimal CMV promoter bearing a tetracycline response element (TRE) up-stream of the C-Intein-C-Cas9 fusion, and in the second rAAV construct a full promoter was used to drive expression of the N-Intein-N-Cas9 fusion and a tet-regulatable-activator (tetA). In the presence of doxycycline, tetA binds to the TRE site allowing inducible expression of the C-Cas9 and thereby temporal regulation of genome editing. Applicants demonstrated functioning of this circuit in both in vitro and in vivo scenarios (FIG. 16c). Taken together, the system above enables robust CRISPR-Cas9 based genome editing, and coupling of tet regulators enables facile regulation of the otherwise persistent gene expression from the AAVs.

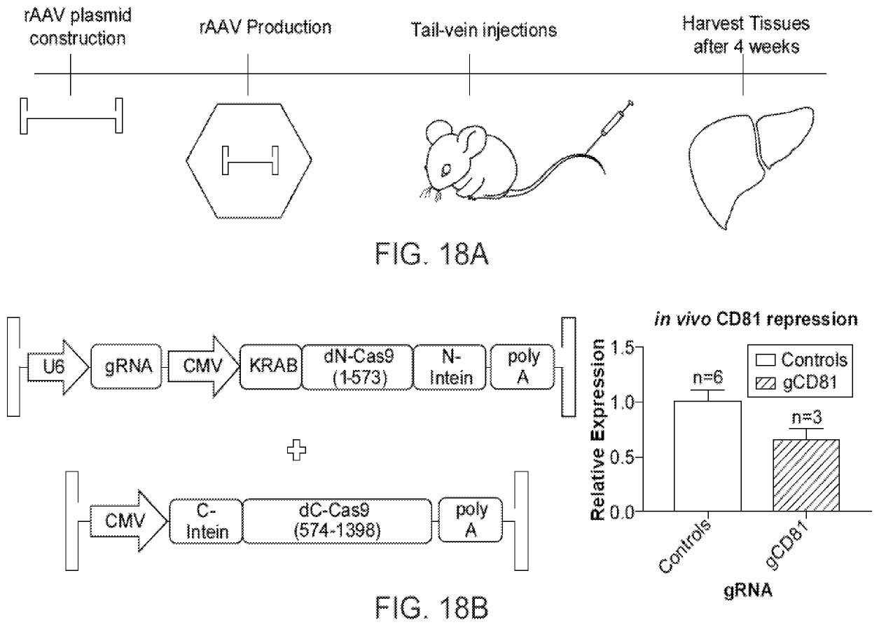

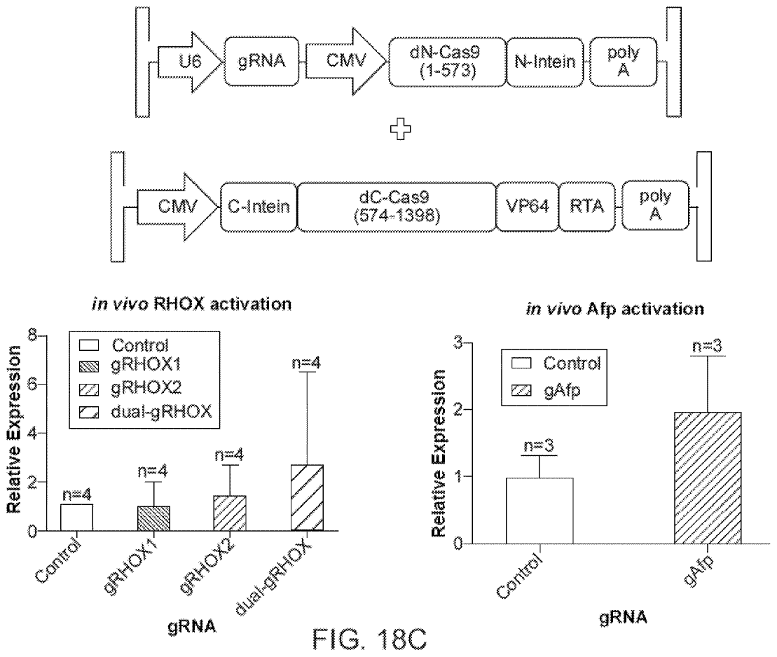

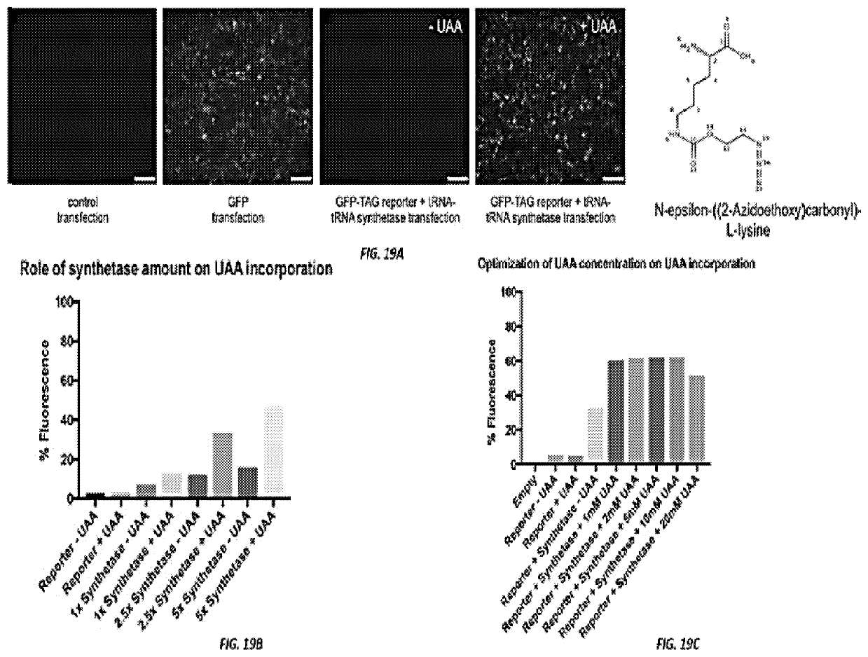

Applicants next utilized dead split-Cas9 proteins to engineer targeted genome repression via fusion of a KRAB domain, and targeted genome activation via fusion of VP64 cum rTA domains (FIG. 16d). In vitro experiments were performed in HEK293 Ts utilizing AAVDJ, and in vivo experiments were conducted in C57BL/6J, 10-week old mice with AAV delivery via tail vein injection at titers of 0.5E12-1E12 AAV8 particles per mouse using the AAV8 serotype. Mice were analyzed at 4 weeks post transduction. Applicants confirmed targeted gene repression and activation, as assayed via RNA and immunofluorescence based protein expression, in both in vitro and in vivo scenarios and across multiple genomic loci (FIG. 16e-j, FIG. 18). Notably, Applicants were able to achieve ~80% in vivo repression at the CD81 locus (n=4), and a >2 fold in vivo activation of the Afp locus (n=4). This system thus paves the way for fine control of gene expression and offers a scarless approach for in vivo genome engineering applications.

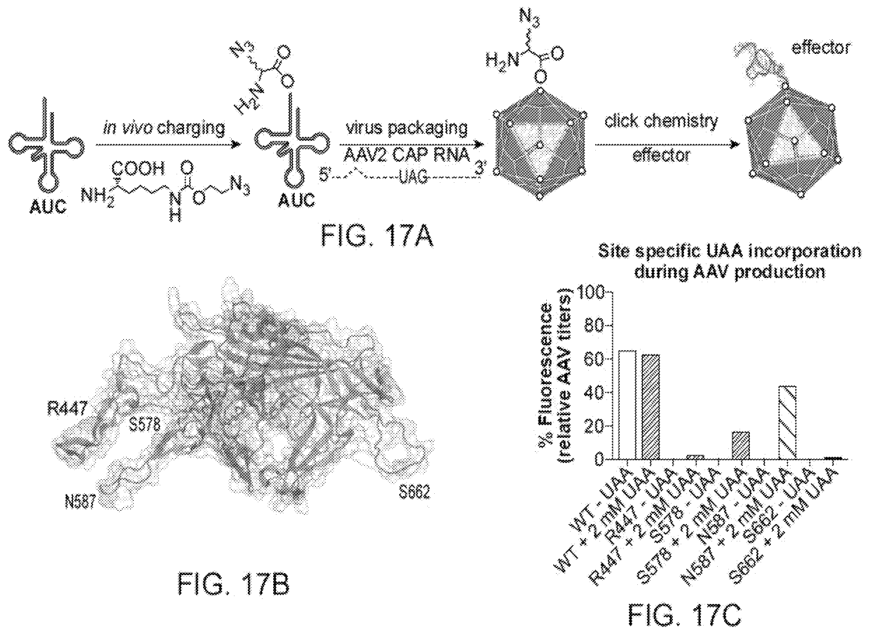

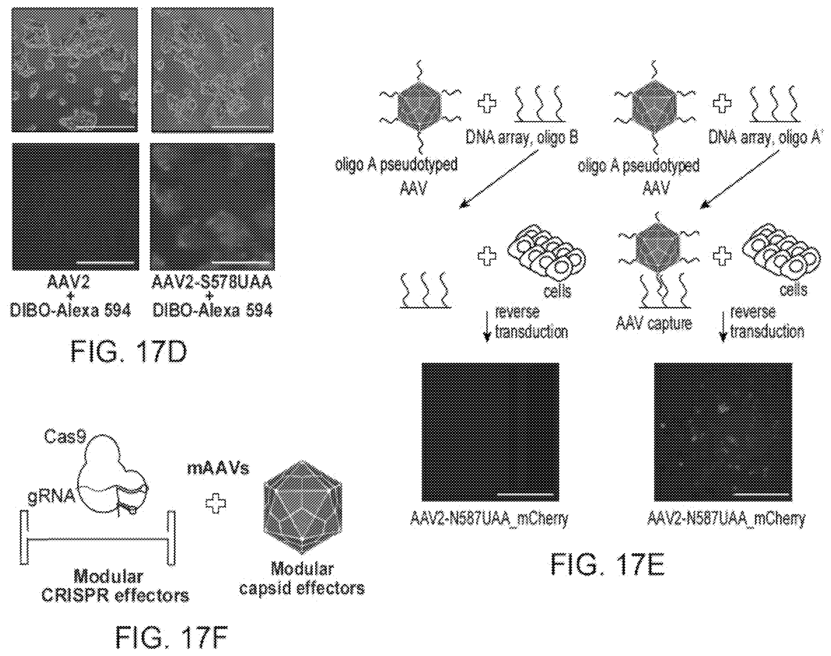

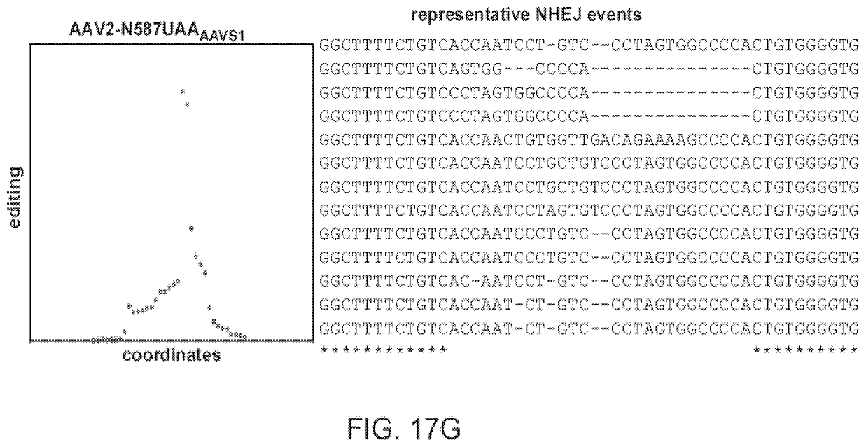

With the establishment of programmability in CRISPR effector incorporation into the AAVs, Applicants next turned their attention to enabling facile programmability in capsid pseudotyping. AAV capsid proteins are typically inflexible to insertion of large peptides or biomolecules (without significant loss of titer or functionality). Applicants thus developed a novel and versatile approach that circumvents this limitation by utilizing unnatural-amino acid (UAA) mediated incorporation of bio-orthogonal click chemistry handles to enable facile capsid modifications. Applicants first computationally mapped accessible amino acid sites on the AAV2 surface and focused their evaluation on R447, N587, S578 and S662 as potential candidate sites (FIG. 17b). The UAA of interest was genetically encoded by a reassigned nonsense codon (TAG) at the corresponding amino acids in the AAV VP1 protein, and co-translationally incorporated into the capsid using an orthogonal UAA specific tRNA/aminoacyl-tRNA synthetase (tRNA/aaRS) pair (FIG. 17a, FIG. 19). Applicants could thence successfully incorporate an azide modified lysine-based amino acid-N-epsilon-((2-Azidoethoxy) carbonyl)-L-lysine on to the AAV2 capsid surface, with N587 and S578 modifications showing highest relative production titers and viral activity (FIG. 17c).

Applicants next demonstrated the ready capsid engineering enabled by UAA incorporation via two independent pseudotyping experiments: one, Applicants performed a click chemistry reaction to link a fluorescent molecule, Alexa 594 DIBO alkyne, onto the virus and successfully visualized modified fluorescent virus via transduction of cells (FIG. 17d); two, Applicants tethered alkyne-tagged oligonucleotides onto the AAV surface via click chemistry and demonstrated their selective capture on DNA array spots bearing corresponding complementary oligonucleotides, as evidence by transduction of cells cultured on top of these (FIG. 17e). Finally, Applicants confirmed that these UAA modified AAVs could incorporate the split-Cas9 based genome engineering payloads (FIG. 17f) and effect robust genome editing (FIG. 17g), thus establishing an integrated mAAV delivery platform.

Taken together, Applicants' approach provides a facile and straightforward method to edit and regulate the expression of endogenous genes using the Cas9 and dCas9 based effectors, and also ready AAV pseudotyping via incorporation of UAAs on their surface. This system has several advantages, including the utilization of a split-Cas9 system, which due to the limited cargo capacity of AAVs (~4.7 kb), is optimal to conduct all desired genome engineering applications, including genome editing and regulation. In addition, another advantage of this system is that one can utilize desired accessory elements of interest to optimize transcription of the payloads. Applicants show that their mAAV-Cas9 system can be utilized to achieve a high level of in vivo transcriptional repression (~80%) (FIG. 16g, 16j) and in vivo transcriptional activation (>2 fold increase) (FIG. 16i). Furthermore, Applicants show that their system can be utilized to edit cells in vitro in HEK293 Ts, CD34+ HSCs cells and in vivo in C57BL/6J mice (FIG. 16b). Given the high therapeutic value in targeting CD34+ HSCs, Applicants believe that their all AAV system can provide a powerful resource for developing versatile delivery agents for these cells. Importantly, Applicants also demonstrate temporal control over genome editing with their inducible synthetic switch, which limits the expression of Cas9 nuclease, and is therefore, of high therapeutic value (FIG. 16c, 16d). This mAAV system, Applicants show, also allows for easy and quick addition of aptamers to the capsid surface via the process of click chemistry. This opens the door to a host of programmable pseudotyping of the capsid surface to both systematically engineer the AAV target cell type specificity, as well as study the basic biology of AAV transduction into cells. Applicants anticipate these vectors will complement other strategies for engineering novel AAV vectors such as those based on directed evolution, molecular shuffling and evolutionary lineage analysis, and further enable a modular parts based systematic evaluation of aptamers and other moieties for modulating AAV activity. Applicants also note some potential limitations of the mAAV system: one, utilizing a split-Cas9 system will have reduced targeting efficiency as both components, C-Cas9 and N-Cas9, have to be co-delivered to the target cell of interest to restore Cas9 activity; and two, modifications of the capsid via UAAs leads to 1.5-5 fold lower viral titers. Applicants expect that with improvements in techniques for localized tissue-specific delivery and optimization of AAV productions parameters, these aspects will be progressively addressed. Taken together Applicants anticipate their versatile mAAV synthetic delivery platform, through its ready programmability in CRISPR effector incorporation and capsid pseudotyping, will have broad utility in basic science and therapeutic applications.

### Example 2—Unnatural Amino Acid Addition onto the AAV2 Capsid

The following is the outline of the protocol:
1. Testing of non-canonical amino acid incorporation
2. Generation of AAV capsid constructs with TAG inserted
3. Generation of AAVs containing the non canonical amino acid in its capsid
4. Testing the hypothesis with MUC-1 aptamer and A549 cells
5 Testing if the AAV2 generated containing the MUC-1 aptamer could be used to selectively transduce A549s in a mixed population of cells
6. Use the AAV2 generated to deliver Cas9 selectively to A549s in a mixed population of cells and check for gene editing
7. In vivo experiments: Using the AAV2 generated delivery mechanism for CRISPR-Cas9 and checking gene editing in the target cells

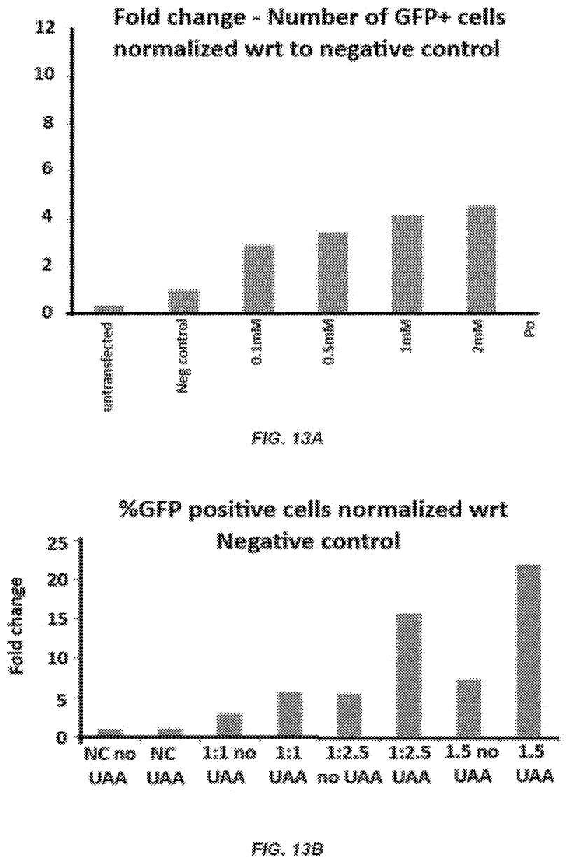

Applicants began by testing the incorporation of the non canonical amino acid into a GFP reporter plasmid containing a TAG stop codon in the middle of the GFP gene. Making use of Amber suppression, in the presence of the tRNA, tRNA synthetase and the non canonical amino acid, the GFP expression was restored (FIG. 13A). Applicants also varied the reporter to synthetase ratio (1:1, 1:2.5 and 1:5) and the results are depicted in FIG. 13B.

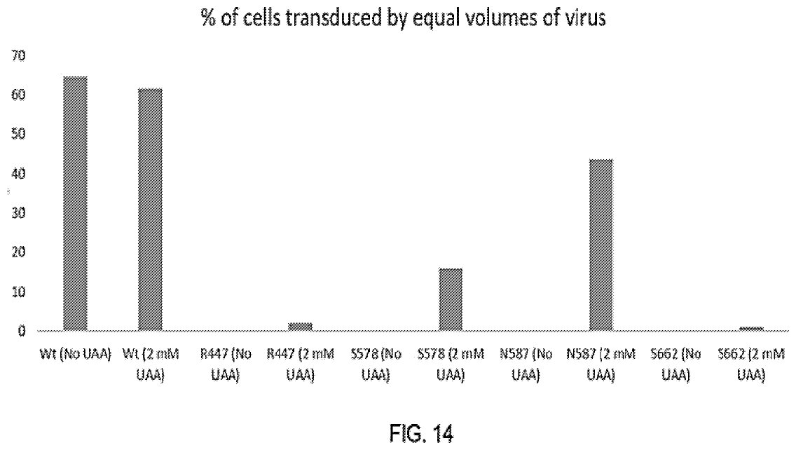

Applicants have added the unnatural amino acid to the virus capsid using the method of amber suppression. Applicants have added incorporated the stop codon TAG in place of surface residues R447, S578, N587 and S662. Applicants hypothesized that the virus would only be produced in the presence of the tRNA/synthetase pair and the unnatural amino acid. The experiments carried out so far seem to show us exactly this. In the absence of the unnatural amino acid the virus titres are extremely low while they are several fold (200×) higher in the case when unnatural amino acids are added. Applicants generated 4 different viruses containing the non canonical amino N-epsilon-((2-Azidoethoxy) carbonyl)-L-lysine at the residues specified (FIG. 14).

Next Applicants designed a MUC-1 aptamer containing an alkyne group and are looking to add it to the non canonical amino via click chemistry since the non canonical amino acid contains an azide group. AAV2 doesn't infect the A549 lung cancer cell line very effectively. A549 cells show an overexpression of MUC-1 on their surface and Applicants believe that the MUC-1 aptamer added onto the AAV2 would help improve the specificity of the virus towards the A549 cells.

### Example 3—AAV2-SpyTag

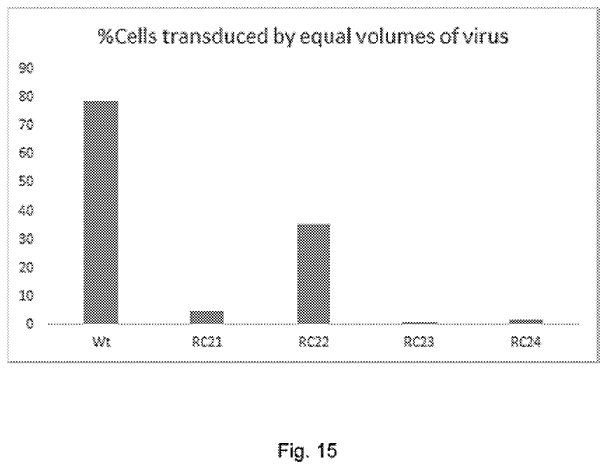

SpyTags and SpyTags with linker peptides have been introduced at the residue N587 of the AAV2 capsid both with and without the HSPG binding peptide creating 4 versions of the AAV2 (FIG. 15).

### Example 4—AAV-DJ

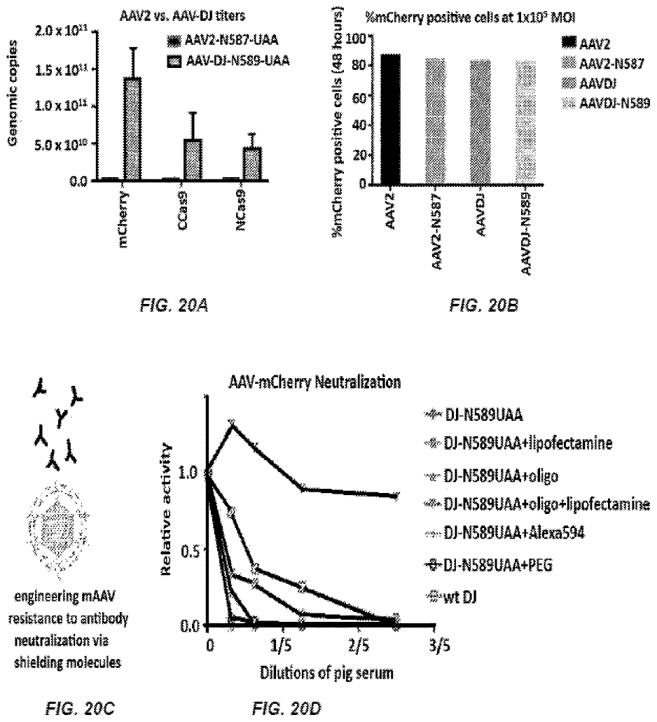

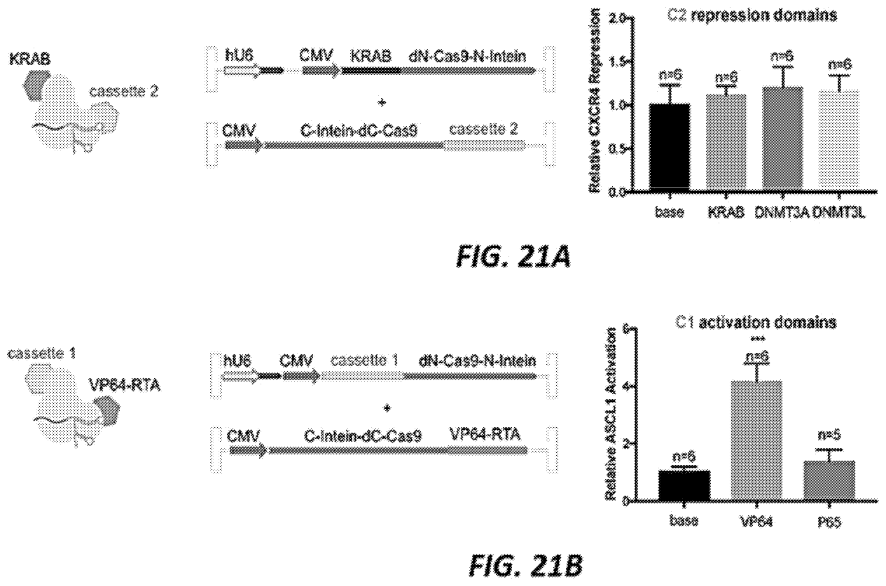

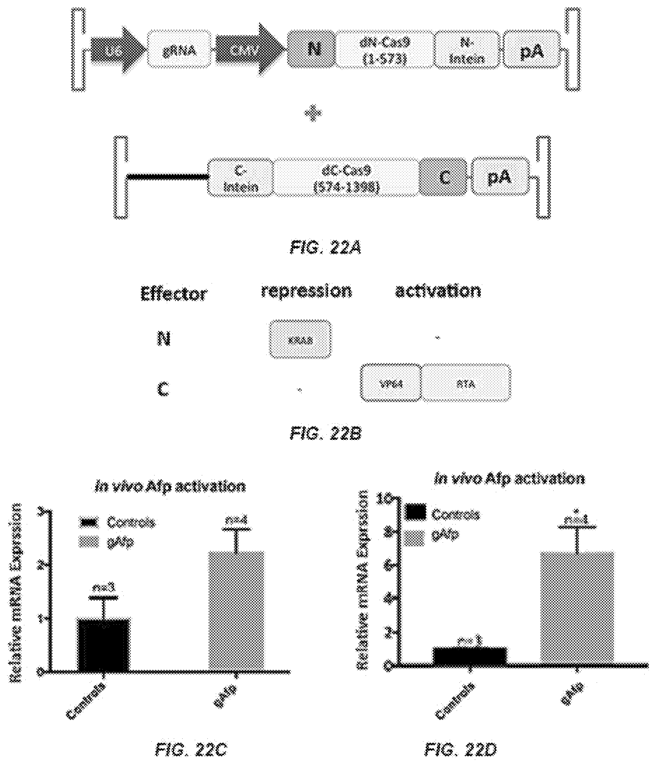

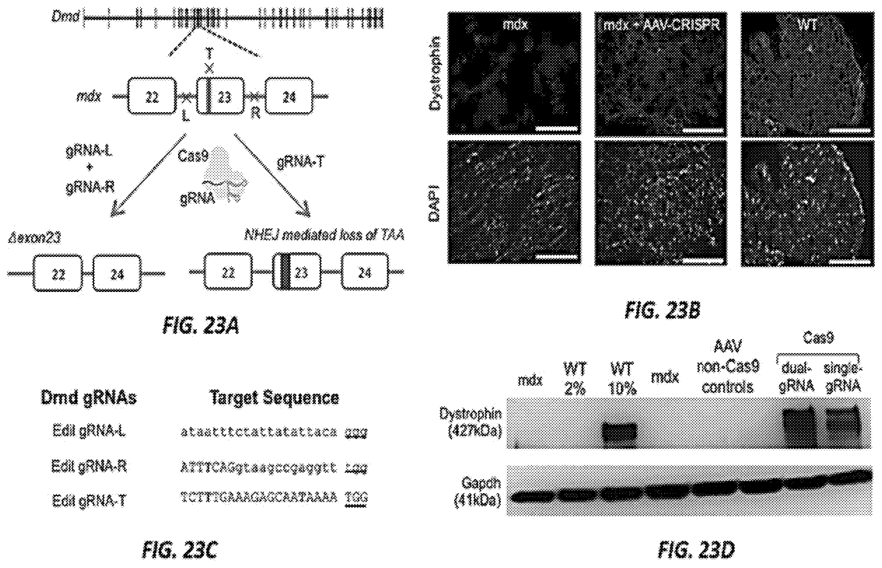

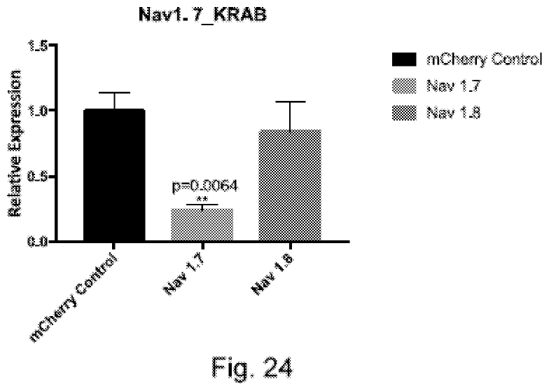

To facilitate broader usage of this system, Applicants also engineered the AAV-DJ serotype to similarly incorporate UAAs. Towards this, based on protein alignments, N589 in AAV-DJ was chosen as the equivalent site to N587 in AAV2. Applicants observed that the AAV-DJ-N589UAA virus had 5-15 fold higher titers than the AAV2-N587UAA virus (FIG. 20a), and confirmed that the incorporation of the UAA in place of residues N587 and N589 on the AAV2 and AAV-DJ respectively does not negatively affect the activity of the virus (FIG. 20b).

The prevalence of AAV neutralizing antibodies in the serum is a major obstacle to their effective use in in vivo studies and therapeutic applications. Applicants thus surmised if, utilizing the programmability of this system, it was possible to confer novel surface properties to the AAV capsids that could enable a degree of shielding of AAVs to neutralization by AAV antibodies (FIG. 20c). Towards engineering such a ‘stealth’ AAV we screened a host of small molecule and polymer moieties by tethering these onto the AAV capsid surface and assaying the resultant AAV transduction ability post exposure to pig serum (FIG. 20d) that is known to bear neutralizing AAV antibodies⁴⁸⁻⁵⁰. Interestingly Applicants observed that shielding via lipids resulted in near complete resistance of AAVs to pig serum-based neutralization. Applicants achieved this via tethering of oligonucleotides onto the AAV surface, which in turn were used to bind the commercial lipid polymer formulation lipofectamine. Notably, Applicants observed activity of the lipid-coated virus even under conditions where the wt AAV-DJ and AAV-DJ-N589 viruses are completely neutralized (FIG. 20d). Applicants further confirmed these engineered viruses retain full genome editing functionality, and notably in the presence of the lipofectamine coat displayed enhanced editing rates compared to unmodified viruses. This approach, thus, paves the way for programmable control of AAV capsid surface properties thereby enabling a systematic evaluation of small molecules and polymers for modulating AAV activity.

### Example 5—miRNA for Tissue Specificity

Applicants assessed the specificity and delivery of this exemplary system by using TK-GFP (Thymidine kinase GFP fusion protein) as a reporter gene. TK-GFP allows for real time in vivo imaging of the whole animal using PET/SPECT, which provides spatial information as to which tissues the virus infects while providing quantitative information as qPCR would.

### Example 6—Pain Management

Applicants test their pain management system in C57BL/6J mice, with 9 mice utilized total. Three mice are injected with the pAAV9_gSCN9a_dCas9 system, 3 mice are injected with an empty vector, pAAV9_gempty_dCas9, and 3 SNC9a mutant mice (Scn9atm1Dgen) are used as positive controls. Applicants also utilize human neuronal cells to test the human gRNAs in vitro.

### Example 7—CD81 Repression

Applicants have designed the split-Cas9 and split-dCas9 systems to target three malarial host genes in the liver, CD81, Sr-b1, and MUC13, in order to repress and edit them. These are host factors required for the plasmodium sporozoite infection of hepatocytes. Applicants have tested the repression of CD81 in vivo, and have detected a repression of 35%. (FIGS. 8 and 9). FIG. 8 represents the relative expression of CD81 in 3 mice that have been treated with AAV8_gCD81_KRAB_dCas9 and 6 control mice. FIG. 9 represents three sets of histology samples: the first which has no primary antibody, the second is the positive control which shows relatively high expression of CD81, and the third is the set that was delivered AAV8_gCD81_KRAB_dCas9, which shows a decreased expression of CD81.

### Example 8—Pain Management

There are three main characteristics to pain: duration (acute to chronic), location (e.g. muscle, orofacial), as well as cause (e.g. nerve injury, inflammation). Applicants utilize four primary kinds of pain models (burn models, inflammatory, postoperative, and neuropathic) to further understand 1) what kinds of pain our therapy targets and 2) whether our treatment shows similar results or improvement from traditional methods for pain management, e.g. opioids. These pain models are summarized in the table below. For the acute nociception burn models, Applicants utilize two commonly utilized models: the hot plate test and the “Hargreaves” test, which usually are utilized to assess nociceptive processing as an assay to screen for the analgesic activity of a drug or physiological manipulation. For the first model, an animal is placed on a 55° C. until the animal elicits known behaviors following a noxious thermal stimulus, such as jumping or licking of its paw. If the animal does not respond before 45 seconds, it is removed from the hot plate to avoid tissue damage. The mechanical thresholds are then measured utilizing von Frey filaments, nylon fibers with logarithmically incremental stiffness (0.41, 0.70, 1.20, 2.00 g), which measures withdrawal response. Thermal nociceptive responses are then tested in a different experiment, known as Hargreaves. Briefly, mice are placed in a Plexiglas cubicle on a heated (30° C.) glass surface, and the light from a focused projection bulb, located below the glass, is directed at the plantar surface of one hind paw. Thermal withdrawal responses are measured every 30 min for 3 h post injury. The time interval between the application of the light and the hind paw withdrawal response, defined as the paw withdrawal latency (PWL: s), is then measured. For the inflammatory pain model, Applicants inject serum from arthritic transgenic K/B×N mice into wildtype mice in order to produce mice with robust and high mechanical allodynia with onset that correlates with joint/paw inflammation lasting 2-3 weeks. The mechanical thresholds via von Frey filaments as described before will also be measured. The next postoperative model, an incision is made through the skin, fascia, and muscle of the plantar aspect of the hindpaw of mice under anesthesia. Withdrawal responses are measured using von Frey filaments at distinct areas around the wound for 6 days post-surgery.

| Type of Pain Model | Insult | References |
|---|---|---|
| Acute nociception: | Hot plate and | Nozaki-Taguchi and Yaksh |
| Burn models | “Hargreaves” | (1998) Neurosci. Lett. |
| | | 254(1): 25-8 |
| Inflammatory Pain | Arthritis (K/BxN | Christianson et al., (2012) |
| Model | serum injected | Methods Mol. Biol. |
| | into mice) | 851: 249-260 |
| Postoperative Pain | Incision model | Brennan et al. (1996) Pain |
| model | (hyperalgesia) | 64(3): 493-501 |
| Neuropathic Pain | Spinal nerve | Kim and Chung (1992) Pain |
| Models | ligation/transection | 50(3): 355-363 |
| | Chemotherapy | Balayssac et al. (2009) |
| | (Cisplatin) | Neurosci. Lett. |
| | | 465(1): 108-1112 |

Lastly, we will utilize two neuropathic pain models: spinal nerve ligation and chemotherapy utilizing Cisplatin. In the first model, spinal nerve ligation (SNL), also known as the Chung model, L5 and L6 spinal nerves are dissected from the L4 spinal nerve and tightly ligated distal to the dorsal root ganglia (DRG). For the chemotherapy model, mice will receive dosages of Cisplatin at 5 mg/kg per week during 8 weeks. Neuropathic models are known to have behavioral alterations, such as mechanical allodynia, cold allodynia, and thermal hyperalgesia. For this reason, both the Hargreaves test to test for withdrawal latencies due to application of radiant heat as well as the von Frey test to test for mechanical stimulation are utilized.

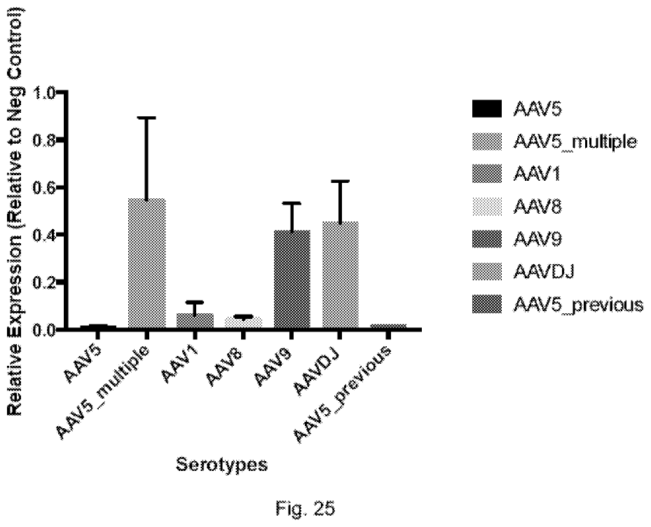

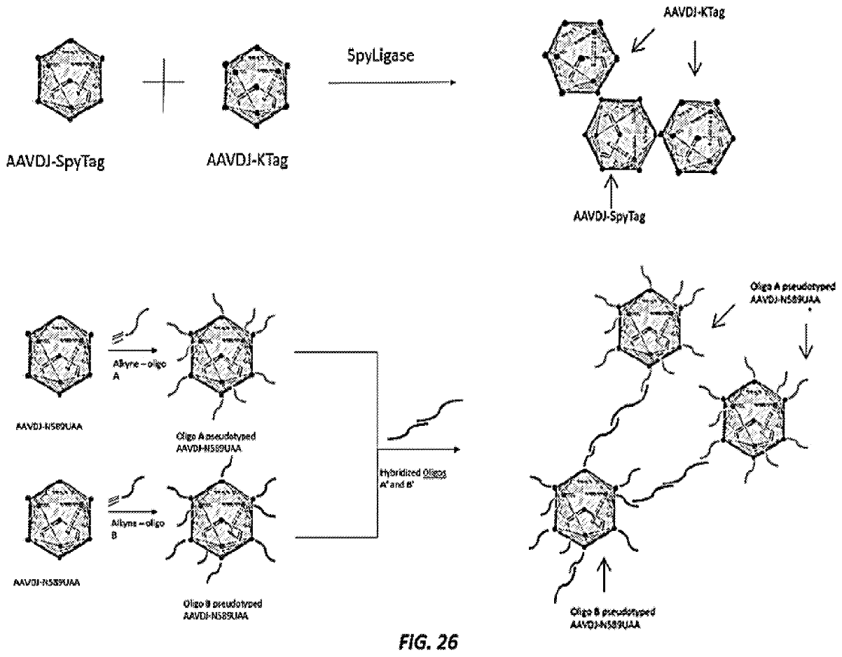

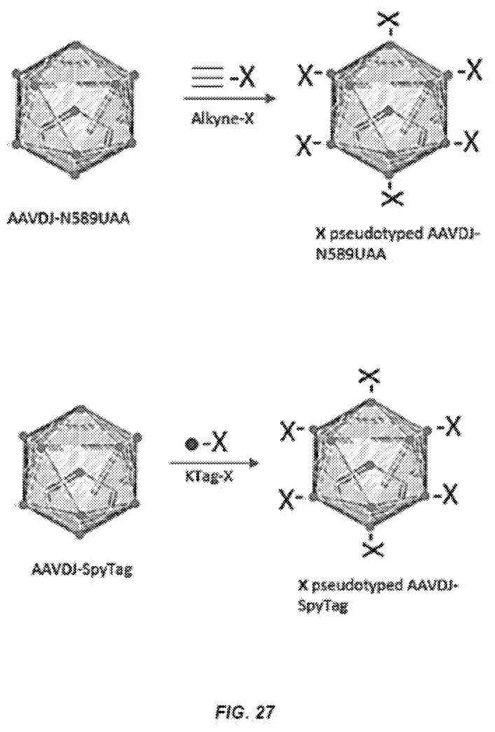

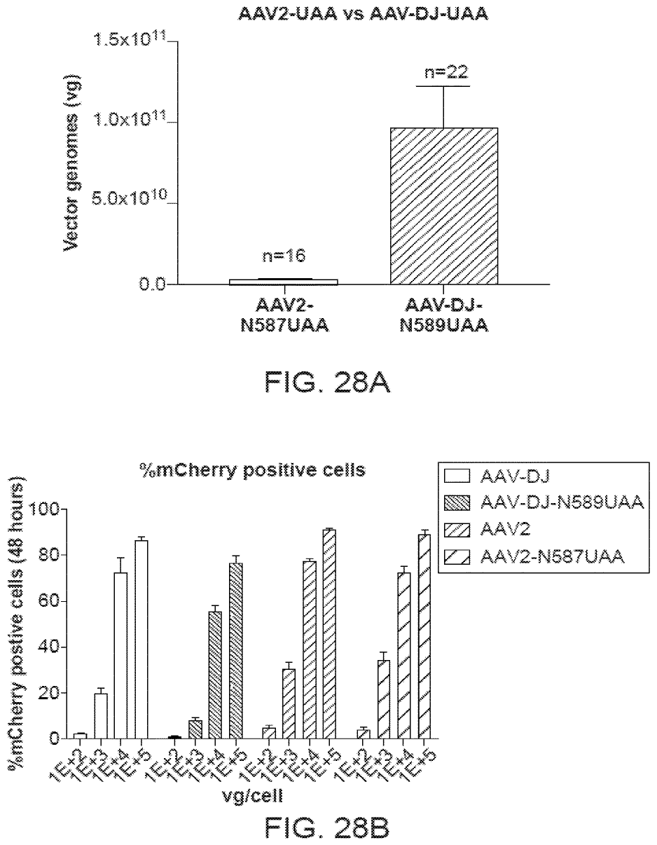

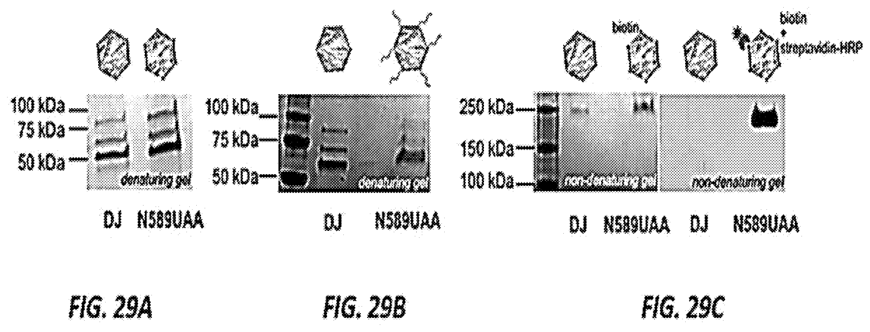

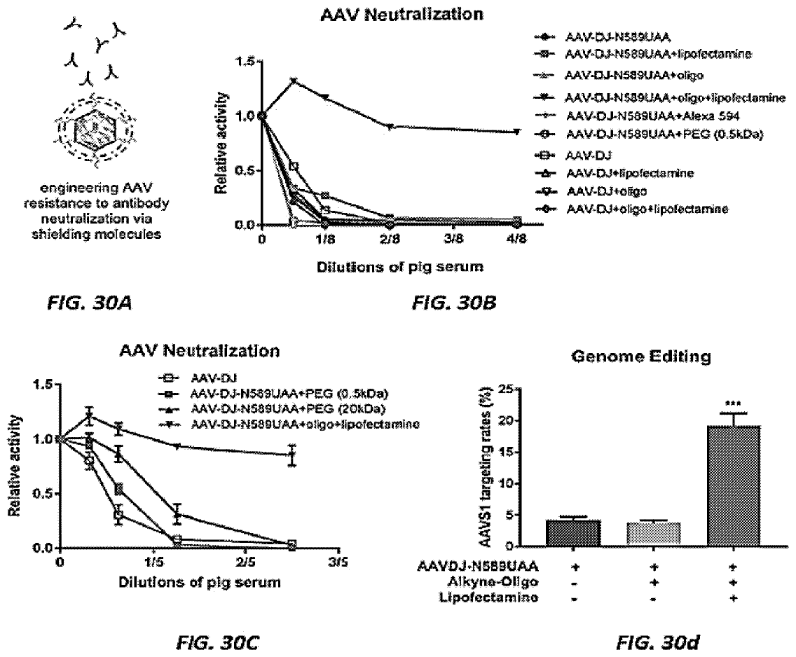

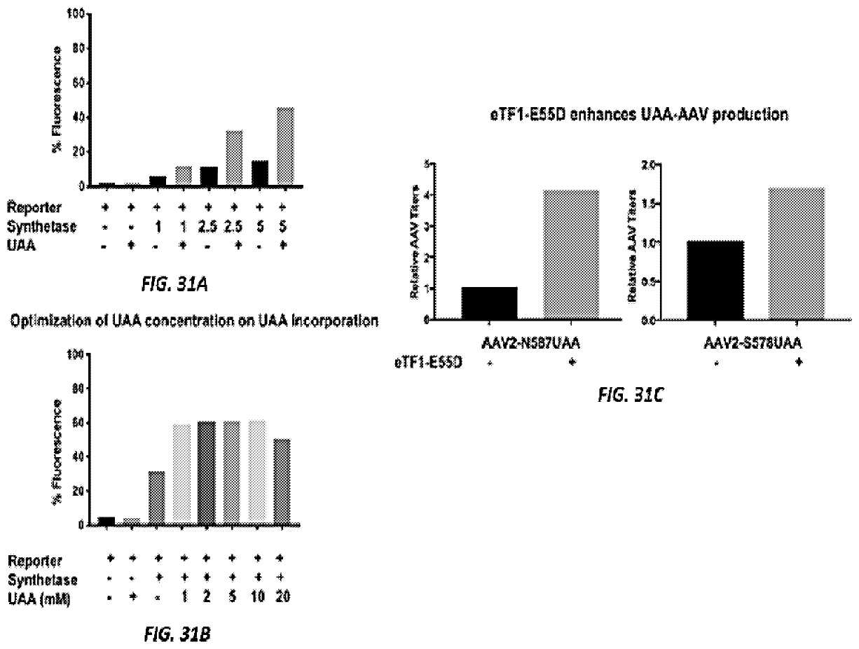

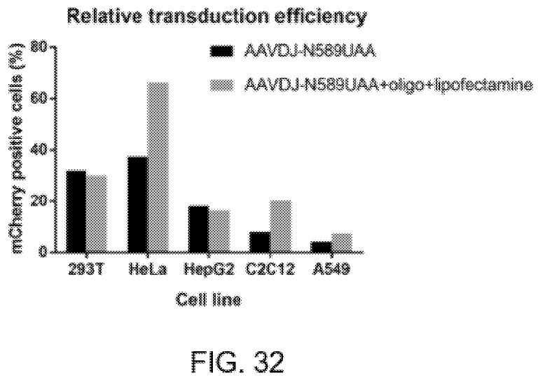

After having determined (FIG. 25) which AAV serotype is optimal for targeting the DRG (dorsal root ganglion), Applicants conduct experiments targeting several genes.

| | |
|---|---|
| Nav 1.3 (SCN3A) | Repress/KO |
| Nav 1.7 (SCN9A) | Repress/KO |
| Nav 1.8 (SCN10A) | Repress/KO |
| Nav 1.9 (SCN11A) | Repress/KO |
| SHANK3 | Repress/KO |
| NMDA receptor antagonists | Repress/KO |
| (including NR2B) | |
| IL-10 | Activate (overexpress) |
| Penk | Activate (overexpress) |
| Pomc | Activate (overexpress) |
| MVIIA-PC | Activate (overexpress) |

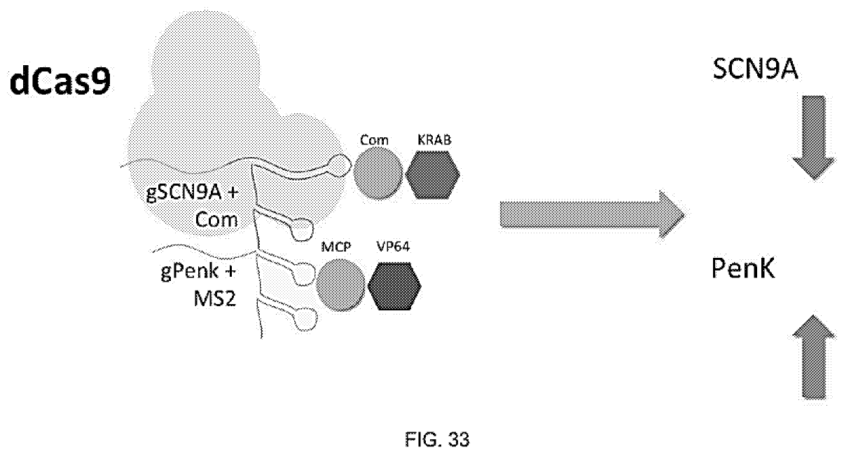

In the first round of experiments, Applicants first edit the SCN9A gene. Applicants inject C57BL/6J mice intrathecally with ~1E11-1E12 vg/mouse of AAV with the split-Cas9 targeting the SCN9A gene. Applicants then separate other mice into 5 groups to test the different pain models, with WT mice injected with opioids as the positive control, and mice injected with PBS as the negative control. At the end of 8 weeks, Applicants sacrifice the mice, extract gDNA from the DRGs and sequence the targeted region of interest (150 bp surrounding the cut site), via next generation sequencing. Because a permanent loss of pain might not be desirable, Applicants also target SCN9A via dCas9 and the optimized repression domains (FIG. 33). Applicants again test this set of mice with the pain models. Additionally, Applicants harvest the mice DRG neurons at 8 weeks and will conduct RNA-sequencing to determine the changes in gene expression post therapy. Some additional genes that Applicants are targeting include other sodium channels such as Nav 1.8 (SCN10A gene), 1.9 (SCN11A gene) and 1.3 (SCN3A gene), as well as the transient receptor potential cation channel subfamily V member 1 (TrpV1), also known as the capsaicin receptor and the vanilloid receptor 1, SHANK3, and NMDA receptor antagonists. Because gene repression might not suffice to achieve a pain-free state, Applicants also conduct gene activation (or overexpression).

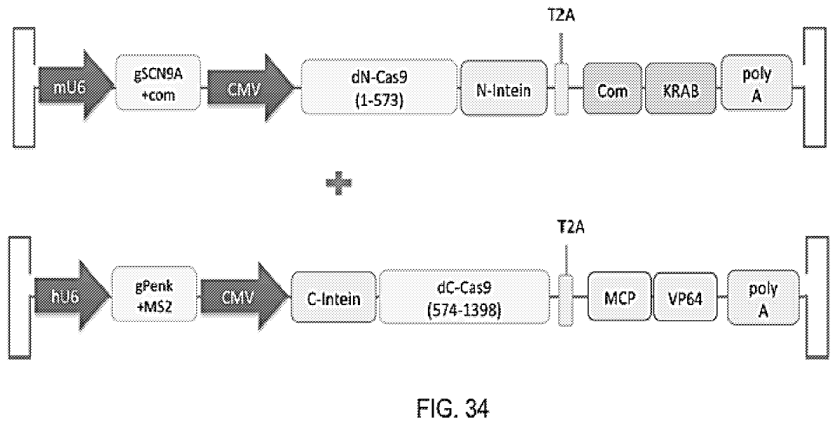

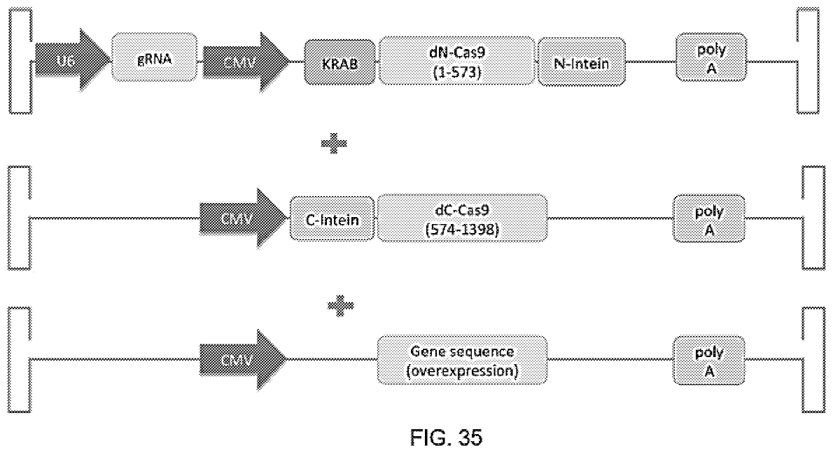

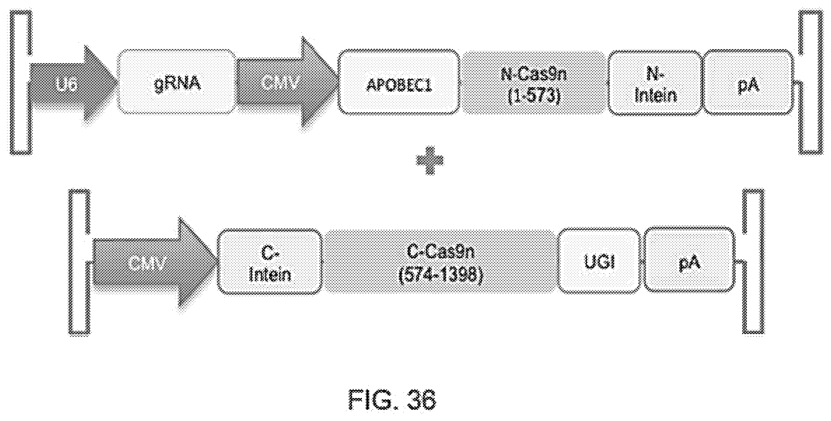

Previous research has shown that a simultaneous repression of SCN9A and upregulation of the enkephalin precursor Penk might be necessary for a pain-free phenotype. For this reason, Applicants utilize gRNA constructs with RNA hairpins (MS2, PP7, Com) and fuse their cognate RNA-binding proteins onto the activation/repression domains. For activation of Penk, Applicants construct gRNA-MS2 construct on the dN-Cas9 plasmid and fuse the MS2 RNA cognate, MCP onto the VP64 activation site. Similarly, Applicants add the SCN9A specific gRNA-Com onto the dN-Cas9 and its RNA cognate, COM is fused onto a KRAB. Applicants can therefore utilize the dual-AAV dCas9 system with RNA hairpins attached to gRNAs that will recruit the activation/repression of choice to the specific location, allowing simultaneous activation and repression. (FIGS. 33 and 34). Therefore, Applicants inject mice with AAVs that simultaneously activate Penk and repress SCN9A, to determine whether there is any difference in the mice's pain phenotype and will against do an RNA-seq to determine the extent of activation/repression. In addition to SCN9A for repression and Penk for activation, Applicants are targeting other genes for simultaneous activation/repression. Furthermore, in addition to doing simultaneous activation and repression via CRISPR, Applicants are conducting repression via the dCas9-KRAB-gRNA split-AAV constructs and simultaneous activation via overexpression of a gene. (FIG. 35).

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Charpentier, E. & Doudna, J. A. Biotechnology: Rewriting a genome. Nature 495, 50-51 (2013).
2. Hwang, W. Y. et al. Efficient genome editing in zebrafish using a CRISPR-Cas system. Nat Biotechnol 31, 227-229 (2013).
3. Li, D. et al. Heritable gene targeting in the mouse and rat using a CRISPR-Cas system. Nat Biotechnol 31, 681-683 (2013).
4. Mali, P., Esvelt, K. M. & Church, G. M. Cas9 as a versatile tool for engineering biology. Nat Methods 10, 957-963 (2013).
5. Mali, P. et al. RNA-guided human genome engineering via Cas9. Science 339, 823-826 (2013).
6. Nakayama, T. et al. Simple and efficient CRISPR/Cas9-mediated targeted mutagenesis in Xenopus tropicalis. Genesis 51, 835-843 (2013).
7. Shan, Q. et al. Targeted genome modification of crop plants using a CRISPR-Cas system. Nat Biotechnol 31, 686-688 (2013).
8. Yang, D. et al. Effective gene targeting in rabbits using RNA-guided Cas9 nucleases. J Mol Cell Biol 6, 97-99 (2014).
9. Yu, Z. et al. Highly efficient genome modifications mediated by CRISPR/Cas9 in Drosophila. Genetics 195, 289-291 (2013).
10. DiCarlo, J. E. et al. Genome engineering in Saccharomyces cerevisiae using CRISPR-Cas systems. Nucleic Acids Res 41, 4336-4343 (2013).
11. Wang, J. et al. Homology-driven genome editing in hematopoietic stem and progenitor cells using ZFN mRNA and AAV6 donors. Nat Biotechnol 33, 1256-1263 (2015).
12. Yang, Y. et al. A dual AAV system enables the Cas9-mediated correction of a metabolic liver disease in newborn mice. Nat Biotechnol 34, 334-338 (2016).
13. Long, C. et al. Postnatal genome editing partially restores dystrophin expression in a mouse model of muscular dystrophy. Science 351, 400-403 (2016).
14. Nelson, C. E. et al. In vivo genome editing improves muscle function in a mouse model of Duchenne muscular dystrophy. Science 351, 403-407 (2016).
15. Tabebordbar, M. et al. In vivo gene editing in dystrophic mouse muscle and muscle stem cells. Science 351, 407-411 (2016).
16. Ran, F. A. et al. In vivo genome editing using Staphylococcus aureus Cas9. Nature 520, 186-191 (2015).
17. Zuris, J. A. et al. Cationic lipid-mediated delivery of proteins enables efficient protein-based genome editing in vitro and in vivo. Nat Biotechnol 33, 73-80 (2015).
18. Hsu, P. D., Lander, E. S. & Zhang, F. Development and applications of CRISPR-Cas9 for genome engineering. Cell 157, 1262-1278 (2014).
19. Truong, D. J. et al. Development of an intein-mediated split-Cas9 system for gene therapy. Nucleic Acids Res 43, 6450-6458 (2015).
20. Wright, A. V. et al. Rational design of a split-Cas9 enzyme complex. Proc Natl Acad Sci US A 112, 2984-2989 (2015).
21. Zetsche, B., Volz, S. E. & Zhang, F. A split-Cas9 architecture for inducible genome editing and transcription modulation. Nat Biotechnol 33, 139-142 (2015).
22. Davis, K. M., Pattanayak, V., Thompson, D. B., Zuris, J. A. & Liu, D. R. Small molecule-triggered Cas9 protein with improved genome-editing specificity. Nat Chem Biol 11, 316-318 (2015).
23. Chavez, A. et al. Highly efficient Cas9-mediated transcriptional programming. Nat Methods 12, 326-328 (2015).
24. Gilbert, L. A. et al. CRISPR-mediated modular RNA-guided regulation of transcription in eukaryotes. Cell 154, 442-451 (2013).
25. Hilton, I. B. et al. Epigenome editing by a CRISPR-Cas9-based acetyltransferase activates genes from promoters and enhancers. Nat Biotechnol 33, 510-517 (2015).
26. Mali, P. et al. CAS9 transcriptional activators for target specificity screening and paired nickases for cooperative genome engineering. Nat Biotechnol 31, 833-838 (2013).
27. Qi, L. S. et al. Repurposing CRISPR as an RNA-guided platform for sequence-specific control of gene expression. Cell 152, 1173-1183 (2013).
28. Maeder, M. L. et al. CRISPR RNA-guided activation of endogenous human genes. Nat Methods 10, 977-979 (2013).
29. Aslanidi, G. V. et al. Optimization of the capsid of recombinant adeno-associated virus 2 (AAV2) vectors: the final threshold? PLOS One 8, e59142 (2013).
30. Ried, M. U., Girod, A., Leike, K., Buning, H. & Hallek, M. Adeno-associated virus capsids displaying immunoglobulin-binding domains permit antibody-mediated vector retargeting to specific cell surface receptors. J Virol 76, 4559-4566 (2002).
31. Shi, W., Arnold, G. S. & Bartlett, J. S. Insertional mutagenesis of the adeno-associated virus type 2 (AAV2) capsid gene and generation of AAV2 vectors targeted to alternative cell-surface receptors. Hum Gene Ther 12, 1697-1711 (2001).
32. Wu, P. et al. Mutational analysis of the adeno-associated virus type 2 (AAV2) capsid gene and construction of AAV2 vectors with altered tropism. J Virol 74, 8635-8647 (2000).
33. Xie, Q. et al. The atomic structure of adeno-associated virus (AAV-2), a vector for human gene therapy. Proc Natl Acad Sci USA 99, 10405-10410 (2002).
34. Chatterjee, A., Xiao, H., Bollong, M., Ai, H. W. & Schultz, P. G. Efficient viral delivery system for unnatural amino acid mutagenesis in mammalian cells. Proc Natl Acad Sci US A 110, 11803-11808 (2013).
35. Schmied, W. H., Elsasser, S. J., Uttamapinant, C. & Chin, J. W. Efficient multisite unnatural amino acid incorporation in mammalian cells via optimized pyrrolysyl tRNA synthetase/tRNA expression and engineered eRF1. J Am Chem Soc 136, 15577-15583 (2014).
36. Elsasser, S. J., Ernst, R. J., Walker, O. S. & Chin, J. W. Genetic code expansion in stable cell lines enables encoded chromatin modification. Nat Methods 13, 158-164 (2016).
37. Zheng, Y. et al. Broadening the versatility of lentiviral vectors as a tool in nucleic acid research via genetic code expansion. Nucleic Acids Res 43, e73 (2015).
38. Deverman, B. E. et al. Cre-dependent selection yields AAV variants for widespread gene transfer to the adult brain. Nat Biotechnol 34, 204-209 (2016).
39. Grimm, D. et al. In vitro and in vivo gene therapy vector evolution via multispecies interbreeding and retargeting of adeno-associated viruses. J Virol 82, 5887-5911 (2008).
40. Maheshri, N., Koerber, J. T., Kaspar, B. K. & Schaffer, D. V. Directed evolution of adeno-associated virus yields enhanced gene delivery vectors. Nat Biotechnol 24, 198-204 (2006).
41. Zinn, E. et al. In Silico Reconstruction of the Viral Evolutionary Lineage Yields a Potent Gene Therapy Vector. Cell Rep 12, 1056-1068 (2015).
42. Guenther, C. M. et al. Synthetic virology: engineering viruses for gene delivery. Wiley Interdiscip Rev Nanomed Nanobiotechnol 6, 548-558 (2014).
43. Endy, D. Foundations for engineering biology. Nature 438, 449-453 (2005).
44. Guell, M., Yang, L. & Church, G. M. Genome editing assessment using CRISPR Genome Analyzer (CRISPR-GA). Bioinformatics 30, 2968-2970 (2014).
45. Mali, P. et al. Barcoding cells using cell-surface programmable DNA-binding domains. Nat Methods 10, 403-406 (2013).
46. Rapti, K. et al. Neutralizing Antibodies Against AAV Serotypes 1, 2, 6, and 9 in Sera of Commonly Used Animal Models. Mol. Ther. 20, 73-83 (2009).
47. Lee, G. K., Maheshri, N., Kaspar, B. & Schaffer, D. V. PEG Conjugation Moderately Protects Adeno-Associated Viral Vectors Against Antibody Neutralization. (2005). doi:10.1002/bit.20562
48. Fitzpatrick, Z., Crommentuijn, M. H. W., Mu, D. & Maguire, C. A. Biomaterials Naturally enveloped AAV vectors for shielding neutralizing antibodies and robust gene delivery in vivo. 35, 7598-7609 (2014).
49. Lerch, T. F. et al. Structure of AAV-DJ, a Retargeted Gene Therapy Vector: Cryo-Electron Microscopy at 4.5 Å resolution. NIH Public Access. 20, 1310-1320 (2013).
50. Chew, W. L. et al. A multifunctional AAV-CRISPR-Cas9 and its host response. 13, (2016).
51. Kelemen et al. A Precise Chemical Strategy To Alter the Receptor Specificity of the Adeno-Associated Virus. 10-15 (2016). doi: 10.1002/anie.201604067.

What is claimed is:

1. A recombinant expression system for CRISPR-based genome or epigenome editing comprising:
(a) a first expression vector comprising (i) a polynucleotide encoding C-intein, (ii) a polynucleotide encoding C-Cas9, and (iii) a promoter sequence for the first vector; and
(b) a second expression vector comprising (i) a polynucleotide encoding N-Cas9, (ii) a polynucleotide encoding N-intein, and (iii) a promoter sequence for the second vector, wherein optionally, both the first and second expression vectors are adeno-associated virus (AAV) or lentivirus vectors, and
wherein co-expression of the first and second expression vectors results in the expression of a whole Cas9 protein.

2. The recombinant expression system of claim 1, wherein the promoter sequence of the second vector comprises a first promoter operatively linked to an gRNA sequence, optionally an sgRNA, and a second promoter.

3. The recombinant expression system of claim 1, wherein both the first and second expression vectors further comprise a poly-A tail.

4. The recombinant expression system of claim 1, wherein: the first expression vector further comprises a tetracycline response element and/or the second expression vector further comprises a tetracycline regulatable activator, or wherein the first expression vector further comprises a tetracycline regulatable activator and/or the second expression vector further comprises a tetracycline response element.

5. The recombinant expression of claim 4, wherein the tetracycline response element comprises one or more repeats of tetO.

6. The recombinant expression system of claim 1, wherein the C-Cas9 is dC-Cas9 and the N-Cas9 is dN-Cas9.

7. The recombinant expression system of claim 6, wherein the first expression vector and/or second expression vector further comprises one or more of KRAB, DNMT3A, or DNMT3L.

8. The recombinant expression system of claim 6, wherein the first expression vector and/or second expression vector further comprises one or more of VP64, RtA, or P65.

9. The recombinant expression system of claim 7, further comprising a gRNA for a gene targeted for repression, silencing, or downregulation.

10. The recombinant expression system of claim 8, further comprising a gRNA for a gene targeted for expression, activation, or upregulation.

11. The recombinant expression system of claim 10, further comprising a third expression vector encoding the gene targeted for expression, activation, or upregulation and, optionally, a promoter.

12. The recombinant expression system of claim 1, wherein the first expression vector and/or the second expression vector further comprises an miRNA circuit.

13. A composition comprising the recombinant expression system of claim 1, wherein the first expression vector is encapsulated in a first viral capsid and the second expression vector is encapsulated in a second viral capsid, and optionally, wherein the first viral capsid and/or the second viral capsid is an AAV or lentivirus capsid.

14. A method of pain management in a subject in need thereof, comprising administering an effective amount of the composition of claim 13 to the subject, wherein the composition comprises a vector encoding a gRNA targeting one or more of SCN9A, SCN10A, SCN11A, SCN3A, TrpV1, SHANK3, NR2B, IL-10, PENK, POMC, or MVIIA-PC.


SEQUENCE LISTING (346 sequences)

SEQ ID NO: 1 - DNA, 701 bp, Cytomegalovirus
  source 1..701: mol_type = other DNA
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaaccctt

SEQ ID NO: 2 - DNA, 249 bp, synthetic construct
  misc_feature 1..249: note = Description of Unknown: U6 promoter sequence
  source 1..249: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 3 - protein, 830 aa, synthetic construct
  REGION 1..830: note = Description of Artificial Sequence: Synthetic polypeptide
MIKIATRKYLGKQNVYDIGVERDHNFALKNGFIASCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 830 aa, synthetic construct
  REGION 1..830: note = Description of Artificial Sequence: Synthetic polypeptide
MIKIATRKYLGKQNVYDIGVERDHNFALKNGFIASCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 702 aa, synthetic construct
  REGION 1..702: note = Description of Artificial Sequence: Synthetic polypeptide
MGPKKKRKVAAADYKDDDDKGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 6 - protein, 702 aa, synthetic construct
  REGION 1..702: note = Description of Artificial Sequence: Synthetic polypeptide
MGPKKKRKVAAADYKDDDDKGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 7 - DNA, 22 bp, synthetic construct
  misc_feature 1..22: note = Description of Artificial Sequence: Synthetic oligonucleotide
  source 1..22: mol_type = other DNA
actccctatcagtgatagagaa

SEQ ID NO: 8 - DNA, 376 bp, synthetic construct
  misc_feature 1..376: note = Description of Artificial Sequence: Synthetic polynucleotide
  source 1..376: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 9 - DNA, 248 bp, synthetic construct
  misc_feature 1..248: note = Description of Artificial Sequence: Synthetic polynucleotide
  source 1..248: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataa

SEQ ID NO: 10 - protein, 270 aa, synthetic construct
  REGION 1..270: note = Description of Artificial Sequence: Synthetic polypeptide
GSGATNFSLLKQAGDVEENPGPMSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 11 - RNA, 22 nt, Homo sapiens
  source 1..22: mol_type = other RNA
tagcttatcagactgatgttga

SEQ ID NO: 12 - DNA, 29 bp, Homo sapiens
  source 1..29: mol_type = other DNA
tcaacatcagtctgataagctaagatcta

SEQ ID NO: 13 - RNA, 22 nt, Homo sapiens
  source 1..22: mol_type = other RNA
tcgtaccgtgagtaataatgcg

SEQ ID NO: 14 - DNA, 29 bp, Homo sapiens
  source 1..29: mol_type = other DNA
cgcattattactcacggtacgaagatcac

SEQ ID NO: 15 - RNA, 22 nt, unidentified
  misc_feature 1..22: note = Description of Unknown: miR-1a-3p sequence
  source 1..22: mol_type = other RNA
tggaatgtaaagaagtatgtat

SEQ ID NO: 16 - DNA, 29 bp, unidentified
  misc_feature 1..29: note = Description of Unknown: Heart target sequence
  source 1..29: mol_type = other DNA
atacatacttctttacattccaagatcac

SEQ ID NO: 17 - RNA, 22 nt, unidentified
  misc_feature 1..22: note = Description of Unknown: miR-122a-5p sequence
  source 1..22: mol_type = other RNA
tggagtgtgacaatggtgtttg

SEQ ID NO: 18 - DNA, 29 bp, unidentified
  misc_feature 1..29: note = Description of Unknown: Liver target sequence
  source 1..29: mol_type = other DNA
caaacaccattgtcacactccaagatcac

SEQ ID NO: 19 - protein, 1710 aa, synthetic construct
  REGION 1..1710: note = Description of Artificial Sequence: Synthetic polypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 20 - protein, 71 aa, Homo sapiens
DAKSLTAWSRTLVTFKDVFVDFTREEWKLLDTAQQIVYRNVMLENYKNLVSLGYQLTKPDVILRLEKGEEP

SEQ ID NO: 21 - protein, 57 aa, synthetic construct
  REGION 1..57: note = Description of Artificial Sequence: Synthetic polypeptide
GSGRADALDDFDLDMLGSDALDDFDLDMLGSDALDDFDLDMLGSDALDDFDLDMLIN

SEQ ID NO: 22 - protein, 150 aa, unidentified
  REGION 1..150: note = Description of Unknown: RTa sequence
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLN

SEQ ID NO: 23 - protein, 261 aa, unidentified
  REGION 1..261: note = Description of Unknown: P65 sequence
SQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 24 - protein, 322 aa, synthetic construct
  REGION 1..322: note = Description of Artificial Sequence: Synthetic polypeptide
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 25 - protein, 366 aa, synthetic construct
  REGION 1..366: note = Description of Artificial Sequence: Synthetic polypeptide
GSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 26 - DNA, 20 bp, synthetic construct
  misc_feature 1..20: note = Description of Artificial Sequence: Synthetic oligonucleotide
  source 1..20: mol_type = other DNA
ggaaagccgacagccgccgc

SEQ ID NO: 27 - DNA, 20 bp, synthetic construct
  misc_feature 1..20: note = Description of Artificial Sequence: Synthetic oligonucleotide
  source 1..20: mol_type = other DNA
ggcgcgggcctctccttccc

SEQ ID NO: 28 - DNA, 20 bp, synthetic construct
  misc_feature 1..20: note = Description of Artificial Sequence: Synthetic oligonucleotide
  source 1..20: mol_type = other DNA
gagcacgggcgaaagaccga

SEQ ID NO: 29 - DNA, 20 bp, synthetic construct
  misc_feature 1..20: note = Description of Artificial Sequence: Synthetic oligonucleotide
  source 1..20: mol_type = other DNA
gtgtgctcttaaggggtgcg

SEQ ID NO: 30 - DNA, 20 bp, synthetic construct
  misc_feature 1..20: note = Description of Artificial Sequence: Synthetic oligonucleotide
  source 1..20: mol_type = other DNA
gtggcggttgaggcgagcac

SEQ ID NO: 31 - DNA, 20 bp, synthetic construct
  misc_feature 1..20: note = Description of Artificial Sequence: Synthetic oligonucleotide
  source 1..20: mol_type = other DNA
gacccatgtaacaactccac

SEQ ID NO: 32 - DNA, 20 bp, synthetic construct
  misc_feature 1..20: note = Description of Artificial Sequence: Synthetic oligonucleotide
  source 1..20: mol_type = other DNA
gtgtatattgttgaacccgt

SEQ ID NO: 33 - DNA, 20 bp, synthetic construct
  misc_feature 1..20: note = Description of Artificial Sequence: Synthetic oligonucleotide
  source 1..20: mol_type = other DNA
aacaactccactggagtaga

SEQ ID NO: 34 - DNA, 20 bp, synthetic construct
  misc_feature 1..20: note = Description of Artificial Sequence: Synthetic oligonucleotide
  source 1..20: mol_type = other DNA
caaactgttaagaaacgggc

SEQ ID NO: 35 - DNA, 20 bp, synthetic construct
  misc_feature 1..20: note = Description of Artificial Sequence: Synthetic oligonucleotide
  source 1..20: mol_type = other DNA
ggttctggcaaaattgctgt

SEQ ID NO: 36 - DNA, 20 bp, synthetic construct
  misc_feature 1..20: note = Description of Artificial Sequence: Synthetic oligonucleotide
  source 1..20: mol_type = other DNA
tcgtggatttctatcacttt

SEQ ID NO: 37 - DNA, 20 bp, synthetic construct
  misc_feature 1..20: note = Description of Artificial Sequence: Synthetic oligonucleotide
  source 1..20: mol_type = other DNA
cttggtaacgtcttctcttg

SEQ ID NO: 38 - DNA, 20 bp, synthetic construct
  misc_feature 1..20: note = Description of Artificial Sequence: Synthetic oligonucleotide
  source 1..20: mol_type = other DNA
cgatggttccacgtgcaata

SEQ ID NO: 39 - DNA, 20 bp, synthetic construct
  misc_feature 1..20: note = Description of Artificial Sequence: Synthetic oligonucleotide
  source 1..20: mol_type = other DNA
taagctgaataacaccgttg

SEQ ID NO: 40 - DNA, 20 bp, synthetic construct
  misc_feature 1..20: note = Description of Artificial Sequence: Synthetic oligonucleotide
  source 1..20: mol_type = other DNA
ccgcttcctgttctgagatc

SEQ ID NO: 41 - DNA, 20 bp, synthetic construct
  misc_feature 1..20: note = Description of Artificial Sequence: Synthetic oligonucleotide
  source 1..20: mol_type = other DNA
gtcacgagttccaccctgcc

SEQ ID NO: 42 - DNA, 20 bp, synthetic construct
  misc_feature 1..20: note = Description of Artificial Sequence: Synthetic oligonucleotide
  source 1..20: mol_type = other DNA
cagcctggatggcttacctc

SEQ ID NO: 43 - DNA, 20 bp, synthetic construct
  misc_feature 1..20: note = Description of Artificial Sequence: Synthetic oligonucleotide
  source 1..20: mol_type = other DNA
gggacttaccagctaggtgc

SEQ ID NO: 44 - DNA, 20 bp, synthetic construct
  misc_feature 1..20: note = Description of Artificial Sequence: Synthetic oligonucleotide
  source 1..20: mol_type = other DNA
gatctcagaacaggaagcgg

SEQ ID NO: 45 - DNA, 20 bp, synthetic construct
  misc_feature 1..20: note = Description of Artificial Sequence: Synthetic oligonucleotide
  source 1..20: mol_type = other DNA
gtgtaaattacaggaaccaa

SEQ ID NO: 46 - DNA, 20 bp, synthetic construct
  misc_feature 1..20: note = Description of Artificial Sequence: Synthetic oligonucleotide
  source 1..20: mol_type = other DNA
gacctggtagctaggttcta

SEQ ID NO: 47 - DNA, 20 bp, synthetic construct
  misc_feature 1..20: note = Description of Artificial Sequence: Synthetic oligonucleotide
  source 1..20: mol_type = other DNA
gatagagtgaatctcagaac

SEQ ID NO: 48 - DNA, 20 bp, synthetic construct
  misc_feature 1..20: note = Description of Artificial Sequence: Synthetic oligonucleotide
  source 1..20: mol_type = other DNA
gaatagagcctgtctggaaa

SEQ ID NO: 49 - DNA, 20 bp, synthetic construct
  misc_feature 1..20: note = Description of Artificial Sequence: Synthetic oligonucleotide
  source 1..20: mol_type = other DNA
gtgttatgctgtaattcata

SEQ ID NO: 50 - DNA, 20 bp, synthetic construct
  misc_feature 1..20: note = Description of Artificial Sequence: Synthetic oligonucleotide
  source 1..20: mol_type = other DNA
ggtctggaaatggtgattta

SEQ ID NO: 51 - DNA, 20 bp, synthetic construct
  misc_feature 1..20: note = Description of Artificial Sequence: Synthetic oligonucleotide
  source 1..20: mol_type = other DNA
gaaagaaaatagagcctgtc

SEQ ID NO: 52 - DNA, 20 bp, synthetic construct
  misc_feature 1..20: note = Description of Artificial Sequence: Synthetic oligonucleotide
  source 1..20: mol_type = other DNA
gcctaaccatcttggatgct

SEQ ID NO: 53 - DNA, 20 bp, synthetic construct
  misc_feature 1..20: note = Description of Artificial Sequence: Synthetic oligonucleotide
  source 1..20: mol_type = other DNA
gaccatagaacctagctacc

SEQ ID NO: 54 - DNA, 20 bp, synthetic construct
  misc_feature 1..20: note = Description of Artificial Sequence: Synthetic oligonucleotide
  source 1..20: mol_type = other DNA
ggcggtcgccagcgctccag

SEQ ID NO: 55 - DNA, 20 bp, synthetic construct
  misc_feature 1..20: note = Description of Artificial Sequence: Synthetic oligonucleotide
  source 1..20: mol_type = other DNA
gccacctggaaagaagagag

SEQ ID NO: 56 - DNA, 20 bp, synthetic construct
  misc_feature 1..20: note = Description of Artificial Sequence: Synthetic oligonucleotide
  source 1..20: mol_type = other DNA
ggtcgccagcgctccagcgg

SEQ ID NO: 57 - DNA, 20 bp, synthetic construct
  misc_feature 1..20: note = Description of Artificial Sequence: Synthetic oligonucleotide
  source 1..20: mol_type = other DNA
gccagcaatgggaggaagaa

SEQ ID NO: 58 - DNA, 20 bp, synthetic construct
  misc_feature 1..20: note = Description of Artificial Sequence: Synthetic oligonucleotide
  source 1..20: mol_type = other DNA
gttccaggtggcgtaataca

SEQ ID NO: 59 - DNA, 20 bp, synthetic construct
  misc_feature 1..20: note = Description of Artificial Sequence: Synthetic oligonucleotide
  source 1..20: mol_type = other DNA
ggcggggctgctacctccac

SEQ ID NO: 60 - DNA, 20 bp, synthetic construct
  misc_feature 1..20: note = Description of Artificial Sequence: Synthetic oligonucleotide
  source 1..20: mol_type = other DNA
gggcgcagtctgcttgcagg

SEQ ID NO: 61 - DNA, 20 bp, synthetic construct
  misc_feature 1..20: note = Description of Artificial Sequence: Synthetic oligonucleotide
  source 1..20: mol_type = other DNA
ggcgctccagcggcggctgt

SEQ ID NO: 62 - DNA, 20 bp, synthetic construct
  misc_feature 1..20: note = Description of Artificial Sequence: Synthetic oligonucleotide
  source 1..20: mol_type = other DNA
gaccgggtggttccagcaat

SEQ ID NO: 63 - DNA, 20 bp, synthetic construct
  misc_feature 1..20: note = Description of Artificial Sequence: Synthetic oligonucleotide
  source 1..20: mol_type = other DNA
ggggtggttccagcaatggg

SEQ ID NO: 64 - DNA, 20 bp, synthetic construct
  misc_feature 1..20: note = Description of Artificial Sequence: Synthetic oligonucleotide
  source 1..20: mol_type = other DNA
gtgactccggagtaaagcga

SEQ ID NO: 65 - DNA, 20 bp, synthetic construct
  misc_feature 1..20: note = Description of Artificial Sequence: Synthetic oligonucleotide
  source 1..20: mol_type = other DNA
gggagctcaccatagaactt

SEQ ID NO: 66 - DNA, 20 bp, synthetic construct
  misc_feature 1..20: note = Description of Artificial Sequence: Synthetic oligonucleotide
  source 1..20: mol_type = other DNA
gacggatctagatcctccag

SEQ ID NO: 67 - DNA, 20 bp, synthetic construct
  misc_feature 1..20: note = Description of Artificial Sequence: Synthetic oligonucleotide
  source 1..20: mol_type = other DNA
gccgggtaagagctactagt

SEQ ID NO: 68 - DNA, 20 bp, synthetic construct
  misc_feature 1..20: note = Description of Artificial Sequence: Synthetic oligonucleotide
  source 1..20: mol_type = other DNA
gcccggtgtgtgctgtagaa

SEQ ID NO: 69 - DNA, 20 bp, synthetic construct
  misc_feature 1..20: note = Description of Artificial Sequence: Synthetic oligonucleotide
  source 1..20: mol_type = other DNA
gtttactccggagtcactgg

SEQ ID NO: 70 - DNA, 20 bp, synthetic construct
  misc_feature 1..20: note = Description of Artificial Sequence: Synthetic oligonucleotide
  source 1..20: mol_type = other DNA
gctatctccaccagtgactc

SEQ ID NO: 71 - DNA, 20 bp, synthetic construct
  misc_feature 1..20: note = Description of Artificial Sequence: Synthetic oligonucleotide
  source 1..20: mol_type = other DNA
gacatcacccagggccaagg

SEQ ID NO: 72 - DNA, 20 bp, synthetic construct
  misc_feature 1..20: note = Description of Artificial Sequence: Synthetic oligonucleotide
  source 1..20: mol_type = other DNA
gtagtttcgagggatccaat

SEQ ID NO: 73 - DNA, 20 bp, synthetic construct
  misc_feature 1..20: note = Description of Artificial Sequence: Synthetic oligonucleotide
  source 1..20: mol_type = other DNA
gctcccagcagaactgatcg

SEQ ID NO: 74 - DNA, 20 bp, synthetic construct
  misc_feature 1..20: note = Description of Artificial Sequence: Synthetic oligonucleotide
  source 1..20: mol_type = other DNA
gatgggtccaagtcttccag

SEQ ID NO: 75 - DNA, 20 bp, synthetic construct
  misc_feature 1..20: note = Description of Artificial Sequence: Synthetic oligonucleotide
  source 1..20: mol_type = other DNA
ggttcctgctatacccacag

SEQ ID NO: 76 - DNA, 20 bp, synthetic construct
  misc_feature 1..20: note = Description of Artificial Sequence: Synthetic oligonucleotide
  source 1..20: mol_type = other DNA
gccagagagtcggaagtgaa

SEQ ID NO: 77 - DNA, 20 bp, synthetic construct
  misc_feature 1..20: note = Description of Artificial Sequence: Synthetic oligonucleotide
  source 1..20: mol_type = other DNA
gcctgctatacccacagtgg

SEQ ID NO: 78 - DNA, 20 bp, synthetic construct
  misc_feature 1..20: note = Description of Artificial Sequence: Synthetic oligonucleotide
  source 1..20: mol_type = other DNA
gggaaagcctctggaagact

SEQ ID NO: 79 - DNA, 20 bp, synthetic construct
  misc_feature 1..20: note = Description of Artificial Sequence: Synthetic oligonucleotide
  source 1..20: mol_type = other DNA
ggaagagatgaccaccactg

SEQ ID NO: 80 - DNA, 20 bp, synthetic construct
  misc_feature 1..20: note = Description of Artificial Sequence: Synthetic oligonucleotide
  source 1..20: mol_type = other DNA
ggaatgtcgccatagagctt

SEQ ID NO: 81 - DNA, 20 bp, synthetic construct
  misc_feature 1..20: note = Description of Artificial Sequence: Synthetic oligonucleotide
  source 1..20: mol_type = other DNA
ggagctcataggaaagcctc

SEQ ID NO: 82 - DNA, 20 bp, synthetic construct
  misc_feature 1..20: note = Description of Artificial Sequence: Synthetic oligonucleotide
  source 1..20: mol_type = other DNA
gctttaagactggaatccta

SEQ ID NO: 83 - DNA, 20 bp, synthetic construct
  misc_feature 1..20: note = Description of Artificial Sequence: Synthetic oligonucleotide
  source 1..20: mol_type = other DNA
gggaagttgcccaagctcta

SEQ ID NO: 84 - DNA, 20 bp, synthetic construct
  misc_feature 1..20: note = Description of Artificial Sequence: Synthetic oligonucleotide
  source 1..20: mol_type = other DNA
ggaattcgaatacagctcct

SEQ ID NO: 85 - DNA, 20 bp, synthetic construct
  misc_feature 1..20: note = Description of Artificial Sequence: Synthetic oligonucleotide
  source 1..20: mol_type = other DNA
gcttcaggcagagacccccg

SEQ ID NO: 86 - DNA, 20 bp, synthetic construct
  misc_feature 1..20: note = Description of Artificial Sequence: Synthetic oligonucleotide
  source 1..20: mol_type = other DNA
ggagcctccgtggtgacaca

SEQ ID NO: 87 - DNA, 20 bp, synthetic construct
  misc_feature 1..20: note = Description of Artificial Sequence: Synthetic oligonucleotide
  source 1..20: mol_type = other DNA
gcacggcaggaaccttcccc

SEQ ID NO: 88 - DNA, 20 bp, synthetic construct
  misc_feature 1..20: note = Description of Artificial Sequence: Synthetic oligonucleotide
  source 1..20: mol_type = other DNA
gagcaccggagggacccgca

SEQ ID NO: 89 - DNA, 20 bp, synthetic construct
  misc_feature 1..20: note = Description of Artificial Sequence: Synthetic oligonucleotide
  source 1..20: mol_type = other DNA
ggcccggaacgacagagcac

SEQ ID NO: 90 - DNA, 20 bp, synthetic construct
  misc_feature 1..20: note = Description of Artificial Sequence: Synthetic oligonucleotide
  source 1..20: mol_type = other DNA
gggaacgacagagcaccgga

SEQ ID NO: 91 - DNA, 20 bp, synthetic construct
  misc_feature 1..20: note = Description of Artificial Sequence: Synthetic oligonucleotide
  source 1..20: mol_type = other DNA
gaccgcggcgaggccgtgaa

SEQ ID NO: 92 - DNA, 20 bp, synthetic construct
  misc_feature 1..20: note = Description of Artificial Sequence: Synthetic oligonucleotide
  source 1..20: mol_type = other DNA
gcctgccgtgcgggtccctc

SEQ ID NO: 93 - DNA, 20 bp, synthetic construct
  misc_feature 1..20: note = Description of Artificial Sequence: Synthetic oligonucleotide
  source 1..20: mol_type = other DNA
gtacagctcctgggcgcgcc

SEQ ID NO: 94 - DNA, 20 bp, synthetic construct
  misc_feature 1..20: note = Description of Artificial Sequence: Synthetic oligonucleotide
  source 1..20: mol_type = other DNA
gagcgactcctgctagtgca

SEQ ID NO: 95 - DNA, 20 bp, synthetic construct
  misc_feature 1..20: note = Description of Artificial Sequence: Synthetic oligonucleotide
  source 1..20: mol_type = other DNA
gcgggcccgggaccccacgg

SEQ ID NO: 96 - DNA, 20 bp, synthetic construct
  misc_feature 1..20: note = Description of Artificial Sequence: Synthetic oligonucleotide
  source 1..20: mol_type = other DNA
gctccttggaagcacctggg

SEQ ID NO: 97 - DNA, 20 bp, synthetic construct
  misc_feature 1..20: note = Description of Artificial Sequence: Synthetic oligonucleotide
  source 1..20: mol_type = other DNA
gagtcgctgtggacgccctt

SEQ ID NO: 98 - DNA, 20 bp, synthetic construct
  misc_feature 1..20: note = Description of Artificial Sequence: Synthetic oligonucleotide
  source 1..20: mol_type = other DNA
gggactcaccagctagacgc

SEQ ID NO: 99 - DNA, 20 bp, synthetic construct
  misc_feature 1..20: note = Description of Artificial Sequence: Synthetic oligonucleotide
  source 1..20: mol_type = other DNA
gtggtctccccgcctccgtg

SEQ ID NO: 100 - DNA, 20 bp, synthetic construct
  misc_feature 1..20: note = Description of Artificial Sequence: Synthetic oligonucleotide
  source 1..20: mol_type = other DNA
ggggagagctgggctcgtgt

SEQ ID NO: 101 - DNA, 20 bp, synthetic construct
  misc_feature 1..20: note = Description of Artificial Sequence: Synthetic oligonucleotide
  source 1..20: mol_type = other DNA
gtgcctcaaaggtggtcgtg

SEQ ID NO: 102 - DNA, 20 bp, synthetic construct
  misc_feature 1..20: note = Description of Artificial Sequence: Synthetic oligonucleotide
  source 1..20: mol_type = other DNA
gctgcatcagccgtcctcgg

SEQ ID NO: 103 - DNA, 20 bp, synthetic construct
  misc_feature 1..20: note = Description of Artificial Sequence: Synthetic oligonucleotide
  source 1..20: mol_type = other DNA
gggacgcccttcggcactca

SEQ ID NO: 104 - DNA, 20 bp, synthetic construct
  misc_feature 1..20: note = Description of Artificial Sequence: Synthetic oligonucleotide
  source 1..20: mol_type = other DNA
ggattcgcgtgtcccccgga

SEQ ID NO: 105 - DNA, 20 bp, synthetic construct
  misc_feature 1..20: note = Description of Artificial Sequence: Synthetic oligonucleotide
  source 1..20: mol_type = other DNA
ggatatgcaagcgagaagaa

SEQ ID NO: 106 - DNA, 20 bp, synthetic construct
  misc_feature 1..20: note = Description of Artificial Sequence: Synthetic oligonucleotide
  source 1..20: mol_type = other DNA
gctctagacggacagattaa

SEQ ID NO: 107 - DNA, 20 bp, synthetic construct
  misc_feature 1..20: note = Description of Artificial Sequence: Synthetic oligonucleotide
  source 1..20: mol_type = other DNA
gggggaaaaagaggcggtca

SEQ ID NO: 108 - DNA, 20 bp, synthetic construct
  misc_feature 1..20: note = Description of Artificial Sequence: Synthetic oligonucleotide
  source 1..20: mol_type = other DNA
ggcaagcgagaagaagggac

SEQ ID NO: 109 - DNA, 20 bp, synthetic construct
  misc_feature 1..20: note = Description of Artificial Sequence: Synthetic oligonucleotide
  source 1..20: mol_type = other DNA
gccaaagcgtccccttccta

SEQ ID NO: 110 - DNA, 20 bp, synthetic construct
  misc_feature 1..20: note = Description of Artificial Sequence: Synthetic oligonucleotide
  source 1..20: mol_type = other DNA
gaagcgtccccttcctaagg

SEQ ID NO: 111 - DNA, 20 bp, synthetic construct
  misc_feature 1..20: note = Description of Artificial Sequence: Synthetic oligonucleotide
  source 1..20: mol_type = other DNA
ggcttctacaaaccaaggta

SEQ ID NO: 112 - DNA, 20 bp, synthetic construct
  misc_feature 1..20: note = Description of Artificial Sequence: Synthetic oligonucleotide
  source 1..20: mol_type = other DNA
gaccatgctccaccgaggga

SEQ ID NO: 113 - DNA, 20 bp, synthetic construct
  misc_feature 1..20: note = Description of Artificial Sequence: Synthetic oligonucleotide
  source 1..20: mol_type = other DNA
ggaatgaccatgctccaccg

SEQ ID NO: 114 - DNA, 20 bp, synthetic construct
  misc_feature 1..20: note = Description of Artificial Sequence: Synthetic oligonucleotide
  source 1..20: mol_type = other DNA
gtgaatctcagaacaggaag

SEQ ID NO: 115 - DNA, 20 bp, synthetic construct
  misc_feature 1..20: note = Description of Artificial Sequence: Synthetic oligonucleotide
  source 1..20: mol_type = other DNA
gagcggaggcataagcagaa

SEQ ID NO: 116 - DNA, 20 bp, synthetic construct
  misc_feature 1..20: note = Description of Artificial Sequence: Synthetic oligonucleotide
  source 1..20: mol_type = other DNA
gatctggtggctagattcta

SEQ ID NO: 117 - DNA, 20 bp, synthetic construct
  misc_feature 1..20: note = Description of Artificial Sequence: Synthetic oligonucleotide
  source 1..20: mol_type = other DNA
gaggaatcacagctcaacaa

SEQ ID NO: 118 - DNA, 20 bp, synthetic construct
  misc_feature 1..20: note = Description of Artificial Sequence: Synthetic oligonucleotide
  source 1..20: mol_type = other DNA
gatcagaaaacggccctgga

SEQ ID NO: 119 - DNA, 20 bp, synthetic construct
  misc_feature 1..20: note = Description of Artificial Sequence: Synthetic oligonucleotide
  source 1..20: mol_type = other DNA
ggttttgtcagcttacctga

SEQ ID NO: 120 - DNA, 20 bp, synthetic construct
  misc_feature 1..20: note = Description of Artificial Sequence: Synthetic oligonucleotide
  source 1..20: mol_type = other DNA
ggcatccaagatggttagaa

SEQ ID NO: 121 - DNA, 20 bp, synthetic construct
  misc_feature 1..20: note = Description of Artificial Sequence: Synthetic oligonucleotide
  source 1..20: mol_type = other DNA
gattcctaaggctctccatc

SEQ ID NO: 122 - DNA, 20 bp, synthetic construct
  misc_feature 1..20: note = Description of Artificial Sequence: Synthetic oligonucleotide
  source 1..20: mol_type = other DNA
gcaatacagactaggaatta

SEQ ID NO: 123 - DNA, 20 bp, synthetic construct
  misc_feature 1..20: note = Description of Artificial Sequence: Synthetic oligonucleotide
  source 1..20: mol_type = other DNA
gagctcagggagcatcgagg

SEQ ID NO: 124 - DNA, 20 bp, synthetic construct
  misc_feature 1..20: note = Description of Artificial Sequence: Synthetic oligonucleotide
  source 1..20: mol_type = other DNA
gagagtcgcaattggagcgc

SEQ ID NO: 125 - DNA, 20 bp, synthetic construct
  misc_feature 1..20: note = Description of Artificial Sequence: Synthetic oligonucleotide
  source 1..20: mol_type = other DNA
gccagaccagcctgcacagt

SEQ ID NO: 126 - DNA, 20 bp, synthetic construct
  misc_feature 1..20: note = Description of Artificial Sequence: Synthetic oligonucleotide
  source 1..20: mol_type = other DNA
gagcgcaggctaggcctgca

SEQ ID NO: 127 - DNA, 20 bp, synthetic construct
  misc_feature 1..20: note = Description of Artificial Sequence: Synthetic oligonucleotide
  source 1..20: mol_type = other DNA
gctaggagtccgggataccc

SEQ ID NO: 128 - DNA, 20 bp, synthetic construct
  misc_feature 1..20: note = Description of Artificial Sequence: Synthetic oligonucleotide
  source 1..20: mol_type = other DNA
gaatccgcaggtgcactcac

SEQ ID NO: 129 - DNA, 20 bp, synthetic construct
  misc_feature 1..20: note = Description of Artificial Sequence: Synthetic oligonucleotide
  source 1..20: mol_type = other DNA
gaccagcctgcacagtgggc

SEQ ID NO: 130 - DNA, 20 bp, synthetic construct
  misc_feature 1..20: note = Description of Artificial Sequence: Synthetic oligonucleotide
  source 1..20: mol_type = other DNA
gcgacgcggttggcagccga

SEQ ID NO: 131 - DNA, 20 bp, synthetic construct
  misc_feature 1..20: note = Description of Artificial Sequence: Synthetic oligonucleotide
  source 1..20: mol_type = other DNA
ggcagggtggaactcgtgac

SEQ ID NO: 132 - DNA, 20 bp, synthetic construct
  misc_feature 1..20: note = Description of Artificial Sequence: Synthetic oligonucleotide
  source 1..20: mol_type = other DNA
gcaccatccagcaagcaggg

SEQ ID NO: 133 - DNA, 20 bp, synthetic construct
  misc_feature 1..20: note = Description of Artificial Sequence: Synthetic oligonucleotide
  source 1..20: mol_type = other DNA
gcgtcactcaaggatctaca

SEQ ID NO: 134 - DNA, 20 bp, synthetic construct
  misc_feature 1..20: note = Description of Artificial Sequence: Synthetic oligonucleotide
  source 1..20: mol_type = other DNA
gatgggaatggcacccacga

SEQ ID NO: 135 - DNA, 20 bp, synthetic construct
  misc_feature 1..20: note = Description of Artificial Sequence: Synthetic oligonucleotide
  source 1..20: mol_type = other DNA
gcctttagacggagaacaga

SEQ ID NO: 136 - DNA, 20 bp, synthetic construct
  misc_feature 1..20: note = Description of Artificial Sequence: Synthetic oligonucleotide
  source 1..20: mol_type = other DNA
gagatccttgagtgacggac

SEQ ID NO: 137 - DNA, 20 bp, synthetic construct
  misc_feature 1..20: note = Description of Artificial Sequence: Synthetic oligonucleotide
  source 1..20: mol_type = other DNA
gcggggctcctccacgaagg

SEQ ID NO: 138 - DNA, 20 bp, synthetic construct
  misc_feature 1..20: note = Description of Artificial Sequence: Synthetic oligonucleotide
  source 1..20: mol_type = other DNA
gcaaggaatcacgccttcgt

SEQ ID NO: 139 - DNA, 20 bp, synthetic construct
  misc_feature 1..20: note = Description of Artificial Sequence: Synthetic oligonucleotide
  source 1..20: mol_type = other DNA
ggccatgcgcgaatgctgag

SEQ ID NO: 140 - DNA, 20 bp, synthetic construct
  misc_feature 1..20: note = Description of Artificial Sequence: Synthetic oligonucleotide
  source 1..20: mol_type = other DNA
ggcaagcccagccaccttcg

SEQ ID NO: 141 - DNA, 20 bp, synthetic construct
  misc_feature 1..20: note = Description of Artificial Sequence: Synthetic oligonucleotide
  source 1..20: mol_type = other DNA
gaggtaagccatccaggctg

SEQ ID NO: 142 - DNA, 20 bp, synthetic construct
  misc_feature 1..20: note = Description of Artificial Sequence: Synthetic oligonucleotide
  source 1..20: mol_type = other DNA
gttcctgctagggaggctca

SEQ ID NO: 143 - DNA, 20 bp, synthetic construct
  misc_feature 1..20: note = Description of Artificial Sequence: Synthetic oligonucleotide
  source 1..20: mol_type = other DNA
gcctgaaacgacagaggatg

SEQ ID NO: 144 - DNA, 20 bp, synthetic construct
  misc_feature 1..20: note = Description of Artificial Sequence: Synthetic oligonucleotide
  source 1..20: mol_type = other DNA
gtcagaggtggagaccaggt

SEQ ID NO: 145 - DNA, 20 bp, synthetic construct
  misc_feature 1..20: note = Description of Artificial Sequence: Synthetic oligonucleotide
  source 1..20: mol_type = other DNA
gccccagcctgaaacgacag

SEQ ID NO: 146 - DNA, 20 bp, synthetic construct
  misc_feature 1..20: note = Description of Artificial Sequence: Synthetic oligonucleotide
  source 1..20: mol_type = other DNA
ggccaagagcgagaatctcc

SEQ ID NO: 147 - DNA, 20 bp, synthetic construct
  misc_feature 1..20: note = Description of Artificial Sequence: Synthetic oligonucleotide
  source 1..20: mol_type = other DNA
ggtcaggtgtcagagcccat

SEQ ID NO: 148 - DNA, 20 bp, synthetic construct
  misc_feature 1..20: note = Description of Artificial Sequence: Synthetic oligonucleotide
  source 1..20: mol_type = other DNA
gggtgtcagagcccatcggt

SEQ ID NO: 149 - DNA, 20 bp, synthetic construct
  misc_feature 1..20: note = Description of Artificial Sequence: Synthetic oligonucleotide
  source 1..20: mol_type = other DNA
gtgccctgagcctccctagc

SEQ ID NO: 150 - DNA, 20 bp, synthetic construct
  misc_feature 1..20: note = Description of Artificial Sequence: Synthetic oligonucleotide
  source 1..20: mol_type = other DNA
gtctgtgagaaccgaccgat

SEQ ID NO: 151 - DNA, 20 bp, synthetic construct
  misc_feature 1..20: note = Description of Artificial Sequence: Synthetic oligonucleotide
  source 1..20: mol_type = other DNA
gggctccgcaggcgcagcgg

SEQ ID NO: 152 - DNA, 20 bp, synthetic construct
  misc_feature 1..20: note = Description of Artificial Sequence: Synthetic oligonucleotide
  source 1..20: mol_type = other DNA
ggggccagcgcgggggacag

SEQ ID NO: 153 - DNA, 20 bp, synthetic construct
  misc_feature 1..20: note = Description of Artificial Sequence: Synthetic oligonucleotide
  source 1..20: mol_type = other DNA
gccgctagcgggccacacag

SEQ ID NO: 154 - DNA, 20 bp, synthetic construct
  misc_feature 1..20: note = Description of Artificial Sequence: Synthetic oligonucleotide
  source 1..20: mol_type = other DNA
gcgggggacagcggctccgg

SEQ ID NO: 155 - DNA, 20 bp, synthetic construct
  misc_feature 1..20: note = Description of Artificial Sequence: Synthetic oligonucleotide
  source 1..20: mol_type = other DNA
gcatcggccccggcttcgag

SEQ ID NO: 156 - DNA, 20 bp, synthetic construct
  misc_feature 1..20: note = Description of Artificial Sequence: Synthetic oligonucleotide
  source 1..20: mol_type = other DNA
ggggtacggcgagatcgcaa

SEQ ID NO: 157 - DNA, 20 bp, synthetic construct
  misc_feature 1..20: note = Description of Artificial Sequence: Synthetic oligonucleotide
  source 1..20: mol_type = other DNA
gatgccgacgcgcacgacca

SEQ ID NO: 158 - DNA, 20 bp, synthetic construct
  misc_feature 1..20: note = Description of Artificial Sequence: Synthetic oligonucleotide
  source 1..20: mol_type = other DNA
ggccgccgccgctgcgcctg

SEQ ID NO: 159 - DNA, 20 bp, synthetic construct
  misc_feature 1..20: note = Description of Artificial Sequence: Synthetic oligonucleotide
  source 1..20: mol_type = other DNA
ggggcccggactgttcccgg

SEQ ID NO: 160 - DNA, 20 bp, synthetic construct
  misc_feature 1..20: note = Description of Artificial Sequence: Synthetic oligonucleotide
  source 1..20: mol_type = other DNA
gagcgggccacacaggggta

SEQ ID NO: 161 - DNA, 20 bp, synthetic construct
  misc_feature 1..20: note = Description of Artificial Sequence: Synthetic oligonucleotide
  source 1..20: mol_type = other DNA
gggacttaccagctaggtgc

SEQ ID NO: 162 - DNA, 20 bp, synthetic construct
  misc_feature 1..20: note = Description of Artificial Sequence: Synthetic oligonucleotide
  source 1..20: mol_type = other DNA
gcccacaaagaacagctcca

SEQ ID NO: 163 - DNA, 20 bp, synthetic construct
  misc_feature 1..20: note = Description of Artificial Sequence: Synthetic oligonucleotide
  source 1..20: mol_type = other DNA
ggctggtaagtccttctcat

SEQ ID NO: 164 - DNA, 20 bp, synthetic construct
  misc_feature 1..20: note = Description of Artificial Sequence: Synthetic oligonucleotide
  source 1..20: mol_type = other DNA
gggtgcaggcacactccaaa

SEQ ID NO: 165 - DNA, 20 bp, synthetic construct
  misc_feature 1..20: note = Description of Artificial Sequence: Synthetic oligonucleotide
  source 1..20: mol_type = other DNA
gacttaacttggctgactgt

SEQ ID NO: 166 - DNA, 20 bp, synthetic construct
  misc_feature 1..20: note = Description of Artificial Sequence: Synthetic oligonucleotide
  source 1..20: mol_type = other DNA
gtcagcctcccagaagtcca

SEQ ID NO: 167 - DNA, 20 bp, synthetic construct
  misc_feature 1..20: note = Description of Artificial Sequence: Synthetic oligonucleotide
  source 1..20: mol_type = other DNA
ggctgccttggacttctggg

SEQ ID NO: 168 - DNA, 20 bp, synthetic construct
  misc_feature 1..20: note = Description of Artificial Sequence: Synthetic oligonucleotide
  source 1..20: mol_type = other DNA
gccacggaaggcctccagat

SEQ ID NO: 169 - DNA, 20 bp, synthetic construct
  misc_feature 1..20: note = Description of Artificial Sequence: Synthetic oligonucleotide
  source 1..20: mol_type = other DNA
gccaaggcacttgctccatt

SEQ ID NO: 170 - DNA, 20 bp, synthetic construct
  misc_feature 1..20: note = Description of Artificial Sequence: Synthetic oligonucleotide
  source 1..20: mol_type = other DNA
gggctgctgtgtggtaagag

SEQ ID NO: 171 - DNA, 20 bp, synthetic construct
  misc_feature 1..20: note = Description of Artificial Sequence: Synthetic oligonucleotide
  source 1..20: mol_type = other DNA
gccaacctgaatggaagaga

SEQ ID NO: 172 - DNA, 20 bp, synthetic construct
  misc_feature 1..20: note = Description of Artificial Sequence: Synthetic oligonucleotide
  source 1..20: mol_type = other DNA
gagggaagtggaaagcaagg

SEQ ID NO: 173 - DNA, 20 bp, synthetic construct
  misc_feature 1..20: note = Description of Artificial Sequence: Synthetic oligonucleotide
  source 1..20: mol_type = other DNA
gtgggacaggcatggatgaa

SEQ ID NO: 174 - DNA, 20 bp, synthetic construct
  misc_feature 1..20: note = Description of Artificial Sequence: Synthetic oligonucleotide
  source 1..20: mol_type = other DNA
gcctgtcccaggaacggcat

SEQ ID NO: 175 - DNA, 20 bp, synthetic construct
  misc_feature 1..20: note = Description of Artificial Sequence: Synthetic oligonucleotide
  source 1..20: mol_type = other DNA
gtgagaaaagccaacctgaa

SEQ ID NO: 176 - DNA, 20 bp, synthetic construct
  misc_feature 1..20: note = Description of Artificial Sequence: Synthetic oligonucleotide
  source 1..20: mol_type = other DNA
ggattcgagtgtctcccgga

SEQ ID NO: 177 - DNA, 20 bp, synthetic construct
  misc_feature 1..20: note = Description of Artificial Sequence: Synthetic oligonucleotide
  source 1..20: mol_type = other DNA
gaccaagtcgttataaggaa

SEQ ID NO: 178 - DNA, 20 bp, synthetic construct
  misc_feature 1..20: note = Description of Artificial Sequence: Synthetic oligonucleotide
  source 1..20: mol_type = other DNA
gaagtcgttataaggaaagg

SEQ ID NO: 179 - DNA, 20 bp, synthetic construct
  misc_feature 1..20: note = Description of Artificial Sequence: Synthetic oligonucleotide
  source 1..20: mol_type = other DNA
ggaatgaccacgctccacgg

SEQ ID NO: 180 - DNA, 20 bp, synthetic construct
  misc_feature 1..20: note = Description of Artificial Sequence: Synthetic oligonucleotide
  source 1..20: mol_type = other DNA
gcctctggtgtgtactctgt

SEQ ID NO: 181 - DNA, 20 bp, Mus sp.
  source 1..20: mol_type = other DNA
aaagtgatagaaatccacga

SEQ ID NO: 182 - DNA, 20 bp, Mus sp.
  source 1..20: mol_type = other DNA
gtgtgtttgcaagatcaatg

SEQ ID NO: 183 - DNA, 20 bp, Mus sp.
  source 1..20: mol_type = other DNA
ctggatgggaacccgctgag

SEQ ID NO: 184 - DNA, 20 bp, Mus sp.
  source 1..20: mol_type = other DNA
tatcctgaccaacacgatgg

SEQ ID NO: 185 - DNA, 20 bp, Mus sp.
  source 1..20: mol_type = other DNA
gccagttccaagggtcacgg

SEQ ID NO: 186 - DNA, 20 bp, Mus sp.
  source 1..20: mol_type = other DNA
gtgtccgtagagatttaatg

SEQ ID NO: 187 - DNA, 20 bp, Mus sp.
  source 1..20: mol_type = other DNA
tatctcaaaccgtacccttg

SEQ ID NO: 188 - DNA, 20 bp, Mus sp.
  source 1..20: mol_type = other DNA
ctgagtacacgagtttaggg

SEQ ID NO: 189 - DNA, 20 bp, Mus sp.
  source 1..20: mol_type = other DNA
caagagaagacgttaccaag

SEQ ID NO: 190 - DNA, 20 bp, Mus sp.
  source 1..20: mol_type = other DNA
gatccattgccacacaacaa

SEQ ID NO: 191 - DNA, 20 bp, Mus sp.
  source 1..20: mol_type = other DNA
ccagcaatatggaacttcga

SEQ ID NO: 192 - DNA, 20 bp, Mus sp.
  source 1..20: mol_type = other DNA
catcactgatcctaacgtgt

SEQ ID NO: 193 - DNA, 20 bp, Mus sp.
  source 1..20: mol_type = other DNA
tattgcacgtggaaccatcg

SEQ ID NO: 194 - DNA, 20 bp, Mus sp.
  source 1..20: mol_type = other DNA
gaggacgatatggaatgttg

SEQ ID NO: 195 - DNA, 20 bp, Mus sp.
  source 1..20: mol_type = other DNA
tttgtttgctcaaggagttg

SEQ ID NO: 196 - DNA, 20 bp, Mus sp.
  source 1..20: mol_type = other DNA
cttaatgagagtgtttaatg

SEQ ID NO: 197 - DNA, 20 bp, Mus sp.
  source 1..20: mol_type = other DNA
gaaccctctccgacgcaccg

SEQ ID NO: 198 - DNA, 20 bp, Mus sp.
  source 1..20: mol_type = other DNA
agatgcgacagtatgacacc

SEQ ID NO: 199 - DNA, 20 bp, Mus sp.
  source 1..20: mol_type = other DNA
cgtgctcggatcatacaggc

SEQ ID NO: 200 - DNA, 20 bp, Mus sp.
  source 1..20: mol_type = other DNA
gtacctacagatttggtccg

SEQ ID NO: 201 - DNA, 20 bp, Mus sp.
  source 1..20: mol_type = other DNA
taagctgaataacaccgttg

SEQ ID NO: 202 - DNA, 20 bp, Mus sp.
  source 1..20: mol_type = other DNA
aagccacatactccttgcga

SEQ ID NO: 203 - DNA, 20 bp, Mus sp.
  source 1..20: mol_type = other DNA
cctgcgatcatagagccttg

SEQ ID NO: 204 - DNA, 20 bp, Mus sp.
  source 1..20: mol_type = other DNA
gctccacgagaagcatgtcg

SEQ ID NO: 205 - DNA, 20 bp, Mus sp.
  source 1..20: mol_type = other DNA
tatcctacgcttgctccgaa

SEQ ID NO: 206 - DNA, 20 bp, Mus sp.
  source 1..20: mol_type = other DNA
ggcaccggttgtaacccaca

SEQ ID NO: 207 - DNA, 20 bp, Mus sp.
  source 1..20: mol_type = other DNA
acatcatggaagaatacgac

SEQ ID NO: 208 - DNA, 20 bp, Mus sp.
  source 1..20: mol_type = other DNA
tgactggctacggctacaca

SEQ ID NO: 209 - DNA, 30 bp, Mus sp.
  source 1..30: mol_type = other DNA
gccgaaagtgatagaaatccacgaagggaa

SEQ ID NO: 210 - DNA, 30 bp, Mus sp.
  source 1..30: mol_type = other DNA
aggagtgtgtttgcaagatcaatgaggact

SEQ ID NO: 211 - DNA, 30 bp, Mus sp.
  source 1..30: mol_type = other DNA
ctccctggatgggaacccgctgagcggcga

SEQ ID NO: 212 - DNA, 30 bp, Mus sp.
  source 1..30: mol_type = other DNA
ccagtatcctgaccaacacgatggagggta

SEQ ID NO: 213 - DNA, 30 bp, Mus sp.
  source 1..30: mol_type = other DNA
tccagccagttccaagggtcacggaggaag

SEQ ID NO: 214 - DNA, 30 bp, Mus sp.
  source 1..30: mol_type = other DNA
ctcagtgtccgtagagatttaatggggcca

SEQ ID NO: 215 - DNA, 30 bp, Mus sp.
  source 1..30: mol_type = other DNA
actatatctcaaaccgtacccttgcggaga

SEQ ID NO: 216 - DNA, 30 bp, Mus sp.
  source 1..30: mol_type = other DNA
gctgctgagtacacgagtttagggcggagc

SEQ ID NO: 217 - DNA, 30 bp, Mus sp.
  source 1..30: mol_type = other DNA
tggccaagagaagacgttaccaagcggaag

SEQ ID NO: 218 - DNA, 30 bp, Mus sp.
  source 1..30: mol_type = other DNA
atcagatccattgccacacaacaagggatc

SEQ ID NO: 219 - DNA, 30 bp, Mus sp.
  source 1..30: mol_type = other DNA
ctgcccagcaatatggaacttcgacggctt

SEQ ID NO: 220 - DNA, 30 bp, Mus sp.
  source 1..30: mol_type = other DNA
acttcatcactgatcctaacgtgtgggtct

SEQ ID NO: 221 - DNA, 30 bp, Mus sp.
  source 1..30: mol_type = other DNA
gttttattgcacgtggaaccatcggggcag

SEQ ID NO: 222 - DNA, 30 bp, Mus sp.
  source 1..30: mol_type = other DNA
agaagaggacgatatggaatgttgtggtga

SEQ ID NO: 223 - DNA, 30 bp, Mus sp.
  source 1..30: mol_type = other DNA
tcgttttgtttgctcaaggagttgtggctg

SEQ ID NO: 224 - DNA, 30 bp, Mus sp.
  source 1..30: mol_type = other DNA
tgatcttaatgagagtgtttaatgtgggcc

SEQ ID NO: 225 - DNA, 30 bp, Mus sp.
  source 1..30: mol_type = other DNA
acgagaaccctctccgacgcaccgcgggcc

SEQ ID NO: 226 - DNA, 30 bp, Mus sp.
  source 1..30: mol_type = other DNA
gtgcagatgcgacagtatgacacccggcat

SEQ ID NO: 227 - DNA, 30 bp, Mus sp.
  source 1..30: mol_type = other DNA
gaggcgtgctcggatcatacaggccggcgg

SEQ ID NO: 228 - DNA, 30 bp, Mus sp.
  source 1..30: mol_type = other DNA
agccgtacctacagatttggtccgtggaat

SEQ ID NO: 229 - DNA, 30 bp, Mus sp.
  source 1..30: mol_type = other DNA
cctataagctgaataacaccgttggggact

SEQ ID NO: 230 - DNA, 30 bp, Mus sp.
  source 1..30: mol_type = other DNA
atggaagccacatactccttgcgatggctg

SEQ ID NO: 231 - DNA, 30 bp, Mus sp.
  source 1..30: mol_type = other DNA
tgctcctgcgatcatagagccttgggggcg

SEQ ID NO: 232 - DNA, 30 bp, Mus sp.
  source 1..30: mol_type = other DNA
aagggctccacgagaagcatgtcgtggcgg

SEQ ID NO: 233 - DNA, 30 bp, Mus sp.
  source 1..30: mol_type = other DNA
ccaatatcctacgcttgctccgaacggcca

SEQ ID NO: 234 - DNA, 30 bp, Mus sp.
  source 1..30: mol_type = other DNA
gctaggcaccggttgtaacccacagggctg

SEQ ID NO: 235 - DNA, 30 bp, Mus sp.
  source 1..30: mol_type = other DNA
ctcaacatcatggaagaatacgactggtac

SEQ ID NO: 236 - DNA, 30 bp, Mus sp.
  source 1..30: mol_type = other DNA
gggctgactggctacggctacacatggatc

SEQ ID NO: 237 - DNA, 595 bp, synthetic construct
  misc_feature 1..595: note = Description of Artificial Sequence: Synthetic polynucleotide
  source 1..595: mol_type = other DNA
gcagagctctctggctaactaccggtgccaccatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaagaattcctagagctcgctgatcagcc

SEQ ID NO: 238 - DNA, 865 bp, synthetic construct
  misc_feature 1..865: note = Description of Artificial Sequence: Synthetic polynucleotide
  source 1..865: mol_type = other DNA
gcagagctctctggctaactaccggtgccaccatggcgcggttcctgaggctttgcacctggctgctggcgcttgggtcctgcctcctggctacagtgcaggcggaatgcagccaggactgcgctaaatgcagctaccgcctggttcgcccaggcgacatcaatttcctggcgtgcacactggaatgtgaaggacagctgccttctttcaaaatctgggagacctgcaaggatctcctgcaggtgtccaggcccgagttcccttgggataacatcgacatgtacaaagacagcagcaaacaggatgagagccacttgctagccaagaagtacggaggcttcatgaaacggtacggaggcttcatgaagaagatggacgagctatatcccatggagccagaagaagaagcgaacggaggagagatccttgccaagaggtatggcggcttcatgaagaaggatgcagatgagggagacaccttggccaactcctccgatctgctgaaagagctactgggaacgggagacaaccgtgcgaaagacagccaccaacaagagagcaccaacaatgacgaagacatgagcaagaggtatgggggcttcatgagaagcctcaaaagaagcccccaactggaagatgaagcaaaagagctgcagaagcgctacgggggcttcatgagaagggtgggacgccccgagtggtggatggactaccagaagaggtatgggggcttcctgaagcgctttgctgagtctctgccctccgatgaagaaggcgaaaattactcgaaagaagttcctgagatagagaaaagatacgggggctttatgcggttctgagaattcctagagctcgctgatcagcc

SEQ ID NO: 239 - DNA, 766 bp, synthetic construct
  misc_feature 1..766: note = Description of Artificial Sequence: Synthetic polynucleotide
  source 1..766: mol_type = other DNA
gcagagctctctggctaactaccggtgccaccatgccgagattctgctacagtcgctcaggggccctgttgctggccctcctgcttcagacctccatagatgtgtggagctggtgcctggagagcagccagtgccaggacctcaccacggagagcaacctgctggcttgcatccgggcttgcaaactcgacctctcgctggagacgcccgtgtttcctggcaacggagatgaacagcccctgactgaaaacccccggaagtacgtcatgggtcacttccgctgggaccgcttcggccccaggaacagcagcagtgctggcagcgcggcgcagaggcgtgcggaggaagaggcggtgtggggagatggcagtccagagccgagtccacgcgagggcaagcgctcctactccatggagcacttccgctggggcaagccggtgggcaagaaacggcgcccggtgaaggtgtaccccaacgttgctgagaacgagtcggcggaggcctttcccctagagttcaagagggagctggaaggcgagcggccattaggcttggagcaggtcctggagtccgacgcggagaaggacgacgggccctaccgggtggagcacttccgctggagcaacccgcccaaggacaagcgttacggtggcttcatgacctccgagaagagccagacgcccctggtgacgctcttcaagaacgccatcatcaagaacgcgcacaagaagggccagtgagaattcctagagctcgctgatcagcc

SEQ ID NO: 240 - DNA, 1144 bp, synthetic construct
  misc_feature 1..1144: note = Description of Artificial Sequence: Synthetic polynucleotide
  source 1..1144: mol_type = other DNA
gcagagctctctggctaactaccggtgccaccatgagtgcattgctcatcctggccctggtcggggctgccgtggcttgtaaaggcaaaggagctaaatgcagtagacttatgtatgattgttgcacgggttcatgtagatcagggaagtgcatcgactataaagacgacgatgacaaactggcagctgccggtaacggtaatgggaatgggaacggcaacgggaacggtaacggagacggcacgagggtagcagtaggacaggacacgcaagaggtaatcgttgtaccgcatagtctccccttcaaggtagtagtgatcagtgctatactggcgctggtggttctcacaattattagtctgataattttgataatgctgtggcaaaaaaagccccggagaatccgaatggtcagtaagggtgaagaagacaatatggccataattaaggagttcatgcgattcaaggtacatatggagggtagcgtcaatggtcacgagttcgaaatagaaggcgaaggcgaggggagaccctatgaaggaacacagacagctaaacttaaggtaacgaaaggcggcccactcccgttcgcctgggatattcttagtccgcagttcatgtacggttcaaaggcgtatgtcaaacatccagcggacatccccgattacctgaaattgagcttcccagagggatttaaatgggagcgggtcatgaatttcgaagatgggggagttgtgacagtaactcaagactccagtctccaggatggtgaattcatatacaaagtcaaactcaggggcaccaatttccccagcgacggccccgtcatgcaaaagaaaaccatgggatgggaggccagctccgagcgcatgtatcctgaggatggagctcttaaaggagagatcaaacagcgcctgaagttgaaggatggaggccactacgatgccgaggttaagacaacctataaggccaaaaagccagtgcagcttccgggagcgtacaatgtaaacatcaagctggatattacgagccacaacgaggactacacgatagtagaacagtacgagagagcagagggacggcactccactggtggtatggacgaattgtataagtaagaattcctagagctcgctgatcagcc

SEQ ID NO: 241 - DNA, 20 bp, synthetic construct
  misc_feature 1..20: note = Description of Artificial Sequence: Synthetic oligonucleotide
  source 1..20: mol_type = other DNA
cgaaattgaagacgaagagc

SEQ ID NO: 242 - DNA, 20 bp, synthetic construct
  misc_feature 1..20: note = Description of Artificial Sequence: Synthetic oligonucleotide
  source 1..20: mol_type = other DNA
ggagactgagagagagaagc

SEQ ID NO: 243 - DNA, 20 bp, synthetic construct
  misc_feature 1..20: note = Description of Artificial Sequence: Synthetic oligonucleotide
  source 1..20: mol_type = other DNA
tgatgagggagggcaccatg

SEQ ID NO: 244 - DNA, 20 bp, synthetic construct
  misc_feature 1..20: note = Description of Artificial Sequence: Synthetic oligonucleotide
  source 1..20: mol_type = other DNA
ggtcctgccgctgcttgtca

SEQ ID NO: 245 - DNA, 20 bp, synthetic construct
  misc_feature 1..20: note = Description of Artificial Sequence: Synthetic oligonucleotide
  source 1..20: mol_type = other DNA
agccggccagttccaaaccc

SEQ ID NO: 246 - DNA, 20 bp, synthetic construct
  misc_feature 1..20: note = Description of Artificial Sequence: Synthetic oligonucleotide
  source 1..20: mol_type = other DNA
agggcccggcgcaatgacag

SEQ ID NO: 247 - DNA, 20 bp, synthetic construct
  misc_feature 1..20: note = Description of Artificial Sequence: Synthetic oligonucleotide
  source 1..20: mol_type = other DNA
tcttcaaataaccactcctg

SEQ ID NO: 248 - DNA, 20 bp, synthetic construct
  misc_feature 1..20: note = Description of Artificial Sequence: Synthetic oligonucleotide
  source 1..20: mol_type = other DNA
tcagcaacaatgtcaacacc

SEQ ID NO: 249 - DNA, 20 bp, synthetic construct
  misc_feature 1..20: note = Description of Artificial Sequence: Synthetic oligonucleotide
  source 1..20: mol_type = other DNA
ggcaatctccataatgccgt

SEQ ID NO: 250 - DNA, 20 bp, synthetic construct
  misc_feature 1..20: note = Description of Artificial Sequence: Synthetic oligonucleotide
  source 1..20: mol_type = other DNA
tatccacagagcctaaccca

SEQ ID NO: 251 - DNA, 20 bp, synthetic construct
  misc_feature 1..20: note = Description of Artificial Sequence: Synthetic oligonucleotide
  source 1..20: mol_type = other DNA
tgtacgaaaagccagtgatg

SEQ ID NO: 252 - DNA, 20 bp, synthetic construct
  misc_feature 1..20: note = Description of Artificial Sequence: Synthetic oligonucleotide
  source 1..20: mol_type = other DNA
gggttcactccagacctgtg

SEQ ID NO: 253 - DNA, 20 bp, synthetic construct
  misc_feature 1..20: note = Description of Artificial Sequence: Synthetic oligonucleotide
  source 1..20: mol_type = other DNA
aaggtctgagaatcgcgaag

SEQ ID NO: 254 - DNA, 20 bp, synthetic construct
  misc_feature 1..20: note = Description of Artificial Sequence: Synthetic oligonucleotide
  source 1..20: mol_type = other DNA
cattctggcagagttagcag

SEQ ID NO: 255 - protein, 446 aa, Homo sapiens
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 256 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 257 - protein, 437 aa, synthetic construct
  REGION 1..437: note = Description of Artificial Sequence: Synthetic polypeptide
MADDPSAADRNVEIWKIKKLIKSLEAARGNGTSMISLIIPPKDQISRVAKMLADDFGTASNIKSRVNRLSVLGAITSVQQRLKLYNKVPPNGLVVYCGTIVTEEGKEKKVNIDFEPFKPINTSLYLCDNKFHTEALTALLSDDSKFGFIVIDGSGALFGTLQGNTREVLHKFTVDLPKKHGRGGQSALRFARLRMEKRHNYVRKVAETAVQLFISGDKVNVAGLVLAGSADFKTELSQSDMFDQRLQSKVLKLVDISYGGENGFNQAIELSTEVLSNVKFIQEKKLIGRYFDEISQDTGKYCFGVEDTLKALEMGAVEILIVYENLDIMRYVLHCQGTEEEKILYLTPEQEKDKSHFTDKETGQEHELIESMPLLEWFANNYKKFGATLEIVTDKSQEGSQFVKGFGGIGGILRYRVDFQGMEYQGGDDEFFDLDDY

SEQ ID NO: 258 - protein, 13 aa, synthetic construct
  REGION 1..13: note = Description of Artificial Sequence: Synthetic peptide
AHIVMVDAYKPTK

SEQ ID NO: 259 - protein, 10 aa, synthetic construct
  REGION 1..10: note = Description of Artificial Sequence: Synthetic peptide
ATHIKFSKRD

SEQ ID NO: 260 - protein, 735 aa, Adeno-associated virus 2 [sequence omitted]

SEQ ID NO: 261 - protein, 737 aa, synthetic construct
  REGION 1..737: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 262 - protein, 27 aa, synthetic construct
  REGION 1..27: note = Description of Artificial Sequence: Synthetic peptide
ATHIKFSKRDGSGSGSRPKPQQFFGLM

SEQ ID NO: 263 - protein, 27 aa, synthetic construct
  REGION 1..27: note = Description of Artificial Sequence: Synthetic peptide
RPKPQQFFGLMGSGSGSATHIKFSKRD

SEQ ID NO: 264 - protein, 50 aa, synthetic construct
  REGION 1..50: note = Description of Artificial Sequence: Synthetic polypeptide
YTIWMPENPRPGTPCDIFTNSRGKRASNGGGKGGGSGSGSATHIKFSKRD

SEQ ID NO: 265 - protein, 50 aa, synthetic construct
  REGION 1..50: note = Description of Artificial Sequence: Synthetic polypeptide
ATHIKFSKRDGSGSGSGGKGGYTIWMPENPRPGTPCDIFTNSRGKRASNG

SEQ ID NO: 266 - DNA, 16 bp, synthetic construct
  misc_feature 1..16: note = Description of Artificial Sequence: Synthetic primer
  source 1..16: mol_type = other DNA
cggcctcagtgagcga

SEQ ID NO: 267 - DNA, 21 bp, synthetic construct
  misc_feature 1..21: note = Description of Artificial Sequence: Synthetic primer
  source 1..21: mol_type = other DNA
ggaacccctagtgatggagtt

SEQ ID NO: 268 - DNA, 20 bp, synthetic construct
  misc_feature 1..20: note = Description of Artificial Sequence: Synthetic oligonucleotide
  source 1..20: mol_type = other DNA
ggggccactagggacaggat

SEQ ID NO: 269 - DNA, 20 bp, synthetic construct
  misc_feature 1..20: note = Description of Artificial Sequence: Synthetic oligonucleotide
  source 1..20: mol_type = other DNA
gagtccgagcagaagaagaa

SEQ ID NO: 270 - DNA, 20 bp, synthetic construct
  misc_feature 1..20: note = Description of Artificial Sequence: Synthetic oligonucleotide
  source 1..20: mol_type = other DNA
ggaatcccttctgcagcacc

SEQ ID NO: 271 - DNA, 20 bp, synthetic construct
  misc_feature 1..20: note = Description of Artificial Sequence: Synthetic oligonucleotide
  source 1..20: mol_type = other DNA
cagcccaagatagttaagtg

SEQ ID NO: 272 - DNA, 20 bp, synthetic construct
  misc_feature 1..20: note = Description of Artificial Sequence: Synthetic oligonucleotide
  source 1..20: mol_type = other DNA
cgggtggtcggtagtgagtc

SEQ ID NO: 273 - DNA, 22 bp, synthetic construct
  misc_feature 1..22: note = Description of Artificial Sequence: Synthetic oligonucleotide
  source 1..22: mol_type = other DNA
cagacgcgaggaaggagggcgc

SEQ ID NO: 274 - DNA, 20 bp, synthetic construct
  misc_feature 1..20: note = Description of Artificial Sequence: Synthetic oligonucleotide
  source 1..20: mol_type = other DNA
cgggagaaaggaacgggagg

SEQ ID NO: 275 - DNA, 20 bp, synthetic construct
  misc_feature 1..20: note = Description of Artificial Sequence: Synthetic oligonucleotide
  source 1..20: mol_type = other DNA
gacgcgtgctctccctcatc

SEQ ID NO: 276 - DNA, 20 bp, synthetic construct
  misc_feature 1..20: note = Description of Artificial Sequence: Synthetic oligonucleotide
  source 1..20: mol_type = other DNA
gctgtgggttgggcctgctg

SEQ ID NO: 277 - DNA, 20 bp, synthetic construct
  misc_feature 1..20: note = Description of Artificial Sequence: Synthetic oligonucleotide
  source 1..20: mol_type = other DNA
accccaccatccatccgcca

SEQ ID NO: 278 - DNA, 20 bp, synthetic construct
  misc_feature 1..20: note = Description of Artificial Sequence: Synthetic oligonucleotide
  source 1..20: mol_type = other DNA
cgaaattgaagacgaagagc

SEQ ID NO: 279 - DNA, 20 bp, synthetic construct
  misc_feature 1..20: note = Description of Artificial Sequence: Synthetic oligonucleotide
  source 1..20: mol_type = other DNA
ggacaaagaccacttcagag

SEQ ID NO: 280 - DNA, 20 bp, synthetic construct
  misc_feature 1..20: note = Description of Artificial Sequence: Synthetic oligonucleotide
  source 1..20: mol_type = other DNA
atttcaggtaagccgaggtt

SEQ ID NO: 281 - DNA, 20 bp, synthetic construct
  misc_feature 1..20: note = Description of Artificial Sequence: Synthetic oligonucleotide
  source 1..20: mol_type = other DNA
ataatttctattatattaca

SEQ ID NO: 282 - DNA, 20 bp, synthetic construct
  misc_feature 1..20: note = Description of Artificial Sequence: Synthetic primer
  source 1..20: mol_type = other DNA
gaagctgttggctgaaaagg

SEQ ID NO: 283 - DNA, 27 bp, synthetic construct
  misc_feature 1..27: note = Description of Artificial Sequence: Synthetic primer
  source 1..27: mol_type = other DNA
ggagatttaggaagtatggggttagtg

SEQ ID NO: 284 - DNA, 19 bp, synthetic construct
  misc_feature 1..19: note = Description of Artificial Sequence: Synthetic primer
  source 1..19: mol_type = other DNA
cgcggccaacaagaagatg

SEQ ID NO: 285 - DNA, 20 bp, synthetic construct
  misc_feature 1..20: note = Description of Artificial Sequence: Synthetic primer
  source 1..20: mol_type = other DNA
acagtcagccgcatcttctt

SEQ ID NO: 286 - DNA, 21 bp, synthetic construct
  misc_feature 1..21: note = Description of Artificial Sequence: Synthetic primer
  source 1..21: mol_type = other DNA
catgtacgttgctatccaggc

SEQ ID NO: 287 - DNA, 21 bp, synthetic construct
  misc_feature 1..21: note = Description of Artificial Sequence: Synthetic primer
  source 1..21: mol_type = other DNA
gctcaactcaggttaccgtga

SEQ ID NO: 288 - DNA, 21 bp, synthetic construct
  misc_feature 1..21: note = Description of Artificial Sequence: Synthetic primer
  source 1..21: mol_type = other DNA
cttccctcatcctcctgctac

SEQ ID NO: 289 - DNA, 23 bp, synthetic construct
  misc_feature 1..23: note = Description of Artificial Sequence: Synthetic primer
  source 1..23: mol_type = other DNA
gctcttcgtcttcaatttcgtct

SEQ ID NO: 290 - DNA, 19 bp, synthetic construct
  misc_feature 1..19: note = Description of Artificial Sequence: Synthetic primer
  source 1..19: mol_type = other DNA
tggccttccgtgttcctac

SEQ ID NO: 291 - DNA, 22 bp, synthetic construct
  misc_feature 1..22: note = Description of Artificial Sequence: Synthetic primer
  source 1..22: mol_type = other DNA
gtgacgttgacatccgtaaaga

SEQ ID NO: 292 - DNA, 20 bp, synthetic construct
  misc_feature 1..20: note = Description of Artificial Sequence: Synthetic primer
  source 1..20: mol_type = other DNA
ctcactgacgttggcaaaga

SEQ ID NO: 293 - DNA, 28 bp, synthetic construct
  misc_feature 1..28: note = Description of Artificial Sequence: Synthetic primer
  source 1..28: mol_type = other DNA
aaaacctcctctcttacttttctacttc

SEQ ID NO: 294 - DNA, 20 bp, synthetic construct
  misc_feature 1..20: note = Description of Artificial Sequence: Synthetic primer
  source 1..20: mol_type = other DNA
cgacgagtaggatgagaccg

SEQ ID NO: 295 - DNA, 20 bp, synthetic construct
  misc_feature 1..20: note = Description of Artificial Sequence: Synthetic primer
  source 1..20: mol_type = other DNA
acgaccaaatccgttgactc

SEQ ID NO: 296 - DNA, 21 bp, synthetic construct
  misc_feature 1..21: note = Description of Artificial Sequence: Synthetic primer
  source 1..21: mol_type = other DNA
ctccttaatgtcacgcacgat

SEQ ID NO: 297 - DNA, 21 bp, synthetic construct
  misc_feature 1..21: note = Description of Artificial Sequence: Synthetic primer
  source 1..21: mol_type = other DNA
agggtgtactggcaagtttgg

SEQ ID NO: 298 - DNA, 22 bp, synthetic construct
  misc_feature 1..22: note = Description of Artificial Sequence: Synthetic primer
  source 1..22: mol_type = other DNA
acaaactgggtaaaggtgatgg

SEQ ID NO: 299 - DNA, 19 bp, synthetic construct
  misc_feature 1..19: note = Description of Artificial Sequence: Synthetic primer
  source 1..19: mol_type = other DNA
tgttgggtgccggtttgtt

SEQ ID NO: 300 - DNA, 20 bp, synthetic construct
  misc_feature 1..20: note = Description of Artificial Sequence: Synthetic primer
  source 1..20: mol_type = other DNA
gagttgctgttgaagtcgca

SEQ ID NO: 301 - DNA, 19 bp, synthetic construct
  misc_feature 1..19: note = Description of Artificial Sequence: Synthetic primer
  source 1..19: mol_type = other DNA
gccggactcatcgtactcc

SEQ ID NO: 302 - DNA, 54 bp, synthetic construct
  misc_feature 1..54: note = Description of Artificial Sequence: Synthetic primer
  source 1..54: mol_type = other DNA
acactctttccctacacgacgctcttccgatctagtgctgcttgctgctggcca

SEQ ID NO: 303 - DNA, 56 bp, synthetic construct
  misc_feature 1..56: note = Description of Artificial Sequence: Synthetic primer
  source 1..56: mol_type = other DNA
gactggagttcagacgtgtgctcttccgatctttgcttgtccctctgtcaatggcg

SEQ ID NO: 304 - DNA, 57 bp, synthetic construct
  misc_feature 1..57: note = Description of Artificial Sequence: Synthetic primer
  source 1..57: mol_type = other DNA
acactctttccctacacgacgctcttccgatctcggttaatgtggctctggttctgg

SEQ ID NO: 305 - DNA, 60 bp, synthetic construct
  misc_feature 1..60: note = Description of Artificial Sequence: Synthetic primer
  source 1..60: mol_type = other DNA
gactggagttcagacgtgtgctcttccgatctggggttagacccaatatcaggagactag

SEQ ID NO: 306 - DNA, 51 bp, synthetic construct
  misc_feature 1..51: note = Description of Artificial Sequence: Synthetic primer
  source 1..51: mol_type = other DNA
acactctttccctacacgacgctcttccgatctatgagtatgcctgccgtg

SEQ ID NO: 307 - DNA, 51 bp, synthetic construct
  misc_feature 1..51: note = Description of Artificial Sequence: Synthetic primer
  source 1..51: mol_type = other DNA
gactggagttcagacgtgtgctcttccgatctgggactcattcagggtagt

SEQ ID NO: 308 - DNA, 53 bp, synthetic construct
  misc_feature 1..53: note = Description of Artificial Sequence: Synthetic primer
  source 1..53: mol_type = other DNA
acactctttccctacacgacgctcttccgatctaggaccaatccaagctccgc

SEQ ID NO: 309 - DNA, 51 bp, synthetic construct
  misc_feature 1..51: note = Description of Artificial Sequence: Synthetic primer
  source 1..51: mol_type = other DNA
gactggagttcagacgtgtgctcttccgatctttgcgctgcgccttctcag

SEQ ID NO: 310 - DNA, 54 bp, synthetic construct
  misc_feature 1..54: note = Description of Artificial Sequence: Synthetic primer
  source 1..54: mol_type = other DNA
acactctttccctacacgacgctcttccgatcttgtagagcaagcagcaggggc

SEQ ID NO: 311 - DNA, 57 bp, synthetic construct
  misc_feature 1..57: note = Description of Artificial Sequence: Synthetic primer
  source 1..57: mol_type = other DNA
gactggagttcagacgtgtgctcttccgatctggtgtccaagaacagtagcaggaac

SEQ ID NO: 312 - DNA, 35 bp, synthetic construct
  misc_feature 1..35: note = Description of Artificial Sequence: Synthetic oligonucleotide
  source 1..35: mol_type = other DNA
aaaaactatattaccctgttatccctagcgtaact

SEQ ID NO: 313 - DNA, 30 bp, synthetic construct
  misc_feature 1..30: note = Description of Artificial Sequence: Synthetic oligonucleotide
  source 1..30: mol_type = other DNA
aaaaatataagcgggagattcgtcctcata

SEQ ID NO: 314 - DNA, 30 bp, synthetic construct
  misc_feature 1..30: note = Description of Artificial Sequence: Synthetic oligonucleotide
  source 1..30: mol_type = other DNA
agttacgctagggataacagggtaatatag

SEQ ID NO: 315 - DNA, 25 bp, synthetic construct
  misc_feature 1..25: note = Description of Artificial Sequence: Synthetic oligonucleotide
  source 1..25: mol_type = other DNA
tatgaggacgaatctcccgcttata

SEQ ID NO: 316 - DNA, 50 bp, synthetic construct
  misc_feature 1..50: note = Description of Artificial Sequence: Synthetic oligonucleotide
  source 1..50: mol_type = other DNA
ggggcttttctgtcaccaatcctgtccctagtggccccactgtggggtgg

SEQ ID NO: 317 - DNA, 35 bp, synthetic construct
  misc_feature 1..35: note = Description of Artificial Sequence: Synthetic oligonucleotide
  source 1..35: mol_type = other DNA
ggggcttttctgtcagtggccccactgtggggtgg

SEQ ID NO: 318 - DNA, 38 bp, synthetic construct
  misc_feature 1..38: note = Description of Artificial Sequence: Synthetic oligonucleotide
  source 1..38: mol_type = other DNA
ggggcttttctgtccctagtggccccactgtggggtgg

SEQ ID NO: 319 - DNA, 38 bp, synthetic construct; identical to SEQ ID NO: 318
  misc_feature 1..38: note = Description of Artificial Sequence: Synthetic oligonucleotide
  source 1..38: mol_type = other DNA

SEQ ID NO: 320 - DNA, 53 bp, synthetic construct
  misc_feature 1..53: note = Description of Artificial Sequence: Synthetic oligonucleotide
  source 1..53: mol_type = other DNA
ggggcttttctgtcaccaactgtggttgacagaaaagccccactgtggggtgg

SEQ ID NO: 321 - DNA, 53 bp, synthetic construct
  misc_feature 1..53: note = Description of Artificial Sequence: Synthetic oligonucleotide
  source 1..53: mol_type = other DNA
ggggcttttctgtcaccaatcctgctgtccctagtggccccactgtggggtgg

SEQ ID NO: 322 - DNA, 53 bp, synthetic construct; identical to SEQ ID NO: 321
  misc_feature 1..53: note = Description of Artificial Sequence: Synthetic oligonucleotide
  source 1..53: mol_type = other DNA

SEQ ID NO: 323 - DNA, 53 bp, synthetic construct
  misc_feature 1..53: note = Description of Artificial Sequence: Synthetic oligonucleotide
  source 1..53: mol_type = other DNA
ggggcttttctgtcaccaatcctagtgtccctagtggccccactgtggggtgg

SEQ ID NO: 324 - DNA, 51 bp, synthetic construct
  misc_feature 1..51: note = Description of Artificial Sequence: Synthetic oligonucleotide
  source 1..51: mol_type = other DNA
ggggcttttctgtcaccaatccctgtccctagtggccccactgtggggtgg

SEQ ID NO: 325 - DNA, 51 bp, synthetic construct; identical to SEQ ID NO: 324
  misc_feature 1..51: note = Description of Artificial Sequence: Synthetic oligonucleotide
  source 1..51: mol_type = other DNA

SEQ ID NO: 326 - DNA, 49 bp, synthetic construct
  misc_feature 1..49: note = Description of Artificial Sequence: Synthetic oligonucleotide
  source 1..49: mol_type = other DNA
ggggcttttctgtcacaatcctgtccctagtggccccactgtggggtgg

SEQ ID NO: 327 - DNA, 49 bp, synthetic construct
  misc_feature 1..49: note = Description of Artificial Sequence: Synthetic oligonucleotide
  source 1..49: mol_type = other DNA
ggggcttttctgtcaccaatctgtccctagtggccccactgtggggtgg

SEQ ID NO: 328 - DNA, 49 bp, synthetic construct; identical to SEQ ID NO: 327
  misc_feature 1..49: note = Description of Artificial Sequence: Synthetic oligonucleotide
  source 1..49: mol_type = other DNA

SEQ ID NO: 329 - DNA, 23 bp, unidentified
  misc_feature 1..23: note = Description of Unknown: Target sequence
  source 1..23: mol_type = unassigned DNA
ataatttctattatattacaggg

SEQ ID NO: 330 - DNA, 23 bp, unidentified
  misc_feature 1..23: note = Description of Unknown: Target sequence
  source 1..23: mol_type = unassigned DNA
atttcaggtaagccgaggtttgg

SEQ ID NO: 331 - DNA, 23 bp, unidentified
  misc_feature 1..23: note = Description of Unknown: Target sequence
  source 1..23: mol_type = unassigned DNA
tctttgaaagagcaataaaatgg

SEQ ID NO: 332 - DNA, 6588 bp, synthetic construct
  misc_feature 1..6588: note = Description of Artificial Sequence: Synthetic polynucleotide
  source 1..6588: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgattaagatcgcaacccgaaaatacctgggaaagcagaacgtctacgatattggtgtagagagagaccataactttgctctgaagaacggctttattgcctcatgcttcgacagcgttgagatttccggcgtggaggatagattcaacgcttctctcggcacttatcacgaccttctgaagattatcaaggataaggatttcctggacaacgaagagaatgaagacatcctggaggacatcgtcctgaccttgaccctgttcgaggacagagagatgatcgaggagaggcttaagacctacgcccacctgtttgatgacaaagtgatgaaacagctgaaacggagacggtatactggttggggcaggctgtcccggaagcttattaacggaatacgggataagcaaagtggaaagacaatacttgacttcctgaagtctgatggttttgctaacaggaatttcatgcagctgattcacgacgactcccttacatttaaggaggacattcagaaggcccaggtgtctggacaaggggactctctccatgagcacatcgccaacctggccggcagcccagccatcaaaaaaggaattcttcaaactgtaaaggtggtggatgagctggttaaagtcatgggacggcacaagcctgagaatatcgtcattgagatggccagggagaatcagacgacacagaaaggacagaagaactcacgcgagaggatgaagagaattgaggaagggataaaggagctgggaagtcagattctgaaggaacacccagttgaaaatacccagctgcagaatgaaaagctgtatctgtactatctgcagaatggacgagacatgtatgttgatcaggagctggacattaaccgactctcagattatgacgtggatgctatagtccctcagagtttcctcaaggacgattcaatcgataataaagtgttgacccgcagcgacaaaaacaggggcaaaagcgataatgtgccctcagaggaagtggtcaagaaaatgaagaattactggagacagctgctcaacgctaagcttattacccagaggaaattcgataatttgacaaaagctgaaaggggtgggcttagcgagctggataaagcaggattcatcaagcggcagcttgtcgagacgcgccagatcacaaagcacgtggcacagattttggattcccgcatgaacactaagtatgacgagaacgataagctgatccgcgaggtgaaggtgatcacgctgaagtccaagctggtaagtgatttccggaaagatttccagttctacaaagtgagggagattaacaactatcaccacgcccacgacgcttacttgaatgccgttgtgggtacagcattgatcaaaaaatatccaaagctggaaagtgagtttgtttacggagactataaagtctatgacgtgcggaagatgatcgccaagagcgagcaggagatcgggaaagcaacagctaaatatttcttctattccaatatcatgaattttttcaaaactgagataacacttgctaatggtgagataagaaagcgaccgctgatagagacgaatggcgagactggcgagatcgtgtgggacaaagggagggacttcgcaaccgtccgcaaggtcttgagcatgccgcaggtgaatatagttaagaaaaccgaagtgcaaacaggcggcttcagtaaggagtccatattgccgaagaggaactctgacaagctgatcgctaggaaaaaggattgggatccaaaaaaatacggcgggttcgactcccctaccgttgcatacagcgtgcttgtggtcgcgaaggtcgaaaagggcaagtctaagaagctcaagagtgtcaaagaattgctgggtatcacaattatggagcgcagtagtttcgagaagaatccgatagattttctggaggcaaagggatacaaggaggtgaagaaggatctgatcatcaaactgcctaagtactccctgttcgagcttgagaatggtagaaagcgcatgcttgcctcagccggcgaattgcagaagggcaatgagctcgccctgccttcaaaatacgtgaacttcctgtacttggcatcacactacgaaaagctgaaaggatcccctgaggataatgagcaaaaacaactttttgtggagcagcataagcactatctcgatgaaattattgagcagatttctgaattcagcaagcgcgtcatcctcgcggacgccaatctggataaagtgctgagcgcctacaataaacaccgagacaagcccattcgggaacaggccgagaacatcattcacctcttcactctgactaatctcggggccccggccgcattcaaatacttcgacactactatcgacaggaaacgctatacttcaacgaaggaggtgctggacgctactttgatccaccagtccattacggggctctatgagacacgaatcgatctttctcaacttggaggtgatgcctacccatatgacgtgcctgactatgcctccctgggctctgggagccctaagaaaaagaggaaggtagaggatccaaaaaaaaagcgaaaagtcgatgatggcggttccggcggagggtcggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccatctaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 333 - DNA, 7533 bp, synthetic construct
  misc_feature 1..7533: note = Description of Artificial Sequence: Synthetic polynucleotide
  source 1..7533: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgattaagatcgcaacccgaaaatacctgggaaagcagaacgtctacgatattggtgtagagagagaccataactttgctctgaagaacggctttattgcctcatgcttcgacagcgttgagatttccggcgtggaggatagattcaacgcttctctcggcacttatcacgaccttctgaagattatcaaggataaggatttcctggacaacgaagagaatgaagacatcctggaggacatcgtcctgaccttgaccctgttcgaggacagagagatgatcgaggagaggcttaagacctacgcccacctgtttgatgacaaagtgatgaaacagctgaaacggagacggtatactggttggggcaggctgtcccggaagcttattaacggaatacgggataagcaaagtggaaagacaatacttgacttcctgaagtctgatggttttgctaacaggaatttcatgcagctgattcacgacgactcccttacatttaaggaggacattcagaaggcccaggtgtctggacaaggggactctctccatgagcacatcgccaacctggccggcagcccagccatcaaaaaaggaattcttcaaactgtaaaggtggtggatgagctggttaaagtcatgggacggcacaagcctgagaatatcgtcattgagatggccagggagaatcagacgacacagaaaggacagaagaactcacgcgagaggatgaagagaattgaggaagggataaaggagctgggaagtcagattctgaaggaacacccagttgaaaatacccagctgcagaatgaaaagctgtatctgtactatctgcagaatggacgagacatgtatgttgatcaggagctggacattaaccgactctcagattatgacgtggatgctatagtccctcagagtttcctcaaggacgattcaatcgataataaagtgttgacccgcagcgacaaaaacaggggcaaaagcgataatgtgccctcagaggaagtggtcaagaaaatgaagaattactggagacagctgctcaacgctaagcttattacccagaggaaattcgataatttgacaaaagctgaaaggggtgggcttagcgagctggataaagcaggattcatcaagcggcagcttgtcgagacgcgccagatcacaaagcacgtggcacagattttggattcccgcatgaacactaagtatgacgagaacgataagctgatccgcgaggtgaaggtgatcacgctgaagtccaagctggtaagtgatttccggaaagatttccagttctacaaagtgagggagattaacaactatcaccacgcccacgacgcttacttgaatgccgttgtgggtacagcattgatcaaaaaatatccaaagctggaaagtgagtttgtttacggagactataaagtctatgacgtgcggaagatgatcgccaagagcgagcaggagatcgggaaagcaacagctaaatatttcttctattccaatatcatgaattttttcaaaactgagataacacttgctaatggtgagataagaaagcgaccgctgatagagacgaatggcgagactggcgagatcgtgtgggacaaagggagggacttcgcaaccgtccgcaaggtcttgagcatgccgcaggtgaatatagttaagaaaaccgaagtgcaaacaggcggcttcagtaaggagtccatattgccgaagaggaactctgacaagctgatcgctaggaaaaaggattgggatccaaaaaaatacggcgggttcgactcccctaccgttgcatacagcgtgcttgtggtcgcgaaggtcgaaaagggcaagtctaagaagctcaagagtgtcaaagaattgctgggtatcacaattatggagcgcagtagtttcgagaagaatccgatagattttctggaggcaaagggatacaaggaggtgaagaaggatctgatcatcaaactgcctaagtactccctgttcgagcttgagaatggtagaaagcgcatgcttgcctcagccggcgaattgcagaagggcaatgagctcgccctgccttcaaaatacgtgaacttcctgtacttggcatcacactacgaaaagctgaaaggatcccctgaggataatgagcaaaaacaactttttgtggagcagcataagcactatctcgatgaaattattgagcagatttctgaattcagcaagcgcgtcatcctcgcggacgccaatctggataaagtgctgagcgcctacaataaacaccgagacaagcccattcgggaacaggccgagaacatcattcacctcttcactctgactaatctcggggccccggccgcattcaaatacttcgacactactatcgacaggaaacgctatacttcaacgaaggaggtgctggacgctactttgatccaccagtccattacggggctctatgagacacgaatcgatctttctcaacttggaggtgatgcctacccatatgacgtgcctgactatgcctccctgggctctgggagccctaagaaaaagaggaaggtagaggatccaaaaaaaaagcgaaaagtcgatgatggcggttccggcggagggtcgatggcagctatacctgcactggatcccgaagctgaacctagcatggatgtcatccttgtcggcagcagtgagctgtcatctagtgtctccccaggtacagggcgagacttgatcgcgtatgaggttaaagccaaccaacggaacattgaggacatttgcatttgttgcggttccttgcaagtccacacccaacacccactctttgagggtggcatctgcgctccttgtaaggataaattcctggacgccctgttcctttatgatgacgacggataccagagctactgttctatatgttgttccggggagactctccttatctgtggaaatcctgactgcacacggtgctactgctttgagtgtgttgattcattggttggtcccggcacaagcggcaaggtacatgctatgtctaattgggtatgttatctgtgcctccccagctcacgaagtggcctgttgcaacgcagacggaagtggcgaagtcaacttaaagccttttatgacagagaatctgagaatcctctggagatgtttgagactgtaccagtctggcgaagacaacccgtgcgggtgttgagcctgtttgaggatatcaagaaggagttgacttccctcggtttcctggaatcaggaagtgatcccggccagctcaaacatgtagtcgatgtgactgacacggtgcggaaagatgtcgaggagtggggccctttcgatctggtgtatggggctacaccccccttgggccacacttgtgacaggcccccgtcatggtatctgttccaatttcaccgcctccttcaatatgcgcgacccaagccaggttccccgaggccatttttctggatgttcgtggacaacctggtgcttaacaaagaggatttggacgttgcctctagattcttggaaatggagcctgttactattccggacgtccatggcggcagcctccaaaacgcagtgcgagtctggtctaacataccagcgattcgctcacgccattgggctttggtgtccgaagaagaattgagccttcttgcccagaataagcaaagcagtaaactggccgccaaatggcccacaaaattggtaaagaactgtttcctcccattgcgggagtacttcaagtacttcagcacagaattgacgtcttcattgatctaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 334 - DNA, 7341 bp, synthetic construct
  misc_feature 1..7341: note = Description of Artificial Sequence: Synthetic polynucleotide
  source 1..7341: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgattaagatcgcaacccgaaaatacctgggaaagcagaacgtctacgatattggtgtagagagagaccataactttgctctgaagaacggctttattgcctcatgcttcgacagcgttgagatttccggcgtggaggatagattcaacgcttctctcggcacttatcacgaccttctgaagattatcaaggataaggatttcctggacaacgaagagaatgaagacatcctggaggacatcgtcctgaccttgaccctgttcgaggacagagagatgatcgaggagaggcttaagacctacgcccacctgtttgatgacaaagtgatgaaacagctgaaacggagacggtatactggttggggcaggctgtcccggaagcttattaacggaatacgggataagcaaagtggaaagacaatacttgacttcctgaagtctgatggttttgctaacaggaatttcatgcagctgattcacgacgactcccttacatttaaggaggacattcagaaggcccaggtgtctggacaaggggactctctccatgagcacatcgccaacctggccggcagcccagccatcaaaaaaggaattcttcaaactgtaaaggtggtggatgagctggttaaagtcatgggacggcacaagcctgagaatatcgtcattgagatggccagggagaatcagacgacacagaaaggacagaagaactcacgcgagaggatgaagagaattgaggaagggataaaggagctgggaagtcagattctgaaggaacacccagttgaaaatacccagctgcagaatgaaaagctgtatctgtactatctgcagaatggacgagacatgtatgttgatcaggagctggacattaaccgactctcagattatgacgtggatgctatagtccctcagagtttcctcaaggacgattcaatcgataataaagtgttgacccgcagcgacaaaaacaggggcaaaagcgataatgtgccctcagaggaagtggtcaagaaaatgaagaattactggagacagctgctcaacgctaagcttattacccagaggaaattcgataatttgacaaaagctgaaaggggtgggcttagcgagctggataaagcaggattcatcaagcggcagcttgtcgagacgcgccagatcacaaagcacgtggcacagattttggattcccgcatgaacactaagtatgacgagaacgataagctgatccgcgaggtgaaggtgatcacgctgaagtccaagctggtaagtgatttccggaaagatttccagttctacaaagtgagggagattaacaactatcaccacgcccacgacgcttacttgaatgccgttgtgggtacagcattgatcaaaaaatatccaaagctggaaagtgagtttgtttacggagactataaagtctatgacgtgcggaagatgatcgccaagagcgagcaggagatcgggaaagcaacagctaaatatttcttctattccaatatcatgaattttttcaaaactgagataacacttgctaatggtgagataagaaagcgaccgctgatagagacgaatggcgagactggcgagatcgtgtgggacaaagggagggacttcgcaaccgtccgcaaggtcttgagcatgccgcaggtgaatatagttaagaaaaccgaagtgcaaacaggcggcttcagtaaggagtccatattgccgaagaggaactctgacaagctgatcgctaggaaaaaggattgggatccaaaaaaatacggcgggttcgactcccctaccgttgcatacagcgtgcttgtggtcgcgaaggtcgaaaagggcaagtctaagaagctcaagagtgtcaaagaattgctgggtatcacaattatggagcgcagtagtttcgagaagaatccgatagattttctggaggcaaagggatacaaggaggtgaagaaggatctgatcatcaaactgcctaagtactccctgttcgagcttgagaatggtagaaagcgcatgcttgcctcagccggcgaattgcagaagggcaatgagctcgccctgccttcaaaatacgtgaacttcctgtacttggcatcacactacgaaaagctgaaaggatcccctgaggataatgagcaaaaacaactttttgtggagcagcataagcactatctcgatgaaattattgagcagatttctgaattcagcaagcgcgtcatcctcgcggacgccaatctggataaagtgctgagcgcctacaataaacaccgagacaagcccattcgggaacaggccgagaacatcattcacctcttcactctgactaatctcggggccccggccgcattcaaatacttcgacactactatcgacaggaaacgctatacttcaacgaaggaggtgctggacgctactttgatccaccagtccattacggggctctatgagacacgaatcgatctttctcaacttggaggtgatgcctacccatatgacgtgcctgactatgcctccctgggctctgggagccctaagaaaaagaggaaggtagaggatccaaaaaaaaagcgaaaagtcgatgatggcggttccggcggagggtcgacctatggtcttcttaggagaagagaagactggccctctcggctccaaatgttcttcgctaataatcacgatcaagaattcgacccgcctaaggtctacccaccggtgccagcagagaaacgaaagccgatcagagtattgtctttgttcgatggcatagccacgggactcctggtgctgaaagatctgggaatccaggttgatcgctacatcgcctcagaggtttgtgaagactctataaccgtagggatggtacgacaccagggtaagataatgtatgtcggtgatgtacggtccgtgacacaaaaacacatacaggagtggggaccctttgaccttgtgataggcggatctccatgcaatgacctttccattgttaatcctgcccgcaaaggactttacgaaggaaccggccgactcttttttgaattttatcggttgctccatgatgctcggccgaaggagggcgatgaccgcccctttttctggcttttcgagaacgtcgtcgctatgggcgtttccgataagagagacataagccgattccttgagagcaacccagtaatgattgatgcaaaagaagtttctgccgcccacagggctaggtacttctggggaaatttgccaggcatgaaccgcccactggcatccaccgttaacgataagctggaacttcaggaatgtttggagcacggtagaatcgcaaaattctcaaaagtaagaacgatcacgacaagaagtaattctatcaagcaagggaaagatcagcacttccccgtctttatgaatgaaaaggaggacattctttggtgcactgaaatggagcgcgtgttcggatttcctgttcactatacggacgtcagcaatatgtctcgcctcgccaggcagcgattgttgggccgctcttggagtgttccagtcatacgacatctttttgcgccacttaaagaatactttgcctgtgtgatctaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 335 - DNA, 6759 bp, synthetic construct
  misc_feature 1..6759: note = Description of Artificial Sequence: Synthetic polynucleotide
  source 1..6759: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttgctagcctagacccagctttcttgtacaaagttggcattaatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaaggccaccatggatgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtggcggttccggcggagggtcgatcatgggccccaagaaaaaacgcaaggtggccgcagcagactataaggatgacgacgataaggggatccatggtgtgcctgctgcagataaaaaatacagcatcggcctggctatcggaactaactccgtcggctgggccgtcattaccgacgaatacaaagtacctagcaaaaagttcaaggtgcttggcaacacagatcgccactcaatcaagaaaaaccttatcggagccctgctgtttgactcaggcgaaaccgccgaggctacacgcctgaaaagaacagctagacggcggtacaccagaaggaagaaccggatctgttatcttcaggagattttctccaatgagatggctaaggtggacgattctttcttccatcgactcgaagaatctttcttggtggaggaagataagaaacacgagaggcatcctattttcggaaacattgtcgatgaagtggcctatcatgagaaataccccacgatctaccatctgcgaaaaaagttggttgactctaccgacaaggcggacctgaggcttatttatctggccctggcccatatgatcaaattcagggggcacttcttgatcgagggggaccttaatcccgacaactctgacgtggataagttgttcatacagcttgtgcagacctacaaccagctgttcgaggagaatccaatcaacgccagcggagtggacgctaaagccattctgagcgcgagattgagcaagtctagaagattggaaaaccttatagcccagctgccaggtgagaagaagaacggactgtttggcaatctcattgcgcttagcctcggactcaccccgaacttcaaatccaacttcgacctcgccgaagatgccaaattgcagctcagtaaggatacgtatgacgatgatcttgacaatctgctggcgcagatcggggaccagtacgccgatcttttcttggcagcaaaaaatctctcagatgcaatactcttgtcagacatactgcgagttaataccgagattactaaggctccgctttctgcctccatgatcaagcgctacgatgagcatcaccaggatctgacactgttgaaagccctggtgcgccaacagctgccagagaaatacaaggaaatcttttttgaccagtccaagaatggctacgcaggatacatcgatggaggagccagtcaggaggaattttacaagtttattaagcctatcctggagaagatggatggtaccgaagaactcctggtcaagctcaaccgagaagatttgcttcgcaagcaaaggacttttgacaacggctccattccgcatcagattcatctgggcgagctgcatgccattctgcgaagacaggaggatttttacccatttctgaaggacaaccgagagaagatcgagaaaatactgacattcaggataccatattacgtgggtccactcgccaggggcaactcccgattcgcctggatgacaaggaaaagcgaagagacgatcactccatggaacttcgaggaggtcgtggacaagggggcctccgcgcagagctttatcgagaggatgacgaactttgacaaaaatctccctaacgagaaggtgctgccaaaacattctctgctctacgagtatttcaccgtttataatgagctcacaaaggtgaagtacgtgaccgaagggatgcggaagcccgcttttctgtccggagagcagaagaaggctatcgtggatttgctctttaagactaaccgcaaggtaacagtcaagcagctgaaggaagactacttcaagaagatcgaatgcttgtcctacgaaacggaaatcttgacagttgagtacgggctcctgccaatcgggaagatagtagagaagaggattgaatgtaccgtctattctgttgataacaacggtaacatatacacccagcccgtcgcccaatggcacgatcgcggtgagcaggaggtgttcgaatactgtctggaggacgggtcattgattcgggcgactaaggaccataagtttatgacggtagacggccagatgttgcccatagatgagatctttgagcgggaactcgacttgatgagagtcgataatcttcctaattagcttaagggttcgatccctactggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 336 - DNA, 7341 bp, synthetic construct
  misc_feature 1..7341: note = Description of Artificial Sequence: Synthetic polynucleotide
  source 1..7341: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttgctagcctagacccagctttcttgtacaaagttggcattaatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaaggccaccatggatccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcatcatgggccccaagaaaaaacgcaaggtggccgcagcagactataaggatgacgacgataaggggatccatggtgtgcctgctgcagataaaaaatacagcatcggcctggctatcggaactaactccgtcggctgggccgtcattaccgacgaatacaaagtacctagcaaaaagttcaaggtgcttggcaacacagatcgccactcaatcaagaaaaaccttatcggagccctgctgtttgactcaggcgaaaccgccgaggctacacgcctgaaaagaacagctagacggcggtacaccagaaggaagaaccggatctgttatcttcaggagattttctccaatgagatggctaaggtggacgattctttcttccatcgactcgaagaatctttcttggtggaggaagataagaaacacgagaggcatcctattttcggaaacattgtcgatgaagtggcctatcatgagaaataccccacgatctaccatctgcgaaaaaagttggttgactctaccgacaaggcggacctgaggcttatttatctggccctggcccatatgatcaaattcagggggcacttcttgatcgagggggaccttaatcccgacaactctgacgtggataagttgttcatacagcttgtgcagacctacaaccagctgttcgaggagaatccaatcaacgccagcggagtggacgctaaagccattctgagcgcgagattgagcaagtctagaagattggaaaaccttatagcccagctgccaggtgagaagaagaacggactgtttggcaatctcattgcgcttagcctcggactcaccccgaacttcaaatccaacttcgacctcgccgaagatgccaaattgcagctcagtaaggatacgtatgacgatgatcttgacaatctgctggcgcagatcggggaccagtacgccgatcttttcttggcagcaaaaaatctctcagatgcaatactcttgtcagacatactgcgagttaataccgagattactaaggctccgctttctgcctccatgatcaagcgctacgatgagcatcaccaggatctgacactgttgaaagccctggtgcgccaacagctgccagagaaatacaaggaaatcttttttgaccagtccaagaatggctacgcaggatacatcgatggaggagccagtcaggaggaattttacaagtttattaagcctatcctggagaagatggatggtaccgaagaactcctggtcaagctcaaccgagaagatttgcttcgcaagcaaaggacttttgacaacggctccattccgcatcagattcatctgggcgagctgcatgccattctgcgaagacaggaggatttttacccatttctgaaggacaaccgagagaagatcgagaaaatactgacattcaggataccatattacgtgggtccactcgccaggggcaactcccgattcgcctggatgacaaggaaaagcgaagagacgatcactccatggaacttcgaggaggtcgtggacaagggggcctccgcgcagagctttatcgagaggatgacgaactttgacaaaaatctccctaacgagaaggtgctgccaaaacattctctgctctacgagtatttcaccgtttataatgagctcacaaaggtgaagtacgtgaccgaagggatgcggaagcccgcttttctgtccggagagcagaagaaggctatcgtggatttgctctttaagactaaccgcaaggtaacagtcaagcagctgaaggaagactacttcaagaagatcgaatgcttgtcctacgaaacggaaatcttgacagttgagtacgggctcctgccaatcgggaagatagtagagaagaggattgaatgtaccgtctattctgttgataacaacggtaacatatacacccagcccgtcgcccaatggcacgatcgcggtgagcaggaggtgttcgaatactgtctggaggacgggtcattgattcgggcgactaaggaccataagtttatgacggtagacggccagatgttgcccatagatgagatctttgagcgggaactcgacttgatgagagtcgataatcttcctaattagcttaagggttcgatccctactggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 337 - DNA, 5751 bp, synthetic construct
  misc_feature 1..5751: note = Description of Artificial Sequence: Synthetic polynucleotide
  source 1..5751: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaaggcctccacggccactagtctttcgtcttcaagaattcctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagtactctggaaacagaatctactaaaacaaggcaaaatgccgtgtttatctcgtcaacttgttggcgagatttttgaattctcgacctcgagacaaatggcagcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccacctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactcttaaggatatcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttgaggatccagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 338 - DNA, 7317 bp, synthetic construct
  misc_feature 1..7317: note = Description of Artificial Sequence: Synthetic polynucleotide
  source 1..7317: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttgctagcctagacccagctttcttgtacaaagttggcattaatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaaggccaccatgggccccaagaaaaaacgcaaggtggccgcagcagactataaggatgacgacgataaggggatccatggtgtgcctgctgcagataaaaaatacagcatcggcctggatatcggaactaactccgtcggctgggccgtcattaccgacgaatacaaagtacctagcaaaaagttcaaggtgcttggcaacacagatcgccactcaatcaagaaaaaccttatcggagccctgctgtttgactcaggcgaaaccgccgaggctacacgcctgaaaagaacagctagacggcggtacaccagaaggaagaaccggatctgttatcttcaggagattttctccaatgagatggctaaggtggacgattctttcttccatcgactcgaagaatctttcttggtggaggaagataagaaacacgagaggcatcctattttcggaaacattgtcgatgaagtggcctatcatgagaaataccccacgatctaccatctgcgaaaaaagttggttgactctaccgacaaggcggacctgaggcttatttatctggccctggcccatatgatcaaattcagggggcacttcttgatcgagggggaccttaatcccgacaactctgacgtggataagttgttcatacagcttgtgcagacctacaaccagctgttcgaggagaatccaatcaacgccagcggagtggacgctaaagccattctgagcgcgagattgagcaagtctagaagattggaaaaccttatagcccagctgccaggtgagaagaagaacggactgtttggcaatctcattgcgcttagcctcggactcaccccgaacttcaaatccaacttcgacctcgccgaagatgccaaattgcagctcagtaaggatacgtatgacgatgatcttgacaatctgctggcgcagatcggggaccagtacgccgatcttttcttggcagcaaaaaatctctcagatgcaatactcttgtcagacatactgcgagttaataccgagattactaaggctccgctttctgcctccatgatcaagcgctacgatgagcatcaccaggatctgacactgttgaaagccctggtgcgccaacagctgccagagaaatacaaggaaatcttttttgaccagtccaagaatggctacgcaggatacatcgatggaggagccagtcaggaggaattttacaagtttattaagcctatcctggagaagatggatggtaccgaagaactcctggtcaagctcaaccgagaagatttgcttcgcaagcaaaggacttttgacaacggctccattccgcatcagattcatctgggcgagctgcatgccattctgcgaagacaggaggatttttacccatttctgaaggacaaccgagagaagatcgagaaaatactgacattcaggataccatattacgtgggtccactcgccaggggcaactcccgattcgcctggatgacaaggaaaagcgaagagacgatcactccatggaacttcgaggaggtcgtggacaagggggcctccgcgcagagctttatcgagaggatgacgaactttgacaaaaatctccctaacgagaaggtgctgccaaaacattctctgctctacgagtatttcaccgtttataatgagctcacaaaggtgaagtacgtgaccgaagggatgcggaagcccgcttttctgtccggagagcagaagaaggctatcgtggatttgctctttaagactaaccgcaaggtaacagtcaagcagctgaaggaagactacttcaagaagatcgaatgcttgtcctacgaaacggaaatcttgacagttgagtacgggctcctgccaatcgggaagatagtagagaagaggattgaatgtaccgtctattctgttgataacaacggtaacatatacacccagcccgtcgcccaatggcacgatcgcggtgagcaggaggtgttcgaatactgtctggaggacgggtcattgattcgggcgactaaggaccataagtttatgacggtagacggccagatgttgcccatagatgagatctttgagcgggaactcgacttgatgagagtcgataatcttcctaatggatccggcgcaacaaacttctctctgctgaaacaagccggagatgtcgaagagaatcctggaccgatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtagcttaagggttcgatccctactggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 339 - DNA, 6192 bp, synthetic construct
  misc_feature 1..6192: note = Description of Artificial Sequence: Synthetic polynucleotide
  source 1..6192: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaaaaggcctccacggccactagtctttcgtcttcaagaattcctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggtctagagctagcgaattcgaatttgccaccatgattaagatcgcaacccgaaaatacctgggaaagcagaacgtctacgatattggtgtagagagagaccataactttgctctgaagaacggctttattgcctcatgcttcgacagcgttgagatatccggcgtggaggatagattcaacgcttctctcggcacttatcacgaccttctgaagattatcaaggataaggatttcctggacaacgaagagaatgaagacatcctggaggacatcgtcctgaccttgaccctgttcgaggacagagagatgatcgaggagaggcttaagacctacgcccacctgtttgatgacaaagtgatgaaacagctgaaacggagacggtatactggttggggcaggctgtcccggaagcttattaacggaatacgggataagcaaagtggaaagacaatacttgacttcctgaagtctgatggttttgctaacaggaatttcatgcagctgattcacgacgactcccttacatttaaggaggacattcagaaggcccaggtgtctggacaaggggactctctccatgagcacatcgccaacctggccggcagcccagccatcaaaaaaggaattcttcaaactgtaaaggtggtggatgagctggttaaagtcatgggacggcacaagcctgagaatatcgtcattgagatggccagggagaatcagacgacacagaaaggacagaagaactcacgcgagaggatgaagagaattgaggaagggataaaggagctgggaagtcagattctgaaggaacacccagttgaaaatacccagctgcagaatgaaaagctgtatctgtactatctgcagaatggacgagacatgtatgttgatcaggagctggacattaaccgactctcagattatgacgtggatcatatagtccctcagagtttcctcaaggacgattcaatcgataataaagtgttgacccgcagcgacaaaaacaggggcaaaagcgataatgtgccctcagaggaagtggtcaagaaaatgaagaattactggagacagctgctcaacgctaagcttattacccagaggaaattcgataatttgacaaaagctgaaaggggtgggcttagcgagctggataaagcaggattcatcaagcggcagcttgtcgagacgcgccagatcacaaagcacgtggcacagattttggattcccgcatgaacactaagtatgacgagaacgataagctgatccgcgaggtgaaggtgatcacgctgaagtccaagctggtaagtgatttccggaaagatttccagttctacaaagtgagggagattaacaactatcaccacgcccacgacgcttacttgaatgccgttgtgggtacagcattgatcaaaaaatatccaaagctggaaagtgagtttgtttacggagactataaagtctatgacgtgcggaagatgatcgccaagagcgagcaggagatcgggaaagcaacagctaaatatttcttctattccaatatcatgaattttttcaaaactgagataacacttgctaatggtgagataagaaagcgaccgctgatagagacgaatggcgagactggcgagatcgtgtgggacaaagggagggacttcgcaaccgtccgcaaggtcttgagcatgccgcaggtgaatatagttaagaaaaccgaagtgcaaacaggcggcttcagtaaggagtccatattgccgaagaggaactctgacaagctgatcgctaggaaaaaggattgggatccaaaaaaatacggcgggttcgactcccctaccgttgcatacagcgtgcttgtggtcgcgaaggtcgaaaagggcaagtctaagaagctcaagagtgtcaaagaattgctgggtatcacaattatggagcgcagtagtttcgagaagaatccgatagattttctggaggcaaagggatacaaggaggtgaagaaggatctgatcatcaaactgcctaagtactccctgttcgagcttgagaatggtagaaagcgcatgcttgcctcagccggcgaattgcagaagggcaatgagctcgccctgccttcaaaatacgtgaacttcctgtacttggcatcacactacgaaaagctgaaaggatcccctgaggataatgagcaaaaacaactttttgtggagcagcataagcactatctcgatgaaattattgagcagatttctgaattcagcaagcgcgtcatcctcgcggacgccaatctggataaagtgctgagcgcctacaataaacaccgagacaagcccattcgggaacaggccgagaacatcattcacctcttcactctgactaatctcggggccccggccgcattcaaatacttcgacactactatcgacaggaaacgctatacttcaacgaaggaggtgctggacgctactttgatccaccagtccattacggggctctatgagacacgaatcgatctttctcaacttggaggtgatgcctacccatatgacgtgcctgactatgcctctctgggctctgggagccctaagaaaaagaggaaggtagaggatccaaaaaaaaagcgaaaagtcgattagagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 340 - DNA, 6642 bp, synthetic construct
  misc_feature 1..6642: note = Description of Artificial Sequence: Synthetic polynucleotide
  source 1..6642: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgattaagatcgcaacccgaaaatacctgggaaagcagaacgtctacgatattggtgtagagagagaccataactttgctctgaagaacggctttattgcctcatgcttcgacagcgttgagatatccggcgtggaggatagattcaacgcttctctcggcacttatcacgaccttctgaagattatcaaggataaggatttcctggacaacgaagagaatgaagacatcctggaggacatcgtcctgaccttgaccctgttcgaggacagagagatgatcgaggagaggcttaagacctacgcccacctgtttgatgacaaagtgatgaaacagctgaaacggagacggtatactggttggggcaggctgtcccggaagcttattaacggaatacgggataagcaaagtggaaagacaatacttgacttcctgaagtctgatggttttgctaacaggaatttcatgcagctgattcacgacgactcccttacatttaaggaggacattcagaaggcccaggtgtctggacaaggggactctctccatgagcacatcgccaacctggccggcagcccagccatcaaaaaaggaattcttcaaactgtaaaggtggtggatgagctggttaaagtcatgggacggcacaagcctgagaatatcgtcattgagatggccagggagaatcagacgacacagaaaggacagaagaactcacgcgagaggatgaagagaattgaggaagggataaaggagctgggaagtcagattctgaaggaacacccagttgaaaatacccagctgcagaatgaaaagctgtatctgtactatctgcagaatggacgagacatgtatgttgatcaggagctggacattaaccgactctcagattatgacgtggatcatatagtccctcagagtttcctcaaggacgattcaatcgataataaagtgttgacccgcagcgacaaaaacaggggcaaaagcgataatgtgccctcagaggaagtggtcaagaaaatgaagaattactggagacagctgctcaacgctaagcttattacccagaggaaattcgataatttgacaaaagctgaaaggggtgggcttagcgagctggataaagcaggattcatcaagcggcagcttgtcgagacgcgccagatcacaaagcacgtggcacagattttggattcccgcatgaacactaagtatgacgagaacgataagctgatccgcgaggtgaaggtgatcacgctgaagtccaagctggtaagtgatttccggaaagatttccagttctacaaagtgagggagattaacaactatcaccacgcccacgacgcttacttgaatgccgttgtgggtacagcattgatcaaaaaatatccaaagctggaaagtgagtttgtttacggagactataaagtctatgacgtgcggaagatgatcgccaagagcgagcaggagatcgggaaagcaacagctaaatatttcttctattccaatatcatgaattttttcaaaactgagataacacttgctaatggtgagataagaaagcgaccgctgatagagacgaatggcgagactggcgagatcgtgtgggacaaagggagggacttcgcaaccgtccgcaaggtcttgagcatgccgcaggtgaatatagttaagaaaaccgaagtgcaaacaggcggcttcagtaaggagtccatattgccgaagaggaactctgacaagctgatcgctaggaaaaaggattgggatccaaaaaaatacggcgggttcgactcccctaccgttgcatacagcgtgcttgtggtcgcgaaggtcgaaaagggcaagtctaagaagctcaagagtgtcaaagaattgctgggtatcacaattatggagcgcagtagtttcgagaagaatccgatagattttctggaggcaaagggatacaaggaggtgaagaaggatctgatcatcaaactgcctaagtactccctgttcgagcttgagaatggtagaaagcgcatgcttgcctcagccggcgaattgcagaagggcaatgagctcgccctgccttcaaaatacgtgaacttcctgtacttggcatcacactacgaaaagctgaaaggatcccctgaggataatgagcaaaaacaactttttgtggagcagcataagcactatctcgatgaaattattgagcagatttctgaattcagcaagcgcgtcatcctcgcggacgccaatctggataaagtgctgagcgcctacaataaacaccgagacaagcccattcgggaacaggccgagaacatcattcacctcttcactctgactaatctcggggccccggccgcattcaaatacttcgacactactatcgacaggaaacgctatacttcaacgaaggaggtgctggacgctactttgatccaccagtccattacggggctctatgagacacgaatcgatctttctcaacttggaggtgattctggcggctctacaaatctgtctgacataatagaaaaggaaactgggaagcaacttgtcatccaagaatccatacttatgttgccggaagaggttgaagaggtcattggtaataagccggagagcgatattctcgtacacacagcatacgatgaatcaaccgatgaaaacgtaatgttgcttacttcagatgctcccgagtacaagccctgggcattggtaatccaggattccaacggcgaaaacaaaattaagatgctttctggagggagtcccaagaaaaagcggaaggtagcgtacccgtatgatgtcccagattacgcgagtcttggtagcgggtccccgaagaaaaagcgaaaggtggaagatccgaagaaaaagagaaaagttgattaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 341 - DNA, 7203 bp, synthetic construct
  misc_feature 1..7203: note = Description of Artificial Sequence: Synthetic polynucleotide
  source 1..7203: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttgctagcctagacccagctttcttgtacaaagttggcattaatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaaggccaccatgggaccgaaaaaaaagaggaaggtcgcggctggaagcggttccatgtccagcgagaccggacccgttgccgtcgatcctactttgaggagaagaatcgaaccacatgaatttgaagtatttttcgaccctagagagctgcgaaaagaaacctgcttgctgtatgaaataaattggggcggtcgccacagtatatggaggcacacctctcagaatacaaacaagcacgtagaggtgaactttattgaaaaattcaccacagagagatatttctgcccgaatacgagatgttccattacgtggtttctttcttggtccccatgcggtgagtgttcccgggccatcacagagtttttgtcacgataccctcacgtcacgctttttatctacatagcgcgactgtatcaccatgccgaccccaggaataggcaaggcttgcgcgatttgattagtagcggggttaccatccagattatgacggagcaagagtcagggtactgttggcggaactttgtaaactactccccgagcaatgaggcgcactggcctcgctacccacacctgtgggtccgactttacgtcttggaattgtattgcatcatcctcggcctcccgccgtgtctgaacatcctgcggcgcaagcagccccaattgacattttttacaatcgccctgcaatcatgccattatcagcggttgccgccacacatactttgggccacgggtttgaaaagcggatccgagacgcctggcaccagcgagtccgcaacccccgagagcgacaaaaagtatagtataggtttggctattggaactaattccgtaggttgggctgtgataacagatgaatacaaagtacctagcaaaaagttcaaggtgcttggcaacacagatcgccactcaatcaagaaaaaccttatcggagccctgctgtttgactcaggcgaaaccgccgaggctacacgcctgaaaagaacagctagacggcggtacaccagaaggaagaaccggatctgttatcttcaggagattttctccaatgagatggctaaggtggacgattctttcttccatcgactcgaagaatctttcttggtggaggaagataagaaacacgagaggcatcctattttcggaaacattgtcgatgaagtggcctatcatgagaaataccccacgatctaccatctgcgaaaaaagttggttgactctaccgacaaggcggacctgaggcttatttatctggccctggcccatatgatcaaattcagggggcacttcttgatcgagggggaccttaatcccgacaactctgacgtggataagttgttcatacagcttgtgcagacctacaaccagctgttcgaggagaatccaatcaacgccagcggagtggacgctaaagccattctgagcgcgagattgagcaagtctagaagattggaaaaccttatagcccagctgccaggtgagaagaagaacggactgtttggcaatctcattgcgcttagcctcggactcaccccgaacttcaaatccaacttcgacctcgccgaagatgccaaattgcagctcagtaaggatacgtatgacgatgatcttgacaatctgctggcgcagatcggggaccagtacgccgatcttttcttggcagcaaaaaatctctcagatgcaatactcttgtcagacatactgcgagttaataccgagattactaaggctccgctttctgcctccatgatcaagcgctacgatgagcatcaccaggatctgacactgttgaaagccctggtgcgccaacagctgccagagaaatacaaggaaatcttttttgaccagtccaagaatggctacgcaggatacatcgatggaggagccagtcaggaggaattttacaagtttattaagcctatcctggagaagatggatggtaccgaagaactcctggtcaagctcaaccgagaagatttgcttcgcaagcaaaggacttttgacaacggctccattccgcatcagattcatctgggcgagctgcatgccattctgcgaagacaggaggatttttacccatttctgaaggacaaccgagagaagatcgagaaaatactgacattcaggataccatattacgtgggtccactcgccaggggcaactcccgattcgcctggatgacaaggaaaagcgaagagacgatcactccatggaacttcgaggaggtcgtggacaagggggcctccgcgcagagctttatcgagaggatgacgaactttgacaaaaatctccctaacgagaaggtgctgccaaaacattctctgctctacgagtatttcaccgtttataatgagctcacaaaggtgaagtacgtgaccgaagggatgcggaagcccgcttttctgtccggagagcagaagaaggctatcgtggatttgctctttaagactaaccgcaaggtaacagtcaagcagctgaaggaagactacttcaagaagatcgaatgcttgtcctacgaaacggaaatcttgacagttgagtacgggctcctgccaatcgggaagatagtagagaagaggattgaatgtaccgtctattctgttgataacaacggtaacatatacacccagcccgtcgcccaatggcacgatcgcggtgagcaggaggtgttcgaatactgtctggaggacgggtcattgattcgggcgactaaggaccataagtttatgacggtagacggccagatgttgcccatagatgagatctttgagcgggaactcgacttgatgagagtcgataatcttcctaattagcttaagggttcgatccctactggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 342 - DNA, 7447 bp, synthetic construct
  misc_feature 1..7447: note = Description of Artificial Sequence: Synthetic polynucleotide
  source 1..7447: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtacctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 343 - DNA, 7146 bp, synthetic construct
  misc_feature 1..7146: note = Description of Artificial Sequence: Synthetic polynucleotide
  source 1..7146: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgattaagatcgcaacccgaaaatacctgggaaagcagaacgtctacgatattggtgtagagagagaccataactttgctctgaagaacggctttattgcctcatgcttcgacagcgttgagatttccggcgtggaggatagattcaacgcttctctcggcacttatcacgaccttctgaagattatcaaggataaggatttcctggacaacgaagagaatgaagacatcctggaggacatcgtcctgaccttgaccctgttcgaggacagagagatgatcgaggagaggcttaagacctacgcccacctgtttgatgacaaagtgatgaaacagctgaaacggagacggtatactggttggggcaggctgtcccggaagcttattaacggaatacgggataagcaaagtggaaagacaatacttgacttcctgaagtctgatggttttgctaacaggaatttcatgcagctgattcacgacgactcccttacatttaaggaggacattcagaaggcccaggtgtctggacaaggggactctctccatgagcacatcgccaacctggccggcagcccagccatcaaaaaaggaattcttcaaactgtaaaggtggtggatgagctggttaaagtcatgggacggcacaagcctgagaatatcgtcattgagatggccagggagaatcagacgacacagaaaggacagaagaactcacgcgagaggatgaagagaattgaggaagggataaaggagctgggaagtcagattctgaaggaacacccagttgaaaatacccagctgcagaatgaaaagctgtatctgtactatctgcagaatggacgagacatgtatgttgatcaggagctggacattaaccgactctcagattatgacgtggatgctatagtccctcagagtttcctcaaggacgattcaatcgataataaagtgttgacccgcagcgacaaaaacaggggcaaaagcgataatgtgccctcagaggaagtggtcaagaaaatgaagaattactggagacagctgctcaacgctaagcttattacccagaggaaattcgataatttgacaaaagctgaaaggggtgggcttagcgagctggataaagcaggattcatcaagcggcagcttgtcgagacgcgccagatcacaaagcacgtggcacagattttggattcccgcatgaacactaagtatgacgagaacgataagctgatccgcgaggtgaaggtgatcacgctgaagtccaagctggtaagtgatttccggaaagatttccagttctacaaagtgagggagattaacaactatcaccacgcccacgacgcttacttgaatgccgttgtgggtacagcattgatcaaaaaatatccaaagctggaaagtgagtttgtttacggagactataaagtctatgacgtgcggaagatgatcgccaagagcgagcaggagatcgggaaagcaacagctaaatatttcttctattccaatatcatgaattttttcaaaactgagataacacttgctaatggtgagataagaaagcgaccgctgatagagacgaatggcgagactggcgagatcgtgtgggacaaagggagggacttcgcaaccgtccgcaaggtcttgagcatgccgcaggtgaatatagttaagaaaaccgaagtgcaaacaggcggcttcagtaaggagtccatattgccgaagaggaactctgacaagctgatcgctaggaaaaaggattgggatccaaaaaaatacggcgggttcgactcccctaccgttgcatacagcgtgcttgtggtcgcgaaggtcgaaaagggcaagtctaagaagctcaagagtgtcaaagaattgctgggtatcacaattatggagcgcagtagtttcgagaagaatccgatagattttctggaggcaaagggatacaaggaggtgaagaaggatctgatcatcaaactgcctaagtactccctgttcgagcttgagaatggtagaaagcgcatgcttgcctcagccggcgaattgcagaagggcaatgagctcgccctgccttcaaaatacgtgaacttcctgtacttggcatcacactacgaaaagctgaaaggatcccctgaggataatgagcaaaaacaactttttgtggagcagcataagcactatctcgatgaaattattgagcagatttctgaattcagcaagcgcgtcatcctcgcggacgccaatctggataaagtgctgagcgcctacaataaacaccgagacaagcccattcgggaacaggccgagaacatcattcacctcttcactctgactaatctcggggccccggccgcattcaaatacttcgacactactatcgacaggaaacgctatacttcaacgaaggaggtgctggacgctactttgatccaccagtccattacggggctctatgagacacgaatcgatctttctcaacttggaggtgatgcctacccatatgacgtgcctgactatgcctccctgggctctgggagccctaagaaaaagaggaaggtagaggatccaaaaaaaaagcgaaaagtcgatgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 344 - DNA, 6354 bp, synthetic construct
  misc_feature 1..6354: note = Description of Artificial Sequence: Synthetic polynucleotide
  source 1..6354: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgattaagatcgcaacccgaaaatacctgggaaagcagaacgtctacgatattggtgtagagagagaccataactttgctctgaagaacggctttattgcctcatgcttcgacagcgttgagatttccggcgtggaggatagattcaacgcttctctcggcacttatcacgaccttctgaagattatcaaggataaggatttcctggacaacgaagagaatgaagacatcctggaggacatcgtcctgaccttgaccctgttcgaggacagagagatgatcgaggagaggcttaagacctacgcccacctgtttgatgacaaagtgatgaaacagctgaaacggagacggtatactggttggggcaggctgtcccggaagcttattaacggaatacgggataagcaaagtggaaagacaatacttgacttcctgaagtctgatggttttgctaacaggaatttcatgcagctgattcacgacgactcccttacatttaaggaggacattcagaaggcccaggtgtctggacaaggggactctctccatgagcacatcgccaacctggccggcagcccagccatcaaaaaaggaattcttcaaactgtaaaggtggtggatgagctggttaaagtcatgggacggcacaagcctgagaatatcgtcattgagatggccagggagaatcagacgacacagaaaggacagaagaactcacgcgagaggatgaagagaattgaggaagggataaaggagctgggaagtcagattctgaaggaacacccagttgaaaatacccagctgcagaatgaaaagctgtatctgtactatctgcagaatggacgagacatgtatgttgatcaggagctggacattaaccgactctcagattatgacgtggatgctatagtccctcagagtttcctcaaggacgattcaatcgataataaagtgttgacccgcagcgacaaaaacaggggcaaaagcgataatgtgccctcagaggaagtggtcaagaaaatgaagaattactggagacagctgctcaacgctaagcttattacccagaggaaattcgataatttgacaaaagctgaaaggggtgggcttagcgagctggataaagcaggattcatcaagcggcagcttgtcgagacgcgccagatcacaaagcacgtggcacagattttggattcccgcatgaacactaagtatgacgagaacgataagctgatccgcgaggtgaaggtgatcacgctgaagtccaagctggtaagtgatttccggaaagatttccagttctacaaagtgagggagattaacaactatcaccacgcccacgacgcttacttgaatgccgttgtgggtacagcattgatcaaaaaatatccaaagctggaaagtgagtttgtttacggagactataaagtctatgacgtgcggaagatgatcgccaagagcgagcaggagatcgggaaagcaacagctaaatatttcttctattccaatatcatgaattttttcaaaactgagataacacttgctaatggtgagataagaaagcgaccgctgatagagacgaatggcgagactggcgagatcgtgtgggacaaagggagggacttcgcaaccgtccgcaaggtcttgagcatgccgcaggtgaatatagttaagaaaaccgaagtgcaaacaggcggcttcagtaaggagtccatattgccgaagaggaactctgacaagctgatcgctaggaaaaaggattgggatccaaaaaaatacggcgggttcgactcccctaccgttgcatacagcgtgcttgtggtcgcgaaggtcgaaaagggcaagtctaagaagctcaagagtgtcaaagaattgctgggtatcacaattatggagcgcagtagtttcgagaagaatccgatagattttctggaggcaaagggatacaaggaggtgaagaaggatctgatcatcaaactgcctaagtactccctgttcgagcttgagaatggtagaaagcgcatgcttgcctcagccggcgaattgcagaagggcaatgagctcgccctgccttcaaaatacgtgaacttcctgtacttggcatcacactacgaaaagctgaaaggatcccctgaggataatgagcaaaaacaactttttgtggagcagcataagcactatctcgatgaaattattgagcagatttctgaattcagcaagcgcgtcatcctcgcggacgccaatctggataaagtgctgagcgcctacaataaacaccgagacaagcccattcgggaacaggccgagaacatcattcacctcttcactctgactaatctcggggccccggccgcattcaaatacttcgacactactatcgacaggaaacgctatacttcaacgaaggaggtgctggacgctactttgatccaccagtccattacggggctctatgagacacgaatcgatctttctcaacttggaggtgatgcctacccatatgacgtgcctgactatgcctccctgggctctgggagccctaagaaaaagaggaaggtagaggatccaaaaaaaaagcgaaaagtcgatgatatctaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 345 - DNA, 6744 bp, synthetic construct
  misc_feature 1..6744: note = Description of Artificial Sequence: Synthetic polynucleotide
  source 1..6744: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttgctagcctagacccagctttcttgtacaaagttggcattaatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaaggccaccatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggcggttccggcggagggtcgatgggccccaagaaaaaacgcaaggtggccgcagcagactataaggatgacgacgataaggggatccatggtgtgcctgctgcagataaaaaatacagcatcggcctggctatcggaactaactccgtcggctgggccgtcattaccgacgaatacaaagtacctagcaaaaagttcaaggtgcttggcaacacagatcgccactcaatcaagaaaaaccttatcggagccctgctgtttgactcaggcgaaaccgccgaggctacacgcctgaaaagaacagctagacggcggtacaccagaaggaagaaccggatctgttatcttcaggagattttctccaatgagatggctaaggtggacgattctttcttccatcgactcgaagaatctttcttggtggaggaagataagaaacacgagaggcatcctattttcggaaacattgtcgatgaagtggcctatcatgagaaataccccacgatctaccatctgcgaaaaaagttggttgactctaccgacaaggcggacctgaggcttatttatctggccctggcccatatgatcaaattcagggggcacttcttgatcgagggggaccttaatcccgacaactctgacgtggataagttgttcatacagcttgtgcagacctacaaccagctgttcgaggagaatccaatcaacgccagcggagtggacgctaaagccattctgagcgcgagattgagcaagtctagaagattggaaaaccttatagcccagctgccaggtgagaagaagaacggactgtttggcaatctcattgcgcttagcctcggactcaccccgaacttcaaatccaacttcgacctcgccgaagatgccaaattgcagctcagtaaggatacgtatgacgatgatcttgacaatctgctggcgcagatcggggaccagtacgccgatcttttcttggcagcaaaaaatctctcagatgcaatactcttgtcagacatactgcgagttaataccgagattactaaggctccgctttctgcctccatgatcaagcgctacgatgagcatcaccaggatctgacactgttgaaagccctggtgcgccaacagctgccagagaaatacaaggaaatcttttttgaccagtccaagaatggctacgcaggatacatcgatggaggagccagtcaggaggaattttacaagtttattaagcctatcctggagaagatggatggtaccgaagaactcctggtcaagctcaaccgagaagatttgcttcgcaagcaaaggacttttgacaacggctccattccgcatcagattcatctgggcgagctgcatgccattctgcgaagacaggaggatttttacccatttctgaaggacaaccgagagaagatcgagaaaatactgacattcaggataccatattacgtgggtccactcgccaggggcaactcccgattcgcctggatgacaaggaaaagcgaagagacgatcactccatggaacttcgaggaggtcgtggacaagggggcctccgcgcagagctttatcgagaggatgacgaactttgacaaaaatctccctaacgagaaggtgctgccaaaacattctctgctctacgagtatttcaccgtttataatgagctcacaaaggtgaagtacgtgaccgaagggatgcggaagcccgcttttctgtccggagagcagaagaaggctatcgtggatttgctctttaagactaaccgcaaggtaacagtcaagcagctgaaggaagactacttcaagaagatcgaatgcttgtcctacgaaacggaaatcttgacagttgagtacgggctcctgccaatcgggaagatagtagagaagaggattgaatgtaccgtctattctgttgataacaacggtaacatatacacccagcccgtcgcccaatggcacgatcgcggtgagcaggaggtgttcgaatactgtctggaggacgggtcattgattcgggcgactaaggaccataagtttatgacggtagacggccagatgttgcccatagatgagatctttgagcgggaactcgacttgatgagagtcgataatcttcctaattagcttaagggttcgatccctactggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 346 - DNA, 6516 bp, synthetic construct
  misc_feature 1..6516: note = Description of Artificial Sequence: Synthetic polynucleotide
  source 1..6516: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttgctagcctagacccagctttcttgtacaaagttggcattaatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaaggccaccatggatatcatgggccccaagaaaaaacgcaaggtggccgcagcagactataaggatgacgacgataaggggatccatggtgtgcctgctgcagataaaaaatacagcatcggcctggctatcggaactaactccgtcggctgggccgtcattaccgacgaatacaaagtacctagcaaaaagttcaaggtgcttggcaacacagatcgccactcaatcaagaaaaaccttatcggagccctgctgtttgactcaggcgaaaccgccgaggctacacgcctgaaaagaacagctagacggcggtacaccagaaggaagaaccggatctgttatcttcaggagattttctccaatgagatggctaaggtggacgattctttcttccatcgactcgaagaatctttcttggtggaggaagataagaaacacgagaggcatcctattttcggaaacattgtcgatgaagtggcctatcatgagaaataccccacgatctaccatctgcgaaaaaagttggttgactctaccgacaaggcggacctgaggcttatttatctggccctggcccatatgatcaaattcagggggcacttcttgatcgagggggaccttaatcccgacaactctgacgtggataagttgttcatacagcttgtgcagacctacaaccagctgttcgaggagaatccaatcaacgccagcggagtggacgctaaagccattctgagcgcgagattgagcaagtctagaagattggaaaaccttatagcccagctgccaggtgagaagaagaacggactgtttggcaatctcattgcgcttagcctcggactcaccccgaacttcaaatccaacttcgacctcgccgaagatgccaaattgcagctcagtaaggatacgtatgacgatgatcttgacaatctgctggcgcagatcggggaccagtacgccgatcttttcttggcagcaaaaaatctctcagatgcaatactcttgtcagacatactgcgagttaataccgagattactaaggctccgctttctgcctccatgatcaagcgctacgatgagcatcaccaggatctgacactgttgaaagccctggtgcgccaacagctgccagagaaatacaaggaaatcttttttgaccagtccaagaatggctacgcaggatacatcgatggaggagccagtcaggaggaattttacaagtttattaagcctatcctggagaagatggatggtaccgaagaactcctggtcaagctcaaccgagaagatttgcttcgcaagcaaaggacttttgacaacggctccattccgcatcagattcatctgggcgagctgcatgccattctgcgaagacaggaggatttttacccatttctgaaggacaaccgagagaagatcgagaaaatactgacattcaggataccatattacgtgggtccactcgccaggggcaactcccgattcgcctggatgacaaggaaaagcgaagagacgatcactccatggaacttcgaggaggtcgtggacaagggggcctccgcgcagagctttatcgagaggatgacgaactttgacaaaaatctccctaacgagaaggtgctgccaaaacattctctgctctacgagtatttcaccgtttataatgagctcacaaaggtgaagtacgtgaccgaagggatgcggaagcccgcttttctgtccggagagcagaagaaggctatcgtggatttgctctttaagactaaccgcaaggtaacagtcaagcagctgaaggaagactacttcaagaagatcgaatgcttgtcctacgaaacggaaatcttgacagttgagtacgggctcctgccaatcgggaagatagtagagaagaggattgaatgtaccgtctattctgttgataacaacggtaacatatacacccagcccgtcgcccaatggcacgatcgcggtgagcaggaggtgttcgaatactgtctggaggacgggtcattgattcgggcgactaaggaccataagtttatgacggtagacggccagatgttgcccatagatgagatctttgagcgggaactcgacttgatgagagtcgataatcttcctaattagcttaagggttcgatccctactggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc